(19) United States
(12) Patent Application Publication
(10) Pub. No.: US 2025/0171813 A1
(43) Pub. Date: May 29, 2025

(54) FLAVIN-DEPENDENT OXIDASES HAVING CANNABINOID SYNTHASE ACTIVITY

(71) Applicant: GENOMATICA, INC., San Diego, CA (US)

(72) Inventors: Jamison Parker HUDDLESTON, San Diego, CA (US); Andreas SCHIRMER, San Diego, CA (US); Trevor Nelson PURDY, San Diego, CA (US)

(21) Appl. No.: 18/842,653

(22) PCT Filed: Mar. 1, 2023
(86) PCT No.: PCT/US2023/063485

§ 371 Date: Aug. 29, 2024

Related U.S. Application Data
(60) Provisional application No. 63/315,864, filed on Mar. 2, 2022.

(51) Int. Cl. C12P 7/40; C12N 9/06; C12P 7/22

(52) U.S. Cl. CPC C12P 7/40; C12N 9/0022; C12P 7/22; C12Y 104/03023

(57) ABSTRACT

The disclosure relates to flavin-dependent oxidases having cannabinoid synthase activity. The flavindependent oxidase comprises: (i) a first amino acid sequence comprising a His residue, wherein an FAD cofactor is covalently attached to the His residue; and (ii) a second amino acid sequence comprising a peptide motif of Formula (I): X₁-Gly-X₂-Cys-X₃—X₄—X₅—X₆—X₇—X₈-Gly-X₉—X₁₀—X₁₁-Gly-Gly-Gly-X₁₂-Gly, wherein each X is any amino acid; and wherein the FAD cofactor is covalently attached to the Cys residue, wherein the flavin-dependent oxidase is capable of oxidative cyclization of a prenylated aromatic compound into a cannabinoid, and wherein the flavin-dependent oxidase is a bacterial protein or a fungal protein.

### SEQUENCE LISTING

The instant application contains a Sequence Listing which has been submitted electronically in XML format and is hereby incorporated by reference in its entirety. Said Sequence Listing XML, created on Mar. 1, 2023, is named 0171-0008WO1_SL.xml and is 17,996 bytes in size.

### FIELD OF THE INVENTION

The disclosure relates to flavin-dependent oxidases having cannabinoid synthase activity, wherein the flavin-dependent oxidase comprises: (i) a first amino acid sequence comprising a His residue, wherein an FAD cofactor is covalently attached to the His residue; and (ii) a second amino acid sequence comprising a peptide motif of Formula I:

[Formula I]
X₁-Gly-X₂-Cys-X₃-X₄-X₅-X₆-X₇-X₈-Gly-X₉-X₁₀-X₁₁-Gly-
Gly-Gly-X₁₂-Gly,

wherein each X is any amino acid; and wherein the FAD cofactor is covalently attached to the Cys residue, wherein the flavin-dependent oxidase is capable of oxidative cyclization of a prenylated aromatic compound into a cannabinoid, and wherein the flavin-dependent oxidase is a bacterial protein or a fungal protein. The disclosure further provides an engineered cell comprising a heterologous polynucleotide encoding the flavin-dependent oxidase described herein. Also provided herein are cell extracts and cell culture media comprising a cannabinoid derived from the engineered cell; methods of making cannabinoids; and compositions comprising a cannabinoid obtained from the engineered cell, the cell extract or cell culture medium, or the method; and compositions comprising the flavin-dependent oxidase and a cannabinoid and/or a prenylated aromatic compound. In some embodiments, the flavin-dependent oxidase comprises any of the proteins in Table 1.

### BACKGROUND

Cannabinoids constitute a varied class of chemicals, typically prenylated polyketides derived from fatty acid and isoprenoid precursors, that bind to cellular cannabinoid receptors. Modulation of these receptors has been associated with different types of physiological processes including pain-sensation, memory, mood, and appetite. Endocannabinoids, which occur in the body, phytocannabinoids, which are found in plants such as cannabis, and synthetic cannabinoids, can have activity on cannabinoid receptors and elicit biological responses. Recently, cannabinoids have drawn significant scientific interest in their potential to treat a wide array of disorders, including insomnia, chronic pain, epilepsy, and post-traumatic stress disorder (Babson et al. (2017), Curr Psychiatry Rep 19:23; Romero-Sandoval et al. (2017) Curr Rheumatol Rep 19:67; O'Connell et al. (2017) Epilepsy Behav 70:341-348; Zir-Aviv et al. (2016) Behav Pharmacol 27:561-569). The use of cannabinoids as therapeutics requires their production in large quantities and at high purity. However, purifying individual cannabinoid compounds from C. sativa can be time-consuming and costly, and it can be difficult to isolate a pure sample of a compound of interest. Thus, engineered cells can be a useful alternative for the production of a specific cannabinoid or cannabinoid precursor.

### SUMMARY OF THE INVENTION

The present disclosure relates to flavin-dependent oxidases that have cannabinoid synthase activity.

In some embodiments, the disclosure provides a flavin-dependent oxidase comprising: (i) a first amino acid sequence comprising a His residue, wherein an FAD cofactor is covalently attached to the His residue; and (ii) a second amino acid sequence comprising a peptide motif of Formula I:

[Formula I]
X₁-Gly-X₂-Cys-X₃-X₄-X₅-X₆-X₇-X₈-Gly-X₉-X₁₀-X₁₁-Gly-
Gly-Gly-X₁₂-Gly,

wherein each X is any amino acid; and wherein the FAD cofactor is covalently attached to the Cys residue, wherein the flavin-dependent oxidase is capable of oxidative cyclization of a prenylated aromatic compound into a cannabinoid, and wherein the flavin-dependent oxidase is a bacterial protein or a fungal protein.

In some embodiments, the non-natural flavin-dependent oxidase comprises: Ala, Gly, Ser, Thr. or His at position X₁; Thr, Ser, Arg, Val, Gly, Phe, or Asn at position X₂; Pro, Ala, Gly, Tyr, or Phe at position X₃; Thr, Ser, Ala, Asp, Gly, Asn, or Arg at position X₄; Val or Ile at position X₅; Gly, Ala, Cys, Arg, or Asn at position X₆; Ile, Val. Ala, Leu, Met, or Pro at position X₇; Ala, Gly, Ser, Thr, or Tyr at position X₈; Leu, His, Phe, Tyr, Ile, Val, or Trp at position X₉; Thr, Val, Leu, Ile, or Ala at position X₁₀; Leu, Gln, Ser, Thr, Cys, or Met at position X₁₁; Ile, Tyr, Leu, Trp, Val, Phe, Met, His, or Gln at position X₂; or any combination thereof.

In some embodiments, the peptide motif comprises:

X₁-Gly-X₂-Cys-Pro-Thr-Val-Gly-X₇-Xx-Gly-Leu-Thr-
Leu-Gly-Gly-Gly-X₁₂-Gly.

In some embodiments, X₂ is Thr or Ser; X₇ is Ile or Val; X₈ is Ala, Gly. or Ser; and X₁₂ is Ile, Tyr, or Leu.

In some embodiments, the peptide motif comprises any one of SEQ ID NOs:1-14. In some embodiments, the flavin-dependent oxidase is isolated or derived from an organism according to Table 1. In some embodiments, the flavin-dependent oxidase is not glycosylated. In some embodiments, the flavin-dependent oxidase does not comprise a disulfide bond. In some embodiments, the prenylated aromatic compound is cannabigerolic acid (CBGA), cannabigerorcinic acid (CBGOA), cannabigerovarinic acid (CBGVA), cannabigerorcinol (CBGO), cannabigerivarinol (CBGV), or cannabigerol (CBG). In some embodiments, the flavin-dependent oxidase comprises at least one amino acid variation as compared to a wild-type flavin-dependent oxidase.

In some embodiments, the disclosure provides an engineered cell comprising a heterologous polynucleotide encoding the flavin-dependent oxidase described herein. In some embodiments, the engineered cell is capable of producing a cannabinoid. In some embodiments, the cannabinoid comprises CBCA, CBDA, THCA, CBCOA, CBDOA, THCOA, CBCVA, CBDVA, THCVA, CBC, CBD, THC, CBCO, CBDO, THCO, CBCV, CBDV, THCV, or combinations thereof. In some embodiments, the engineered cell further comprises a cannabinoid biosynthesis pathway enzyme. In some embodiments, the cannabinoid biosynthesis pathway enzyme comprises olivetol synthase (OLS), olivetolic acid cyclase (OAC), prenyltransferase, a geranyl pyrophosphate (GPP) biosynthesis pathway enzyme, or combinations thereof. In some embodiments, the cell is a bacterial cell or a fungal cell. In some embodiments, the cell is an Escherichia coli cell.

In some embodiments, the disclosure provides a cell extract or cell culture medium comprising CBGA, CBCA, CBDA, THCA, CBG, CBC, CBD, THC, CBGOA, CBCOA, CBDOA, THCOA, CBGVA, CBCVA, CBDVA, THCVA, CBGO, CBCO, CBDO, THCO, CBGV, CBCV, CBDV, THCV, an isomer, analog or derivative thereof, or combinations thereof, derived from the engineered cell described herein.

In some embodiments, the disclosure provides a method of making a cannabinoid comprising: contacting a prenylated aromatic compound with the flavin-dependent oxidase described herein; culturing the engineered cell described herein; isolating the cannabinoid from the cell extract or cell culture medium described herein; or a combination thereof. In some embodiments, the prenylated aromatic compound comprises CBGA, CBG, CBGOA, CBGO, CBGVA, CBGV, or a combination thereof. In some embodiments, the cannabinoid comprises CBCA, CBC, CBCOA, CBCO, CBCVA, CBCV, CBDA, CBD, CBDOA, CBDO, CBDVA, CBDV, THCA, THC, THCOA, THCO, THCVA, THCV, an isomer, analog or derivative thereof, or combinations thereof.

In some embodiments, the disclosure provides a composition comprising a cannabinoid or an isomer, analog or derivative thereof obtained from the engineered cell described herein, the cell extract or cell culture medium described herein, or the method described herein. In some embodiments, the cannabinoid is CBCA, CBC, CBCOA, CBCO, CBCVA, CBCV, CBDA, CBD, CBDOA, CBDO, CBDVA, CBDV, THCA, THC, THCOA, THCO, THCVA, THCV, an isomer, analog or derivative thereof, or combinations thereof. In some embodiments, the cannabinoid is 50% or greater, 60% or greater, 70% or greater, 80% or greater, 85% or greater, 90% or greater, 91% or greater, 92% or greater, 93% or greater, 94% or greater, 95% or greater, 96% or greater, 97% or greater, 98% or greater, 99% or greater, 99.2% or greater, 99.4% or greater, 99.5% or greater, 99.6% or greater, 99.7% or greater, 99.8% or greater, or 99.9% or greater of total cannabinoid compound(s) in the composition. In some embodiments, the composition is a therapeutic or medicinal composition; a topical composition; an edible composition; or combinations thereof.

In some embodiments, the disclosure provides a composition comprising: (a) the flavin-dependent oxidase described herein; and (b) a prenylated aromatic compound, a cannabinoid, or both. In some embodiments, the prenylated aromatic compound comprises CBGA, CBG, CBGOA, CBGO, CBGVA, CBGV, or a combination thereof; and wherein the cannabinoid comprises CBCA, CBC, CBCOA, CBCO, CBCVA, CBCV, CBDA, CBD, CBDOA, CBDO, CBDVA, CBDV, THCA, THC, THCOA, THCO, THCVA, THCV, an isomer, analog or derivative thereof, or combinations thereof. In some embodiments, the composition further comprises an enzyme in a cannabinoid biosynthesis pathway. In some embodiments, the cannabinoid biosynthesis pathway enzyme comprises olivetol synthase (OLS), olivetolic acid cyclase (OAC), an enzyme in a geranyl pyrophosphate (GPP) pathway, prenyltransferase, or combinations thereof.

### BRIEF DESCRIPTION OF THE DRAWINGS

The following drawings form part of the present specification and are included to further demonstrate exemplary embodiments of certain aspects of the present disclosure.

FIG. 1 shows the consensus sequence of a peptide motif of Formula I, as described in embodiments herein.

FIG. 2 shows a sequence alignment of four enzymes in Table 1, as described in embodiments herein. Asterisk symbols (*) indicate the amino acid positions that have a single, fully conserved residue. Colon symbols (:) indicate conservation between amino acid groups of highly similar properties. Period symbols (.) indicate conservation between amino acid groups of weakly similar properties. The peptide motif of Formula I is marked in the box.

FIG. 3 shows a sequence alignment of 10 enzymes in Table 1 that had cannabinoid synthase activity plus Clz9, as described in embodiments herein. Asterisks (*), colons (:), and periods (.) are as described for FIG. 2. The peptide motif of Formula I is marked in the box.

FIG. 4 shows a percent identity matrix table of the 11 enzymes from FIG. 3, as described in embodiments herein. Clz9 is marked with a box.

FIG. 5A shows a chromatogram of the reaction of the protein with UniProt ID A0A1Q5S5E2 from Bradyrhizobium sp. NAS96 (“A0A1Q5S5E2”), with CBGA at pH 5.0 for 96 hours. FIG. 5B shows the LC/MS/MS fragmentation patterns of the cannabinoid products in the chromatogram of FIG. 5A (from left to right: CBCA-B, THCA-A, an unknown cannabinoid, and CBCA-A).

FIG. 6A shows a chromatogram of the reaction of a Clz9 variant comprising the amino acid mutations D404A T438F N400W V323Y Q275R C285L E370Q V3721 L296M I271H A338N A272C E159A T442D (“Clz9-var4”), with CBGA at pH 5.0 for 96 hours. FIG. 6B shows the LC/MS/MS fragmentation patterns of the cannabinoid products in the chromatogram of FIG. 6A (from left to right: CBCA-B, THCA-A, an unknown cannabinoid, and CBCA-A). FIG. 6C shows a summary of the cannabinoid products shown in the chromatograms of FIGS. 5A-6B.

FIG. 7 shows a table summarizing the cannabinoid synthase activity of 165 enzymes from Table 1. The relative amount of CBCA formed as compared with an empty vector (F.I.O.E.V.=fold-improvement over empty vector) at pH 7.4 and pH 5.0 are shown. Percent identity to Clz9 is also shown.

FIG. 8A shows a list of enzymes from Table 1 that have greater than 75% sequence identity to one or more of the 11 enzymes shown to be active, as listed in FIGS. 3 and 4. FIG. 8B shows a list of enzymes from Table 1 that have greater than 80% sequence identity to one or more of the 11 enzymes shown to be active, as listed in FIGS. 3 and 4. FIG. 8C shows a list of enzymes from Table 1 that have greater than 90% sequence identity to one or more of the 11 enzymes shown to be active, as listed in FIGS. 3 and 4.

### DETAILED DESCRIPTION OF THE INVENTION

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” means that the specific terms recited are representative examples and embodiments of the disclosure that are not intended to be limited to the specific examples referenced or cited unless explicitly stated otherwise.

As used herein, “about” can mean plus or minus 10% of the provided value. Where ranges are provided, they are inclusive of the boundary values. “About” can additionally or alternately mean either within 10% of the stated value, or within 5% of the stated value, or in some cases within 2.5% of the stated value; or, “about” can mean rounded to the nearest significant digit.

As used herein, “between” is a range inclusive of the ends of the range. For example, a number between x and y explicitly includes the numbers x and y, and any numbers that fall within the interval bounded by x and y.

A “nucleic acid,” “nucleic acid molecule,” “nucleic acid sequence,” “nucleotide sequence,” “oligonucleotide,” or “polynucleotide” means a polymeric compound including covalently linked nucleotides. The term “nucleic acid” includes ribonucleic acid (RNA) and deoxyribonucleic acid (DNA), both of which may be single- or double-stranded. DNA includes, but is not limited to, complementary DNA (cDNA), genomic DNA, plasmid or vector DNA, and synthetic DNA. In some embodiments, the disclosure provides a nucleic acid encoding any one of the polypeptides disclosed herein, e.g., is directed to a polynucleotide encoding a flavin-dependent oxidase or a variant thereof.

A “gene” refers to an assembly of nucleotides that encode a polypeptide and includes cDNA and genomic DNA nucleic acid molecules. In some embodiments, “gene” also refers to a non-coding nucleic acid fragment that can act as a regulatory sequence preceding (i.e., 5′) and following (i.e., 3′) the coding sequence.

As used herein, the term “operably linked” means that a polynucleotide of interest, e.g., the polynucleotide encoding an oxidase, is linked to the regulatory element in a manner that allows for expression of the polynucleotide. In some embodiments, the regulatory element is a promoter. In some embodiments, a nucleic acid expressing the polypeptide of interest is operably linked to a promoter on an expression vector.

As used herein, “promoter,” “promoter sequence,” or “promoter region” refers to a DNA regulatory region or polynucleotide capable of binding RNA polymerase and involved in initiating transcription of a downstream coding or non-coding sequence. In some embodiments, the promoter sequence includes the transcription initiation site and extends upstream to include the minimum number of bases or elements used to initiate transcription at levels detectable above background. In some embodiments, the promoter sequence includes a transcription initiation site, as well as protein binding domains responsible for the binding of RNA polymerase. Eukaryotic promoters typically contain “TATA” boxes and “CAT” boxes. Various promoters, including inducible promoters, may be used to drive expression of the various vectors of the present disclosure.

An “expression vector” or vectors (“an expression construct”) can be constructed to include one or more protein of interest-encoding nucleic acids (e.g., nucleic acid encoding a THCAS described herein) operably linked to expression control sequences functional in the host organism. Expression vectors applicable for use in the microbial host organisms provided include, for example, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, and the like), P1-based artificial chromosomes, yeast plasmids, yeast artificial chromosomes, and any other vectors specific for specific hosts of interest (such as E. coli and yeast). In some embodiments, the expression vector comprises a nucleic acid encoding a protein described herein, e.g., a flavin-dependent oxidase.

Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like. When two or more exogenous encoding nucleic acids (e.g., a gene encoding a flavin-dependent oxidase and an additional gene encoding another enzyme in a cannabinoid biosynthesis pathway such as, e.g., OLS, OAC, prenyltransferase, and/or an enzyme in the GPP pathway as described herein) are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein. The following vectors are provided by way of example; for bacterial host cells: pQE vectors (Qiagen), pBluescript plasmids, pNH vectors, lambda-ZAP vectors (Stratagene); pTrc99a, pKK223-3, pDR540, and pRIT2T (Pharmacia); for eukaryotic host cells: pXT1, pSG5 (Stratagene), pSVK3, pBPV, pMSG, and pSVLSV40 (Pharmacia). However, any other plasmid or other vector may be used so long as it is compatible with the host cell.

The term “host cell” refers to a cell into which a recombinant expression vector has been introduced, or “host cell” may also refer to the progeny of such a cell. Because modifications may occur in succeeding generations, for example, due to mutation or environmental influences, the progeny may not be identical to the parent cell, but are still included within the scope of the term “host cell.” In some embodiments, the present disclosure provides a host cell comprising an expression vector that comprises a nucleic acid encoding a flavin-dependent oxidase or variant thereof. In some embodiments, the host cell is a bacterial cell, a fungal cell, an algal cell, a cyanobacterial cell, or a plant cell.

A genetic alteration that makes an organism or cell non-natural can include, for example, modifications introducing expressible nucleic acids encoding metabolic polypeptides, other nucleic acid additions, nucleic acid deletions and/or other functional disruption of the organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon.

A host cell, organism, or microorganism engineered to express or overexpress a gene, a nucleic acid, nucleic acid sequence, or nucleic acid molecule, or to overexpress an enzyme or polypeptide has been genetically engineered through recombinant DNA technology to include a gene or nucleic acid sequence that it does not naturally include that encodes the enzyme or polypeptide or to express an endogenous gene at a level that exceeds its level of expression in a non-altered cell. As non-limiting examples, a host cell, organism, or microorganism engineered to express or overexpress a gene, a nucleic acid, nucleic acid sequence, or nucleic acid molecule, or to overexpress an enzyme or polypeptide can have any modifications that affect a coding sequence of a gene, the position of a gene on a chromosome or episome, or regulatory elements associated with a gene. A gene can also be overexpressed by increasing the copy number of a gene in the cell or organism. In some embodiments, overexpression of an endogenous gene comprises replacing the native promoter of the gene with a constitutive promoter that increases expression of the gene relative to expression in a control cell with the native promoter. In some embodiments, the constitutive promoter is heterologous.

Similarly, a host cell, organism, or microorganism engineered to under-express (or to have reduced expression of) a gene, nucleic acid, nucleic acid sequence, or nucleic acid molecule, or to under-express an enzyme or polypeptide can have any modifications that affect a coding sequence of a gene, the position of a gene on a chromosome or episome, or regulatory elements associated with a gene. Specifically included are gene disruptions, which include any insertions, deletions, or sequence mutations into or of the gene or a portion of the gene that affect its expression or the activity of the encoded polypeptide. Gene disruptions include “knockout” mutations that eliminate expression of the gene. Modifications to under-express or down-regulate a gene also include modifications to regulatory regions of the gene that can reduce its expression.

The term “exogenous” is intended to mean that the referenced molecule or the referenced activity is introduced into the host cell or host organism. The molecule can be introduced, for example, by introduction of an encoding nucleic acid into the host genetic material such as by integration into a host chromosome or as non-chromosomal genetic material that may be introduced on a vehicle such as a plasmid. The term “exogenous nucleic acid” means a nucleic acid that is not naturally-occurring within the host cell or host organism. Exogenous nucleic acids may be derived from or identical to a naturally-occurring nucleic acid or it may be a heterologous nucleic acid. For example, a non-natural duplication of a naturally-occurring gene is considered to be an exogenous nucleic acid sequence. An exogenous nucleic acid can be introduced in an expressible form into the host cell or host organism. The term “exogenous activity” refers to an activity that is introduced into the host cell or host organism. The source can be, for example, a homologous or heterologous encoding nucleic acid that expresses the referenced activity following introduction into the host cell or host organism.

Accordingly, the term “endogenous” refers to a referenced molecule or activity that is naturally present in the host cell or host organism. Similarly, the term when used in reference to expression of an encoding nucleic acid refers to expression of an encoding nucleic acid contained within the host cell or host organism.

The term “heterologous” refers to a molecule or activity derived from a source other than the referenced species, whereas “homologous” refers to a molecule or activity derived from the host microbial organism/species. Accordingly, exogenous expression of an encoding nucleic acid can utilize either or both of a heterologous or homologous encoding nucleic acid.

When used to refer to a genetic regulatory element, such as a promoter, operably linked to a gene, the term “homologous” refers to a regulatory element that is naturally operably linked to the referenced gene. In contrast, a “heterologous” regulatory element is not naturally found operably linked to the referenced gene, regardless of whether the regulatory element is naturally found in the host cell or host organism.

It is understood that more than one exogenous nucleic acid(s) can be introduced into the host cell or host organism on separate nucleic acid molecules, on polycistronic nucleic acid molecules, or combinations thereof, and still be considered as more than one exogenous nucleic acid. For example, as disclosed herein, a host cell or host organism can be engineered to express at least two, three, four, five, six, seven, eight, nine, ten or more exogenous nucleic acids encoding a desired pathway enzyme or protein. In the case where two or more exogenous nucleic acids encoding a desired activity are introduced into a host cell or host organism, it is understood that the two or more exogenous nucleic acids can be introduced as a single nucleic acid, for example, on a single plasmid, on separate plasmids, can be integrated into the host chromosome at a single site or multiple sites, and still be considered as two or more exogenous nucleic acids. Similarly, it is understood that more than two exogenous nucleic acids can be introduced into a host cell or host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host cell or host organism.

Genes or nucleic acid sequences can be introduced stably or transiently into a host cell host cell or host organism using techniques well known in the art including, but not limited to, conjugation, electroporation, chemical transformation, transduction, transfection, and ultrasound transformation. Optionally, for exogenous expression in E. coli or other prokaryotic host cells, some nucleic acid sequences in the genes or cDNAs of eukaryotic nucleic acids can encode targeting signals such as an N-terminal mitochondrial or other targeting signal, which can be removed before transformation into the prokaryotic host cells, if desired. For example, removal of a mitochondrial leader sequence led to increased expression in E. coli (Hoffmeister et al. (2005), J Biol Chem 280: 4329-4338). For exogenous expression in yeast or other eukaryotic host cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques known in the art to achieve optimized expression of the proteins.

In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Computer algorithms for codon optimizing a particular sequence for expression in a particular host cell are available and include, e.g., Integrated DNA Technologies' Codon Optimization tool, Entelechon's Codon Usage Table Analysis Tool, GenScript's OptimumGene tool, and the like. In some embodiments, the disclosure provides codon optimized polynucleotides expressing a flavin-dependent oxidase or variant thereof.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The start of the protein or polypeptide is known as the “N-terminus” (and also referred to as the amino-terminus, NH₂-terminus, N-terminal end or amine-terminus), referring to the free amine (—NH₂) group of the first amino acid residue of the protein or polypeptide. The end of the protein or polypeptide is known as the “C-terminus” (and also referred to as the carboxy-terminus, carboxyl-terminus, C-terminal end, or COOH-terminus), referring to the free carboxyl group (—COOH) of the last amino acid residue of the protein or polypeptide. Unless otherwise specified, sequences of polypeptides throughout the present disclosure are listed from N-terminus to C-terminus, and sequences of polynucleotides throughout the present disclosure are listed from the 5′ end to the 3′ end.

An “amino acid” as used herein refers to a compound including both a carboxyl (—COOH) and amino (—NH₂) group. “Amino acid” refers to both natural and unnatural, i.e., synthetic, amino acids. Natural amino acids, with their three-letter and single-letter abbreviations, include: alanine (Ala; A); arginine (Arg, R); asparagine (Asn; N); aspartic acid (Asp; D); cysteine (Cys; C); glutamine (Gln; Q); glutamic acid (Glu; E); glycine (Gly; G); histidine (His; H); isoleucine (Ile; I); leucine (Leu; L); lysine (Lys; K); methionine (Met; M); phenylalanine (Phe; F); proline (Pro; P); serine (Ser; S); threonine (Thr; T); tryptophan (Trp; W); tyrosine (Tyr; Y); and valine (Val; V). Unnatural or synthetic amino acids include a side chain that is distinct from the natural amino acids provided above and may include, e.g., fluorophores, post-translational modifications, metal ion chelators, photocaged and photo-cross-linked moieties, uniquely reactive functional groups, and NMR, IR, and x-ray crystallographic probes. Exemplary unnatural or synthetic amino acids are provided in, e.g., Mitra et al. (2013), Mater Methods 3:204 and Wals et al. (2014), Front Chem 2:15. Unnatural amino acids may also include naturally-occurring compounds that are not typically incorporated into a protein or polypeptide, such as, e.g., citrulline (Cit), selenocysteine (See), and pyrrolysine (Pyl).

As used herein, the terms “non-natural,” “non-naturally occurring,” “variant,” and “mutant” are used interchangeably in the context of an organism, polypeptide, or nucleic acid. The terms “non-natural,”“non-naturally occurring,” “variant,” and “mutant” in this context refer to a polypeptide or nucleic acid sequence having at least one variation or mutation at an amino acid position or nucleic acid position as compared to a wild-type polypeptide or nucleic acid sequence. The at least one variation can be, e.g., an insertion of one or more amino acids or nucleotides, a deletion of one or more amino acids or nucleotides, or a substitution of one or more amino acids or nucleotides. A “variant” protein or polypeptide is also referred to as a “non-natural” protein or polypeptide.

Naturally-occurring organisms, nucleic acids, and polypeptides can be referred to as “wild-type,” “wild type” or “original” or “natural” such as wild type strains of the referenced species, or a wild-type protein or nucleic acid sequence. Likewise, amino acids found in polypeptides of the wild type organism can be referred to as “original” or “natural” with regards to any amino acid position.

An “amino acid substitution” refers to a polypeptide or protein including one or more substitutions of wild-type or naturally occurring amino acid with a different amino acid relative to the wild-type or naturally occurring amino acid at that amino acid residue. The substituted amino acid may be a synthetic or naturally occurring amino acid. In some embodiments, the substituted amino acid is a naturally occurring amino acid selected from the group consisting of: A, R, N, D, C, Q, E, G, H, I, L, K, M, F, P, S, T, W, Y, and V. In some embodiments, the substituted amino acid is an unnaturally or synthetic amino acid. Substitution mutants may be described using an abbreviated system. For example, a substitution mutation in which the fifth (5^{th}) amino acid residue is substituted may be abbreviated as “X5Y,” wherein “X” is the wild-type or naturally occurring amino acid to be replaced, “5” is the amino acid residue position within the amino acid sequence of the protein or polypeptide, and “Y” is the substituted, or non-wild-type or non-naturally occurring, amino acid.

An “isolated” polypeptide, protein, peptide, or nucleic acid is a molecule that has been removed from its natural environment. It is also understood that “isolated” polypeptides, proteins, peptides, or nucleic acids may be formulated with excipients such as diluents or adjuvants and still be considered isolated. As used herein, “isolated” does not necessarily imply any particular level purity of the polypeptide, protein, peptide, or nucleic acid.

The term “recombinant” when used in reference to a nucleic acid molecule, peptide, polypeptide, or protein means of, or resulting from, a new combination of genetic material that is not known to exist in nature. A recombinant molecule can be produced by any of the techniques available in the field of recombinant technology, including, but not limited to, polymerase chain reaction (PCR), gene splicing (e.g., using restriction endonucleases), and solid-phase synthesis of nucleic acid molecules, peptides, or proteins.

The term “domain” when used in reference to a polypeptide or protein means a distinct functional and/or structural unit in a protein. Domains are sometimes responsible for a particular function or interaction, contributing to the overall role of a protein. Domains may exist in a variety of biological contexts. Similar domains may be found in proteins with different functions. Alternatively, domains with low sequence identity (i.e., less than about 50%, less than about 40%, less than about 30%, less than about 20%, less than about 10%, less than about 5%, or less than about 1% sequence identity) may have the same function.

As used herein, the term “sequence similarity” (% similarity) refers to the degree of identity or correspondence between nucleic acid sequences or amino acid sequences. In the context of polynucleotides, “sequence similarity” may refer to nucleic acid sequences wherein changes in one or more nucleotide bases results in substitution of one or more amino acids, but do not affect the functional properties of the protein encoded by the polynucleotide. “Sequence similarity” may also refer to modifications of the polynucleotide, such as deletion or insertion of one or more nucleotide bases, that do not substantially affect the functional properties of the resulting transcript. It is therefore understood that the present disclosure encompasses more than the specific exemplary sequences. Methods of making nucleotide base substitutions are known, as are methods of determining the retention of biological activity of the encoded polypeptide.

In the context of polypeptides, “sequence similarity” refers to two or more polypeptides wherein greater than about 40% of the amino acids are identical, or greater than about 60% of the amino acids are functionally identical. “Functionally identical” or “functionally similar” amino acids have chemically similar side chains. For example, amino acids can be grouped in the following manner according to functional similarity: Positively-charged side chains: Arg, His, Lys; Negatively-charged side chains: Asp, Glu; Polar, uncharged side chains: Scr, Thr, Asn, Gln; Hydrophobic side chains: Ala, Val, Ile, Leu, Met, Phe, Tyr, Trp; Other: Cys, Gly, Pro.

In some embodiments, similar polypeptides of the present disclosure have about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% functionally identical amino acids.

The “percent identity” (% identity) between two polynucleotide or polypeptide sequences is determined when sequences are aligned for maximum homology, and generally not including gaps or truncations. Additional sequences added to a polypeptide sequence, such as but not limited to immunodetection tags, purification tags, localization sequences (presence or absence), etc., do not affect the % identity.

Algorithms known to those skilled in the art, such as Align, BLAST, ClustalW and others compare and determine a raw sequence similarity or identity, and also determine the presence or significance of gaps in the sequence which can be assigned a weight or score. Such algorithms also are known in the art and are similarly applicable for determining nucleotide or amino acid sequence similarity or identity, and can be useful in identifying orthologs of genes of interest.

In some embodiments, similar polynucleotides of the present disclosure have about 40%, at least about 40%, about 45%, at least about 45%, about 50%, at least about 50%, about 55%, at least about 55%, about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% identical nucleic acid sequence. In some embodiments, similar polypeptides of the present disclosure have about 40%, at least about 40%, about 45%, at least about 45%, about 50%, at least about 50%, about 55%, at least about 55%, about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% identical amino acid sequence.

A homolog is a gene or genes that are related by vertical descent and are responsible for substantially the same or identical functions in different organisms. Genes are related by vertical descent when, for example, they share sequence similarity of sufficient amount to indicate they are related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Paralogs are genes related by duplication within a genome, and can evolve new functions, even if these are related to the original one.

An amino acid position (or simply, amino acid) “corresponding to” an amino acid position in another polypeptide sequence is the position that is aligned with the referenced amino acid position when the polypeptides are aligned for maximum homology, for example, as determined by BLAST, which allows for gaps in sequence homology within protein sequences to align related sequences and domains. Alternatively, in some instances, when polypeptide sequences are aligned for maximum homology, a corresponding amino acid may be the nearest amino acid to the identified amino acid that is within the same amino acid biochemical grouping—i.e., the nearest acidic amino acid, the nearest basic amino acid, the nearest aromatic amino acid, etc. to the identified amino acid.

By “substantially identical,” with reference to a nucleic acid sequence (e.g., a gene, RNA, or cDNA) or amino acid sequence (e.g., a protein or polypeptide) is meant one that has at least at least 80%, at least 85%, at least 90%, at least 95%, at least 96%, at least 97% at least 98%, or at least 99% nucleotide or amino acid identity, respectively, to a reference sequence.

As used in the context of proteins, the term “structural similarity” indicates the degree of homology between the overall shape, fold, and/or topology of the proteins. It should be understood that two proteins do not necessarily need to have high sequence similarity to achieve structural similarity. Protein structural similarity is often measured by root mean squared deviation (RMSD), global distance test score (GDT-score), and template modeling score (TM-score); see, e.g., Xu and Zhang (2010), Bioinformatics 26(7):889-895. Structural similarity can be determined, e.g., by superimposing protein structures obtained from, e.g., x-ray crystallography, NMR spectroscopy, cryogenic electron microscopy (cryo-EM), mass spectrometry, or any combination thereof, and calculating the RMSD, GDT-score, and/or TM-score based on the superimposed structures. In some embodiments, two proteins have substantially similar tertiary structures when the TM-score is greater than about 0.5, greater than about 0.6, greater than about 0.7, greater than about 0.8, or greater than about 0.9. In some embodiments, two proteins have substantially identical tertiary structures when the TM-score is about 1.0. Structurally-similar proteins may also be identified computationally using algorithms such as, e.g., TM-align (Zhang and Skolnick, Nucleic Acids Res 33(7):2302-2309, 2005); DALI (Holm and Sander, J Mol Biol 233(1):123-138, 1993); STRUCTAL (Gerstein and Levitt, Proc Int Conf Intell Syst Mol Biol 4:59-69, 1996); MINRMS (Jewett et al., Bioinformatics 19(5):625-634, 2003); Combinatorial Extension (CE) (Shindyalov and Bourne. Protein Eng 11(9):739-747, 1998); ProtDex (Aung et al., DASFAA 2003, Proceedings); VAST (Gibrat et al., Curr Opin Struct Biol 6:377-385, 1996); LOCK (Singh and Brutlag, Proc Int Conf Intell Syst Mol Biol 5:284-293, 1997); SSM (Krissinel and Henrick, Acta Cryst D60:2256-2268, 2004), and the like.

### Flavin-Dependent Oxidase

Cannabinoid synthases are enzymes responsible for the biosynthesis of cannabinoids, e.g., cannabinoid compounds described herein. The only naturally-occurring cannabinoid synthase enzymes currently known to convert cannabigerolic acid (CBGA) or its analogs to cannabinoids such as A9-tetrahydrocannabinolic acid (THCA) by THCA synthase (THCAS, EC 1.21.3.7), cannabidiolic acid (CBDA) by CBDA synthase (CBDAS, EC 1.21.3.8) or cannabichromenic acid (CBCA) by CBCA synthase (CBCAS) or their analogs are from the plant Cannabis sativa (Onofri et al. (2015), J Mol Biol 423:96; Laverty et al. (2019), Genome Research 29:146-156). It is challenging to utilize these enzymes from C. sativa for heterologous cannabinoid production in microorganisms such as bacteria because they are typically secreted proteins that require a disulfide bond and glycosylation, are poorly active, and require low pH for optimal activity (Zirpel et al. (2018), J Biotechnol 284:17-26). Thus, cannabinoid synthase enzymes from C. sativa are not conducive for standard microbial fermentation processes that typically use media with a neutral or near neutral pH of 6 to 8.

The present inventors have discovered alternative enzymes for the improved microbial production of cannabinoids. The enzymes described herein may be suitable for soluble and active expression in a microbial host under standard fermentation conditions. In some embodiments, the enzyme is a bacterial or a fungal enzyme. In some embodiments, the enzyme is a flavin-dependent oxidase.

In some embodiments, the present disclosure provides a bacterial or a fungal flavin-dependent oxidase, wherein the flavin-dependent oxidase is capable of oxidative cyclization of a prenylated aromatic compound into a cannabinoid.

As used herein, “cannabinoid” refers to a prenylated polyketide or terpenophenolic compound derived from fatty acid or isoprenoid precursors. In general, cannabinoids are produced via a multi-step biosynthesis pathway, with the final precursor being a prenylated aromatic compound. In some embodiments, the prenylated aromatic compound is cannabigerolic acid (CBGA), cannabigerorcinic acid (CBGOA), cannabigerovarinic acid (CBGVA), cannabigerorcinol (CBGO), cannabigerivarinol (CBGV), or cannabigerol (CBG). In some embodiments, the prenylated aromatic compound is converted into a cannabinoid by oxidative cyclization. In some embodiments, the flavin-dependent oxidase converts one or more of CBGA. CBGOA, CBGVA, CBGO, CBGV, and CBG into a cannabinoid. In some embodiments, the flavin-dependent oxidase converts CBGA into one or more of CBCA, CBDA, or THCA. In some embodiments, the flavin-dependent oxidase converts CBGOA into one or more of CBCOA, CBDOA, or THCOA. In some embodiments, the flavin-dependent oxidase converts CBGVA into one or more of CBCVA, CBDVA, or THCVA. In some embodiments, the flavin-dependent oxidase converts CBG into one or more of CBC, CBD, or THC. In some embodiments, the flavin-dependent oxidase converts CBG into one or more of CBC. In some embodiments, the flavin-dependent oxidase converts CBGO into one or more of CBCO, CBDO, or THCO. In some embodiments, the flavin-dependent oxidase converts CBGV into one or more of CBCV, CBDV, or THCV.

Different cannabinoids can be produced based on the way that a precursor is cyclized. For example, THCA, CBDA, and CBCA are produced by oxidative cyclization of CBGA. Further examples of cannabinoids include, but are not limited to, THCA, THCV, THCO, THCVA, THCOA. THC, CBDA, CBDV, CBDO, CBDVA, CBDOA, CBD, CBCA, CBCV, CBCO, CBCVA, CBCOA, CBC, cannabinolic acid (CBNA), cannabinol (CBN), cannabicyclol (CBL), cannabivarin (CBV), cannabielsoin (CBE), cannabicitran, and isomers, analogs or derivatives thereof. As used herein, an “isomer” of a reference compound has the same molecular formula as the reference compound, but with a different arrangement of the atoms in the molecule. As used herein, an “analog” or “structural analog” of a reference compound has a similar structure as the reference compound, but differs in a certain component such as an atom, a functional group, or a substructure. An analog can be imagined to be formed from the reference compound, but not necessarily synthesized from the reference compound. As used herein, a “derivative” of a reference compound is derived from a similar compound by a similar reaction. Methods of identifying isomers, analogs or derivatives of the cannabinoids described herein are known to one of ordinary skill in the art.

In some embodiments, the flavin-dependent oxidase is a berberine bridge enzyme (BBE-like enzyme). BBE-like enzymes are described, e.g., in Daniel et al. (2017), Arch Biochem Biophys 632:88-103 and include protein family domains (Pfams) PF08031 (berberine-bridge domain) and PF01564 (flavin adenine dinucleotide (FAD)-binding domain). In general, a BBE-like enzyme comprises a FAD binding module that is formed by the N- and C-terminal portions of the protein, and a central substrate binding domain that, together with the FAD cofactor, provides the environment for efficient substrate binding, oxidation and cyclization. It will be understood by one of ordinary skill in the art that, in some embodiments, a BBE-like enzyme binds a flavin mononucleotide (FM) in addition to or instead of FAD.

In some embodiments, the flavin-dependent oxidase has substantial structural similarity with a cannabinoid synthase from C. sativa, e.g., A9-tetrahydrocannabinolic acid synthase (THCAS). THCAS utilizes a FAD cofactor when catalyzing the conversion of substrate CBGA to THCA. In some embodiments, the flavin-dependent oxidase comprises a structurally similar active site as a cannabinoid synthase from C. sativa, e.g., THCAS. As used herein, the term “active site” refers to one or more regions in an enzyme that are important for catalysis, substrate binding, and/or cofactor binding.

In some embodiments, the present disclosure provides a flavin-dependent oxidase comprising: (i) a first amino acid sequence comprising a His residue, wherein an FAD cofactor is covalently attached to the His residue; and (ii) a second amino acid sequence comprising a peptide motif of Formula I:

[Formula I]
X₁-Gly-X₂-Cys-X₃-X₄-X₅-X₆-X₇-X₈-Gly-X₉-X₁₀-X₁₁-Gly-
Gly-Gly-X₁₂-Gly,

wherein each X is any amino acid; and wherein the FAD cofactor is covalently attached to the Cys residue, wherein the flavin-dependent oxidase is capable of oxidative cyclization of a prenylated aromatic compound into a cannabinoid, and wherein the flavin-dependent oxidase is a bacterial protein or a fungal protein.

The present disclosure provides that, while flavin-dependent oxidases may be monovalently bound or bivalently bound to an FAD cofactor, the enzymes that are capable of oxidizing CBGA into a cannabinoid, e.g., CBCA, CBDA, and/or THCA, comprise a bivalent binding to FAD. As used herein, “monovalent” binding means that the FAD is covalently bound to one amino acid residue of the referenced protein, e.g., the flavin-dependent oxidase. As used herein, “bivalent” binding means that the FAD is covalently bound to two amino acid residues of the referenced protein, e.g., flavin-dependent oxidase. In some embodiments, the FAD cofactor is bound to the flavin-dependent oxidase at a histidine (His) residue and a cysteine (Cys) residue. The present disclosure provides that the Cys residue that binds to the FAD cofactor is present in a conserved peptide motif as according to Formula I:

[Formula I]
X₁-Gly-X₂-Cys-X₃-X₄-X₅-X₆-X₇-X₈-Gly-X₉-X₁₀-X₁₁-Gly-
Gly-Gly-X₁₂-Gly,

wherein each X is any amino acid.

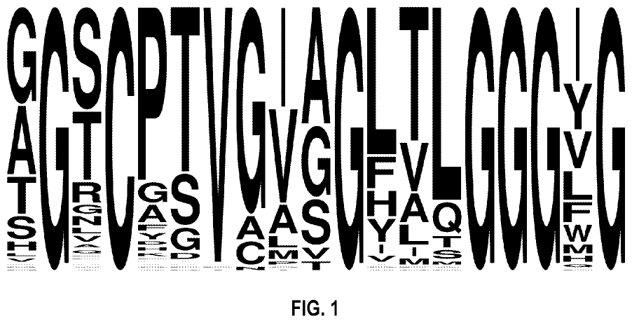

In some embodiments, the flavin-dependent oxidase comprises a peptide motif as shown in FIG. 1. FIG. 1 depicts a peptide motif encompassed by Formula I except without the leading X₁ residue.

In some embodiments, X₁ of Formula I is Ala, Gly, Ser, Thr, or His. In some embodiments, X₂ of Formula I is Thr, Ser, Arg, Val, Gly, Phe, or Asn. In some embodiments, X₃ of Formula I is Pro, Ala, Gly, Tyr, or Phe. In some embodiments, X₄ of Formula I is Thr, Ser, Ala, Asp, Gly, Asn, or Arg. In some embodiments, X₈ of Formula I is Val or Ile. In some embodiments, X₆ of Formula I is Gly, Ala, Cys, Arg, or Asn. In some embodiments, X₇ of Formula I is Ile, Val, Ala, Leu, Met, or Pro. In some embodiments, X₈ of Formula I is Ala, Gly, Ser, Thr, or Tyr. In some embodiments, X₉ of Formula I is Leu, His, Phe, Tyr, Ile. Val, or Trp. In some embodiments, X₁₀ of Formula I is Thr, Val, Leu, Ile, or Ala. In some embodiments, X₈ of Formula I is Leu, Gln, Ser, Thr, Cys, or Met. In some embodiments, X₁₂ of Formula I is Ile, Tyr, Leu, Trp, Val, Phe, Met, His, or Gln.

In some embodiments, the peptide motif of Formula I comprises:

X₁-Gly-X₂-Cys-Pro-Thr-Val-Gly-X₇-X₈-Gly-Leu-Thr-
Leu-Gly-Gly-Gly-X₁₂-Gly,

wherein X₂ is Thr or Ser; X₇ is Ile or Val; X₈ is Ala, Gly, or Ser; and X₁₂ is Ile, Tyr, or Leu.

In some embodiments, the peptide motif of Formula I comprises:

X₁-Gly-Thr-Cys-Pro-Thr-Val-Gly-Ile-Ala-Gly-Leu-
Thr-Leu-Gly-Gly-Gly-Ile-Gly.

In some embodiments, the peptide motif of Formula I comprises

(SEQ ID NO: 1)
AGSCPTVGVAGLTLGGGFG;
(SEQ ID NO: 2)
AGSCGTVAIGGLTLGGGVG;
(SEQ ID NO: 3)
AGSCPTVGIAGLTLGGGIG;
(SEQ ID NO: 4)
AGSCFTVGVAGVTLGGGIG;
(SEQ ID NO: 5)
GGTCPRVAVGGLVLGGGYG;
(SEQ ID NO: 6)
AGVCPDIRIGGHVLGGGVG;
(SEQ ID NO: 7)
AGTCPRIGIGGHVLGGGMG;
(SEQ ID NO: 8)
AGFCPEIGIAGHVLGGGAG
(SEQ ID NO: 9)
TGACGSVCVGGFVQGGGYG;
(SEQ ID NO: 10)
GGSCHDVCVAGFMQGGGFG;
(SEQ ID NO: 11)
SGRCPTVGTSGLVLGGGWG;
(SEQ ID NO: 12)
GGSCPSVGIAGYLLGGGVG;
(SEQ ID NO: 13)
TGNCPTVGMGGYLQGGGVG;
or
(SEQ ID NO: 14)
GGYCPTVAAGGYFAGGGMG.

In some embodiments, SEQ ID NO:1 is a peptide motif according to Formula I in the protein with UniProt ID A0A150PPA5 from Sorangium cellulosum. In some embodiments SEQ ID NO:2 is a peptide motif according to Formula I in the protein with UniProt ID A0A3N1QKT1 from Frondihabitans sp. PhB188. In some embodiments, SEQ ID NO:3 is a peptide motif according to Formula I in the protein with UniProt ID A0A1K1PD14 from Amycolatopsis australiensis. In some embodiments, SEQ ID NO:4 is a peptide motif according to Formula I in the protein with UniProt ID D9XHS6 from Streptomyces viridochromogenes (strain DSM40736/JCM4977/BCRC1201/Tue494).

In some embodiments, SEQ ID NO:5 is a peptide motif according to Formula I in the protein with UniProt ID A0A1H4CL41 from Mycobacterium sp. 283mftsu. In some embodiments, SEQ ID NO:6 is a peptide motif according to Formula I in the protein with Accession ID WP_211768552.1 from Kutzneria sp. CA-103260. In some embodiments, SEQ ID NO:7 is a peptide motif according to Formula I in the protein with Accession ID WP_235454663.1 from Streptomyces olivochromogenes. In some embodiments, SEQ ID NO:8 is a peptide motif according to Formula I in the protein with UniProt ID U6A1G7 from Streptomyces sp. CNH-287 (i.e., “Clz9”).

In some embodiments, SEQ ID NO:9 is a peptide motif according to Formula I in the protein with UniProt ID A0A7X0U8H0 from Acidovorax soli. In some embodiments, SEQ ID NO:10 is a peptide motif according to Formula I in the protein with UniProt ID A0A1Q5S5E2 from Bradyrhizobium sp. NAS96. In some embodiments, SEQ ID NO:11 is a peptide motif according to Formula I in the protein with UniProt ID A0A0Q7FI10 from Massilia sp. Root418.

In some embodiments, SEQ ID NO:12 is a peptide motif according to Formula I in the protein with UniProt ID A0A2E0XWX6 from Phycisphaerae bacterium. In some embodiments, SEQ ID NO:13 is a peptide motif according to Formula I in the protein with UniProt ID A0A0K3BN04 from Kibdelosporangium sp. MJ126-NF4. In some embodiments, SEQ ID NO:14 is a peptide motif according to Formula I in the protein with UniProt ID A0A1U9QQ65 from Streptomyces niveus.

In some embodiments, the peptide motif of Formula I comprises a sequence having at least 80%, at least 85%, at least 90%, at least 91%, at least 92%, at least 93%, at least 94%, at least 95%, at least 96%, at least 97%, at least 98%, at least 99% to any one of SEQ ID NOs:1-14, provided that the amino acid residues at positions 2, 4, 11, 15-17, and 19 of SEQ ID NOs:1-14 remain unchanged.

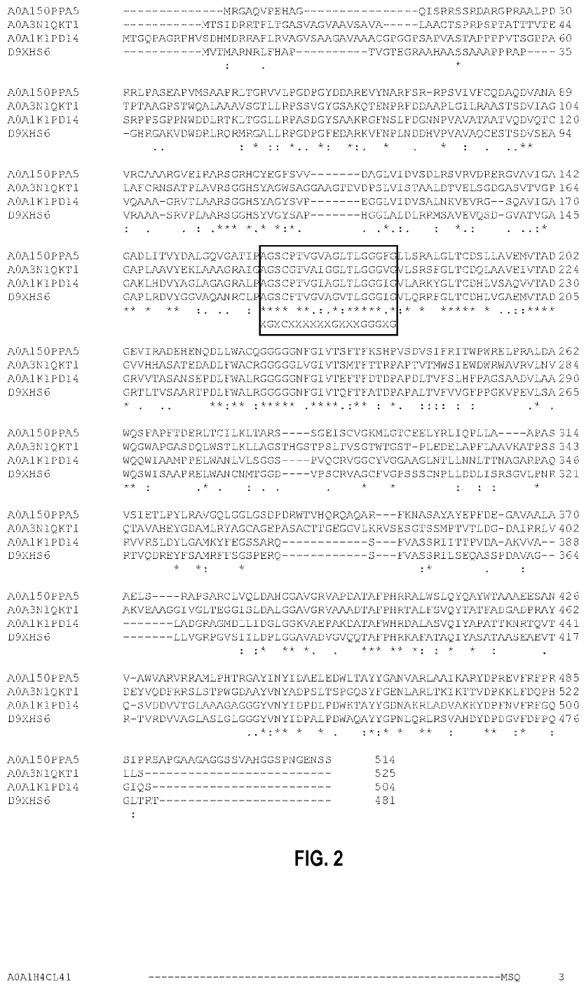

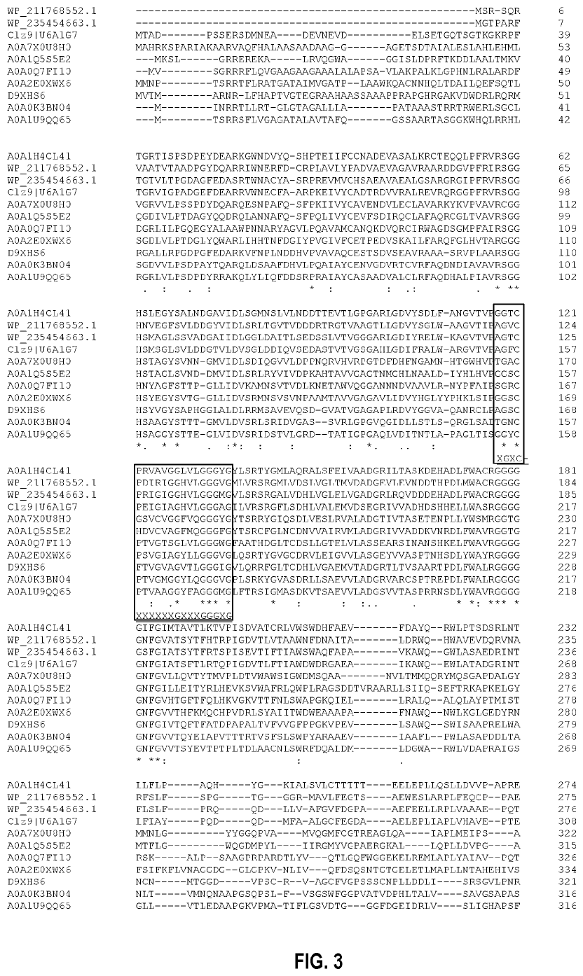

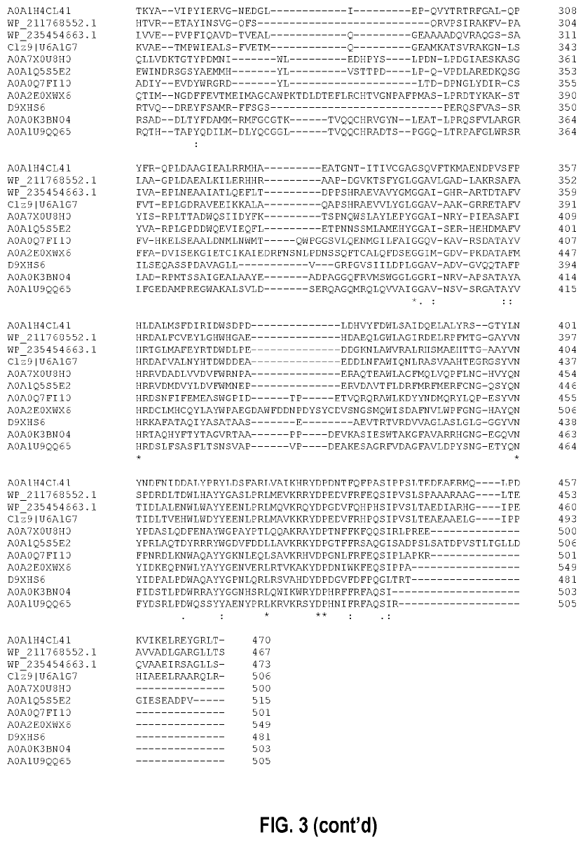

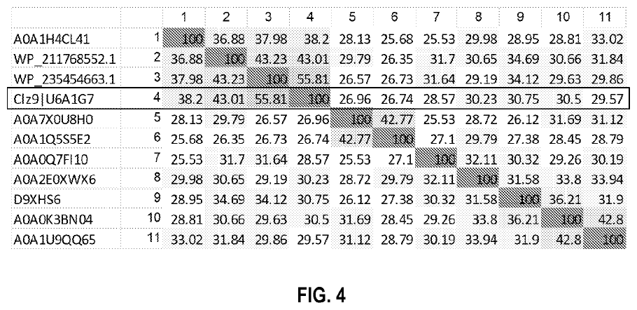

In some embodiments, the flavin-dependent oxidase is a bacterial protein. In some embodiments, the flavin-dependent oxidase is a fungal protein. In some embodiments, the flavin-dependent oxidase is isolated or derived from an organism in Table 1. In some embodiments, the flavin-dependent oxidase comprises a protein in Table 1. Table 1 provides bacterial flavin-dependent oxidases that comprise (i) a His residue bound to an FAD cofactor; and (ii) a peptide motif of Formula T, wherein the FAD cofactor is bound to the Cys residue of the peptide motif, as described herein. A sequence alignment of four of the proteins from Table 1 is shown in FIG. 2.

TABLE 1
Bacterial flavin-dependent oxidases

| UniProt | Organism | UniProt | Organism |
|---|---|---|---|
| Q0PCD7 | Streptomyces galilacus | A0A545AG88 | Cryptosporangium phraense |
| Q7WZ62 | Nonomuraea gerenzanensis | A0A1X1R8K9 | Mycobacterium bohemicum |
| A0A5P8YCI9 | Yersinia pestis | A0A393Y3A4 | Mycolicibacterium porcinum |
| A0A075V680 | Amycolatopsis japonica | A0A0W0N2W5 | Pseudomonas sp. ICMP 19500 |
| A0A1D3DSF8 | Streptomyces thermolilacinus SPC6 | L7V436 | Mycobacterium liflandii (strain 128FXT) |
| A0A191UZV8 | Streptomyces parvulus | A0A1I2AZB5 | Bacillus sp. OV194 |
| A0A1B2HZS0 | Lentzea guizhouensis | A0A1H6EVS1 | Nonomuraea solani |
| A0A1Q5NFS4 | Streptomyces sp. CB02488 | A0A4R5PU84 | Mycobacterium eburneum |
| A0A178WXV2 | Amycolatopsis sp. M39 | A0A1Q8XTN2 | Mycolicibacterium porcinum |
| A0A1X4GRF2 | Streptomyces griscofuscus | A0A318K142 | Nocardia tencrifensis |
| A0A014L9I4 | Streptomyces sp. PRh5 | A0A3N7DKU7 | Variovorax sp. KBW07 |
| A0A1Q5KAW2 | Streptomyces sp. CB02460 | L7V122 | Mycobacterium liflandii (strain 128FXT) |
| A0A1Q4WJJ9 | Streptomyces sp. CB02923 | K0EU35 | Nocardia brasiliensis ATCC 700358 |
| A0A0Q9AGN7 | Streptomyces sp. Root264 | A0A498Q5F0 | Mycobacterium attenuatum |
| A0A1P8XY70 | Streptomyces autolyticus | A0A1H2SRW5 | Amycolatopsis xylanica |
| D6K9C9 | Streptomyces sp. e14 | W5XVC6 | Corynebacterium casei LMG S-19264 |
| A0A4R1TJB9 | Curtobacterium sp. PhB138 | A0A498PQ18 | Mycobacterium attenuatum |
| A0A3R8T190 | Streptomyces sp. RP5T | A0A1H2UY39 | Amycolatopsis xylanica |
| A0A1H1NLZ4 | Jiangella sp. DSM 45060 | A0A1H6DUM2 | Nonomuraea solani |
| A0A5J5KBY3 | Microbispora cellulosiformans | R1G9U0 | Amycolatopsis vancoresmycina DSM 44592 |
| A0A1H3FHW6 | Ruegeria halocynthiae | K0EPJ6 | Nocardia brasiliensis ATCC 700358 |
| A0A349KYY0 | Janthinobacterium sp. | A0A4S2S688 | Streptomyces sp. A1547 |
| A0A5P9YR27 | Nonomuraea nitratireducens | A0A2V8C3P7 | Acidobacteria bacterium |
| A0A3R8SPQ3 | Streptomyces sp. RP5T | A0A0F4W1Z8 | Clostridium sp. IBUN22A |
| A0A6P0HXR1 | Okeania sp. SIO3H1 | A0A2V7VZF5 | Acidobacteria bacterium |
| A0A6P0HSF9 | Okeania sp. SIO3H1 | A0A2V2R8L5 | Acidobacteria bacterium |
| A0A0Q7HM85 | Streptomyces sp. Root1295 | A0A249BX15 | Mycobacterium intracellulare subsp. chimaera |
| A0A1H4ZEZ5 | Streptomyces sp. 2231.1 | A0A1A2PMP2 | Mycobacterium sp. E1319 |
| A0A1S8SAY5 | Clostridium beijerinckii | A0A3D9NJN0 | Streptomyces sp. 75 |
| A0A3M9MH60 | Flexivirga caeni | A0A3D9NBR0 | Streptomyces sp. 75 |
| A0A429EYV8 | Amycolatopsis sp. WAC 04182 | A0A5P9YL04 | Nonomuraea nitratireducens |
| A0A1I4HNL7 | Pelosinus propionicus DSM 13327 | A0A0R2YHB8 | Pseudomonas libanensis |
| A0A366D2Y0 | Nocardia puris | A0A5J5K193 | Microbispora cellulosiformans |
| A0A1W2CSR7 | Sporomusa malonica | A0A4R2EPK8 | Streptomyces sp. BK387 |
| A0A1B9BKS8 | Clostridium beijerinckii | A0A1G3F5Q0 | Pseudomonadales bacterium RIFCSPLOWO2_12_FULL_59_450 |
| A0A3N1J721 | Curtobacterium sp. ZW137 | A0A2P8B8T0 | Streptomyces sp. CS149 |
| A0A024YXM0 | Streptomyces sp. PCS3-D2 | A0A427BQY2 | Empedobacter falsenii |
| A0A0Q8FNK4 | Caulobacter sp. Root655 | A0A3B6VZX5 | Burkholderia pseudomallei |
| A0A2N5D7Z1 | Caulobacter zeae | A0A1Q5C912 | Streptomyces sp. TSRI0395 |
| A0A0C5FZW6 | Streptomyces cyaneogriseus subsp. noncyanogenus | A0A4P2R5W7 | Sorangium cellulosum |
| A0A2U1YVN1 | Azospirillum sp. TSO5 | A0A376G133 | Empedobacter falsenii |
| A0A1W1ZAM4 | Sporomusa malonica | A0A150P8H3 | Sorangium cellulosum |
| A0A4S2TK27 | Streptomyces sp. A0958 | B1HAC8 | Burkholderia pseudomallei S13 |
| A0A2N3VZ92 | Streptomyces sp. TLI_146 | A0A7K2S9M8 | Streptomyces sp. SID8364 |
| A0A640Q969 | Tenacibaculum sp. KUL118 | A6M0E9 | Clostridium beijerinckii (strain ATCC 51743/NCIMB 8052) |
| A0A1X0IT37 | Mycolicibacterium rhodesiae | A0A429DXV5 | Amycolatopsis sp. WAC 04197 |
| A0A429F7N9 | Amycolatopsis sp. WAC 04182 | A0A4R2IEW9 | Kribbella sp. VKM Ac-2541 |
| A0A366D778 | Nocardia puris | A0A3N4QPS7 | Burkholderia pseudomallei |
| A0A022LU98 | Curtobacterium flaccumfaciens UCD-AKU | A0A150PPA5 | Sorangium cellulosum |
| A0A1H3LP80 | Amycolatopsis xylanica | A0A7K2PC88 | Streptomyces sp. SID5926 |
| R1I8C8 | Amycolatopsis vancoresmycina DSM 44592 | A0A525J4R5 | Phenylobacterium sp. |
| A0A3N1GXJ7 | Saccharothrix texasensis | A0A437JN67 | Rheinheimera sp. YQF-1 |
| A0A2S1XMY7 | Azospirillum sp. TSH58 | A0A4Q9HN09 | Streptomyces kasugaensis |
| A0A3Q9K712 | Streptomyces lydicus | A0A243B7J8 | Bacillus thuringiensis serovar poloniensis |
| A0A0M8SEG1 | Streptomyces sp. WM6378 | A0A2S8L905 | Mycobacterium sp. ITM-2016-00318 |
| A0A0D4DNV6 | Streptomyces lydicus | A0A2P2G295 | Amycolatopsis lurida NRRL 2430 |
| A0A1D7VXC3 | Streptomyces lydicus | A0A3G7ABT9 | Pseudomonas sp. CMR12a |
| A0A3Q9JZJ3 | Streptomyces lydicus | A0A525JJ92 | Phenylobacterium sp. |
| A0A0M9YAZ7 | Streptomyces sp. WM6378 | A0A143PGI6 | Luteitalea pratensis |
| A0A1H3FWT1 | Amycolatopsis xylanica | A0A285P9M3 | Pseudomonas sp. URIL14HWK12:I9 |
| M4U8L7 | Ralstonia solanacearum FQY_4 | A0A0I9UDC3 | Mycobacterium haemophilus |
| A0A1H0E4W1 | Afipia sp. GAS231 | A0A0Q9J075 | Bosea sp. Root381 |
| A0A178XF06 | Amycolatopsis sp. M39 | A0A1F2SW14 | Acidobacteria bacterium RIFCSPLOWO2_02_FULL_67_21 |
| A0A7L5SY55 | Streptomyces sp. Rer75 | A0A318RSR0 | Williamsia limnetica |
| A0A7M3LRJ6 | Streptomyces sp. SAJ15 | A0A1E3YX54 | Acidovorax sp. SCN 65-108 |
| A0A6I5HAK2 | Streptomyces sp. SID486 | A0A0I9UNN2 | Mycobacterium haemophilus |
| A0A379BKT6 | Nocardia brasiliensis | A0A2P2FYK8 | Amycolatopsis lurida NRRL 2430 |
| A0A7L5T9P6 | Streptomyces sp. Rer75 | A0A525KMP6 | Phenylobacterium sp. |
| A0A379BYX7 | Nocardia brasiliensis | A0A1Q5LE25 | Streptomyces sp. CB03234 |
| B1R467 | Clostridium perfringens B str. ATCC 3626 | X8FFI1 | Mycobacterium ulcerans str. Harvey |
| A0A1A0TZA1 | Mycobacterium sp. 852014-50255_SCH5639931 | A0A0Q3P660 | Chryseobacterium aquaticum |
| A0A495FEF4 | Acidovorax sp. 93 | A0A3S0DSE4 | Streptomyces sp. WAC05950 |
| A0A1H4IY44 | Terriglobus roseus | A0A1A9HZV7 | Niabella ginsenosidivorans |
| A0A1D7YCY3 | Streptomyces fodineus | A0A1Q5MQH6 | Streptomyces sp. CB02488 |
| A0A7H8TAP2 | Streptomyces chartreusis | A0A1I2J7S3 | Streptomyces alni |
| A0A429EM31 | Amycolatopsis sp. WAC 04197 | A0A1K2B6J4 | Pseudomonas sp. NFPP09 |
| A0A1C1WFJ8 | Pseudomonas sp. S3E12 | A0A1X2AIW3 | Mycobacterium paraense |
| A0A235EP46 | Acidovorax kalamii | A0A6B2TQX0 | Streptomyces sp. SID 13031 |
| A0A175MD92 | Clostridium botulinum B2 433 | A0A388T5S6 | Streptomyces spongiicola |
| A0A150TX40 | Sorangium cellulosum | A0A1Q5S5E2 | Bradyrhizobium sp. NAS96.2 |
| A6LZ39 | Clostridium beijerinckii (strain ATCC 51743/NCIMB 8052) | A0A1H8XT46 | Propionispora vibrioides |
| A0A1A3SIG8 | Mycobacterium sp. 1274761.0 | A0A0F7W2I2 | Streptomyces leeuwenhoekii |
| A0A380Q3E2 | Yersinia pseudotuberculosis | C6BI45 | Ralstonia pickettii (strain 12D) |
| A0A3N1IBT1 | Streptomyces sp. PanSC9 | A0A109K238 | Bradyrhizobium macuxiense |
| A0A7K3BAE4 | Streptomyces sp. SID8377 | A0A4S2DMY9 | Clostridium sartagoforme |
| A0A6G5RH86 | Streptomyces hawaiiensis | A0A0B4W868 | Clostridium botulinum Prevot_594 |
| A0A0W7X5P9 | Streptomyces silvensis | A0A6G2MAJ6 | Streptomyces sp. SID4923 |
| A0A0W7X8I3 | Streptomyces silvensis | A0A1S1LYG5 | Mycobacteroides chelonae |
| A0A2K8YI74 | Bradyrhizobium sp. SK17 | A0A4Y6IW70 | Mycobacteroides chelonae |
| A0A0W7XAU0 | Streptomyces silvensis | A0A1H9SQQ7 | Streptomyces sp. yr375 |
| A0A175RGG1 | Curtobacterium luteum | A0A386U2J2 | Mycobacterium paragordonae |
| A0A239NXI7 | Streptosporangium subroseum | A0A4R5WXQ2 | Mycobacterium paragordonae |
| A0A062WT07 | Frankia sp. BMG5.23 | A0A2V5UBP0 | Verrucomicrobia bacterium |
| A0A495XN27 | Saccharothrix variisporea | A0A4R3UYU7 | Massilia sp. GV097 |
| A0A1C2GIL6 | Mucilaginibacter sp. PPCGB 2223 | A0A1H9VJF9 | Streptomyces sp. yr375 |
| A0A495X6G2 | Saccharothrix variisporea | A0A386TZ44 | Mycobacterium paragordonae |
| A0A031J4M9 | Pseudomonas sp. RIT357 | A0A1S1KYE1 | Mycobacteroides chelonae |
| A0A5P9WPP9 | Streptomyces sp. SYP-A7193 | A0A2V5R7N6 | Verrucomicrobia bacterium |
| A0A650LS33 | Clostridium neonatale | A0A7K2MT80 | Streptomyces sp. SID6013 |
| A0A062X6Y0 | Frankia sp. BMG5.23 | J7WKW4 | Bacillus cereus VD022 |
| A0A1H0A8E6 | Streptomyces wuyuanensis | A0A1S1M4V4 | Mycobacteroides chelonae |
| A0A0G3AKJ5 | Streptomyces incarnatus | A0A2V5VFY1 | Verrucomicrobia bacterium |
| A0A1Q3J4S7 | Acidovorax sp. 65-7 | F2JYC8 | Marinomonas mediterranea (strain ATCC 700492/JCM 21426/NBRC 103028/MMB-1) |
| A0A3R8V3Q1 | Stenotrophomonas sp. 278 | A0A2V5NZ56 | Verrucomicrobia bacterium |
| A0A161XKZ1 | Labrenzia sp. OB1 | A0A0E3TTS0 | Mycobacteroides chelonae |
| A0A0G3UTG8 | Streptomyces sp. Mg1 | A0A2V5SLJ0 | Verrucomicrobia bacterium |
| A0A209BMM0 | Streptomyces sp. CS113 | A0A543FB67 | Nocardia bhagyanarayanae |
| A0A2A7MN17 | Clostridium neonatale | A0A2U8V1D5 | Streptomyces sp. NEAU-S7GS2 |
| A0A165J470 | Acidovorax sp. GW101-3H11 | A0A2U8V0P0 | Streptomyces sp. NEAU-S7GS2 |
| A0A1B4I595 | Burkholderia metallica | A0A5S3XTT1 | Pseudoalteromonas citrea |
| A0A051UKS3 | Mycobacterium [tuberculosis] TKK-01-0051 | A0A218DKE9 | Achromobacter sp. AONIH1 |
| A0A165YG52 | Pseudovibrio axinellae | A0A5C5NHN8 | Pseudomonas marginalis |
| A0A1I2AV58 | Methylobacterium sp. yr596 | A0A643JS78 | Streptomyces sp. HIT-DPA4 |
| A0A1Q4XKC3 | Saccharothrix sp. CB00851 | A0A4R1IBI9 | Ancylobacter aquaticus |
| A0A1H5G6R7 | Streptomyces sp. 3213.3 | A0A1A2JTM8 | Mycobacterium sp. E3339 |
| T2GM88 | Mycobacterium avium subsp. hominissuis (strain TH135) | A0A1X0FYW8 | Mycobacterium mantenii |
| A0A1H7KI02 | Streptacidiphilus jiangxiensis | A0A6I5FTK9 | Streptomyces sp. SID8374 |
| Q5YR72 | Nocardia farcinica (strain IFM 10152) | A0A4Q3BAV7 | Verrucomicrobiaceae bacterium |
| A0A560WYE1 | Streptomyces sp. T12 | A0A4R1IGJ0 | Ancylobacter aquaticus |
| A0A562GH64 | Sporomusa sp. KB1 | A0A0U1QZH1 | Yersinia pseudotuberculosis serotype O:1b (strain IP 31758) |
| A0A6G9ENC7 | Streptomyces sp. VN1 | A0A561TT83 | Kitasatospora viridis |
| A0A525J083 | Bradyrhizobium sp. | A0A1A2TG96 | Mycobacterium mantenii |
| A0A6G9EWC0 | Streptomyces sp. VN1 | A0A1G6JYS1 | Actinokineospora iranica |
| A0A345H405 | Kordia sp. SMS9 | A0A4Q2UC08 | Lichenibacterium minor |
| A0A3N1V0A9 | Streptomyces sp. 2132.2 | A0A561S9A3 | Kitasatospora viridis |
| A0A223S835 | Nocardiopsis gilva YIM 90087 | A0A5S3XTL5 | Pseudoalteromonas citrea |
| A0A1T4PGZ2 | Enhydrobacter aerosaccus | A0A2M8XX51 | Flavobacterium sp. 1 |
| M3BLG9 | Streptomyces gancidicus BKS 13-15 | A0A0A3XLF7 | Bradyrhizobium japonicum |
| A0A7I9YYQ7 | Mycobacterium bourgelatii | A0A1I2KRA1 | Streptomyces alni |
| A0A7H8IPI7 | Streptomyces sp. NA02950 | A0A1N6HIU5 | Paraburkholderia phenazinium |
| A0A6G4AMC7 | Streptomyces rhizosphaericus | A0A7I7L1W4 | Mycobacterium cookii |
| A0A1V4A9A8 | Streptomyces tsukubensis | A0A7I7M0Y9 | Mycobacterium saskatchewanense |
| A0A7I9YJI4 | Mycobacterium bourgelatii | V8CZE1 | Williamsia sp. D3 |
| A0A5H2UQU4 | Streptomyces tsukubensis | A0A3N2GPG7 | Amycolatopsis thermoflava |
| A0A6L9F1Q6 | Flavobacteriaceae bacterium R38 | A0A1E3TIG4 | Mycobacterium shimoidei |
| A0A5Q2J661 | Pseudomonas sp. CFSAN084952 | A0A2S1Z3M3 | Streptomyces spongiicola |
| A0A429BRP2 | Streptomyces sp. WAC 05977 | A0A1H0UR84 | Clostridium gasigenes |
| A0A167FQS0 | Pseudoalteromonas luteoviolacea H33 | A0A4S2DIE3 | Clostridium sartagoforme |
| A0A6I8MAP1 | Amycolatopsis sp. A23 | A0A2H5ASY5 | Kitasatospora sp. MMS16-BH015 |
| A0A4P7Z2V7 | Azospirillum sp. TSA2s | A0A6H1JKM2 | Streptomyces sp. RPA4-5 |
| A0A429B6Z2 | Streptomyces sp. WAC 05977 | A0A536WX81 | Betaproteobacteria bacterium |
| A0A243RL61 | Streptosporangium minutum | A0A4R7R7R2 | Paraburkholderia phenazinium |
| Q2STN7 | Burkholderia thailandensis (strain ATCC 700388/DSM 13276/CIP 106301/E264) | A0A6H1K5C3 | Streptomyces sp. RPA4-5 |
| A0A7K2YC13 | Streptomyces sp. SID1046 | A0A7J5D8A9 | Streptomyces triticiradicis |
| A0A7K2Y6C2 | Streptomyces sp. SID1046 | A0A2G6Z8L8 | Streptomyces sp. 2233.5 |
| A0A1A2YIE5 | Mycobacterium colombiense | A0A1Q5AD90 | Streptomyces sp. CB00072 |
| W0LKM6 | Chania multitudinisentens RB-25 | A0A536VNZ8 | Betaproteobacteria bacterium |
| Q21NE7 | Saccharophagus degradans (strain 2-40/ATCC 43961/DSM 17024) | A0A1C4S342 | Streptomyces sp. Termitarium-T10T-6 |
| A0A6G4A7Y4 | Streptomyces rhizosphaericus | A0A2T0SRN9 | Geodermatophilus tzadiensis |
| A0A243AIE1 | Bacillus thuringiensis serovar navarrensis | B2HDS1 | Mycobacterium marinum (strain ATCC BAA-535/M) |
| A0A2K8QXE5 | Streptomyces sp. M56 | A0A1A2NBQ2 | Mycobacterium sp. E1747 |
| A0A1A2YS30 | Mycobacterium colombiense | A0A2T6L889 | Pseudomonas sp. GV105 |
| A0A329K4J3 | Mycobacterium colombiense | A0A221P089 | Streptomyces pluripotens |
| A0A1A0VTN8 | Mycobacterium colombiense | A0A6H1JJ66 | Streptomyces sp. RPA4-5 |
| A0A2R7NM60 | Acidovorax sp. HMWF018 | B2HJB2 | Mycobacterium marinum (strain ATCC BAA-535/M) |
| A0A329M2F5 | Mycobacterium colombiense | A0A1C4P9W3 | Streptomyces sp. ScaeMP-e83 |
| A0A1R0UME5 | Mycobacterium colombiense | A0A1N6IS34 | Paraburkholderia phenazinium |
| A0A1K0GUC1 | Couchioplanes caeruleus subsp. caeruleus | A0A2H5B989 | Kitasatospora sp. MMS16-BH015 |
| A0A353BUC8 | Firmicutes bacterium | A0A1A3LQA9 | Mycobacterium sp. 1245852.3 |
| A0A4R2ABV7 | Bacillus sp. BK006 | A0A367FEH3 | Sphaerisporangium album |
| A0A5N8WJB7 | Streptomyces phyllanthi | S4YAL0 | Sorangium cellulosum So0157-2 |
| A0A7K3QG23 | Streptomyces diastaticus | A0A1A9J766 | Streptomyces sp. SAT1 |
| U3GC37 | Ralstonia sp. 5_2_56FAA | A0A7C9Q0J8 | Oscillatoria sp. SIO1A7 |
| A0A538JXW1 | Actinobacteria bacterium | M2Z9V8 | Amycolatopsis decaplanina DSM 44594 |
| A0A358SK05 | Actinobacteria bacterium | A0A561H8V3 | Luteibacter yeojuensis |
| A0A7K0ZHE3 | Actinobacteria bacterium | A0A060DV77 | Azospirillum brasilense |
| C8XHY9 | Nakamurella multipartita (strain ATCC 700099/DSM 44233/CIP 104796/JCM | A0A3N4WF52 | Curtobacterium sp. PhB137 |
| | 9543/NBRC 105858/Y-104) | | |
| A0A4R1D692 | Streptomyces sp. KM273126 | A0A1B6AB90 | Streptomyces sp. NBRC 110611 |
| A0A538MCS5 | Actinobacteria bacterium | M2YCG0 | Amycolatopsis decaplanina DSM 44594 |
| A0A538HLR5 | Actinobacteria bacterium | A0A2M8XK63 | Streptomyces sp. 2333.5 |
| A0A6I2Y5M8 | Actinobacteria bacterium | A0A2T7KP69 | Streptomyces sp. CS090A |
| A0A538DG67 | Actinobacteria bacterium | A0A0D6BBC5 | Pseudomonas sp. Os17 |
| A0A6L6R549 | Actinobacteria bacterium | M2Z285 | Amycolatopsis decaplanina DSM 44594 |
| A0A7K0XIX2 | Actinobacteria bacterium | T4VHK4 | Paraclostridium bifermentans ATCC 638 |
| A0A2J7Z0X2 | Streptomyces malaysiensis | A0A7J9VKB2 | Propionibacteriales bacterium |
| A0A538A285 | Actinobacteria bacterium | Q4KJV0 | Pseudomonas fluorescens (strain ATCC BAA-477/NRRL B-23932/Pf-5) |
| A0A537WD39 | Actinobacteria bacterium | A0A147KJC5 | Thermobifida cellulosilytica TB100 |
| A0A6L5ZL01 | Actinobacteria bacterium | A0A3P3QS72 | Pararheinheimera mesophila |
| A0A612YL26 | Actinobacteria bacterium | A0A2A3IKL5 | Streptomyces sp. 2321.6 |
| A0A024KFH5 | Devosia sp. DBB001 | A0A3E0GNM5 | Streptomyces sp. 2221.1 |
| A0A0M8YS18 | Streptomyces sp. NRRL F-7442 | A0A2R4F750 | Acidovorax avenae subsp. avenae |
| A0A1W5XII6 | Streptomyces sp. MOE7 | A0A0C7R0R9 | Paeniclostridium sordellii |
| A0A1W5Y106 | Streptomyces sp. MOE7 | A0A4R1TXA3 | Curtobacterium sp. PhB134 |
| A0A174E437 | Clostridium disporicum | A0A1I2QEY6 | Mycobacterium sp. 455mf |
| W5THF5 | Nocardia nova SH22a | A0A1V2QHB6 | Saccharothrix sp. ALI-22-I |
| A0A174GXV8 | Clostridium disporicum | A0A2Z5JHB3 | Streptomyces atratus |
| A0A1F1LAZ7 | Clostridium sp. HMSC19A10 | A0A327ZKX5 | Actinoplanes lutulentus |
| A0A2T2XHF6 | Sulfobacillus benefaciens | A0A1K2ENL3 | Streptomyces atratus |
| A0A1R0V9C4 | Mycobacterium sp. GA-1841 | A0A1Z4EDS0 | Mycobacterium shigaense |
| A0A1A2B1Q2 | Mycobacterium sp. 852002-51971_SCH5477799-a | A0A1X0AMC2 | Mycobacterium aquaticum |
| A0A4Q0RC49 | Bradyrhizobium guangzhouense | A0A0M2GDL4 | Streptomyces variegatus |
| A0A1B4FA52 | Burkholderia sp. BDU6 | A0A5J6FEZ0 | Streptomyces nitrosporeus |
| A0A5C8H213 | Streptomyces lavendulae | A0A4R7MTQ0 | Streptomyces sp. 846.5 |
| A0A1H1Y3Y7 | Microlunatus soli | A0A6H1KZ39 | Streptomyces sp. RLB1-33 |
| A0A1Q5I6E3 | Streptomyces sp. CB02261 | A0A1S6RXV3 | Streptomyces hygroscopicus |
| A0A6N0Y773 | Curtobacterium sp. Csp2 | A0A1X0Y6G1 | Mycobacterium simiae |
| F0Q7E3 | Acidovorax avenae (strain ATCC 19860/DSM 7227/JCM 20985/NCPPB 1011) | A0A5B1BIS7 | Mycobacterium simiae |
| A0A1Q4XCR8 | Streptomyces sp. CB03911 | A0A6H1L8M9 | Streptomyces sp. RLB1-33 |
| A0A7K2WNP6 | Streptomyces sp. SID3212 | A0A1S6RNA1 | Streptomyces hygroscopicus |
| A0A7K2WNU3 | Streptomyces sp. SID3212 | A0A1V2QV28 | Saccharothrix sp. ALI-22-I |
| D7AZ71 | Nocardiopsis dassonvillei (strain ATCC 23218/DSM 43111/CIP 107115/JCM | A0A1G4W024 | Pseudomonas sp. NFACC25 |
| | 7437/KCTC 9190/NBRC 14626/NCTC 10488/NRRL B-5397/IMRU 509) | | |
| A0A5B9YJC4 | Cellulosilyticum sp. WCF-2 | A0A0J6SPQ4 | Methylobacterium variabile |
| A0A4Y8XWK6 | Streptomyces sp. 4R-3d | A0A7K0DYH1 | Nocardia sp. RB56 |
| A0A7K2PVE7 | Streptomyces sp. SID5470 | A0A1A3ETJ7 | Mycobacterium sp. 1245801.1 |
| A0A7K2PP54 | Streptomyces sp. SID5470 | A0A1C6N6F2 | Streptomyces sp. AmelKG-E11A |
| A0A286EHU9 | Jatrophihabitans sp. GAS493 | A0A1V3XYN6 | Mycobacterium kansasii |
| A0A4Y8Y737 | Streptomyces sp. 4R-3d | A0A164A4Z5 | Mycobacterium kansasii |
| B7INI0 | Bacillus cereus (strain G9842) | A0A1I2WRS1 | Streptomyces mirabilis |
| A0A0P9HKL1 | Paenibacillus sp. A3 | A0A387HGZ6 | Streptomyces hundungensis |
| A0A1X1UI09 | Mycobacterium florentinum | A0A1A2ZRS3 | Mycobacterium sp. E796 |
| A0A1X1UDI8 | Mycobacterium florentinum | A0A6G9FHZ4 | Streptomyces sp. Tu 2975 |
| A0A0A0NJ25 | Streptomyces rapamycinicus (strain ATCC 29253/DSM 41530/NRRL 5491/AYB-994) | A0A1A0KPI7 | Mycobacterium sp. 1100029.7 |
| A0A0E1NSR7 | Yersinia pestis bv. Antiqua (strain Antiqua) | A0A163WER8 | Mycobacterium kansasii |
| A0A100HSR8 | Ralstonia sp. NT80 | A0A4U1IX92 | Polyangium fumosum |
| A0A255YQA6 | Niveispirillum lacus | A0A4R5H8W5 | Alteromonadaceae bacterium M269 |
| A0A5D0U6Y6 | Actinomadura syzygii | A0A543BZT2 | Actinoallomurus bryophytorum |
| A0AIN6H3D0 | Bradyrhizobium erythrophlei | A0A1X7GHB8 | Streptomyces sp. Amel2xC10 |
| A0A516NKC8 | Nocardia otitidiscaviarum | A0A5S4FQB0 | Nonomuraea turkmeniaca |
| A0A1L3LZB5 | Sinorhizobium americanum | A0A286HVS5 | Streptomyces sp. 2323.1 |
| Q3JY23 | Burkholderia pseudomallei (strain 1710b) | A0A1V4D850 | Streptomyces antioxidans |
| A0A2N7YKQ9 | Pseudomonas sp. GW704-F2 | A0A223RWZ3 | Actinopolyspora erythraea |
| A0A4R2AU73 | Sinorhizobium americanum | A0A495CDC3 | Streptomyces sp. 42 |
| A0A4R3UGM6 | Curtobacterium sp. PhB191 | A0A1Q3WSQ0 | Spirosoma sp. 48-14 |
| A0A1H4VSC7 | Pseudomonas saponiphila | A0A6J5BKG0 | Achromobacter insuavis |
| A0A4Z0HG08 | Streptomyces palmae | A0A2N3ZSC2 | Streptomyces sp. OK885 |
| A0A3C0GV39 | Stenotrophomonas sp. | J4SGS7 | Mycobacterium colombiense CECT 3035 |
| A0A6L6X2L4 | Streptomyces sp. p1417 | A0A1V4D704 | Streptomyces antioxidans |
| A0A1C5GI90 | Micromonospora echinofusca | A0A1A0KTR0 | Mycobacterium sp. 1100029.7 |
| A0A1W5XUU1 | Streptomyces sp. MOE7 | A0A1I2EQU3 | Streptomyces mirabilis |
| G4HTY7 | Mycolicibacterium rhodesiae JS60 | A0A2R4EFS1 | Acidovorax avenae subsp. avenae |
| A0A1X0E1G1 | Mycobacterium malmoense | A0A4R5H288 | Alteromonadaceae bacterium M269 |
| A0A5C0AX84 | Pigmentiphaga aceris | A0A3G9GH94 | Aquitalea magnusonii |
| A0A1S2WRC3 | Mycobacterium malmoense | A0A2P2GUA5 | Streptomyces showdoensis |
| A0A7K2FDD1 | Streptomyces sp. SID4921 | A0A2R4EYI7 | Acidovorax avenae subsp. avenae |
| V6K7L7 | Streptomyces roseochromogenus subsp. oscitans DS 12.976 | A0A2R4ENT6 | Acidovorax avenae subsp. avenae |
| A0A1W5XRR5 | Streptomyces sp. MOE7 | A0A2R4DKW7 | Acidovorax avenae subsp. avenae |
| A0A3N1XB65 | Mobilisporobacter senegalensis | A0A4R8CG71 | Curtobacterium sp. PhB25 |
| A0A1B9CT95 | Mycobacterium malmoense | A0A099CZK7 | Actinopolyspora erythraea |
| A0A1X0E122 | Mycobacterium malmoense | A0A3E0GNN4 | Streptomyces sp. 2221.1 |
| A0A429RJS0 | Streptomyces sp. WAC07061 | A0A1A3FJQ1 | Mycobacterium sp. 1245801.1 |
| B5HLZ9 | Streptomyces sviceus ATCC 29083 | A0A0N0UXK4 | bacterium 336/3 |
| A0A2B4FEN2 | Bacillus sp. AFS059628 | A0A1A3ETW0 | Mycobacterium sp. 1245801.1 |
| A0A6I5GXM9 | Streptomyces sp. SID89 | A0A3M5V505 | Pseudomonas syringae pv. avii |
| A0A447G7Y4 | Mycobacterium basiliense | A0A495CEQ6 | Streptomyces sp. 42 |
| A0A3S4DRN8 | Mycobacterium basiliense | A0A1X0IKA3 | Mycobacteroides saopaulense |
| A0A0H5RPQ5 | Mycolicibacterium neworleansense | A0A4Q7QV16 | Fictibacillus sp. BK138 |
| A0A6G2YTU7 | Streptomyces sp. SID8373 | A0A4D8R6Q5 | Azospirillum brasilense |
| A0A6I5GJE8 | Streptomyces sp. SID89 | A0A372G4X5 | Micromonospora craniellae |
| A0A101NQW8 | Streptomyces cellostaticus | A0A3R9EM40 | Amycolatopsis eburnea |
| A0A101NG92 | Streptomyces cellostaticus | A0A656KTC4 | Mycobacterium avium 10-5560 |
| A0A2U3NMV7 | Mycobacterium rhizamassiliense | A0A176J656 | Bacillus sp. SJS |
| D3DC17 | Frankia sp. EUN1f | A0A4R3SS91 | Bacillus sp. OK077 |
| U1LLS2 | Pseudoalteromonas rubra DSM 6842 | A0A546ZJ30 | Agrobacterium rhizogenes |
| A0A3Q9NSU6 | Brevibacterium aurantiacum | Q66FH3 | Yersinia pseudotuberculosis serotype I (strain IP32953) |
| A0A0Q7X5X7 | Duganella sp. Root1480D1 | A0A560BYN9 | Azospirillum brasilense |
| A0A542Q611 | Streptomyces sp. SLBN-118 | A0A4D8PMB2 | Azospirillum brasilense |
| A0A401Y5W6 | Nocardioides sp. LS1 | A0A2K1FX54 | Azospirillum brasilense |
| A0A542QLS6 | Streptomyces sp. SLBN-118 | A0A2N7N7Y5 | Vibrio sp. 10N.222.52.B12 |
| A0A542Q257 | Streptomyces sp. SLBN-118 | A0A654SRY5 | Paeniclostridium sordellii |
| A0A1A3QEU1 | Mycobacterium sp. 1081908.1 | A0A0M1UT57 | Paeniclostridium sordellii |
| J1H5C3 | Clostridium sp. MSTE9 | A0A3R9F105 | Amycolatopsis eburnea |
| A0A6I6FF78 | Streptomyces ficellus | A0A3R9EKF3 | Amycolatopsis eburnea |
| N9XKP8 | Clostridium thermobutyricum | A0A4D8QDI0 | Azospirillum brasilense |
| A0A2T5NRE4 | Chromobacterium haemolyticum | A0A5B0KY29 | Azospirillum brasilense |
| A0A1S2PF81 | Streptomyces monashensis | A0A562RSY8 | Bradyrhizobium huanghuaihaiense |
| A0A6M0WJC1 | Clostridium botulinum | C3DV57 | Bacillus thuringiensis serovar sotto str. T04001 |
| A0A2I4NMA0 | Clostridium botulinum | K4R8X4 | Streptomyces davaonensis (strain DSM 101723/JCM 4913/KCC S-0913/768) |
| A0A368DZJ3 | Flavobacteriales bacterium | A0A2S6IUV5 | Kineococcus xinjiangensis |
| A0A1H7MU19 | Streplacidiphilus jiangxiensis | A0A6L5SF60 | Paeniclostridium sordellii |
| A0A3N1V2I5 | Streptomyces sp. 2132.2 | A0A3R9FAG7 | Amycolatopsis eburnea |
| A0A0L8LSD6 | Streptomyces decoyicus | A0A235HA30 | Azospirillum brasilense |
| A0A0L8M8Q5 | Streptomyces decoyicus | A0A6G2XUS4 | Streptomyces sp. SID8382 |
| A0A3N1SQI0 | Streptomyces sp. 840.1 | A0A1C4P8K6 | Streptomyces sp. SolWspMP-5a-2 |
| A0A1H4V8X6 | Streptomyces sp. 3213.3 | A0A1Q5KI24 | Streptomyces sp. TSRI0107 |
| A0A1H7XYN0 | Streptacidiphilus jiangxiensis | A0A0NIN8Y6 | Actinobacteria bacterium OK006 |
| A0A223SDK9 | Nocardiopsis gilva YIM 90087 | A0A0K8JG76 | Propionispora sp. 2/2-37 |
| A0A0L8MFU1 | Streptomyces decoyicus | A0A2P8D4A7 | Taibaiella chishuiensis |
| A0A345H3F3 | Kordia sp. SMS9 | A0A4R7IXY2 | Streptomyces sp. BK447 |
| A0A543FPB9 | Pseudonocardia cypriaca | A0A1Y1QEB5 | Thiothrix lacustris |
| A0A2T6C033 | Kordia periserrulae | A0A6B2S926 | Streptomyces sp. SID10115 |
| A0A2T6C443 | Kordia periserrulae | A0A1Y1QLZ2 | Thiothrix lacustris |
| A0A1Z4JF17 | Leptolyngbya boryana NIES-2135 | A0A6G2URS3 | Streptomyces sp. SID4931 |
| A0A7G6X4L7 | Kribbella qitaiheensis | A0A3L7ARW3 | Mycetocola lacteus |
| A0A3B7QLA5 | Streptomyces fradiae | A0A124GZV5 | Streptomyces curacoi |
| A0A7G6WRP1 | Kribbella qitaiheensis | A0A4R5LGH9 | Paraburkholderia guartelaensis |
| A0A081I5H3 | Mycobacterium sp. TKK-01-0059 | A0A2K8M1G2 | Amycolatopsis sp. AA4 |
| A0A3B7QU30 | Streptomyces fradiae | A0A1G5LKS2 | Streptomyces sp. 136MFCol5.1 |
| A0A1V2I5Y0 | Frankia asymbiotica | A0A2W6E2K8 | Pseudonocardiales bacterium |
| A0A6N4T9G3 | Undibacterium sp. KW1 | A0A7K3RV52 | Streptomyces parvus |
| H1Q5T1 | Streptomyces coelicoflavus ZG0656 | A0A1B2HIX0 | Lentzea guizhouensis |
| A0A561WUA4 | Micromonospora palomenae | A0A2W6B621 | Pseudonocardiales bacterium |
| A0A1V4PGD9 | Mycobacterium sp. AT1 | A0A2W6E424 | Pseudonocardiales bacterium |
| A0A7G5P2S7 | Clostridium butyricum | A0A656FKV4 | Yersinia pestis biovar Orientalis str. PEXU2 |
| A0A1W6X798 | Mesorhizobium sp. WSM1497 | F6EGI9 | Hoyosella subflava (strain DSM 45089/JCM 17490/NBRC 109087/DQS3-9A1) |
| A0A4Q1R5U1 | Streptomyces sioyaensis | A0A1E7M023 | Streptomyces nanshensis |
| H1Q9Q6 | Streptomyces coelicoflavus ZG0656 | A0A3M5N7F7 | Pseudomonas azotoformans |
| A0A4Q1R4U8 | Streptomyces sioyaensis | A0A2B8B9Q2 | Azospirillum palustre |
| A0A512TJW7 | Clostridium butyricum | A0A1E7KX67 | Streptomyces nanshensis |
| A0A6B2RHZ5 | Streptomyces sp. SID10362 | A0A1H0Z3C4 | Actinopolyspora saharensis |
| A0A2S4XX85 | Streptomyces sp. Ru73 | A0A1E7L275 | Streptomyces nanshensis |
| A0A1H8B618 | Nonomuraea pusilla | A0A0D0PSK8 | Kitasatospora griseola |
| D3F7G5 | Conexibacter woesei (strain DSM 14684/CIP 108061/JCM 11494/NBRC | A0A514B522 | Pseudomonas azotoformans |
| | 100937/ID131577) | | |
| A0A0F4VRA7 | Clostridium sp. IBUN125C | A0A1U9PRC7 | Pseudomonas azotoformans |
| A0A410Q7D1 | Caproiciproducens sp. NJN-50 | A0A2W6BPT6 | Pseudonocardiales bacterium |
| A0A3D4SLC6 | Pseudomonas sp. | A0A5D4JM58 | Streptomyces parvus |
| A0A6M0H256 | Clostridium senegalense | A0A0X3XR61 | Streptomyces sp. NRRL WC-3605 |
| A0A6V8L618 | Phytohabitans rumicis | A0A0X3XHY7 | Streptomyces sp. NRRL WC-3605 |
| A0A5P2BWJ8 | Streptomyces venezuelae | A0A0D0NG31 | Kitasatospora griseola |
| A0A3B8KJV5 | Pseudomonas sp. | E4N3X7 | Kitasatospora setae (strain ATCC 33774/DSM 43861/JCM 3304/KCC A-0304/NBRC |
| | | | 14216/KM-6054) |
| A0A5P2CJY5 | Streptomyces venezuelae | A0A4Q7WAU2 | Plantactinospora sp. CNZ321 |
| A0A242XW47 | Bacillus thuringiensis serovar novosibirsk | A0A5D4JL66 | Streptomyces parvus |
| A0A4RISWE2 | Curtobacterium sp. PhB142 | A0A173ZZ51 | Turicibacter sanguinis |
| A0A2T2X0H1 | Sulfobacillus thermosulfidooxidans | A0A1V4SWQ2 | Clostridium thermobutyricum DSM 4928 |
| A0A3E0IAK2 | Kutzneria buriramensis | A0A242N9P3 | Caballeronia sordidicola |
| A0A3N1L2Y2 | Streptomyces sp. 844.5 | A0A4V2GE63 | Streptomyces sp. CNZ288 |
| A0A258B1R7 | Verrucomicrobia bacterium 12-59-8 | A0A5M8G9D5 | Pseudomonas lactis |
| A0A1Q4VGF7 | Streptomyces sp. CB02056 | A0A371PQT5 | Streptomyces inhibens |
| A0A562GFF0 | Sporomusa sp. KB1 | A0A1H2XJ08 | Nitrosomonas communis |
| A0A4Y4K702 | Streptomyces sp. 6-11-2 | C1B6J4 | Rhodococcus opacus (strain B4) |
| A0A4Z1DIK8 | Streptomyces griseoluteus | A0A0Q8RN59 | Duganella sp. Root198D2 |
| A0A0J8WW77 | Mycolicibacterium conceptionense | M3BZ21 | Streptomyces mobaraensis NBRC 138 |
| A0A418KSL6 | Jiangella rhizosphaerae | A0A1G8L9U0 | Chryseobacterium taeanense |
| A0A223VKV5 | Pseudomonas sp. NS1(2017) | A0A229TNK7 | Amycolatopsis sp. KNN50.9b |
| A0A2U1XTW6 | Azospirillum sp. TSH64 | A0A371Q6Q6 | Streptomyces inhibens |
| A0A536C4I5 | Chloroflexi bacterium | A0A024K232 | Mycobacterium triplex |
| A0A535D5Z4 | Chloroflexi bacterium | A0A174PTU1 | Turicibacter sanguinis |
| A0A2S6WL67 | Streptomyces sp. 46 | A0A371PXX4 | Streptomyces inhibens |
| A0A0J7I4T4 | Chryseobacterium angstadtii | A0A1I4KTZ4 | Nitrosomonas communis |
| A0A535N6V2 | Chloroflexi bacterium | A0A1V4SLA3 | Clostridium thermobutyricum DSM 4928 |
| A0A0S9F2Z0 | Acidovorax sp. Leaf76 | A0A4R3D675 | Streptomyces sp. BK308 |
| A0A402BNQ7 | Sporomusaceae bacterium | A0A5D3YBN0 | Nitrosomonas communis |
| A0A536IIM8 | Chloroflexi bacterium | A0A371PZH3 | Streptomyces inhibens |
| A0A0E1TSU6 | Burkholderia pseudomallei 576 | A0A242N418 | Caballeronia sordidicola |
| A0A536P8P6 | Chloroflexi bacterium | A0A498QVK8 | Mycobacterium pseudokansasii |
| A0A536S614 | Chloroflexi bacterium | A0A1I6F8A9 | Lentzea waywayandensis |
| A0A2S6WBU0 | Streptomyces sp. 46 | A0A5B0BQ84 | Streptomyces sp. SUN51 |
| A0A1F4J1E7 | Burkholderiales bacterium RIFCSPHIGHO2_12_FULL_65_48 | A0A7I8A4J9 | Mycolicibacterium insubricum |
| A0A535K016 | Chloroflexi bacterium | A0A5B9E579 | Terriglobus albidus |
| A0A0V2F6I8 | Caulobacter vibrioides | A0A1M5NXG2 | Bradyrhizobium erythrophlei |
| A0A4U0NVI4 | Streptomyces piniterrae | A0A4Z0G665 | Streptomyces palmae |
| A0A4V5UL67 | Pseudomonas sp. CFBP13528 | A0A516NVX1 | Nocardia otitidiscaviarum |
| A0A2M9IKN3 | Streptomyces sp. CB01373 | A0A2T0JX66 | Actinoplanes italicus |
| A0A2R5H1I0 | Mycobacterium montefiorense | A0A410UFZ5 | Dyella sp. M7H15-1 |
| A0A1X7FBK9 | Azospirillum oryzae | A0A497VTC9 | Streptomyces sp. 74 |
| A0AIR0UHJ3 | Mycobacterium sp. SP-6446 | A0A429C6W9 | Amycolatopsis sp. WAC 04169 |
| A0A2M9IKT6 | Streptomyces sp. CB01373 | A0A379JK70 | Nocardia otitidiscaviarum |
| A0AIR0UBN0 | Mycobacterium sp. SP-6446 | A3RXB7 | Ralstonia solanacearum UW551 |
| X7ZHU1 | Mycobacterium kansasii 662 | A0A401ZS62 | Dictyobacter aurantiacus |
| A0A615I158 | Streptomyces sp. SID4913 | A0A4Z0FXW6 | Streptomyces palmae |
| A0A2P4RGJ4 | Ralstonia pickettii | A0A2N4SM64 | Variovorax sp. RO1 |
| A0A3Q8VV19 | Streptomyces sp. KPB2 | A0A5D0U873 | Actinomadura syzygii |
| A0A6N1ADJ9 | Azospirillum oryzae | A0A0F4KC52 | Streptomyces sp. NRRL B-1568 |
| A0A5C7YAH0 | Mycolicibacterium mageritense | A0A1Y2SL82 | Xenorhabdus beddingii |
| X7ZMM2 | Mycobacterium kansasii 662 | K0JZW9 | Saccharothrix espanaensis (strain ATCC 51144/DSM 44229/JCM 9112/NBRC |
| | | | 15066/NRRL 15764) |
| A0A0J6XKQ8 | Streptomyces roseus | A0A498RAJ8 | Lucifera butyrica |
| A0A3Q8VSR0 | Streptomyces sp. KPB2 | A0A1H2CB28 | Streptomyces sp. 2114.2 |
| A0A512BJY2 | Segetibacter aerophilus | A0A1A3L1M1 | Mycobacterium gordonae |
| A0A2M9J0N0 | Streptomyces sp. CB01201 | A0A1A3K848 | Mycobacterium gordonae |
| A0A6M0WIX2 | Clostridium botulinum | A0A0Q2MG23 | Mycobacterium gordonae |
| A0A3N1AZ61 | Micromonospora sp. Llam0 | A0A7K3CUR5 | Streptomyces sp. SID337 |
| A0A7C9JGT5 | Herbidospora sp. NEAU-GS84 | A0A3N2GL19 | Curtobacterium sp. PhB78 |
| A0A0F4JIP2 | Streptomyces sp. NRRL S-495 | A0A1H4XC28 | Streptomyces sp. 2131.1 |
| A0A4V2TX02 | Streptomyces sp. BK329 | A8L7P3 | Frankia sp. (strain EAN1pec) |
| A0A543J733 | Saccharothrix saharensis | A0A543J735 | Saccharothrix saharensis |
| A0A1C5EY95 | Streptomyces sp. MnatMP-M17 | A0A4R3C3L8 | Streptomyces sp. BK329 |
| A0A2M9KES8 | Streptomyces sp. CB02120-2 | A0A0F4JD92 | Streptomyces sp. NRRL S-495 |
| A0A6G3R9M9 | Streptomyces sp. SID339 | A0A7L5A6M7 | Granulicella sp. WH15 |
| A0A6G3RFL1 | Streptomyces sp. SID339 | A0A2V1NI77 | Streptomyces sp. V2 |
| A0A6G3R645 | Streptomyces sp. SID339 | A0A1N7ULJ9 | Pseudomonas simiae |
| A0A5H2Q188 | Ralstonia solanacearum | A0A3NIAZS8 | Micromonospora sp. Llam0 |
| A0A1S2QHK7 | Streptomyces monashensis | A0A7K3PNG0 | Streptomyces coelicoflavus |
| H8G477 | Saccharomonospora azurea NA-128 | A0A1C6TPR3 | Micromonospora aurantiaca |
| B0T105 | Caulobacter sp. (strain K31) | A0A495HF47 | Streptomyces sp. 3212.4 |
| G7ZF90 | Azospirillum lipoferum (strain 4B) | A0A5D0NQP8 | Actinomadura chibensis |
| A0A177RKW5 | Ralstonia solanacearum | A0A2T0R6S8 | Kineococcus rhizosphaerae |
| A0A348PHU5 | Phycisphaerales bacterium | A0A1Q5DZZ0 | Streptomyces sp. CB01249 |
| A0A1Q5N6S4 | Streptomyces sp. CB00455 | A0A2V1NF41 | Streptomyces sp. V2 |
| A0A0F4JUT1 | Streptomyces sp. NRRL S-495 | A0A1I3I085 | Nitrosomonas sp. Nm34 |
| A0A2VINYA3 | Streptomyces sp. V2 | A0A085HMK8 | Leminorella grimontii ATCC 339 |
| A0A543JAT7 | Saccharothrix saharensis | A0A518WQW7 | Micromonospora sp. HM134 |
| A0A3N1AS80 | Micromonospora sp. Llam0 | A0A3M9L1E4 | Micromonospora aurantiaca |
| A0A7G5LM42 | Devosia sp. MC521 | A0A543JR56 | Saccharothrix saharensis |
| A0A1Q9LEV8 | Actinokineospora bangkokensis | U5WWX2 | Mycobacterium kansasii ATCC 12478 |
| A0A1W1WB70 | Sulfobacillus thermosulfidooxidans (strain DSM 9293/VKM B-1269/AT-1) | A0A1Q5MJF5 | Streptomyces sp. CB00455 |
| A0A4R1BYK8 | Nocardioides jejuensis | A0A2V1NKM2 | Streptomyces sp. V2 |
| V7MSV3 | Mycobacterium avium subsp. hominissuis 10-5606 | A0A543J9Z8 | Saccharothrix saharensis |
| A0A219AA07 | Pseudomonas lactis | A0A2M9KLG0 | Streptomyces sp. CB02120-2 |
| A0A4Q8CX27 | Streptomyces sp. CNZ288 | U5WKA4 | Mycobacterium kansasii ATCC 12478 |
| A0A2G7E6T7 | Streptomyces sp. CNZ279 | A0A1H2D6P2 | Streptomyces sp. 2114.2 |
| A0A1C4MWW1 | Streptomyces sp. DfronAA-171 | A0A1H2D738 | Streptomyces sp. 2114.2 |
| A0A1A3JJ08 | Mycobacterium marseillense | A0A6G3QIZ0 | Streptomyces sp. SID12488 |
| I4K7L3 | Pseudomonas lactis | A0A2M9KES1 | Streptomyces sp. CB02120-2 |
| R8RWI4 | Bacillus cereus HuB4-4 | A0A2V1NHI7 | Streptomyces sp. V2 |
| A0A717PW53 | Mycobacterium marseillense | A0A1C0UJKI | Ralstonia solanacearum |
| A0A7K2UIA3 | Streptomyces sp. SID7813 | A0A5B7U822 | Ralstonia solanacearum |
| A0A024JUF9 | Mycobacterium triplex | A0A6N9UND2 | Streptomyces coelicoflavus |
| A0A1E7KX70 | Streptomyces nanshensis | A0A2G7AMR9 | Streptomyces sp. 94 |
| A0A0R3CIC9 | Pseudomonas lactis | A0A4S3GKA0 | Streptomyces sp. A1499 |
| A0A4Q8CTW5 | Streptomyces sp. CNZ288 | A0A1H4Z2Q3 | Streptomyces misionensis |
| A0A1E7TSS1 | Variovorax boronicumulans | A0A510TD33 | Streptomyces sp. 1-11 |
| A0A498QJP0 | Mycobacterium pseudokansasii | A0A0Q3WPY1 | Brevibacillus choshinensis |
| A0A4R1L8N1 | Acidipila rosea | A0A2D5B876 | Phycisphaerae bacterium |
| A0A226X1Q2 | Caballeronia sordidicola | A0A7H1B8K6 | Streptomyces sp. CRXT-Y-14 |
| A0A7K2UNP8 | Streptomyces sp. SID7813 | A0A4R7IJ96 | Streptomyces sp. BK208 |
| A0A1V2JJ06 | Pseudomonas azotoformans | A0A0F7N8G1 | Streptomyces sp. CNQ-509 |
| A0A2W6E3P0 | Pseudonocardiales bacterium | A0A0F7N849 | Streptomyces sp. CNQ-509 |
| A0A6L7S2E6 | Boseongicola sp. SB0662_bin 57 | A0A089XDD5 | Streptomyces glaucescens |
| A0A4Q0HTZ4 | Pseudomonas azotoformans | A0A6G2VC78 | Streptomyces sp. SID2563 |
| A0A6G4TZI0 | Streptomyces sp. A7024 | W7ITU2 | Actinokineospora spheciospongiae |
| A0A7K2UNV6 | Streptomyces sp. SID7813 | A0A1V3WP65 | Mycobacterium kansasii |
| A0A6G4TV07 | Streptomyces sp. A7024 | A0A0P4RGQ8 | Streptomyces lydicamycinicus |
| S5VIU2 | Streptomyces collinus (strain DSM 40733/Tue 365) | A0A0H2YSN5 | Clostridium perfringens (strain ATCC 13124/DSM 756/JCM 1290/NCIMB 6125/ |
| | | | NCTC 8237/Type A) |
| A0A2E1GDI4 | Gordonia sp. | G2PBH3 | Streptomyces violaceusniger (strain Tu 4113) |
| A0A2C9SUT7 | Mycobacterium sp. shizuoka-1 | A0A4U0T6M5 | Streptomyces sp. NEAU-C40 |
| A0A0F5VZG3 | Streptomyces sp. WM6386 | A0A3E0I1X1 | Tenacibaculum gallaicum |
| W7SW23 | Kutzneria sp. 744 | A0A0C2BHU2 | Streptomyces sp. 150FB |
| A0A6N7KVE6 | Streptomyces kaniharaensis | A0A5B7UUV3 | Streptomyces sp. YIM 121038 |
| A0A7G3FRB9 | Roseivirga sp. XM-24bin3 | A0A0M8W0P7 | Streptomyces sp. NRRL WC-3723 |
| A0A1G7V7P2 | Lentzea fradiae | A0A5B7UYC0 | Streptomyces sp. YIM 121038 |
| A0A0H3L4D2 | Mycobacterium tuberculosis (strain ATCC 35801/TMC 107/Erdman) | A0A3D9QPJ2 | Streptomyces sp. 3212.3 |
| A0A1K1PD14 | Amycolatopsis australiensis | A0A0B1YIF9 | Ralstonia sp. A12 |
| A0A2S5GQ03 | Achromobacter spanius | A0A2R4JK22 | Streptomyces sp. P3 |
| V6JZZ0 | Streptomyces niveus NCIMB 11891 | B5H5L5 | Streptomyces pristinaespiralis (strain ATCC 25486/DSM 40338/CBS 914.69/ |
| | | | JCM 4507/NBRC 13074/NRRL 2958/5647) |
| V6KLW7 | Streptomyces niveus NCIMB 11891 | A0A0C2B721 | Streptomyces sp. 150FB |
| A0A640T5J1 | Streptomyces glebosus | A0A3D9QFY7 | Streptomyces sp. 3212.3 |
| A0A640T0F0 | Streptomyces glebosus | A0A2N3Y661 | Saccharopolyspora spinosa |
| A0A495KZS4 | Nocardiopsis sp. Huas11 | A0A4U8Q044 | Robinsoniella peoriensis |
| A0A3S0Z2H6 | Bacillus sp. VKPM B-3276 | A0A0C1YE14 | Streptomyces sp. 150FB |
| B9NM43 | Rhodobacteraceae bacterium KLH11 | A0A5J6IZH0 | Streptomyces cinereoruber |
| A0A1B7URG9 | Rheinheimera sp. SA_1 | A0A3A5LZ27 | Arthrobacter sp. Hz2 |
| A0A291CMG0 | Burkholderia thailandensis | A0A1H8P6E3 | Streptomyces rubidus |
| A0A1C6VV07 | Micromonospora peucetia | A0A2R7QM27 | Stenotrophomonas sp. HMWF003 |
| A0A6I1Z3X9 | Streptomyces sp. RB17 | A0A231PRI7 | Streptomyces sp. XY006 |
| A0A640T6Z9 | Streptomyces glebosus | A0A5N8VZ81 | Streptomyces phyllanthi |
| A0A3S4S0P7 | Achromobacter spanius | A0A1C5EW72 | Streptomyces sp. MnatMP-M27 |
| A0A2W6RVR9 | Gordonia sp. | A0A6I0B618 | Bacillus sp. CH140a_4T |
| B9NWM4 | Rhodobacteraceae bacterium KLH11 | A0A365H109 | Actinomadura craniellae |
| A0A640SRI9 | Streptomyces glebosus | A0A2G0WNF8 | Pseudomonas sp. ICMP 564 |
| A0A1K1S7B1 | Amycolatopsis australiensis | K0AWE1 | Gottschalkia acidurici (strain ATCC 7906/DSM 604/BCRC 14475/CIP 104303/KCTC |
| | | | 5404/NCIMB 10678/9a) |
| A0A5A7XRX5 | Mycolicibacterium sp. P1-18 | A0A6I8LXT8 | Amycolatopsis sp. A23 |
| A0A1R0VH33 | Mycobacterium sp. IS-2888 | A0A4Q3VAA9 | bacterium |
| A0A3N1KKX3 | Streptomyces sp. 844.5 | A0A0L6VXY1 | Massilia sp. WF1 |
| A0A3N1KT76 | Streptomyces sp. 844.5 | A0A4R2JW48 | Actinocrispum wychmicini |
| A0A2G7CF31 | Streptomyces sp. 61 | A0A7K1KX27 | Actinomadura litoris |
| A0A5D3F7N6 | Actinomadura decatromicini | A0A2S5W277 | Subtercola sp. Z020 |
| A0A2G7CKQ6 | Streptomyces sp. 61 | A0A7K1L5J1 | Actinomadura litoris |
| A0A5D3FTJ0 | Actinomadura decatromicini | A0A6I8M3G8 | Amycolatopsis sp. A23 |
| A0A1G9YWK3 | Allokutzneria albata | A0A109ZZW5 | Burkholderia sp. PAMC 26561 |
| A0A1Q4WA71 | Streptomyces sp. CB02056 | A0A1Y4YBN0 | Pluralibacter gergoviae |
| A0A1M3KZC8 | ‘Candidatus Kapabacteria’ thiocyanatum | A0A6B3F703 | Streptomyces sp. SID11233 |
| A0A0E1V3J4 | Burkholderia pseudomallei Pakistan 9 | A0A5B1LK32 | Nocardioides sp. BN130099 |
| U5E7N1 | Nocardia asteroides NBRC 15531 | A0A371J2W9 | Romboutsia weinsteinii |
| A0A1Q4W7J0 | Streptomyces sp. CB02056 | A0A5Q0H529 | Saccharothrix syringae |
| D8NAD0 | Ralstonia solanacearum CMR15 | A0A0B6S669 | Burkholderia plantarii |
| A0A1R0VM44 | Mycobacterium sp. IS-2888 | A0A5Q0HD87 | Saccharothrix syringae |
| A0A1T3NL76 | Embleya scabrispora | A0A2G3PP23 | Williamsia marianensis |
| A0A229RJQ5 | Amycolatopsis thailandensis | A0A6L8N5X1 | Streptomyces sp. SID5473 |
| A0A1H8V2A1 | Methylobacterium sp. ap11 | X0MQE5 | Streptomyces albulus PD-1 |
| A0A1R0VG54 | Mycobacterium sp. IS-2888 | A0A6L9QMV4 | Actinomadura bangladeshensis |
| A0A2U1DQA3 | Acidovorax sp. 99 | A0A0C2CUM9 | Enhygromyxa salina |
| A0A2W4XQ91 | Leifsonia xyli | A0A315S9A1 | Williamsia marianensis |
| K2IZQ1 | Celeribacter baekdonensis B30 | A0A542LNP4 | Herbaspirillum sp. SJZ107 |
| R8YX69 | Bacillus cereus TIAC219 | A0A1X1PVC2 | Frankia casuarinae (strain DSM 45818/CECT 9043/CcI3) |
| F8JMN9 | Streptomyces cattleya (strain ATCC 35852/DSM 46488/JCM 4925/NBRC | A0A066TPR0 | Amycolatopsis rifamycinica |
| | 14057/NRRL 8057) | | |
| U0ZQ18 | Pseudogulbenkiania ferrooxidans EGD-HP2 | A0A2S9YC39 | Enhygromyxa salina |
| A0A1S1QJ50 | Frankia sp. Cc1.17 | A0A066TTQ6 | Amycolatopsis rifamycinica |
| A0A6H9V796 | Streptomyces sp. HIT-DPA4 | A0A1X1UR08 | Mycobacterium fragae |
| A0A523IXI1 | Candidatus Dadabacteria bacterium | A0A4R4PE58 | Actinomadura bangladeshensis |
| A0A2A3HAX6 | Streptomyces sp. Tue6028 | A0A076M081 | Streptomyces lividans TK24 |
| A0A6V8K509 | Phytohabitans houttuy neae | A0A6L8NDN9 | Streptomyces sp. SID5473 |
| A0A511MFU3 | Nocardia ninae NBRC 108245 | A0A0M8RR93 | Streptomyces sp. NRRL F-5755 |
| A0A5E4X9J8 | Pandoraea horticolens | L7FGM2 | Streptomyces turgidiscabies Car8 |
| A0A1S1QXY6 | Frankia sp. Cc1.17 | A0A6I6VS67 | Pseudomonas sp. S35 |
| X7UM42 | Mycobacterium sp. MAC_080597 8934 | A0A117IVQ6 | Streptomyces kanasensis |
| A0A2A3H3J9 | Streptomyces sp. Tue6028 | S4NUC0 | Streptomyces afghaniensis 772 |
| A0A511YP15 | Chryseobacterium hagamense | L7F6V4 | Streptomyces turgidiscabies Car8 |
| A0A1S1Q4E5 | Frankia sp. Cc1.17 | A0A101Q3F7 | Streptomyces corchorusii |
| A0A0N0TP55 | Nocardiopsis sp. NRRL B-16309 | Q2JH05 | Frankia casuarinae (strain DSM 45818/CECT 9043/CcI3) |
| A0A1G6Z701 | Actinokineospora iranica | A0A656WTX7 | Streptomyces sp. NRRL WC-3701 |
| A0A1S1Q853 | Frankia sp. Cc1.17 | A0A1T5IRU1 | Okibacterium fritillariae |
| H8III4 | Mycobacterium intracellulare (strain ATCC 13950/DSM 43223/JCM 6384/NCTC | A0A4Q3NG28 | Comamonadaceae bacterium |
| | 13025/3600) | | |
| A0A150X4V7 | Roseivirga spongicola | A0A1S1KJJ9 | Mycobacterium syngnathidarum |
| A0A1C4SBR2 | Streptomyces sp. DvalAA-14 | A0A0E1WCM4 | Burkholderia pseudomallei 1710a |
| A0A1A2JFV8 | Mycobacterium sp. E3339 | A0A1T4KIJ5 | Marinactinospora thermotolerans DSM 45154 |
| A0A544Y8X9 | Microbispora sp. SCL1-1 | W1U1Z3 | Clostridium butyricum DORA_1 |
| A0A1X2JLU4 | Paraclostridium bifermentans | A0A7K3R959 | Streptomyces anulatus |
| A0A2N8NXQ9 | Streptomyces eurocidicus | A0A2V4YLN5 | Pseudomonas sp. GV058 |
| A0A2N8NPY1 | Streptomyces eurocidicus | A0A4D4J438 | Gandjariella thermophila |
| A0A2A7VR36 | Bacillus wiedmannii | A0A0U3HPT5 | Pseudoalteromonas rubra |
| A0A1E3T5C2 | Mycobacterium sherrisii | A0A0N0ZCM9 | Burkholderia sp. ST111 |
| A0A0U1D303 | Mycolicibacterium conceptionense | A0A1Q4USH7 | Streptomyces uncialis |
| A0A0B5S424 | Bacillus mycoides | A0A0Q0XX21 | Frankia sp. ACNlag |
| A0A0L8L4X6 | Streptomyces resistomycificus | A0A1D9LD72 | Chromobacterium vaccinii |
| A0A0M0ACB2 | Clostridium botulinum | A0A1A9DP35 | Streptomyces sp. Ncost-T6T-1 |
| A0A6B4DYX6 | Clostridium botulinum | A0A4R3BN51 | Streptomyces sp. BK205 |
| A0A6B4GY43 | Clostridium botulinum | A0A0R3FH33 | Mycobacteroides sp. H092 |
| A0A6H3MD55 | Mycobacterium tuberculosis | A0A0Q0Y9T8 | Frankia sp. ACNlag |
| A0A6B4I873 | Clostridium botulinum | A0A4V3C9U2 | Kribbella sp. VKM Ac-2571 |
| A0A6B4GUP6 | Clostridium botulinum | A0A559WI06 | Micromonospora sp. CNZ297 |
| A0A501USI0 | Clostridium perfringens | A0A1L6PUX4 | Streptomyces sp. Tue 6075 |
| A0A6M1VLZ3 | Clostridium perfringens | A0A1H4UEH8 | Amycolatopsis tolypomycina |
| A0A133NEX1 | Clostridium perfringens | A0A3N1Y9T0 | Kitasatospora cineracea |
| A0A433L2S4 | Clostridium perfringens | A0A0M1IYK4 | Clostridium sp. L74 |
| A0A6M1WEW9 | Clostridium perfringens | A0A4V2TZJ5 | Streptomyces sp. BK038 |
| A0A6N2HZL8 | Streptomyces sp. sk2.1 | A0A1L6PUW1 | Streptomyces sp. Tue 6075 |
| A0A381IWK8 | Clostridium perfringens | A0A4V3CAY5 | Kribbella sp. VKM Ac-2571 |
| A0A286EY93 | Streptomyces sp. 1222.2 | A0A510D6R1 | Streptomyces rochei |
| A0A2X2XB86 | Clostridium perfringens | A0A5P8K5Y3 | Streptomyces phaeolivaceus |
| A0A7D5ZNL2 | Streptomyces sp. NEAU-sy36 | A0A2N9AZZ9 | Streptomyces chartreusis NRRL 3882 |
| A0A1B7UBB9 | Mycobacterium sp. 852002-40037_SCH5390672 | A0A0E8XPP2 | Yersinia wautersii |
| A0A292D2W8 | Pseudomonas sp. FDAARGOS_380 | A0A0K9XDV2 | Streptomyces caatingaensis |
| A0A1M7RN17 | Cryptosporangium aurantiacum | A0A4R3D486 | Streptomyces sp. BK038 |
| A0A498Q733 | Mycobacterium innocens | A0A161LUG5 | Planomonospora sphaerica |
| A0A1Y6B4J5 | Tistlia consotensis USBA 355 | A0A2N9BBU7 | Streptomyces chartreusis NRRL 3882 |
| A0A1B7UB38 | Mycobacterium sp. 852002-40037_SCH5390672 | A0A2L2BDI0 | Chromobacterium vaccinii |
| A0A498PTX9 | Mycobacterium innocens | A0A1H4IK25 | Amycolatopsis tolypomycina |
| A0A429QKB9 | Streptomyces sp. WAC05858 | A0AIL6PRY8 | Streptomyces sp. Tue 6075 |
| A0A1S2PRY3 | Streptomyces colonosanans | A0A1X1BD05 | Williamsia sp. 1135 |
| A0A370CWN4 | Mycolicibacterium moriokaense | A0A1H3HPF5 | Nitrosomonas sp. Nm33 |
| A0A1A3DS03 | Mycobacterium alsense | A0AIXIUHN0 | Mycobacterium europaeum |
| A0A1B9D5L0 | Mycobacterium vulneris | J9A457 | Bacillus cereus BAG6O-2 |
| A0A2D8SNX3 | Owenweeksia sp. | A0A115ARY2 | Streptomyces sp. cf124 |
| A0A318HET7 | Mycolicibacterium moriokaense | A0A2P8HZX0 | Saccharothrix carnea |
| A0A327U202 | Kitasatospora sp. SolWspMP-SS2h | A0A2P8I5H0 | Saccharothrix carnea |
| A0A1X2L0V4 | Mycobacterium vulneris | A0A3D2J5H4 | Ktedonobacter sp. |
| A0A0N9XPE9 | Mycolicibacterium fortuitum | A0A418L4L5 | Mycobacteroides abscessus |
| A0A2W6CR55 | Pseudonocardiales bacterium | A0A2A6NLM2 | Bradyrhizobium sp. C9 |
| B4V6C6 | Streptomyces sp. Mg1 | A0A250VUY4 | Streptomyces olivochromogenes |
| A0A2B5AXL9 | Bacillus toyonensis | J2V269 | Herbaspirillum sp. YR522 |
| A0A2B3C814 | Bacillus thuringiensis | A0A2U3NMM2 | Mycobacterium rhizamassiliense |
| A0A2B0A751 | Bacillus thuringiensis | A0A7K2J021 | Nocardiopsis alba |
| B4V6E0 | Streptomyces sp. Mg1 | A0A6B2WLL1 | Streptomyces sp. SID 13588 |
| A0A2B5EPQ2 | Bacillus toyonensis | A0A6G2Q6P6 | Streptomyces sp. SID685 |
| A0A4R3ILI3 | Streptomyces sp. BK335 | A0A2P7PNY0 | Streptosporangium nondiastaticum |
| A0A6S7D9U0 | Achromobacter piechaudii | A0A3N4SIA6 | Streptomyces sp. Ag109_O5-1 |
| A0A352P8F1 | Lachnoclostridium sp. | A0A2S6WUY0 | Streptomyces sp. MH60 |
| A0A401W9M1 | Streptomyces rimosus subsp. paromomycinus | A0A7K2J0D9 | Nocardiopsis alba |
| A0A1H4RRA2 | Streptomyces melanosporofaciens | W7SMX5 | Kutzneria sp. 744 |
| A0A3D1FJU3 | Lachnoclostridium sp. | A0A429ACR5 | Nonomuraea sp. WAC 01424 |
| A0A559VEG5 | Streptomyces sp. BK340 | A0A6B2X970 | Streptomyces sp. SID13726 |
| A0A5B8E2B9 | Herbaspirillum seropedicae | A0A6B2XGZ7 | Streptomyces sp. SID13726 |
| W6JZT5 | Tetrasphaera australiensis Ben110 | A0A6N7KH80 | Streptomyces kaniharaensis |
| A0A0Q8ESP9 | Streptomyces sp. Root63 | A0A127QSY8 | Collimonas pratensis |
| A0A1G4W902 | Mycolicibacterium fluoranthenivorans | A0A127Q017 | Collimonas pratensis |
| A0A318T354 | Pseudoroseicyclus aestuarii | A0A4Y3QZX1 | Streptomyces cacaoi subsp. cacaoi |
| A0A401W3G6 | Streptomyces rimosus subsp. paromomycinus | A0A542DEN4 | Amycolatopsis cihanbeyliensis |
| A0A6M4X321 | Streptomyces cacaoi subsp. asoensis | A0A0K1JK68 | Luteipulveratus mongoliensis |
| A0A2G5QJE7 | Caulobacter sp. FWC2 | A0A0M9YYQ2 | Streptomyces sp. XY431 |
| A0A559V1L9 | Streptomyces sp. BK340 | A0A1H9WIJ1 | Actinokineospora terrae |
| A0A1A3DQ73 | Mycobacterium sp. 1482292.6 | A0A6I6SBQ9 | Streptomyces sp. GS7 |
| A0A1A2X5U4 | Mycobacterium sp. E2497 | A0A0M8U1P4 | Streptomyces sp. XY431 |
| G1Y4E6 | Nitrospirillum amazonense Y2 | A0A1X1XZA8 | Mycobacterium kyorinense |
| A0A1W9ZAD8 | Mycobacterium arosiense ATCC BAA-14 | A0A0M8TWL2 | Streptomyces sp. XY431 |
| A0A1A3DUZ7 | Mycobacterium sp. 1482292.6 | F7T8R1 | Achromobacter insuavis AXX-A |
| A0A1A2XAG9 | Mycobacterium sp. E2497 | A0A660LHH2 | Solirubrobacter pauli |
| A0A1H4YV07 | Streptomyces melanosporofaciens | A0A0N6ZMV0 | Streptomyces sp. CCM_MD2014 |
| A0A2G6XY99 | Streptomyces sp. 76 | A0A7D6E0Y4 | Mycobacterium gordonae |
| A0A0B5F274 | Streptomyces albus (strain ATCC 21838/DSM 41398/FERM P-419/JCM 4703/ | A0A238YHU9 | Dokdonia pacifica |
| | NBRC 107858) | | |
| A0A7I7PFS8 | Mycobacterium noviomagense | D3P513 | Azospirillum sp. (strain B510) |
| A0A2G6XUY2 | Streptomyces sp. 76 | A0A7D6E7D0 | Mycobacterium gordonae |
| A0A0D1NSV9 | Bradyrhizobium elkanii | K0I2K2 | Acidovorax sp. KKS102 |
| A0A4R4WU42 | Nonomuraea diastatica | A0A5C8M4D6 | Rheinheimera tangshanensis |
| A0A3S0BUM0 | Streptomyces sp. WAC05858 | A0A1V0VEF6 | Kitasatospora albolonga |
| A0A2K9F6D5 | Streptomyces sp. CMB-StM0423 | A0A147FV12 | Methylobacterium indicum |
| A0A418VMB2 | Azospirillum sp. K2W22B-5 | A0A5R9FJB8 | Streptomyces montanus |
| A0A2G6X5D7 | Variovorax sp. 54 | A0A0F4WI03 | Clostridium sp. IBUN13A |
| A0A317S8X8 | Actinokineospora mzabensis | A0A1X1XBY5 | Mycobacterium gordonae |
| A0A4R4WC48 | Nonomuraea diastatica | U2N8W4 | Clostridium intestinale URNW |
| A0A2K9ETE7 | Streptomyces sp. CMB-StM0423 | A0A498R0Z3 | Lucifera butyrica |
| A0A179SAD2 | Methylobacterium platani | A0A4P7GZE1 | Nocardia sp. CS682 |
| A0A540PB15 | Streptomyces ipomoeae | A0A7K3CTR5 | Streptomyces sp. SID337 |
| A0A2S4Y6Q8 | Streptomyces sp. Ru72 | N9WFJ1 | Clostridium thermobutyricum |
| A0A0R3FH35 | Mycobacteroides sp. H072 | A0A1V3G9A8 | Fictibacillus arsenicus |
| A0A540NV40 | Streptomyces ipomoeae | A0A3N4U1Z1 | Streptomyces sp. SAI-083 |
| A0A4R4ZS80 | Kribbella antibiotica | A0A4P6JNW4 | Ktedonosporobacter rubrisoli |
| A0A2P9FBY2 | Streptomyces sp. MA5143a | A0A2X0IMP3 | Streptacidiphilus pinicola |
| A0A0J9DQ35 | Ralstonia sp. MD27 | U2N0B0 | Clostridium intestinale URNW |
| A0A101R3J2 | Streptomyces longwoodensis | A0A2G7BX01 | Streptomyces sp. 93 |
| A0A101QX10 | Streptomyces longwoodensis | A0A4R0IFW1 | Kribbella sp. YM55 |
| A0A1H8GE61 | Cryobacterium luteum | A0A4R5TVS2 | Arthrobacter crusticola |
| T4VC28 | Paraclostridium bifermentans ATCC 19299 | A0A2G5MXS4 | Pseudomonas sp. 2822-17 |
| A0A3D9SV43 | Thermomonospora umbrina | A0A6M9XN92 | Streptomyces coelicolor |
| A0A0Q4RFL3 | Acidovorax sp. Leaf78 | A0A2G0CHS9 | Lewinella marina |
| A0A1Q7WEB3 | Actinobacteria bacterium 13_1_20CM_3_71_11 | A0A0F0HLP6 | Saccharothrix sp. ST-888 |
| A0A2N7X979 | Trinickia symbiotica | A0A1A0NBW1 | Mycobacteriaceae bacterium 1482268.1 |
| A0A3D9SVP4 | Thermomonospora umbrina | A0A372PFT4 | Microbispora triticiradicis |
| A0A495BQK5 | Edaphobacter dinghuensis | A0A0F0HND5 | Saccharothrix sp. ST-888 |
| A0A1R1JXY9 | Alcaligenes xylosoxydans xylosoxydans | A8M333 | Salinispora arenicola (strain CNS-205) |
| A0A495BPH3 | Edaphobacter dinghuensis | A0A1D2S0H0 | Acidovorax sp. SCN 68-22 |
| A0A1A3TIH3 | Mycobacterium sp. 1165178.9 | A0A511N183 | Deinococcus cellulosilyticus NBRC 1063 |
| A0A3G7HF49 | Pseudomonas chlororaphis | F8JV05 | Streptomyces cattleya (strain ATCC 35852/DSM 46488/JCM 4925/NBRC |
| | | | 14057/NRRL 8057) |
| A0A0L8QGL7 | Streptomyces varsoviensis | A0A3S9IH94 | Streptomyces aquilus |
| A0A0D6HFN5 | Alcaligenes xylosoxydans xylosoxydans | A0A1X2JLV9 | Paraclostridium bifermentans |
| A0A7K2R5C1 | Streptomyces sp. SID5471 | A0A5P3XJR6 | Paraclostridium bifermentans |
| A0A1C6PTK8 | Streptomyces sp. SceaMP-e96 | A0A3Q9C070 | Streptomyces aquilus |
| A0A1X0D6V1 | Mycolicibacterium insubricum | A0A249BHZ4 | Mycobacterium intracellulare |
| A0A0M8WY09 | Streptomyces sp. NRRL B-3648 | A0A1B5EBH6 | Pseudomonas sp. 44 R 15 |
| J7W7T5 | Bacillus cereus VD142 | A0A561CTC8 | Acidovorax radicis |
| A0A1C6MAE7 | Streptomyces sp. SceaMP-e96 | F8JQL7 | Streptomyces cattleya (strain ATCC 35852/DSM 46488/JCM 4925/NBRC |
| | | | 14057/NRRL 8057) |
| M3CDL5 | Streptomyces mobaraensis NBRC 138 | A0A3Q9C3V1 | Streptomyces aquilus |
| A0A5C7R9P2 | Thiothrix sp. | A0A5S4G9F7 | Nonomuraea zeae |
| A0A1A3BYM4 | Mycobacterium sp. 1245805.9 | X8AE93 | Mycobacterium intracellulare |
| A0A1C6P8M0 | Streptomyces sp. SceaMP-e96 | A0A1A2Q805 | Mycobacterium intracellulare |
| M3BNR4 | Streptomyces mobaraensis NBRC 138 | A0A511M603 | Nocardia ninae NBRC 108245 |
| A0A2S6ZFF8 | Xanthomonas theicola | A0A5S4GPH1 | Nonomuraea zeae |
| A0A1A3CAZ2 | Mycobacterium sp. 1245805.9 | A0A4R1PX37 | Anaerospora hongkongensis |
| A0A5C7R6M0 | Thiothrix sp. | A0A1M6EZR5 | Propionispora hippei DSM 15287 |
| A0A7K0VET7 | Actinobacteria bacterium | R8CBJ5 | Bacillus cereus str. Schrouff |
| A0A291SU84 | Streptomyces malaysiensis | C4IBW2 | Clostridium butyricum E4 str. BoNT E BL5262 |
| A0A1E7YI62 | Mycolicibacterium sp. (ex Dasyatis americana) | A0A1V2PE88 | Kribbella sp. ALI-6-A |
| A0A538H0G8 | Actinobacteria bacterium | A0A318QAI0 | Komagataeibacter pomaceti |
| A0A537VFK3 | Actinobacteria bacterium | A0A1C3GI61 | Pseudomonas sp. 1 R 17 |
| A0A2J7YRC7 | Streptomyces malaysiensis | A0A2K4JM40 | Pseudomonas sp. GW531-R1 |
| A0A358SM40 | Actinobacteria bacterium | A0A2K8PAS7 | Streptomyces lavendulae subsp. lavendulae |
| A0A4Y9VBU0 | Micromonospora sp. CNZ285 | A0A6I5BL49 | Streptomyces sp. SID4926 |
| A0A538J4N0 | Actinobacteria bacterium | A0A1G6LSN8 | Acidovorax valerianellae |
| A0A6N9VG36 | Streptomyces fulvissimus | I0WM12 | Rhodococcus imtechensis RKJ3 |
| A0A538CEI2 | Actinobacteria bacterium | A0A176Z4K2 | Bradyrhizobium centrolobii |
| A0A1A2LRI2 | Mycobacterium sp. E3251 | A0A221NQY8 | Arthrobacter sp. YN |
| A0A6I3B8W9 | Actinobacteria bacterium | A0A1V2PEE6 | Kribbella sp. ALI-6-A |
| A0A6N9V2I6 | Streptomyces fulvissimus | A0A4T2C559 | Subtercola vilae |
| A0A612XYR2 | Actinobacteria bacterium | A0A7C4ZUC8 | Anaerolineae bacterium |
| A0A537VNH0 | Actinobacteria bacterium | A0A4R4LUK5 | Actinomadura sp. KC216 |
| A0A538MHQ0 | Actinobacteria bacterium | A0A023Y3G0 | Stenotrophomonas rhizophila |
| A0A6L6R2W7 | Actinobacteria bacterium | A0A4Z1CXY7 | Streptomyces bauhiniae |
| Q63PN4 | Burkholderia pseudomallei (strain K96243) | A0A4R4M3M0 | Actinomadura sp. KC216 |
| A0A538D8J0 | Actinobacteria bacterium | A0AIN6TQT5 | Microbispora rosea |
| A0A4P5S9V1 | Actinobacteria bacterium | A0A218XK90 | Vibrio campbellii |
| A0A291T423 | Streptomyces malaysiensis | A0A2N3UZH1 | Streptomyces sp. GP55 |
| A0A537XIU9 | Actinobacteria bacterium | A0A3N0CYQ7 | Streptomyces sp. I6 |
| A0A538JXN5 | Actinobacteria bacterium | A0A5D4RP67 | Bacillus marisflavi |
| G2G7F8 | Streptomyces zinciresistens K42 | A0A2N3UES8 | Streptomyces sp. GP55 |
| A0A515FV13 | Streptomyces malaysiensis | A0A0F2KVP2 | Azospirillum thiophilum |
| A0A4R1G1A0 | Nocardia alba | A0A5M8UBK5 | Agrobacterium sp. ICMP 7243 |
| A0A1A2LZV3 | Mycobacterium sp. E3251 | A0A4Q1RW96 | Streptomyces sp. TM32 |
| A0A538IKD1 | Actinobacteria bacterium | A0A2N3UED9 | Streptomyces sp. GP55 |
| A0A537X2W4 | Actinobacteria bacterium | A0A2U4GXK0 | Vibrio campbellii |
| A0A1H4ISC3 | Tenacibaculum sp. MAR_2009_124 | A0A2K2RK53 | Streptomyces sp. DH-12 |
| A0A6I2VBL5 | Actinobacteria bacterium | A0A2P8QB64 | Streptomyces dioscori |
| A0A538EQ32 | Actinobacteria bacterium | A0A3N6FKQ5 | Streptomyces sp. ADI95-17 |
| A0A537XII6 | Actinobacteria bacterium | A0A6I6PZU4 | Bacillus marisflavi |
| A0A166VEF5 | Pseudoalteromonas luteoviolacea DSM 6061 | A0A7J5ALI0 | Tenacibaculum aiptasiae |
| A0A538E649 | Actinobacteria bacterium | A0A1M9M6D2 | Mycobacteroides abscessus subsp. bolletii |
| J7XG89 | Bacillus cereus BAG5X1-1 | A0A367FIF5 | Sphaerisporangium album |
| A0A0M8ULK4 | Streptomyces sp. H036 | A0A6B2ZKX6 | Streptomyces sp. SID7909 |
| A0A538L7F9 | Actinobacteria bacterium | A0A1M3DH55 | Sphingomonas sp. 67-36 |
| A0A538HQ57 | Actinobacteria bacterium | A0A1M7PA54 | Streptomyces paucisporeus |
| A0A538BIP7 | Actinobacteria bacterium | A0A1X0ID59 | Mycobacterium paraseoulense |
| A0A0M9CKW1 | Streptomyces sp. XY332 | A0A0M4DSV6 | Streptomyces sp. CFMR 7 |
| A0A7K2Q7Y0 | Streptomyces sp. SID7760 | A0A117PMN2 | Streptomyces pseudovenezuelae |
| A0A7K2QPC4 | Streptomyces sp. SID7760 | A0A4R8SNF2 | Mycobacteroides salmoniphilum |
| A0A7I7YXI4 | Mycobacterium parmense | A0A5S3YFS7 | Pseudoalteromonas sp. S1609 |
| A0A7I7YRA2 | Mycobacterium parmense | A0A7I7NFT2 | Mycolicibacterium boenickei |
| G2G1M3 | Desulfosporosinus sp. OT | A0A0L0KVW9 | Streptomyces stelliscabiei |
| A0A540PKA7 | Streptomyces ipomoeae | A0A4V6QF66 | Mycobacteroides salmoniphilum |
| A0A0X3TEH8 | Ruegeria marisrubri | A0A2H3QMP9 | Bacillus sp. AFS012607 |
| A0A1Q4XJ75 | Nocardiopsis sp. TSRI0078 | A0A4R8SCG8 | Mycobacteroides salmoniphilum |
| A0A3A3CXE1 | Pseudoalteromonas sp. MSK9-3 | A0A1X0IFQ9 | Mycobacterium paraseoulense |
| A0A2G4FFL0 | Actinobacteria bacterium | W5VZ39 | Kutzneria albida DSM 43870 |
| A0A537YK61 | Actinobacteria bacterium | A0A1M7I679 | Streptomyces paucisporeus |
| A0A540PZQ1 | Streptomyces ipomoeae | A0A1A9C2T2 | Streptomyces sp. DI166 |
| A0A3A3CVE3 | Pseudoalteromonas sp. MSK9-3 | A0A366M372 | Sphaerisporangium sp. LHW63015 |
| A0A538I1R4 | Actinobacteria bacterium | A0A2N8P4Y6 | Streptomyces noursei |
| A0A2N8BBG7 | Pseudomonas sp. FW305-25 | A0A221W5C3 | Actinoalloteichus hoggarensis |
| A0A336Q6B9 | Clostridium perfringens | A0A7K2LWK7 | Streptomyces sp. SID5998 |
| A0A381IP21 | Clostridium perfringens | A0A6G7T4Q6 | Streptomyces sp. ID38640 |
| A0A4U9NYX7 | Clostridium perfringens | A0A3E1HC91 | Pseudomonas sp. GL93 |
| M4ZY10 | Bradyrhizobium oligotrophicum S58 | A0A3Q8URF8 | Streptomyces sp. WAC 06738 |
| A0A2X2YCT1 | Clostridium perfringens | A0A6G7T565 | Streptomyces sp. ID38640 |
| A0A2Z3TUG5 | Clostridium perfringens | A0A1M6BDL6 | Clostridium intestinale DSM 6191 |
| A0A1B9EMH7 | Streptomyces sp. PTY08712 | A0A6N0X254 | Sphingomonas sp. CL5.1 |
| A0A1B9C497 | Mycobacterium intracellulare subsp. yongonense | A0A366M4C8 | Sphaerisporangium sp. LHW63015 |
| A0A1D3E1G4 | Streptomyces thermolilacinus SPC6 | A0A1I1IWI5 | Clostridium uliginosum |
| A0A2K9EVM4 | Streptomyces sp. CMB-StM0423 | A0A3S0HF97 | Variovorax gossypii |
| A0A317S6X1 | Actinokineospora mzabensis | A0A561C1B2 | Kribbella amoyensis |
| A0A543VUD3 | Streptomyces cavourensis | A0A1A2DXL8 | Mycobacterium sp. 852014-52450_SCH5900713 |
| A0A1Q2ZKQ7 | Streptomyces acidiscabies | A0A5Q0LEN3 | Streptomyces fagopyri |
| A0A428W7V4 | Amycolatopsis balhimycina DSM 5908 | A0A4Q4DNG8 | Streptomyces sp. L-9-10 |
| A0A1U7MAW5 | Sporomusa sphaeroides DSM 2875 | A0A0U3NU26 | Streptomyces sp. CdTB01 |
| A0A428X4U9 | Amycolatopsis balhimycina DSM 5908 | A0A1I5CVS7 | Amycolatopsis rubida |
| A0A258SB04 | Sphingomonas sp. 28-62-20 | A0A6N9WBX0 | Amycolatopsis rubida |
| A0A6G3S1F8 | Streptomyces sp. SID14478 | A0A5Q0LEM5 | Streptomyces fagopyri |
| A0A150W1Z3 | Streptomyces sp. WAC04657 | A0A7I7P004 | Mycobacterium seoulense |
| A0A1G5UTG5 | Sphingomonas sp. NFR15 | A0A4R7ZUM5 | Kribbella sp. VKM Ac-2570 |
| R4LDE7 | Actinoplanes sp. N902-109 | A0A2G7DY88 | Streptomyces sp. 1 |
| A0A165NAX3 | Pseudovibrio sp. W74 | A0A2M9LXV9 | Streptomyces sp. CB02959 |
| A0A4Q7KJH6 | Herbihabitans rhizosphaerae | A0A1K2FW60 | Streptomyces sp. F-1 |
| A0A2D3UJV6 | Streptomyces peucetius subsp. caesius ATCC 27952 | A0A0B2YT01 | Mycolicibacterium setense |
| A0A428WRZ1 | Amycolatopsis balhimycina DSM 5908 | A0A1G9UQM0 | Streptomyces sp. cf386 |
| A0A4V2ERA3 | Herbihabitans rhizosphaerae | A0A1A0PQ19 | Mycolicibacterium setense |
| A0A0L0JLR6 | Streptomyces acidiscabies | A0A1H0GDN2 | Afipia sp. GAS231 |
| A0A100JDJ0 | Streptomyces acidiscabies | A0A4R7ZU71 | Kribbella sp. VKM Ac-2570 |
| A0A433MME0 | Variovorax guangxiensis | A0A1C6MQ30 | Streptomyces sp. LamerLS-316 |
| A0A1D3JPL5 | Pseudomonas veronii 1YdBTEX2 | A0A7I7P768 | Mycobacterium seoulense |
| A0A1A2P231 | Mycobacterium sp. E3247 | Q0SVW7 | Clostridium perfringens (strain SM101/Type A) |
| W2EZP5 | Microbispora sp. ATCC PTA-5024 | A0A2X3KD40 | Frankia sp. Ea1.12 |
| W2EPN7 | Microbispora sp. ATCC PTA-5024 | A0A5D3G5W1 | Pseudomonas synxantha |
| A0A1M7QXF4 | Streptomyces yunnanensis | A0AIS8R6A0 | Clostridium beijerinckii |
| A0A0M8QLU8 | Streptomyces caelestis | A0A3G7UG94 | Pseudomonas synxantha |
| A0A7K2LYK2 | Streptomyces sp. SID5998 | A0A0B5QJ65 | Clostridium beijerinckii |
| A0A1I7M754 | Bradyrhizobium arachidis | A0AIS9NDC1 | Clostridium beijerinckii |
| A0A7K2LX16 | Streptomyces sp. SID5998 | A0A4R7VXF2 | Actinophytocola oryzae |
| A0A2A9KI29 | Collimonas sp. PA-H2 | A0A1S9N2P5 | Clostridium beijerinckii |
| A0A544W353 | Mycolicibacterium hodleri | A0A3L8KWD8 | Streptomyces griseocarneus |
| A0A4R3SPK8 | Curtobacterium sp. PhB146 | A0A3L8JZ67 | Streptomyces griseocarneus |
| A0A1M5XR35 | Clostridium intestinale DSM 6191 | A0A1W7M0K6 | Clostridium beijerinckii |
| A0A1A2NUK0 | Mycobacterium sp. E3247 | A0A1S8PQG0 | Clostridium beijerinckii |
| A0A6I6N767 | Streptomyces broussonetiae | A0A7K0BQG8 | Actinomadura sp. RB68 |
| A0A7K2M1W7 | Streptomyces sp. SID5998 | A2S6Q7 | Burkholderia mallei (strain NCTC 10229) |
| A0A5P2XWL1 | Streptomyces rimosus | A0A1B9BGZ8 | Clostridium beijerinckii |
| A0A0M8QDF1 | Streptomyces caelestis | A0A429F7P2 | Amycolatopsis sp. WAC 04182 |
| I8UD85 | Fictibacillus macauensis ZFHKF-1 | A0A1S8S9C6 | Clostridium beijerinckii |
| A0A4R7FBK7 | Pseudomonas sp. GV087 | A0A3L8J6M5 | Streptomyces griseocarneus |
| A0A2U0ZP22 | Filimonas sp. YR581 | A0A1W7M067 | Clostridium beijerinckii |
| A0A1I7KXU6 | Bradyrhizobium arachidis | A0A3G7U3C3 | Pseudomonas synxantha |
| A0A3Q8UPP7 | Streptomyces sp. WAC 06738 | A0A4R7W0V0 | Actinophytocola oryzae |
| A0A1M7PEP9 | Streptomyces yunnanensis | A0A024YML3 | Streptomyces sp. PCS3-D2 |
| A0A4U3MB39 | Herbidospora galbida | A0A0B5QE03 | Clostridium beijerinckii |
| A0A3S8VSH8 | Streptomyces sp. WAC 06738 | A0A0R2Z7U1 | Pseudomonas synxantha |
| R0DZ34 | Ralstonia pickettii OR214 | A0A0E3GZ34 | Pseudomonas synxantha |
| I4L3F9 | Pseudomonas synxantha BG33R | A0A5N5ZC47 | Kordia sp. TARA_039_SRF |
| A0A4R8CNK2 | Kribbella sp. VKM Ac-2573 | A0A1S8PNY0 | Clostridium beijerinckii |
| A0A3T1AXJ0 | Actinoplanes sp. OR16 | A0A3L8KVH0 | Streptomyces griseocarneus |
| A0A2B8ANR6 | Streptomyces sp. Ru87 | A0A1A2NLL4 | Mycobacterium sp. E1715 |
| A0A401K6B4 | Ralstonia sp. SET104 | A0A6G2XLQ3 | Streptomyces sp. SID8381 |
| A0A495R057 | Actinomadura pelletieri DSM 43383 | A0A1X2B5S4 | Mycolicibacterium peregrinum |
| A0A4R8CNI0 | Kribbella sp. VKM Ac-2573 | A0A4R2K6J2 | Actinocrispum wychmicini |
| A0A646KGG6 | Streptomyces jumonjinensis | A0A6G7P8A0 | Streptomyces sp. JB150 |
| A0A0X8VCL5 | Anaerotignum propionicum DSM 1682 | A0A246C2U9 | Mycolicibacterium peregrinum |
| A0A2A4KMZ2 | Streptomyces sp. WZ.A104 | A0A505D367 | Streptomyces sporangiiformans |
| A0A0H5CPC6 | Alloactinosynnema sp. L-07 | A0A428XE04 | Amycolatopsis sp. WAC 01376 |
| A0A429DNI5 | Streptomyces sp. WAC 04229 | A0A366X6A9 | Phaeobacter gallaeciensis |
| A0A495QHY9 | Actinomadura pelletieri DSM 43383 | A0A429SVZ6 | Streptomyces sp. WAC07149 |
| A0A2B8AR18 | Streptomyces sp. Ru87 | A0A1A0WGG0 | Mycolicibacterium peregrinum |
| A0A0L0KFN1 | Streptomyces acidiscabies | A0A1H0W206 | Actinopolyspora xinjiangensis |
| A0A229TF91 | Amycolatopsis vastitatis | A0A1H5V8U9 | Thermomonospora echinospora |
| A0A495QT74 | Actinomadura pelletieri DSM 43383 | A0A1A2UJC4 | Mycobacterium sp. E2733 |
| A0A646KK63 | Streptomyces jumonjinensis | Q0RUK8 | Frankia alni (strain ACN14a) |
| A0A4U5WWC1 | Streptomyces galbus | A0A2A2NB36 | Vibrio sp. VIB |
| A0A0Q8C1Y8 | Pseudomonas sp. Root569 | A0A1E4DTT8 | Pelagibacterium sp. SCN 63-126 |
| A0A646KR25 | Streptomyces jumonjinensis | A0A428YEL4 | Amycolatopsis sp. WAC 01376 |
| A0A229T761 | Amycolatopsis vastitatis | A0A6G2XCP5 | Streptomyces sp. SID8381 |
| A0A2S9DXM9 | Pseudomonas cedrina | A0A7K1KST0 | Actinomadura litoris |
| A0A6C6YVH1 | Burkholderia mallei (strain NCTC 10247) | A0A1C5CJP7 | Streptomyces sp. MnatMP-M27 |
| A0A1I2E0Q8 | Nannocystis exedens | M3BF38 | Streptomyces gancidicus BKS 13-15 |
| X5L7B9 | Mycolicibacterium mageritense DSM 444 | A0A2L2MMX4 | Streptomyces dengpaensis |
| A0A1A0MDT1 | Mycobacterium sp. 1164966.3 | A0A6G2XLC6 | Streptomyces sp. SID8381 |
| A0A327VGY6 | Streptomyces sp. PsTaAH-137 | A0A1F4GNS7 | Burkholderiales bacterium GWA2_64_37 |
| A0A1A2YL25 | Mycobacterium sp. E1386 | A0A317N960 | Nocardia neocaledoniensis |
| A0A3G4W5P0 | Streptomyces sp. ADI95-16 | A0A2U1WD12 | Azospirillum sp. TSA6c |
| A0A1A2XLA4 | Mycobacterium sp. E1386 | A0A1B4FXL5 | Burkholderia sp. BDU8 |
| A0A543ITN9 | Thermopolyspora flexuosa | A0A4V2S5R5 | Actinocrispum wychmicini |
| A0A1E5Q2J2 | Streptomyces subrutilus | A0A7K2NJT1 | Streptomyces sp. SID5468 |
| U1JLC7 | Pseudoalteromonas citrea DSM 8771 | A0A4R2IC98 | Kribbella sp. VKM Ac-2541 |
| A0A1J5MV04 | Gammaproteobacteria bacterium MedPE | A0A2M9APM9 | Streptomyces sp. CNZ306 |
| A0A327VZE7 | Streptomyces sp. PsTaAH-137 | A0A656H9D6 | Thiothrix nivea (strain ATCC 35100/DSM 5205/JP2) |
| A0A2C8X9Z4 | Streptomyces sp. OK228 | A0A7K2P612 | Streptomyces sp. SID5926 |
| A0A3G4W7D1 | Streptomyces sp. ADI95-16 | A0A7K2NKK7 | Streptomyces sp. SID5468 |
| A0A1E5PMZ8 | Streptomyces subrutilus | A0A7H5I8L0 | Streptomyces sp. SID4-14 |
| A0A427SDZ1 | Tenacibaculum litoreum | A0A2M9A312 | Streptomyces sp. CNZ306 |
| A0A3G4W6K1 | Streptomyces sp. ADI95-16 | A0A4R7HAS5 | Streptomyces sp. BK141 |
| A0A3G4W533 | Streptomyces sp. ADI95-16 | A0A6I5H283 | Streptomyces sp. SID486 |
| A0A530YTN0 | Mesorhizobium sp. | A0A150QTZ7 | Sorangium cellulosum |
| A0A6G3NVS3 | Streptomyces cyaneofuscatus | A0AIS1R1B1 | Frankia sp. EUN1h |
| A0A3N1NKW8 | Curtobacterium sp. PhB171 | A0A2N5L2P6 | Ralstonia mannitolilytica |
| A0A6D1VFX9 | Cryobacterium sp. TMT1-2-2 | A0A4P2QAV2 | Sorangium cellulosum |
| A0A4P7RXQ0 | Sphingopyxis sp. PAMC25046 | A0A7H8IX87 | Streptomyces sp. NA02950 |
| A0A2N4TUY8 | Ralstonia pickettii | A0A515YAW7 | Streptomyces sp. RLB3-6 |
| A0A0Q6XI25 | Rhizobacter sp. Root1221 | A0A5H2USH0 | Streptomyces tsukubensis |
| A0A291EPL8 | Ralstonia pickettii | A0A515Y9Y2 | Streptomyces sp. RLB3-6 |
| A0A3N6D3J1 | Streptomyces sp. ADI93-02 | A0A0K8PMR9 | Streptomyces azureus |
| A0A0J6XIT5 | Streptomyces roseus | A0A5H2UQJ8 | Streptomyces tsukubensis |
| A0A6I5F1D0 | Streptomyces sp. SID8356 | G7M7X9 | Clostridium sp. DL-VIII |
| A0A291RLG1 | Nocardia terpenica | A0A2Z3UQD6 | Streptosporangium sp. ‘caverna’ |
| A0A0K9XUK8 | Chryseobacterium sp. Hurlbut01 | A0A0U1KV92 | Sporomusa ovata |
| A0A1I0HS89 | Lacrimispora sphenoides | A0A1B4Y082 | Mycobacterium ulcerans subsp. shinshuense |
| A0A5R1P4W2 | Cryobacterium sp. TMT1-66-1 | A0A1Q9UKD1 | Actinomadura sp. CNU-125 |
| A0A7K3BSK5 | Streptomyces sp. SID4919 | A0A0H5AC38 | Pseudomonas trivialis |
| A0A3M0HZM8 | Streptomyces shenzhenensis | A0A209CUP2 | Streptomyces sp. CS057 |
| A0A5M8RYZ3 | Bacillus cereus | A0A3N1TR54 | Streptomyces sp. CEV 2-1 |
| A0A2A7ZQV4 | Bacillus cereus | A0A5C5NH58 | Pseudomonas rhodesiae |
| A0A6C0Q4R1 | Streptomyces sp. S4.7 | A0A552QUJ9 | Streptomyces sp. 130 |
| A0A7K3FLQ8 | Streptomyces sp. SID5477 | A0A120IZ71 | Mycobacterium tuberculosis variant africanum |
| A0A0E4GZW2 | Mycobacterium lentiflavum | C2SIC5 | Bacillus cereus BDRD-ST196 |
| A0A7K3FJS9 | Streptomyces sp. SID5477 | A0A1H5P7Z8 | Jiangella alba |
| A0A653Q405 | Curtobacterium sp. 81-2 | A0A6B1KCA2 | Streptomyces sp. SID5464 |
| A0A1X1UYB9 | Mycobacterium europaeum | A0A1A2N283 | Mycobacterium sp. E1747 |
| A0A0E4GVX8 | Mycobacterium lentiflavum | A0A6M0FAD2 | Okeania sp. SIO1I7 |
| A1TNT9 | Acidovorax citrulli (strain AAC00-1) | A0A4R0K1F1 | Kribbella sp. YM53 |
| A0A0Q9D4Z3 | Acidovorax sp. Root275 | A0A0F4ISN8 | Streptomyces katrae |
| A0A4R4V4G3 | Actinomadura sp. 7K507 | A0A4V6Q8W6 | Curtobacterium sp. PhB42 |
| A0A0U0ZS58 | Mycobacteroides abscessus | A0A6H1K9A8 | Streptomyces sp. RPA4-2 |
| A0A6G2RW98 | Streptomyces sp. SID8354 | A0A1I7C372 | Geodermatophilus amargosae |
| A0A2A6NU87 | Bradyrhizobium sp. C9 | A0A6H1KIP2 | Streptomyces sp. RPA4-2 |
| A0A100JWV4 | Streptomyces scabiei | A0A3N4SDG0 | Streptomyces sp. Ag109_O5-1 |
| A0A4P6FDT4 | Xylanimicrobium sp. FW10M-9 | A0A7C8BEE0 | Roseomonas genomospecies 6 |
| A0A6G2RRU8 | Streptomyces sp. SID8354 | A0A6I6WH31 | Streptomyces sp. NHF165 |
| A0A3N0EII1 | Nocardiopsaceae bacterium YIM 96095 | A0A248JQD5 | Nitrospirillum amazonense CBAmc |
| A0A655L6M0 | Mycobacteroides abscessus | A0A1I3KDV7 | Bradyrhizobium sp. Gha |
| A0A1X2DAU8 | Mycobacterium sp. IEC1808 | A0A2U3P3Y3 | Mycobacterium numidiamassiliense |
| A0A0U1DPH4 | Mycobacterium europaeum | A0A2U3P4H3 | Mycobacterium numidiamassiliense |
| A0A2G6SDQ0 | Acidovorax sp. 59 | A0A1G7N194 | Lentzea fradiae |
| A0A1I4VNZ2 | Streptomyces sp. cf124 | A0A2X2M6L0 | Streptomyces griseus |
| A0A0U1DIV2 | Mycobacterium europaeum | A0A1X1Z583 | Mycobacterium nebraskense |
| A0A086H4Y8 | Streptomyces scabiei | A0A4V2M7R1 | Kribbella sp. YM53 |
| A0A369V444 | Streptomyces parvulus | A0A1G7ZIA1 | Lentzea fradiae |
| A0A4R5K7P9 | Arthrobacter terricola | A0A429BPT8 | Nonomuraea sp. WAC 01424 |
| A0A2P7PGZ4 | Streptosporangium nondiastaticum | W7S966 | Kutzneria sp. 744 |
| A0A165VFD7 | Pseudovibrio sp. Ad26 | A0A429BGZ4 | Nonomuraea sp. WAC 01424 |
| A0A2P7PSU0 | Streptosporangium nondiastaticum | A0A2P7PW45 | Streptosporangium nondiastaticum |
| A0A2P7PMG8 | Streptosporangium nondiastaticum | A0A1H0A6L9 | Fictibacillus solisalsi |
| A0A614Q3L3 | Actinomadura sp. J1-007 | A0A3N4SNI8 | Streptomyces sp. Ag109_O5-1 |
| A0A374PED0 | Hungatella hathewayi | A0A6H1KLB5 | Streptomyces sp. RPA4-2 |
| A0A369D6W5 | Bacillus sp. AG102 | A0A4R4QFL2 | Kribbella albertanoniae |
| A0A2N0EGX9 | Streptomyces sp. 4121.5 | A0A4R4QFC3 | Kribbella albertanoniae |
| A0A3E4UAN6 | Hungatella hathewayi | A0A0T1WL33 | Mycobacterium sp. Root135 |
| A0A365VYS8 | Pseudomonas sp. MWU12-2534b | A0A089XAP3 | Streptomyces glaucescens |
| A0A2G0Y5K3 | Pseudomonas sp. ICMP 8385 | A0A1H0R1M3 | Streptomyces guanduensis |
| A0A4V1L5M6 | Granulicella sibirica | A0A0P4R518 | Streptomyces lydicamycinicus |
| A0A6I4PK73 | Actinomadura sp. J1-007 | A0A0J6NGF1 | Chromobacterium sp. LK11 |
| A0A6G9H0M1 | Streptomyces sp. QMT-12 | A0A1H0H0B0 | Streptomyces guanduensis |
| A0A132MVR8 | Streptomyces thermoautotrophicus | A0A6L9ZUK7 | Okeania sp. SIO3B5 |
| A0A4Q0SYI3 | Granulicella sibirica | E3IYN1 | Frankia inefficax (strain DSM 45817/CECT 9037/EuI1c) |
| A0A6I4PVA6 | Actinomadura sp. J1-007 | A0A2U0XCA4 | Streptomyces sp. 3212.2 |
| A0A2N0EJM9 | Streptomyces sp. 4121.5 | A0A3G7Y463 | Pseudomonas sp. R5-89-07 |
| A0A1V0VJ03 | Kitasatospora albolonga | A0A5D3FLI3 | Actinomadura decatromicini |
| A0A191V435 | Streptomyces parvulus | A0A1Q8CL40 | Actinophytocola xanthii |
| A0A6G4B9L9 | Streptomyces sp. PRKS01-65 | A0A3E2N0G5 | Mycobacterium marinum |
| A0A1A2RNK9 | Mycobacterium sp. E2479 | A0A0M9ZYS6 | Nocardia sp. NRRL S-836 |
| A0A0J6RGS6 | Methylobacterium indicum | A0A4S3GPV7 | Streptomyces sp. Akac8 |
| A0A3S8YHP1 | Streptomyces sp. WISF4 | A0A0M8V4P1 | Streptomyces sp. MMG1121 |
| A0A174DUM8 | Hungatella hathewayi | A0A418KII6 | Jiangella rhizosphaerae |
| A0A3E3DJU5 | Hungatella hathewayi | J0KPG2 | Acidovorax sp. CF316 |
| A0A2S2CXX0 | Azospirillum thermophilum | A0A0L8LUU1 | Streptomyces decoyicus |
| A0A1U9QQ65 | Streptomyces niveus | A0A0F7N5J1 | Streptomyces sp. CNQ-509 |
| A0A5M8SWG5 | Acidobacteria bacterium AB60 | A0A0P4R4K6 | Streptomyces lydicamycinicus |
| A0A562VDK8 | Stackebrandtia albiflava | A0A3A1VVX5 | Acidovorax cavernicola |
| A0A081P157 | Paenibacillus tyrfis | D9V3S9 | Streptomyces sp. AA4 |
| A0A0T9PQK5 | Yersinia pekkanenii | A0A0L9ZW14 | Clostridium botulinum |
| A0A2N7Z8Q2 | Pseudomonas sp. GW456-11-11-14-LB4 | A0A2T7K852 | Streptomyces sp. CS081A |
| F7P1Q2 | Rheinheimera sp. A13L | A0A2M9JAK1 | Streptomyces sp. CB01201 |
| A0A2R7M5W4 | Acidovorax sp. HMWF029 | D9VHB5 | Streptomyces sp. AA4 |
| A0A6M1MBT6 | Methylobacterium sp. DB0501 | A0A2G2E805 | Fluviicola sp. |
| A0A370APP0 | Streptomyces corynorhini | A0A117RV28 | Streptomyces sp. DSM 15324 |
| A0A3A9YSV9 | Streptomyces hoynatensis | A0A0Q6Q486 | Leifsonia sp. Root112D2 |
| A0A3A9YVP8 | Streptomyces hoynatensis | A0A378W381 | Mycolicibacterium senegalense |
| A0A4P7H3D0 | Nocardia sp. CS682 | A0A614WI76 | Actinomadura rayongensis |
| A0A2A7UNY3 | Nocardia sp. FDAARGOS_372 | A0A2M9GDD1 | Pseudomonas sp. S10E 269 |
| S4Z1P6 | Mycobacterium intracellulare subsp. yongonense 05-1390 | A0A2G2DZV7 | Fluviicola sp. |
| A0A3A9Z5E3 | Streptomyces hoynatensis | A0A6B2TJU5 | Streptomyces sp. SID10116 |
| A0A1B1Z107 | Fictibacillus arsenicus | A0A2M9J747 | Streptomyces sp. CB01201 |
| A0A1M3N2S0 | Myxococcales bacterium 68-20 | A0A0F0GZ56 | Streptomyces sp. NRRL F-4428 |
| A0A3A9ZFR9 | Streptomyces hoynatensis | A0A6B2T5I1 | Streptomyces sp. SID10116 |
| A0A1H7A870 | Arthrobacter sp. yr096 | A0A1C5CQV5 | Streptomyces sp. MnatMP-M17 |
| A0A0Q2UJY8 | Mycobacterium gordonae | A0A1C5C567 | Streptomyces sp. MnatMP-M17 |
| A0A7G1P329 | Streptomyces glomeroaurantiacus | A0A6B2TC04 | Streptomyces sp. SID 10116 |
| A0A2N8NYV4 | Streptomyces eurocidicus | A0A2M9J005 | Streptomyces sp. CB01201 |
| A0A517DZ34 | Sporomusa termitida | A0A1A6BAR4 | Mycobacterium gordonae |
| D9XHS6 | Streptomyces viridochromogenes (strain DSM 40736/JCM 4977/BCRC 1201/Tue 494) | A0A3A9YT28 | Streptomyces hoynatensis |
| A0A6B2S841 | Streptomyces sp. SID 10853 | A0A7K3D380 | Streptomyces sp. SID337 |
| A0A372JGE8 | Actinomadura sp. NEAU-G17 | A0A0N0AMX2 | Streptomyces sp. NRRL F-6491 |
| A0A124ED85 | Streptomyces kanasensis | A0A2V4NUA5 | Streptomyces tateyamensis |
| A0A3M2L9J1 | Nocardia stercoris | A0A2G7BYT9 | Streptomyces sp. 93 |
| A0A2M8ZBI1 | [Clostridium] celerecrescens 18A | A0A2X0I946 | Streptacidiphilus pinicola |
| A0A2A7BLE2 | Bacillus wiedmannii | A0A5F0JXX6 | Enterobacter sp. A11 |
| A0A7H0IPW3 | Streptomyces sp. CRXT-G-22 | A0A1E5XR37 | Devosia insulae DS-56 |
| A0A5J6GE85 | Streptomyces kanamyceticus | A0A4R4VLI5 | Nonomuraea deserti |
| A0A2B5NJE1 | Bacillus wiedmannii | A0A0F6ACS4 | Pseudoalteromonas luteoviolacea S4054 |
| A0A3Q9EMP0 | Streptomyces cyaneochromogenes | A0A4P6JND8 | Ktedonosporobacter rubrisoli |
| A0A6I7Z1X3 | Streptomyces sp. me109 | A0A6N9UHF0 | Streptomyces coelicoflavus |
| A0A4V6CSC2 | Nakamurella flava | A0A3D9LQS8 | Streptomyces sp. 62 |
| A0A1V4DXI0 | Streptomyces sp. GKU 895 | A0A1W0CRQ6 | Chromobacterium haemolyticum |
| A0A101S9G3 | Streptomyces canus | A0A419Z210 | Paraburkholderia sp. BL23IIN1 |
| A0A1V4DTA8 | Streptomyces sp. GKU 895 | A0A7C9W0A7 | Lentzea alba |
| A0A7M3NN13 | Streptomyces sp. uw30 | A0A419AC85 | Paracoccus siganidrum |
| A0A0X7JRB5 | Streptomyces albus subsp. albus | A0A5S9BP14 | Chromobacterium haemolyticum |
| A0A0N0K682 | alpha proteobacterium AAP38 | S2XYW3 | Streptomyces sp. HGB0020 |
| A0A0M8SLM7 | Streptomyces sp. WM4235 | A0A2N0IUW0 | Streptomyces sp. 69 |
| A0A6L8N6G8 | Streptomyces sp. SID5473 | A0A7C9RVA4 | Lentzea alba |
| A0A0M8TCZ0 | Streptomyces sp. WM4235 | A0A4R2UI58 | Pseudomonas sp. GV077 |
| A0A7K3EPC4 | Streptomyces sp. SID5606 | V7L782 | Mycobacterium avium subsp. avium 11-4751 |
| A0A5S4V5U0 | Agromyces sp. NEAU-184 | A0A1M7PGH5 | Rhizobacter sp. OV335 |
| A0A2W2F282 | Desertiactinospora gelatinilytica | A0A0T1SN52 | Streptomyces sp. Root1304 |
| A0A437P9C5 | Streptomyces sp. San01 | A0A5C4L804 | Methylobacterium sp. 17Sr1-39 |
| A0A7K3EMV1 | Streptomyces sp. SID5606 | A0A1I2N0E9 | Duganella sp. CF458 |
| A0A076M015 | Streptomyces lividans TK24 | L7N4J5 | Mycobacterium tuberculosis (strain CDC 1551/Oshkosh) |
| A0A076M6E7 | Streptomyces lividans TK24 | A0A3N1LYX4 | Streptomyces ossamyceticus |
| A0A1C5EZ81 | Streptomyces sp. DconLS | A0A5M3Y1P7 | Acrocarpospora pleiomorpha |
| A0A229GZE3 | Streptomyces sp. NBS 14/10 | A0A640URS5 | Streptomyces tubercidicus |
| A0A0M9YI58 | Streptomyces sp. WM4235 | A0A227PFC2 | Flavobacterium araucananum |
| A0A2W2G821 | Desertiactinospora gelatinilytica | A0A640V2W5 | Streptomyces tubercidicus |
| A0A6N9HIL6 | Massilia guangdongensis | A0A614M7M0 | Sphingorhabdus profundilacus |
| A0A429II99 | Streptomyces sp. WAC 06783 | A0A1I6M3Z3 | Granulicella pectinivorans |
| A0A229GK89 | Streptomyces sp. NBS 14/10 | A0A0N0MWB1 | Actinobacteria bacterium OK006 |
| A0A502ISZ1 | Pseudomonas caspiana | A0A075UPT5 | Amycolatopsis japonica |
| A0A192A1U4 | Ralstonia insidiosa | A0A616QQI4 | Pseudomonas sp. J380 |
| A0A2N8P1A1 | Streptomyces eurocidicus | A0A4R7IXH6 | Streptomyces sp. BK447 |
| A0A658L4A9 | Paraburkholderia bannensis | A0A1A3PJ33 | Mycobacterium sp. 1245111.1 |
| R6KY72 | Clostridium sp. CAG:265 | A0A6F8YCZ4 | Phytohabitans suffuscus |
| A0A2N8NZ04 | Streptomyces eurocidicus | A0A6F8YAS6 | Phytohabitans suffuscus |
| A0A617Z2J3 | Streptomyces sp. me109 | A0A1Q5L6T1 | Streptomyces sp. TSRI0107 |
| A0A7K2H415 | Streptomyces sp. SID4936 | A0A6P1EHJ8 | Stenotrophomonas sp. 364 |
| A0A7H0IGB7 | Streptomyces sp. CRXT-G-22 | A0A542H175 | Microbacterium sp. SLBN-1 |
| A0A1S8SYG6 | Clostridium puniceum | A0A4R4XT75 | Actinomadura sp. KC06 |
| A0A3Q9ETY5 | Streptomyces cyaneochromogenes | R6G3H3 | Clostridium sp. CAG:221 |
| A0A101NWM6 | Streptomyces canus | A0A0M9YP88 | Streptomyces sp. MMG1533 |
| A0A372JPS3 | Actinomadura sp. NEAU-G17 | A0A4R4Y5X1 | Actinomadura sp. KC06 |
| A0A5P9PM00 | Amycolatopsis sp. YIM 10 | A0A6G9YGH3 | Nocardia arthritidis |
| A0A1G6V1F2 | Bacillus wiedmannii | A0A428YKH0 | Kibdelosporangium aridum |
| A0A5J6GLA0 | Streptomyces kanamyceticus | A0A2T3VPE3 | Micromonospora sp. RP3T |
| A0A084JR85 | Lacrimispora celerecrescens | A0A2T3VPC9 | Micromonospora sp. RP3T |
| A0A1C7CFP8 | Curtobacterium citreum | A0A212TW72 | Streptomyces sp. 2114.4 |
| A0A542PCY1 | Streptomyces sp. SLBN-109 | A0A0D0UTQ0 | Micromonospora haikouensis |
| A0A542AJD9 | Clostridium sp. KNHs216 | A0A6G6YQW2 | Bradyrhizobium sp. 6(2017) |
| A0A542FFH0 | Curtobacterium citreum | A0A1Y2PST6 | Streptomyces pharetrae CZA14 |
| A0A4U0NRG7 | Streptomyces piniterrae | A0A2N3TSA9 | Streptomyces sp. 73 |
| A0A101V5V4 | Streptomyces dysideae | A0A1B4SCW2 | Burkholderia sp. MSMB617WGS |
| A0A4U0NGM5 | Streptomyces piniterrae | A0A3N1K0V6 | Curtobacterium sp. PhB115 |
| A0A536QPZ9 | Chloroflexi bacterium | A0A3F3HD40 | Pseudomonas sp. BRG-100 |
| A0A172YXZ0 | Pseudomonas antarctica | A0A2I2L0E2 | Frankia canadensis |
| B1MBB6 | Mycobacteroides abscessus (strain ATCC 19977/DSM44196/CIP 104536/JCM | A0A101CFG3 | Streptomyces sp. NRRL F-5122 |
| | 13569/NCTC 13031/TMC 1543) | | |
| A0A6V8SHY4 | Clostridium sp. TW1 | A0A1Q4Z867 | Streptomyces sp. CB03578 |
| A0A2M8Z073 | [Clostridium] celerecrescens 18A | A0A5S4WLV9 | Bradyrhizobium cytisi |
| A0A7M3NMB5 | Streptomyces sp. uw30 | A0A4R5ECU0 | Nonomuraea mesophila |
| A0A0M5J1L9 | Streptomyces pristinaespiralis | A0A6H1N6B8 | Streptomyces sp. SID4-11 |
| A3P0S7 | Burkholderia pseudomallei (strain 1106a) | A0A6H1N9Y4 | Streptomyces sp. SID4-11 |
| A0A2U9SAU5 | Azospirillum ramasamyi | A0A1Y2PV18 | Streptomyces pharetrae CZA14 |
| A0A5P2UHX2 | Streptomyces subrutilus | A0A1Q4Z7X5 | Streptomyces sp. CB03578 |
| D9WXW7 | Streptomyces viridochromogenes (strain DSM 40736/JCM 4977/BCRC 1201/Tue 494) | A0A124E089 | Mycolicibacterium brisbanense |
| A0A2N8KW35 | Paucibacter aquatile | A0A0K2MAA3 | Clostridium beijerinckii NRRL B-598 |
| A0A1A2YSH1 | Mycobacterium sp. E1386 | A0A100VXZ5 | Mycolicibacterium brisbanense |
| A0A2C8X955 | Streptomyces sp. OK228 | A0A1I2Y3Q1 | Actinopolymorpha cephalotaxi |
| A0A0D0RSZ7 | Pseudomonas fluorescens | A0A2G6SZQ2 | Acidovorax sp. 56 |
| A0A5Q2SEW0 | Clostridium butyricum | A0A3F3H6F3 | Pseudomonas sp. BRG-100 |
| A0A2K9CI25 | Burkholderia pseudomallei | A0A124F237 | Streptomyces sp. NRRL F-5122 |
| A0A6B2DVU0 | Amycolatopsis sp. SID8362 | A0A0K2MBA4 | Clostridium beijerinckii NRRL B-598 |
| A0A6B2DD55 | Amycolatopsis sp. SID8362 | A0A7G1KKI7 | Nocardia wallacei |
| A0A0B5DJG5 | Streptomyces nodosus | A0A6I0BG39 | Bacillus sp. CH126_4D |
| A0A2A7NG13 | Mycolicibacterium agri | A0A0L6ZEL4 | Clostridium homopropionicum DSM 5847 |
| A0A347IS65 | Ralstonia solanacearum | A0A1Q5JVQ1 | Streptomyces sp. CB01883 |
| A0A6N3DIN4 | Clostridium butyricum | A0A6F7VRE7 | Streptomyces sp. AgN23 |
| A0A4E7Q3L9 | Bacillus thuringiensis subsp. israelensis | A0A1Q4Y0H1 | Streptomyces sp. CB03578 |
| A0A0S4WI40 | Ralstonia solanacearum | V7J0U8 | Mycobacterium avium 05-4293 |
| A0A7K2X8Z1 | Streptomyces sp. SID2131 | A0A087LKK7 | Devosia sp. 17-2-E-8 |
| A0A6N9VMB1 | Streptomyces fulvissimus | A0A1J0ERR8 | Pseudomonas frederiksbergensis |
| A0A5B7V7K3 | Streptomyces sp. YIM 121038 | A0A401YDK7 | Embleya hyalina |
| A0A6B2DAX0 | Amycolatopsis sp. SID8362 | A0A2M9PBK4 | Amaricoccus sp. HAR-UPW-R2A-40 |
| A0A3D3B596 | Rhizobiales bacterium | A0A433JV32 | Labedella endophytica |
| A0A5B7V7K4 | Streptomyces sp. YIM 121038 | A0A051UIG7 | Mycobacterium [tuberculosis] TKK-01-0051 |
| A0A1I5IT25 | Amycolatopsis rubida | A0A428YDH7 | Amycolatopsis sp. WAC 01376 |
| A0A316A150 | Faecalicatena contorta | A0A4Z1BQM7 | Empedobacter tilapiae |
| A0A1B1MFP0 | Streptomyces lincolnensis | A0A1B2GSV0 | Streptomyces noursei ATCC 11455 |
| A0A2N5CT26 | Caulobacter flavus | A0A0J9E994 | Candidatus Rhodobacter lobularis |
| A0A1H1DDK3 | Pseudomonas grimontii | A0A429S1L2 | Streptomyces sp. WAC06614 |
| A0A6G2CJR8 | Turicibacter sanguinis | A0A1A2UZW8 | Mycobacterium sp. E2733 |
| A0A429TRA3 | Streptomyces sp. WAC05374 | A0A0M9XG87 | Streptomyces rimosus subsp. rimosus |
| A0A1X1XRW4 | Mycobacterium kubicae | A0A2R4LYJ0 | Celeribacter baekdonensis |
| A0A1S1NA00 | Mycobacterium talmoniae | A0A1A2Q2L6 | Mycobacterium sp. E342 |
| A0A173SC06 | Turicibacter sanguinis | A0A1N7SBH2 | Paraburkholderia ribeironis |
| A0A1G6S6D2 | Nocardioides lianchengensis | A0A3N4AYQ1 | Yersinia pestis |
| A0A1S8QGW4 | Clostridium beijerinckii | A0A5P1YSK2 | Streptomyces tendae |
| A0A386PEQ4 | Clostridium septicum | A0A2U8VAB4 | Streptomyces sp. NEAU-S7GS2 |
| A0A1H1I8L0 | Thermostaphylospora chromogena | A0A6B2VYQ4 | Streptomyces sp. SID13666 |
| A0A1S8P4F0 | Clostridium beijerinckii | A0A4R7BRE7 | Streptomyces sp. BK561 |
| A0A1U6JNV2 | Bacillus sp. V-88 | A0A3R8QDW5 | Streptomyces griseofuscus |
| A0A640UC24 | Streptomyces libani subsp. rufus | A0A7H1Q255 | Streptomyces griseofuscus |
| A0A317KLN9 | Micromonospora globispora | A0A426RW96 | Streptomyces griseofuscus |
| A0A1V4IGV0 | Clostridium chromiireducens | A0A5F0EGC2 | Cryobacterium sp. TMT1-3 |
| A0A640TXS0 | Streptomyces libani subsp. rufus | A0A2V5X6B1 | Verrucomicrobia bacterium |
| A0A497XMZ8 | Pedobacter alluvionis | A0A4R5X1C0 | Mycobacterium paragordonae |
| A0A154MC42 | Amycolatopsis regifaucium | A0A7I9WZC9 | Mycobacterium kubicae |
| A0A5S3QR35 | Pseudomonas protegens | A0A1I9ZAG1 | Nocardia seriolae |
| A0A3Q9KPL6 | Streptomyces griseoviridis | A0A1X2M1K2 | Mycobacterium decipiens |
| A0A640TZG9 | Streptomyces libani subsp. rufus | A0A4R1XDI4 | Promicromonospora sp. CF082 |
| A0A640U5U0 | Streptomyces libani subsp. rufus | Q8XND2 | Clostridium perfringens (strain 13/Type A) |
| A0A3S9ZAX6 | Streptomyces griseoviridis | A0A1X2LVD8 | Mycobacterium decipiens |
| A0A4Z0HT47 | Mycolicibacterium peregrinum | A0A1A9IX77 | Streptomyces sp. SAT1 |
| A0A220Y4G3 | Mycobacterium intracellulare subsp. chimaera | A0A022MJ41 | Streptomyces sp. Tu 6176 |
| E4MZ37 | Kitasatospora setae (strain ATCC 33774/DSM 43861/JCM 3304/KCC A-0304/NBRC | A0A1V1W0W7 | Nocardioides sp. PD653 |
| | 14216/KM-6054) | | |
| A0A0B8NPE6 | Nocardia seriolae | D6AEN1 | Streptomyces filamentosus NRRL 15998 |
| A0A5P0YW90 | Streptomyces alkaliterrae | A0A6P2BRP0 | Trebonia kvetii |
| A0A5C4WBY5 | Nonomuraea phyllanthi | A0A2A2YZY2 | Streptomyces sp. SA15 |
| A0A438KZP8 | Pseudomonas asplenii | A0A1A0LM48 | Mycobacterium sp. 1554424.7 |
| A0A5P0YN72 | Streptomyces alkaliterrae | A0A3N7E649 | Acidovorax sp. FJL06 |
| A0A1X1RSS9 | Mycobacterium celatum | A0A5Q0H641 | Saccharothrix syringae |
| A0A5C4WWK2 | Nonomuraea phyllanthi | A0A251ZLX8 | Pluralibacter gergoviae |
| A0A1N0NGA4 | Mycobacteroides abscessus subsp. abscessus | D6AIT3 | Streptomyces filamentosus NRRL 15998 |
| A0A100I679 | Mycobacterium pseudoshottsii JCM 15466 | M7A7B2 | Gordonia sp. NB4-1Y |
| A0A538IWI2 | Actinobacteria bacterium | D6AEM9 | Streptomyces filamentosus NRRL 15998 |
| A0A5S3UUW0 | Pseudoalteromonas rubra | A0A2A8S5D1 | Bacillus sp. AFS018417 |
| A0A132MSV4 | Streptomyces thermoautotrophicus | A0A257FMA6 | Burkholderiales bacterium PBB2 |
| A0A1Z4EE56 | Mycobacterium shigaense | A9VPG6 | Bacillus mycoides (strain KBAB4) |
| A0A1P8TIU5 | Streptomyces alfalfae | A0A1A2TI10 | Mycobacterium mantenii |
| A0A1P8TRJ5 | Streptomyces alfalfae | I9A6H4 | Frankia sp. QA3 |
| A0A100JFR3 | Streptomyces acidiscabies | A0A6I5EDH8 | Streptomyces sp. SID5469 |
| A0A1A6BEW8 | Mycobacterium gordonae | A0A166FI47 | Pseudovibrio sp. WM33 |
| A0A2A3LDH2 | Mycobacterium avium subsp. hominissuis | A0A0H2YMM6 | Yersinia pestis bv. Antiqua (strain Nepal516) |
| A0A1B1MC75 | Streptomyces lincolnensis | A0A1X0FD04 | Mycobacterium mantenii |
| A0A3N2QC49 | Candidatus Cardinium hertigii | A0A397QKW1 | Streptomyces sp. 19 |
| A0A2T4NNK1 | Streptomyces sp. A244 | K0VKH1 | Mycolicibacterium fortuitum subsp. fortuitum DSM 466 |
| A0A560G4X7 | Nitrospirillum amazonense | A0A4R3E0E7 | Streptomyces sp. BK674 |
| A0A7I7L702 | Mycobacterium shottsii | A0A1A1VNY3 | Mycobacterium sp. 852002-10029_SCH5224772 |
| A0A495GAK7 | Paraburkholderia sediminicola | A0A1B2N526 | Stenotrophomonas rhizophila |
| A0A6G3CCQ6 | Streptomyces sp. SID5643 | A0A3E0VPB1 | Subtercola boreus |
| A0A1J0VSX1 | Nocardia mangyaensis | A0A372ZRG3 | Kitasatospora xanthocidica |
| A0A419VKX7 | Streptomyces sp. 2112.5 | A0A1H6DVV2 | Streptomyces yanglinensis |
| A0A560HDK2 | Nitrospirillum amazonense | A0A4U3GQ96 | Stenotrophomonas rhizophila |
| G0FXE6 | Amycolatopsis mediterranei (strain S699) | A0A0T6LRA4 | Streptomyces vitaminophilus |
| A0A7H8P4S5 | Streptomyces sp. NA03103 | A0A3N1KQR1 | Stenotrophomonas rhizophila |
| A0A560K8N6 | Nitrospirillum amazonense | A0A3L7QC50 | Planctomycetes bacterium |
| A0A1V0TN30 | Streptomyces gilvosporeus | A0A1Y6D6N0 | Methylomagnum ishizawai |
| A0A2N3XD88 | Streptomyces sp. 5112.2 | A0A373A3F5 | Kitasatospora xanthocidica |
| A0A0X3UR29 | Streptomyces sp. NRRL F-4489 | A0A3L7PDE9 | Planctomycetes bacterium |
| A0A1T5BMI8 | Luteibacter sp. 22Crub2.1 | A0A1X7ERV7 | Pseudomonas sp. LAMO17WK12:I1 |
| A0A372GMI3 | Actinomadura sp. LHW52907 | A0A6G8YKQ0 | Stenotrophomonas rhizophila |
| A0A7H8P929 | Streptomyces sp. NA03103 | A0A6M4PJ20 | Streptomyces sp. jing01 |
| A0A167IAC0 | Pseudoalteromonas luteoviolacea S4060-1 | A0A1A1W9Y0 | Mycobacterium sp. 852002-10029_SCH5224772 |
| A0A646NV90 | Pseudomonas haemolytica | A0A3L7MM08 | Planctomycetes bacterium |
| A0A0Q7MYC0 | Nocardia sp. Root136 | A0A4Y5YZC1 | Luteibacter pinisoli |
| G0FU77 | Amycolatopsis mediterranei (strain S699) | A0A3E0W1Z9 | Subtercola boreus |
| E8X6Y2 | Granulicella tundricola (strain ATCC BAA-1859/DSM23138/MP5ACTX9) | A0A1M5YYK6 | Streptomyces sp. 3214.6 |
| A0A4R0KTY7 | Kribbella pittospori | A0A3N4UT21 | Streptomyces sp. Ag82_G5-5 |
| A0A0D7QD47 | Bradyrhizobium sp. LTSPM299 | F6DQM1 | Desulfotomaculum ruminis (strain ATCC 23193/DSM 2154/NCIMB 8452/DL) |
| A0A1Q5IG26 | Streptomyces sp. CB02115 | A0A554S695 | Variovorax sp. KBS0712 |
| A0A2P4UCS2 | Actinomadura sp. RB29 | A0A3D8NN14 | Streptomyces sp. IB2014 011-12 |
| G0FTK6 | Amycolatopsis mediterranei (strain S699) | A0A7H0HU07 | Streptomyces sp. CRPJ-33 |
| A0A2A9G1H1 | Amycolatopsis sulphurea | A0A7K2KBG8 | Streptomyces sp. SID4937 |
| A0A2A9G303 | Amycolatopsis sulphurea | D6K450 | Streptomyces sp. e14 |
| A0A6G6WZM7 | Nordella sp. HKS 07 | X8DP37 | Mycobacteroides abscessus subsp. bolletii 1513 |
| D4XFR4 | Achromobacter piechaudii ATCC 43553 | A0A5S3X033 | Pseudoalteromonas rubra |
| A0A0T1UP09 | Streptomyces sp. Root1310 | A0A542UG64 | Streptomyces puniciscabiei |
| A0A1V3C674 | Nocardiopsis sinuspersici | A0A345CQZ2 | Erwinia tracheiphila |
| A0A7I7LED7 | Mycobacterium shottsii | A0A1R0KPF1 | Amycolatopsis coloradensis |
| A0A2P4UD49 | Actinomadura sp. RB29 | A0A4Q7EKU7 | Pseudoalteromonas rubra |
| A0A542J244 | Acidovorax sp. SLBN-42 | A0A6N7ZAT8 | Amycolatopsis pithecelloba |
| A0A5P1DAH7 | Pseudomonas haemolytica | A0A3E0VTX9 | Subtercola boreus |
| A0A7J5CKL7 | Micromonospora sp. ALFpr18c | A0A542SXF7 | Streptomyces puniciscabiei |
| A0A1G9I1W7 | Romboutsia lituseburensis DSM 797 | A0A5S3WPR8 | Pseudoalteromonas rubra |
| A0A1V9IK45 | Clostridium sporogenes | A0A075V600 | Amycolatopsis japonica |
| A0A1A3I562 | Mycobacterium sp. 1423905.2 | A0A6B2SM92 | Streptomyces sp. SID10115 |
| A0A1L3NIS1 | Clostridium sporogenes | A0A6B2SDV9 | Streptomyces sp. SID10115 |
| A0A7K2X5K4 | Streptomyces sp. SID2131 | AVA1L7GSA6 | Streptomyces sp. TN58 |
| A0A1L3NMI5 | Clostridium sporogenes | A0A2A5NPM6 | Curtobacterium sp. ‘Ferrero’ |
| D6AUH2 | Streptomyces filamentosus NRRL 15998 | A0A6B3EDL4 | Streptomyces sp. SID8499 |
| K6TSP1 | Clostridium sp. Maddingley MBC34-26 | A0A6B3E2N7 | Streptomyces sp. SID8499 |
| A0A1J1D1D5 | Clostridium sporogenes | A0A2T1AS36 | Paraburkholderia insulsa |
| A0A1S1MTT5 | Pseudoalteromonas amylolytica | I3ZIW1 | Terriglobus roseus (strain DSM 18391/NRRL B-41598/KBS 63) |
| K6U9G6 | Clostridium sp. Maddingley MBC34-26 | A0A2T5WYK4 | Nitrosospira sp. Nsp37 |
| A0A1A3IQK4 | Mycobacterium sp. 1423905.2 | A0A0M8X6L7 | Streptomyces sp. NRRL B-1140 |
| A0A521FSV5 | Geodermatophilus aquaeductus | A0A6B3DUN5 | Streptomyces sp. SID8499 |
| A0A0K2AUW1 | Streptomyces ambofaciens (strain ATCC 23877/3486/DSM 40053/JCM 4204/NBRC | A0A085VYT6 | Hyalangium minutum |
| | 12836/NRRL B-2516) | | |
| A0A4S2U834 | Streptomyces sp. A0642 | B9JJS1 | Agrobacterium radiobacter (strain K84/ATCC BAA-868) |
| W7S2T6 | Lysinibacillus sphaericus CBAM5 | A0A1A8ZB80 | Micromonospora narathiwatensis |
| A0A553YPV9 | Streptomyces sp. MZ03-48 | A5HYC9 | Clostridium botulinum (strain Hall/ATCC 3502/NCTC 13319/Type A) |
| A0A0X3W348 | Streptomyces violaceusniger | A0A2W1UGE7 | Curtobacterium sp. MCJR17_020 |
| A0A243S1L0 | Streptomyces swartbergensis | A0A1Q5KRG5 | Streptomyces sp. TSRI0107 |
| A0A0Q7C0I1 | Nocardioides sp. Root1257 | A0A6G2YD56 | Streptomyces sp. SID8382 |
| A0A2H1KYW6 | Brevibacterium antiquum CNRZ 918 | A0A7K2JZ17 | Streptomyces sp. SID5910 |
| A0A235FF75 | Fictibacillus aquaticus | A0A1Q5GSN5 | Streptomyces sp. CB01580 |
| A0A4D4LE37 | Streptomyces violaceusniger | A0A5A5TB63 | Dictyobacter sp. Uno17 |
| A0A4R0KQ83 | Kribbella pittospori | A0A1X0AYY8 | Mycobacterium aquaticum |
| A0A4R4PBZ5 | Streptomyces sp. 8K308 | A0A3L9YSV4 | Acidovorax sp. 100 |
| A0A1I0YCR4 | Clostridium frigidicarnis | A0A419YSN1 | Streptomyces sp. TLI_171 |
| Q82G17 | Streptomyces avermitilis (strain ATCC 31267/DSM 46492/JCM 5070/NBRC 14893/ | A0A6N7Q748 | Polyangium spumosum |
| | NCIMB 12804/NRRL 8165/MA-4680) | | |
| A0A235F5Q0 | Fictibacillus aquaticus | A0A419YIZ8 | Streptomyces sp. TLI_171 |
| A0A6B9YNM0 | Edaphobacter sp. 12200R-103 | A0A4D4JI92 | Acidovorax sp. NB1 |
| A0A2T6MIV9 | Pseudomonas sp. GV047 | A0A5B1BMI0 | Mycobacterium simiae |
| L5NBF0 | Halobacillus sp. BAB-2008 | A0A4R4SG13 | Actinomadura sp. GC306 |
| W9D2J6 | Frankia sp. CcI6 | A0A7G7DWD1 | Mycobacterium tuberculosis |
| A0A1M5X856 | Streptomyces sp. 3214.6 | A0A1M6WGD3 | Desulfotomaculum aeronauticum DSM 10349 |
| S9SZ23 | Ralstonia sp. AU12-08 | A0A1A2XBD8 | Mycobacterium sp. E2327 |
| A0A2W2B5B2 | Aestuariivirga litoralis | A0A1U9P6U7 | Streptomyces sp. fd1-xmd |
| A0A1S1PX23 | Frankia soli | A0A4U5WHS9 | Streptomyces lasalocidi |
| A0A2N8N1G5 | Amycolatopsis sp. BJA-103 | A0A2P7ZG90 | Streptomyces sp. 111WW2 |
| A0A4R4RK29 | Jiangella ureilytica | A0A429N5X6 | Streptomyces sp. WAC00469 |
| A0A3N4V5L3 | Streptomyces sp. Ag82_G5-5 | A0A1J4PYH6 | Streptomyces malaysiense |
| A0A0H3CVC2 | Amycolatopsis mediterranei (strain U-32) | A0A370H5R3 | Nocardia mexicana |
| A0A6B9YNP6 | Edaphobacter sp. 12200R-103 | A0A239JDB9 | Actinoplanes regularis |
| A0A0X3WWC1 | Streptomyces violaceusniger | A0A1U9NUY0 | Streptomyces sp. fdl-xmd |
| A0A2C9ZNN6 | Streptomyces swartbergensis | A0A6B3E5R1 | Streptomyces sp. SID8499 |
| A0A0H3DDN6 | Amycolatopsis mediterranei (strain U-32) | Q3EM67 | Bacillus thuringiensis serovar israelensis ATCC 35646 |
| A0A0Q7FI10 | Massilia sp. Root418 | A0A3E0FGQ8 | Pseudomonas sp. GV062 |
| A0A0C6EV19 | Methylobacterium aquaticum | A0A1A2WPR2 | Mycobacterium sp. E2327 |
| A0A0H3DAG4 | Amycolatopsis mediterranei (strain U-32) | A0A2P7ZQQ2 | Streptomyces sp. 111WW2 |
| A0A0J6S2Y1 | Methylobacterium aquaticum | A0A1E4EJR7 | bacterium SCN 62-11 |
| A0A2N8MXJ9 | Amycolatopsis sp. BJA-103 | A0A1V2VLT5 | Herbaspirillum sp. VT-16-41 |
| A0A5J6HF55 | Streptomyces alboniger | A0A370HJ00 | Nocardia mexicana |
| A0A397MVR6 | Streptomyces sp. 3212.5 | A0A3G9J431 | Nocardioides baekrokdamisoli |
| A0A2N8MLX9 | Amycolatopsis sp. BJA-103 | A0A1U9VJG1 | blood disease bacterium A2-HR MARDI |
| A0A0D4DJE1 | Streptomyces lydicus | A0A6B2VA73 | Streptomyces sp. SID10815 |
| A0A2T0Z4U8 | Pseudomonas sp. NFPP22 | A0A6I5D4L0 | Streptomyces sp. SID4982 |
| Q744Y6 | Mycolicibacterium paratuberculosis (strain ATCC BAA-968/K-10) | A0A2N3ZD07 | Streptomyces sp. OK885 |
| A0A1D7VVB9 | Streptomyces lydicus | W7IW47 | Actinokineospora spheciospongiae |
| A0A2R4T459 | Streptomyces lunaelactis | A0A3E2YFK6 | Micromonospora sp. MW-13 |
| A0A3N1GF93 | Couchioplanes caeruleus | A0A1A2ZE77 | Mycobacterium sp. E796 |
| A0A1I5UMT9 | Ralstonia sp. NFACC01 | J5E763 | Mycobacterium colombiense CECT 3035 |
| A0A2M9LDL7 | Kitasatospora sp. CB02891 | A0A2R4E0Z4 | Acidovorax avenae subsp. avenae |
| A0A3Q9KC22 | Streptomyces lydicus | A0A5S4FIQ1 | Nonomuraea turkmeniaca |
| A0A516RK45 | Streptomyces spectabilis | A0A3E0GJK4 | Streptomyces sp. 2221.1 |
| A0A285CEV9 | Streptomyces sp. TLI_55 | A0A286C7A5 | Nitrosovibrio sp. Nv4 |
| A0A0S2P1K1 | Streptomyces hygroscopicus subsp. limoneus | A0A286CIX7 | Streptomyces sp. Ag82_G6-1 |
| A0A5P2XIC9 | Streptomyces spectabilis | A0A2U0XD89 | Streptomyces sp. 3212.2 |
| A0A516RCH7 | Streptomyces spectabilis | B2TRK6 | Clostridium botulinum (strain Eklund 17B/Type B) |
| A0A1H4L508 | Pseudomonas panacis | A0A4R1CU12 | Frankia sp. BMG5.11 |
| A0A1D8Y5P0 | Stenotrophomonas sp. LM091 | A0A5R8MNT0 | Nonomuraea sp. KC401 |
| A0A516RL89 | Streptomyces spectabilis | R9C161 | Clostridium sartagoforme AAU1 |
| A0A5M9INZ9 | Pseudomonas panacis | A0A0H3M182 | Mycobacterium bovis (strain BCG/Pasteur 1173P2) |
| A0A1G9PIE6 | Nonomuraea jiangxiensis | A0A4R8H923 | Streptomyces sp. 25 |
| A0A291QBF2 | Streptomyces formicae | A0A4R8HCT7 | Streptomyces sp. 25 |
| A0A5C5CH80 | Buttiauxella sp. B2 | A0A1H5R4D6 | Amycolatopsis pretoriensis |
| A0A2K8LLY8 | Amycolatopsis sp. AA4 | A0A285L638 | Nocardia amikacinitolerans |
| A0A1G8EZM5 | Nonomuraea jiangxiensis | A0A117Q4C8 | Streptomyces yokosukanensis |
| A0A6G2QRC7 | Streptomyces sp. SID7804 | A0A1H5VEJ4 | Saccharopolyspora jiangxiensis |
| A0A1C5EG75 | Streptomyces sp. Ncost-T10-10d | A0A495K4G8 | Williamsia muralis |
| A0A124I4X8 | Streptomyces bungoensis | A0A1A2L2B7 | Mycobacterium sp. E802 |
| A0A4P5VRQ0 | Cyanobium sp. | G0TLU6 | Mycobacterium canettii (strain CIPT 140010059) |
| A0A4Q7XVR2 | Duganella sp. BK054 | A0A6G2UAC5 | Streptomyces sp. SID7805 |
| A0A2N5AV09 | Rhizobium loti | A0A5C8Q9A5 | Streptomyces sp. IB2014 016-6 |
| A0A7I9XVK1 | Mycobacterium botniense | A0A2T5NPZ7 | Chromobacterium sp. Panama |
| A0A1H6A5U8 | Thalassococcus halodurans | A0A4R4M977 | Actinomadura sp. 7K534 |
| A0A495BWC0 | Edaphobacter dinghuensis | A0A353F9G7 | Cryomorphaceae bacterium |
| A0A7G6BQM4 | Paraburkholderia tropica | A0A1S9TFC2 | Bacillus mycoides |
| A0A431V9Z2 | Azospirillum sp. L-25-5w-1 | A0A0A0WUF7 | Bacillus mycoides |
| K6ZV34 | Paraglaciecola polaris LMG 21857 | A0A7H0NQ12 | Streptomyces sp. CB00271 |
| A0A7L6APW4 | Candidatus Thiothrix singaporensis | A0A6G2I3J8 | Streptomyces sp. HUCO-GS316 |
| A0A1V2NKN6 | Leifsonia sp. ALI-44-B | B4CUF2 | Chthoniobacter flavus Ellin428 |
| A0A1Z2L330 | Streptomyces albireticuli | A0A1A3KS33 | Mycobacterium asiaticum |
| A0A4S2USX3 | Streptomyces sp. LRa12 | A0A1A3L2Z8 | Mycobacterium asiaticum |
| A0A6G3DLU1 | Streptomyces sp. SID5914 | A0A550HMA8 | Streptomyces sp. IB201691-2A2 |
| A0A4R1WHN0 | Kribbella sp. VKM Ac-2568 | A0A3N1YDA6 | Curtobacterium sp. JUb34 |
| A0A370VNS4 | Streptomyces sp. M7 | A0A652JSP8 | Streptomyces sp. col6 |
| A0A2C9EES1 | Pseudomonas protegens (strain DSM 19095/LMG 27888/CHA0) | A0A443TBJ1 | Bacillus mycoides |
| A0A4V1DB37 | Pseudomonas veronii | A0A1A3P1F1 | Mycobacterium asiaticum |
| A0A4R3EP20 | Streptomyces sp. BK042 | A0A1A3NNR2 | Mycobacterium asiaticum |
| A0A542TWS1 | Streptomyces sp. SLBN-192 | A0A1H0WRG7 | Acidovorax cattleyae |
| A0A1V6MYT3 | Streptomyces phaeoluteigriseus | A0A1A3BH41 | Mycobacterium asiaticum |
| A0A1Z2KY60 | Streptomyces albireticuli | A0A1W2LXS2 | Amycolatopsis keratiniphila subsp. keratiniphila |
| A0A1F4E943 | Burkholderiales bacterium RIFCSPHIGHO2_01 FULL_64_960 | A0A6H9YPL9 | Actinomadura rudentiformis |
| A0A4R3F8F6 | Streptomyces sp. BK042 | A0A6M4H124 | Usitatibacter rugosus |
| A0A429CD63 | Amycolatopsis sp. WAC 01416 | A0A0H2WHQ1 | Burkholderia mallei (strain ATCC 23344) |
| A0A2A2DAC0 | Streptomyces albireticuli | A0A328LE00 | Bacillus sp. SRB_8 |
| A0A1C5DBR8 | Streptomyces sp. Cmuel-A718b | A0A561WBF6 | Actinoplanes teichomyceticus |
| A0A2D0K0J8 | Xenorhabdus sp. KK7.4 | A0A6H9Z5C2 | Actinomadura rudentiformis |
| A0A4S2VFH2 | Streptomyces sp. LRa12 | A0A498BHZ7 | Streptomyces sp. 57 |
| A0A2A2DGI8 | Streptomyces albireticuli | A0A7H0NF57 | Streptomyces sp. CB00271 |
| A0A429DEV1 | Amycolatopsis sp. WAC 01416 | F6G4K4 | Ralstonia solanacearum (strain Po82) |
| A0A4S2W537 | Streptomyces sp. LRa12 | A0A1M5LYE9 | Streptoalloteichus hindustanus |
| A0A1Z2L3R4 | Streptomyces albireticuli | A0A2U2ZW25 | Streptomyces sp. Act143 |
| A0A2A2DAK5 | Streptomyces albireticuli | A0A657XBB3 | Pseudomonas sp. FW300-E2 |
| A0A1Z2LBH6 | Streptomyces albireticuli | A0A561VI80 | Actinoplanes teichomyceticus |
| A0A0R3AIL0 | Pseudomonas paralactis | A0A3S9PD41 | Streptomyces luteoverticillatus |
| I0S0V4 | Mycobacterium xenopi RIVM700367 | A0A1W2LP70 | Amycolatopsis keratiniphila subsp. keratiniphila |
| A0A4P6TV30 | Streptomyces seoulensis | A0A1W2M134 | Amycolatopsis keratiniphila subsp. keratiniphila |
| A0A1Q3ZHV0 | Flavobacteriia bacterium 40-80 | A0A6H9YTI4 | Actinomadura rudentiformis |
| A0A7H8LIN4 | Streptomyces seoulensis | A0A1H5HAY1 | Streptomyces sp. TLI_105 |
| A0A2U1X855 | Azospirillum sp. TSH20 | A0A542RR34 | Nocardioides sp. SLBN-35 |
| A0A7G6B0R9 | Herbaspirillum frisingense | A0A3M0CFM6 | Eilatimonas milleporae |
| A0A7G3UMX9 | Streptomyces tsukubensis (strain DSM 42081/NBRC 108919/NRRL 18488/9993) | J9W5V8 | Mycobacterium indicus pranii (strain DSM 45239/MTCC 9506) |
| A0A2W5Y7G6 | Candidatus Dormibacteraeota bacterium | A0A2V2QCK9 | Streptomyces sp. ZEA17I |
| A0A7K2VST6 | Streptomyces sp. SID1328 | A0A3M0CKR2 | Eilatimonas milleporae |
| A0A542EKV7 | Kribbella jejuensis | A0A559TWL6 | Streptomyces sp. CNZ289 |
| A0A4R8BLX2 | Curtobacterium sp. PhB190 | A0A386ZCU4 | Nocardia yunnanensis |
| A0A542EL00 | Kribbella jejuensis | A0A1A1X0B1 | Mycobacterium sp. ACS4054 |
| A0A497ZLI2 | Ruegeria conchae | A0A2B4XSA3 | Bacillus mycoides |
| A0A6H1JPL0 | Streptomyces sp. RPA4-5 | A0A550HUB4 | Streptomyces sp. IB201691-2A2 |
| A0A1X2BL69 | Mycobacterium saskatchewanense | A0A1A3NB83 | Mycobacterium asiaticum |
| A0A6B1KGQ9 | Streptomyces sp. SID5464 | A0A1A3NNH0 | Mycobacterium asiaticum |
| A0A4R3DN01 | Streptomyces sp. BK674 | A0A1A3MXR7 | Mycobacterium asiaticum |
| A0A4R3E6M3 | Streptomyces sp. BK674 | A0A653YF38 | Bacillus mycoides |
| A0A6M0FL35 | Okeania sp. SIO117 | A0A1E8BQU4 | Bacillus mycoides |
| A0A397QJY3 | Streptomyces sp. 19 | J8IK54 | Bacillus mycoides |
| A0A397QJ55 | Streptomyces sp. 19 | A0A1A3BEK5 | Mycobacterium asiaticum |
| A0A1I1YDM8 | Acidovorax konjaci | A0A550HEQ2 | Streptomyces sp. IB201691-2A2 |
| A0A6I5DY60 | Streptomyces sp. SID5474 | A0A550HCZ4 | Streptomyces sp. IB201691-2A2 |
| A0A076EYZ0 | Rhodococcus opacus | A0A1X6Q4I1 | Bacillus mycoides |
| A0A6H1RH42 | Saccharopolyspora sp. ASAGF58 | A0A6G2IJU5 | Streptomyces sp. HUCO-GS316 |
| A0A1X0BEC1 | Mycobacterium bouchedurhonense | A0A4Q9ZG27 | Bacillus mycoides |
| A0A4R0ICK3 | Kribbella sp. YM55 | A0A1A3P2P4 | Mycobacterium asiaticum |
| A0A494X021 | Pararobbsia silviterrae | A0A0E1S826 | Burkholderia pseudomallei 305 |
| A0A3A4AWK7 | Bailinhaonella thermotolerans | A0A1A2B2Z3 | Mycobacterium sp. 852002-50816_SCH5313054-b |
| A0A1H4LHZ0 | Terriglobus roseus | A0A1A1X162 | Mycobacterium sp. ACS4054 |
| A0A2P1PMR4 | Ahniella affigens | A0A1A3CKN3 | Mycobacterium asiaticum |
| A0A4Q7WVL4 | Streptomyces sp. BK239 | A0A1C4C7N0 | Bacillus mycoides |
| A0A4Q7WXS3 | Streptomyces sp. BK239 | A0A1D3N5U0 | Bacillus mycoides |
| A0A0F0HQ31 | Saccharothrix sp. ST-888 | A0A1Q5FM51 | Streptomyces sp. CB00316 |
| A0A397RPG8 | Pelomonas sp. BT06 | A0A3S8R950 | Tenacibaculum sp. DSM 106434 |
| A0A143PF33 | Luteitalea pratensis | A0A0N9IB07 | Kibdelosporangium phytohabitans |
| A0A3C1DZ34 | Microbacteriaceae bacterium | A0A239NYR4 | Streptomyces glauciniger |
| A0A090YSC8 | Bacillus clarus | A0A0J7ZJS6 | Streptomyces viridochromogenes |
| A0A6G3AIU4 | Streptomyces sp. SID2999 | A0A0J7ZCT0 | Streptomyces viridochromogenes |
| A0A1H2K7G2 | Jiangella alkaliphila | A0A0N9IJ24 | Kibdelosporangium phytohabitans |
| A0A101QID2 | Streptomyces antibioticus | A0A174WCK3 | Clostridium paraputrificum |
| A0A1H5N159 | Streptomyces sp. Ag109_O5-10 | A0A2G6YBJ9 | Acidovorax sp. 62 |
| A0A3G7X8Q1 | Pseudomonas orientalis | A0A511TGA3 | Myxococcus fulvus |
| A0A520ILY7 | Pedobacter sp. | A0A238L750 | Maliponia aquimaris |
| H2JYQ2 | Streptomyces hygroscopicus subsp. jinggangensis (strain 5008) | A0A0N9IEX7 | Kibdelosporangium phytohabitans |
| A0A4V2DY71 | Pseudomonas orientalis | A0A0N9I6V6 | Kibdelosporangium phytohabitans |
| A0A101Q1J1 | Streptomyces antibioticus | A0A1Q5EC32 | Kitasatospora sp. CB01950 |
| A0A4R6SPE3 | Labedaea rhizosphaerae | A0A7G8BDF9 | Acidobacterium sp. 4Y35 |
| A0A7L4ZE11 | Kordia antarctica | A0A0N9HNU2 | Kibdelosporangium phytohabitans |
| A0A3G7WSV3 | Pseudomonas orientalis | A0A0N7F488 | Kibdelosporangium phytohabitans |
| A0A0R2ZTK8 | Pseudomonas orientalis | A0A248YT66 | Plantactinospora sp. KBS50 |
| A0A428YW25 | Actinoplanes sp. ATCC 53533 | A0A0J7YZY7 | Streptomyces viridochromogenes |
| A0A1S9NR44 | Streptomyces antibioticus | A0A0N9IGF3 | Kibdelosporangium phytohabitans |
| A0A101Q0K3 | Streptomyces antibioticus | A0A1Q5F0C8 | Kitasatospora sp. CB01950 |
| E2PWZ8 | Streptomyces clavuligerus | A0A0N9I8L9 | Kibdelosporangium phytohabitans |
| Q9EX55 | Streptomyces coelicolor (strain ATCC BAA-471/A3(2)/M145) | A0A1Q5EUE5 | Kitasatospora sp. CB01950 |
| A0A7L4ZFS5 | Kordia antarctica | A0A5C7TJC1 | Rheinheimera sp. |
| A0A2SIT177 | Streptomyces tirandamycinicus | A0A3D5BP74 | Rheinheimera sp. |
| A0A419SYH0 | Lacrimispora algidixylanolytica | A0A3S8WB37 | Streptomyces sp. WAC 01529 |
| A0A419T7F0 | Lacrimispora algidixylanolytica | A0A1G5A697 | Variovorax sp. EL159 |
| A0A2R5H9Z0 | Mycobacterium montefiorense | A0A2R4VXX4 | Azospirillum humicireducens |
| A0A0P7BXH8 | Frankia sp. R43 | D3D5C8 | Frankia sp. EUNIf |
| A0A2T9K5I4 | Caulobacter radicis | A0A7K1K5I3 | Mycolicibacterium sp. CBMA 247 |
| A0A4R0HDV2 | Kribbella soli | A0A7G8BN49 | Acidobacterium sp. 4Y35 |
| A0A4R0HAY9 | Kribbella soli | Q8Y3D0 | Ralstonia solanacearum (strain GMI1000) |
| A0A6S6QX45 | Anaerocolumna cellulosilytica | A0A7G6ZB84 | Glaciihabitans sp. INWT7 |
| B2U7W5 | Ralstonia pickettii (strain 12J) | R4US60 | Mycobacteroides abscessus subsp. bolletii 50594 |
| A0A495ICU8 | Frondihabitans australicus | A0A5C6WJE0 | Streptomyces sp. ISID311 |
| A0A4R5EUT2 | Actinomadura sp. 6K520 | A0A378UM96 | Mycolicibacterium aichiense |
| A0A6G9Z5P4 | Nocardia terpenica | A0A3A4JPK5 | Nocardia panacis |
| A0A164IZ76 | Nocardia terpenica | A0A2A9JCV8 | Saccharopolyspora erythraea (strain ATCC 11635/DSM 40517/JCM 4748/NBRC |
| | | | 13426/NCIMB 8594/NRRL 2338) |
| A0A454TQ69 | Ralstonia pseudosolanacearum | A4FGY6 | Saccharopolyspora erythraea (strain ATCC 11635/DSM 40517/JCM 4748/NBRC |
| | | | 13426/NCIMB 8594/NRRL 2338) |
| A0A3L7AJH0 | Xanthobacter tagetidis | A0A4R7EBQ3 | Nitrosospira multiformis |
| B5G877 | Streptomyces sp. (strain SPB074) | A0A328ZAQ9 | Nitrosospira multiformis |
| A0A209CFA0 | Streptomyces sp. CS159 | A0A1I7FAW6 | Nitrosospira multiformis |
| O53608 | Mycobacterium tuberculosis (strain ATCC 25618/H37Rv) | A0A2S7F4X2 | Clostridium butyricum |
| A0A4R5EMA1 | Actinomadura sp. 6K520 | A0A6L9EQ18 | Clostridium butyricum |
| V4HV53 | Pseudoalteromonas luteoviolacea 2ta16 | A0A101CDT0 | Chryseobacterium greenlandense |
| A0A4R5EM01 | Actinomadura sp. 6K520 | A0A5B8JGZ4 | Streptomyces qinzhouensis |
| A0A7H5JVY8 | Streptomyces sp. S1A1-3 | A0A4R0I9T5 | Kribbella sindirgiensis |
| A0A6G2LQ34 | Streptomyces sp. SID4956 | A0A4R0I6J0 | Kribbella sindirgiensis |
| V4I4T2 | Pseudoalteromonas luteoviolacea 2ta16 | A0A2A3HNX0 | Streptomyces sp. TLI_235 |
| A0A2S6HX43 | Hungatella xylanolytica | A0A1H2I3F2 | Amycolatopsis keratiniphila |
| A0A1U9QUH9 | Streptomyces niveus | A0A345SX67 | Streptacidiphilus bronchialis |
| A0A7H5JWQ4 | Streptomyces sp. S1A1-3 | R4SWX3 | Amycolatopsis keratiniphila |
| A0A176L217 | Streptomyces sp. FXJ1.172 | R4SIA3 | Amycolatopsis keratiniphila |
| A0A2S6HN71 | Hungatella xylanolytica | A0A657QMY9 | Mycobacterium persicum |
| A0A6C0QAW3 | Streptomyces sp. S4.7 | A0A2P9IKN9 | Actinomadura parvosata subsp. kistnae |
| A0A327V7X4 | Streptomyces sp. KhCrAH-43 | X7Y9D5 | Mycobacterium kansasii 824 |
| A0A3N7CX84 | Pedobacter sp. KBW06 | A0A1P8K0U3 | Rhodoferax koreense |
| A0A1I0FEU4 | Nonomuraea wenchangensis | A0A657QN36 | Mycobacterium persicum |
| A0A1I3DUS3 | Pseudomonas sp. NFPP04 | A0A2P9I1Q7 | Actinomadura parvosata subsp. kistnae |
| H0BRV8 | Acidovorax sp. NO-1 | A0A1V0QUC2 | Streptomyces sp. Sge12 |
| F7NYL7 | Rheinheimera sp. A13L | A0A657A9K8 | Mycobacterium persicum |
| A0A2W2BZQ9 | Jiangella anatolica | A0A1H9G8E1 | Lentzea flaviverrucosa |
| A0A654A2P7 | Bacillus sp. 349Y | A0A2W0FR75 | Pseudomonas sp. TKO29 |
| A0A0N1NIL9 | Actinobacteria bacterium OK074 | A0A6G2RJF9 | Streptomyces sp. SID4951 |
| A0A5R8Z8L1 | Microbispora fusca | A0A1A3EF04 | Mycobacterium sp. 1465703.0 |
| A0A370IA87 | Nocardia pseudobrasiliensis | A0A1A3DPP1 | Mycobacterium sp. 1465703.0 |
| A0A370HYE5 | Nocardia pseudobrasiliensis | A0A1V0R830 | Streptomyces sp. Sge12 |
| L8EGB6 | Streptomyces rimosus subsp. rimosus (strain ATCC 10970/DSM 40260/JCM | A0A6B2RJ57 | Streptomyces sp. SID 10362 |
| | 4667/NRRL 2234) | | |
| A0A3N0DIH5 | Marmoricola solisilvae | A0A5B8JB07 | Streptomyces qinzhouensis |
| A0A0R3FF03 | Mycobacteroides sp. H002 | I9NUD7 | Pelosinus fermentans JBW45 |
| A0A0N1GVP8 | Actinobacteria bacterium OK074 | A0A1H9D6V7 | Lentzea flaviverrucosa |
| A0A0N1GFY0 | Actinobacteria bacterium OK074 | A0A498PF71 | Mycobacterium persicum |
| A0A4D4K8C9 | Streptomyces antimycoticus | X7Y1F9 | Mycobacterium kansasii 824 |
| A0A6G3W4E2 | Streptomyces sp. SID9944 | A0A657ADX4 | Mycobacterium persicum |
| A0A2C0X252 | Bacillus cereus | A0A2P9I172 | Actinomadura parvosata subsp. kistnae |
| A0A2B9E4N4 | Bacillus cereus | A0A1H2J2U5 | Amycolatopsis keratiniphila |
| A0A1C4XV44 | Micromonospora chokoriensis | A0A498P946 | Mycobacterium persicum |
| A5TYD4 | Mycobacterium tuberculosis (strain ATCC 25177/H37Ra) | A0A6I6KK66 | Streptomyces rimosus R6-500 |
| A0A5J6J9V4 | Streptomyces vinaceus | A0A6G2PBG1 | Streptomyces sp. SID5466 |
| A0A2C2VYJ5 | Bacillus cereus | A0A6G2PBP7 | Streptomyces sp. SID5466 |
| A0A2B4ATX1 | Bacillus thuringiensis | A0A6B0E8B1 | Clostridium chromiireducens |
| A0A511NMD9 | Empedobacter brevis NBRC 149 | A0A0E3M1R6 | uncultured bacterium AR_456 |
| A0A1H3BC65 | Saccharopolyspora shandongensis | L7RX87 | Streptomyces sp. FXJ7.023 |
| A0A1X0K7D6 | Mycobacterium scrofulaceum | A0A6G3QQV3 | Streptomyces sp. SID14436 |
| AVA1I0IX00 | Lacrimispora sphenoides | B0CN23 | Streptomyces lavendulae |
| A0A1A2UVL9 | Mycobacterium scrofulaceum | A0A6B1JX69 | Streptomyces sp. SID6139 |
| A0A209C5Q1 | Streptomyces sp. CS159 | Q7X2G7 | Streptomyces griseoflavus |
| A0A6G3VVA8 | Streptomyces sp. SID9944 | Q9X5Q4 | Streptomyces lavendulae |
| A0A1S9UP94 | Bacillus cereus | A0A212LZ31 | uncultured Sporomusa sp. |
| A0A1Y0TKD7 | Bacillus thuringiensis | A0A6N3FDB9 | Clostridium paraputrificum |
| A0A2C2FAB9 | Bacillus thuringiensis | A0A449H252 | Nocardia farcinica |
| A0A6G3WCE6 | Streptomyces sp. SID9944 | A0A2H4RBY1 | Micromonospora sp. HK160111 |
| A0A5Q4T9L9 | Streptomyces sp. t39 | A0A0E3M0B2 | uncultured bacterium AR_412 |
| A0A5M8SEQ1 | Bacillus cereus | A0A6G2PLU3 | Streptomyces sp. SID5466 |
| A0A2B3LSR1 | Bacillus thuringiensis | A0A2I6SBC7 | Amycolatopsis sp. |
| A0A369BN03 | Bacillus sp. AG236 | M9T245 | Streptomyces sp. SCSIO 01127 |
| A0A2C1SJK6 | Bacillus cereus | B0CN14 | Streptomyces lavendulae |
| A0A150BUC0 | Bacillus cereus | A0A6B3C2P0 | Streptomyces sp. SID 12501 |
| Q5LL21 | Ruegeria pomeroyi (strain ATCC 700808/DSM 15171/DSS-3) | A0A6B3RIH6 | Bacillus megaterium |
| A0A0X3X2P7 | Streptomyces sp. NRRL S-1521 | A0A2P2BRX1 | Romboutsia hominis |
| A0A0N0MSG0 | Actinobacteria bacterium OK074 | A0A5Q4TEW5 | Streptomyces sp. t39 |
| J7TCA6 | Clostridium sporogenes (strain ATCC 15579) | A0A499UXP8 | Streptomyces antimycoticus |
| A0A370ICF1 | Nocardia pseudobrasiliensis | A0A5J6JBT6 | Streptomyces vinaceus |
| A0A0Q7UFE3 | Caulobacter sp. Root1455 | A0A1S9YLE2 | Bacillus cereus |
| A0A2N3WX76 | Nocardia fluminea | A0A150C2X3 | Bacillus cereus |
| A0A2T5BH47 | Streptomyces sp. VMFN-G11Ma | A0A2B1FTR6 | Bacillus cereus |
| A0A0X3WP36 | Streptomyces sp. NRRL S-1521 | A0A3S9T4Y0 | Bacillus thuringiensis |
| A0A1W9ZQH4 | Mycobacterium arosiense ATCC BAA-14 | A0A3N5AGX2 | Streptomyces sp. Ag109_G2-6 |
| B1HNL3 | Lysinibacillus sphaericus (strain C3-41) | A0A0D1AQ68 | Clostridium botulinum B2 450 |
| A0A1M7D826 | Chishuiella changwenlii | A0A1X0KHQ7 | Mycobacterium scrofulaceum |
| A0A1A2XD00 | Mycobacterium sp. E2497 | A0A1S7F807 | Bacillus thuringiensis |
| A0A2W1U8T4 | Curtobacterium sp. MCLR17_039 | A0A1A2W933 | Mycobacterium scrofulaceum |
| A0A2G6WSI9 | Streptomyces sp. 76 | A0A4D4KRE9 | Streptomyces antimycoticus |
| A0A1Z4EXP5 | [Mycobacterium] stephanolepidis | A0A2N8QBH1 | Paraburkholderia fungorum |
| A0A5F0DBY8 | Cryobacterium luteum | A0A6G3WCL1 | Streptomyces sp. SID9944 |
| A0A1A3EKD2 | Mycobacterium sp. 1482292.6 | A0A2B0KDM6 | Bacillus cereus |
| A0A0J1J5Z5 | Robinsoniella sp. RHS | A0A3N4RFP5 | Kitasatospora niigatensis |
| A0A4R4ZS39 | Kribbella antibiotica | A0A559V075 | Streptomyces sp. CNZ287 |
| A0A4R4ZJH0 | Kribbella antibiotica | A0A5N0ITY8 | Mycobacterium sp. |
| A0A2T7T0M5 | Streptomyces scopuliridis RB72 | A0A1A3KKZ9 | Mycobacterium sp. 1245852.3 |
| V9HGZ2 | Clostridium sp. 7_2_43FAA | D5P4C3 | Mycobacterium parascrofulaceum ATCC BAA-614 |
| A0A1U9R2J0 | Streptomyces niveus | D5PCQ8 | Mycobacterium parascrofulaceum ATCC BAA-614 |
| A0A196QEF6 | Sulfitobacter geojensis | A0A508TUY1 | Bradyrhizobium ivorense |
| A0A3S8WWZ8 | Streptomyces sp. WAC 01438 | A0A508TYW0 | Bradyrhizobium ivorense |
| A0A176KV72 | Streptomyces sp. FXJ1.172 | A0A1N0U5P9 | Mycobacteroides abscessus subsp. bolletii |
| A0A2A5HSY8 | Alteromonadaceae bacterium | A0A0C1GRX9 | Ruegeria sp. ANG-R |
| A0A0F5VMR7 | Streptomyces sp. WM6386 | A0A2U3NJY0 | Mycobacterium terramassiliense |
| A0A640SUP3 | Streptomyces glebosus | A0A316I8T2 | Lentzea deserti |
| A0A4R8DEK9 | Kribbella sp. VKM Ac-2566 | A0A343JCB2 | Clostridium isatidis |
| A0A4R8DDF0 | Kribbella sp. VKM Ac-2566 | A0A2I0SAS2 | Streptomyces populi |
| A0A7D6ZEY1 | Nocardia sp. WCH-YHL-001 | A0A5C7WFK9 | Mycobacterium sp. |
| A0A3S5HVM2 | Achromobacter spanius | A0A4V2XYB5 | Actinomadura sp. KC345 |
| A0A1C6BLT8 | uncultured Clostridium sp. | A0A1N6A092 | Mycobacteroides abscessus subsp. bolletii |
| A0A6B4DAW9 | Clostridium botulinum | A0A6B9B4N4 | Mycobacterium avium subsp. avium |
| A0A0S4VME3 | Ralstonia solanacearum | A0A149PR07 | Paraburkholderia monticola |
| A0A1C6GD53 | uncultured Clostridium sp. | A0A433AW80 | Mycobacterium sp. |
| A0A0S4U2J4 | Ralstonia solanacearum | A0A316IDU9 | Lentzea deserti |
| A0A0K3BB22 | Kibdelosporangium sp. MJ126-NF4 | A0A3S0RT84 | Mycobacterium sp. |
| A0A0K3B1X9 | Kibdelosporangium sp. MJ126-NF4 | B1IEC8 | Clostridium botulinum (strain Okra/Type B1) |
| A0A0K3B3N7 | Kibdelosporangium sp. MJ126-NF4 | A0A1S1RDK0 | Frankia sp. BMG5.36 |
| A0A0K3BN04 | Kibdelosporangium sp. MJ126-NF4 | A0A6G6ZT48 | Pseudomonas sp. BIOMIG1BAC |
| A0A7C6FUB6 | Papillibacter sp. | A0A0Q6WT32 | Massilia sp. Root351 |
| A0A6G3XCW2 | Streptomyces sp. SID7499 | A0A553DQS7 | Flavobacterium sp. ZT3R18 |
| A0A6B1M1A7 | Streptomyces sp. SID8361 | A0A3E0W815 | Subtercola boreus |
| A0A0K3B041 | Kibdelosporangium sp. MJ126-NF4 | A0A3G2FTF3 | Acidovorax sp. 1608163 |
| D2XDW8 | Kibdelosporangium sp. MJ126-NF4 | A0A0E9MSN7 | Sphingomonas changbaiensis NBRC 104936 |
| A0A6B3C559 | Streptomyces sp. SID12501 | A0A0F4QXW3 | Pseudoalteromonas rubra |
| A0A7D7WJS2 | Devosia sp. D6-9 | B1IEB4 | Clostridium botulinum (strain Okra/Type B1) |
| D1H0B7 | Streptomyces albaduncus | A0A7K0CVA0 | Nocardia sp. RB20 |
| A0A6B1M8Y1 | Streptomyces sp. SID8361 | A0A0M9ZY76 | Streptomyces sp. NRRL WC-3618 |
| A0A0K3BAZ6 | Kibdelosporangium sp. MJ126-NF4 | A0A1H9SMI5 | Lentzea albida |
| A0A0K3BT81 | Kibdelosporangium sp. MJ126-NF4 | A0A563EU48 | Lentzea sp. FXJ1.1311 |
| A0A6G4ZDQ9 | Clostridium perfringens | A0A4Q6IK40 | Streptomyces sp. F001 |
| A0A0K3AYA2 | Kibdelosporangium sp. MJ126-NF4 | A0A7D7WWP2 | Streptomyces lincolnensis |
| A0A0K3ATV8 | Kibdelosporangium sp. MJ126-NF4 | A0A5N5W999 | Streptomyces mobaraensis |
| A0A6B1PK85 | Streptomyces sp. SID2955 | A0A1R0KZQ9 | Amycolatopsis coloradensis |
| Q2PC35 | Streptomyces achromogenes subsp. rubradiris | A0A553DEW6 | Flavobacterium sp. ZT3R18 |
| A0A6G3U3T3 | Streptomyces sp. SID7958 | A0A2G6PQU7 | Bacteroidetes bacterium |
| A0A5P2F1L9 | Clostridium diolis | A0A6G4WXB2 | Streptomyces boncukensis |
| A0A1H7K5U9 | Variovorax sp. YR750 | A0A4Q1RQQ0 | Streptomyces sp. TM32 |
| A0A4Q7YPD7 | Edaphobacter modestus | A0A7K3QMM3 | Streptomyces bauhiniae |
| A0A5P2F4D9 | Clostridium diolis | A0A1Q3RTM4 | Burkholderiales bacterium 64-34 |
| A0A7H8H7A0 | Streptomyces harbinensis | A0A2P8QDL5 | Streptomyces dioscori |
| A0A2P7Z5X0 | Streptomyces sp. 111WW2 | A0A5S4Z8I2 | Bacillus marisflavi |
| A0A402C6G0 | Rhodococcus wratislaviensis | A0A6G4X807 | Streptomyces boncukensis |
| A0A5P2GD62 | Empedobacter brevis | A0A561UZ50 | Streptomyces brevispora |
| A0A3N4ZM67 | Streptomyces sp. TLI_185 | A0A3N0DD88 | Streptomyces sp. I6 |
| A0A4Y7QQR7 | Bacillus sp. BH2 | A0A0M0G0A0 | Bacillus marisflavi |
| A0A5P2EZJ5 | Clostridium diolis | A0A3N6F2H5 | Streptomyces sp. ADI95-17 |
| A0A344L2Q7 | Amycolatopsis albispora | A0A522NTJ6 | Mycobacterium sp. |
| A0A2Z5K708 | Streptomyces sp. Go-475 | A0A559VUG9 | Streptomyces sp. CNZ287 |
| A0A3M3ZYE7 | Pseudomonas marginalis pv. marginalis | A0A4V6CLX6 | Mycobacteroides abscessus subsp. bolletii |
| A0A1M3EBI9 | Bacteroidetes bacterium 43-16 | A0A559VYK4 | Streptomyces sp. CNZ287 |
| A0A3M5QLM3 | Pseudomonas marginalis pv. marginalis | A0A4R4S050 | Actinomadura sp. KC345 |
| A0A4R2Q341 | Streptomyces sp. BK438 | A0A3P6KQT7 | Clostridium carnis |
| A0A049DU94 | Mycobacterium avium XTB13-223 | A0A5A9F4G5 | Azospirillum sp. B21 |
| A0A4Q7YQJ2 | Edaphobacter modestus | A0A316I0N7 | Lentzea deserti |
| A0A6P1FZZ3 | Empedobacter brevis | A0A0Q8SUN9 | Acidovorax sp. Root217 |
| A0A0D7CLI5 | Streptomyces natalensis ATCC 27448 | A0A1V2MV74 | Streptomyces sp. IB2014 011-1 |
| A0A1C4L7P8 | Streptomyces sp. DvalAA-43 | A0A1A3MPQ0 | Mycobacterium sp. 1245852.3 |
| A0A5J6III2 | Streptomyces coeruleorubidus | A0A5J6U2K0 | Actinomadura sp. WMMB 499 |
| A0A100WMG8 | Mycolicibacterium fortuitum subsp. acetamidolyticum | A0A0D7PA98 | Bradyrhizobium sp. LTSP885 |
| A0A2Z5K333 | Streptomyces sp. Go-475 | A0A1V2P561 | Actinosynnema sp. ALI-1.44 |
| A0A561XY46 | Acidovorax delafieldii | B1BNQ3 | Clostridium perfringens E str. JGS1987 |
| A0A4R2Z760 | Pedobacter sp. CF074 | A0A0N0H152 | Streptomyces chattanoogensis |
| A0A5J6I6P1 | Streptomyces coeruleorubidus | A0A3N6C325 | Streptomyces sp. ADI91-18 |
| A0A1M6NM90 | Anaerocolumna jejuensis DSM 15929 | A0A4Q0WTW9 | Bacillus albus |
| A0A0D7CD15 | Streptomyces natalensis ATCC 27448 | A0A2S9PQK7 | Streptomyces sp. ST5x |
| A0A6A0B520 | Streptomyces sp. CWH03 | A0A3N6EY34 | Streptomyces sp. ADI91-18 |
| Q0RGV8 | Frankia alni (strain ACN14a) | A0A1V2PQ96 | Actinosynnema sp. ALI-1.44 |
| A0A7H0HFQ9 | Acidovorax monticola | A0A1U1AFD1 | Mycobacteroides abscessus subsp. massiliense |
| A0A5A9EQI3 | Azospirillum sp. Sh1 | A0A0Q6YCQ9 | Streptomyces sp. Root369 |
| A0A2L2MHE3 | Streptomyces dengpaensis | A0A286B703 | Variovorax sp. YR752 |
| A0A1A0QPW3 | Mycolicibacterium peregrinum | A0A1A0WSC0 | Mycobacterium sp. 852013-50091_SCH5140682 |
| A0A255PJJ7 | Streptomyces sp. FBKL.4005 | A0A1V2P4D4 | Actinosynnema sp. ALI-1.44 |
| A0A4R2JZ27 | Actinocrispum wychmicini | A0A1U0WW46 | Mycobacteroides abscessus subsp. massiliense |
| A0A1A2PRR0 | Mycobacterium sp. E1715 | A0A4R5B6D3 | Actinomadura darangshiensis |
| A0A6G2XNA4 | Streptomyces sp. SID8381 | A0A0Q6YN71 | Streptomyces sp. Root369 |
| A0A4R2JZH1 | Actinocrispum wychmicini | A0A5N8X022 | Streptomyces acidicola |
| A0A0M7MK67 | Achromobacter sp. | A0A1V2PJV7 | Actinosynnema sp. ALI-1.44 |
| A0A0J6T988 | Methylobacterium tarhaniae | A0A4R7SW83 | Kribbella sp. VKM Ac-2575 |
| A0A2S8KVQ4 | Mycolicibacterium austroafricanum | A0A2Z4V1U4 | Streptomyces sp. ICC1 |
| A0A1A2PPH2 | Mycobacterium sp. E342 | A0A3N6E6L3 | Streptomyces sp. ADI91-18 |
| A0A495X4E2 | Saccharothrix variisporea | A0A0L6CH89 | Luteipulveratus halotolerans |
| A0A1I0QC00 | Luteibacter sp. 329MFSha | A0A2S9PUV9 | Streptomyces sp. ST5x |
| A0A2S0K1Q5 | Lysinibacillus sphaericus | A0A259UVJ9 | Sporomusa silvacetica DSM 10669 |
| A0A1W2FLB6 | Kibdelosporangium aridum | A0A1E7NEU8 | Kitasatospora aureofaciens |
| A0A428ZEU1 | Kibdelosporangium aridum | A0A0S6TXE5 | Clostridium botulinum B str. Osaka05 |
| A0A3S0BGX8 | Streptomyces sp. WAC08241 | A0A084JS47 | Lacrimispora celerecrescens |
| A0A1A3SMU6 | Mycobacterium sp. 1274761.0 | A0A1S2NYP2 | Streptomyces sp. MUSC 14 |
| A0A6G9Y7F7 | Nocardia arthritidis | A0A124IF76 | Streptomyces dysideae |
| A0A1Y5XQS4 | Kibdelosporangium aridum | A0A229GL02 | Streptomyces sp. NBS 14/10 |
| A0A3R9V6U4 | Streptomyces sp. WAC08241 | A0A0N0SKS3 | Streptomyces sp. WM4235 |
| C4AQN2 | Burkholderia mallei GB8 horse 4 | A0A437P444 | Streptomyces sp. San01 |
| A0A077MG14 | Tetrasphaera jenkinsii Ben 74 | A0A229HKN4 | Streptomyces sp. NBS 14/10 |
| A0A1I3F7W0 | Streptosporangium canum | A0A1A2D151 | Mycobacterium sp. 852002-51163_SCH5372311 |
| A0A428Z4A4 | Kibdelosporangium aridum | A0A0M8RQL1 | Streptomyces rimosus subsp. pseudoverticillatus |
| A0A428ZEM5 | Kibdelosporangium aridum | A0A438MA47 | Nonomuraea polychroma |
| A0A428ZDF8 | Kibdelosporangium aridum | A0A3N1DMH4 | Streptomyces sp. SAI-097 |
| A0A4S2RF69 | Streptomyces sp. A1277 | A0A7K1XK08 | Streptomyces sp. XHT-2 |
| A0A5J4LW64 | Streptomyces angustmyceticus | A0A346C3L2 | Streptomyces sp. CB09001 |
| A0A1Y5XFQ7 | Kibdelosporangium aridum | A0A7K2TF93 | Streptomyces sp. SID8352 |
| A0A3B0AZY2 | Streptomyces klenkii | D6TU22 | Ktedonobacter racemifer DSM 44963 |
| A0A3B0BKF5 | Streptomyces klenkii | A0A6B3BIU1 | Streptomyces sp. SID9727 |
| A0A4Y3VVM6 | Streptomyces spinoverrucosus | A0A2A2D8B2 | Streptomyces albireticuli |
| A0A4Y3VUS0 | Streptomyces spinoverrucosus | W5WLQ4 | Kutzneria albida DSM 43870 |
| A0A1W2AE97 | Kibdelosporangium aridum | H0E4V7 | Patulibacter medicamentivorans |
| A0A1Y5XLZ8 | Kibdelosporangium aridum | A0A2N8TE55 | Streptomyces sp. 13K301 |
| A0A7H8L404 | Kitasatospora sp. NA04385 | A0A2V3X3V3 | Paraburkholderia tropica |
| A0A3B0BVU8 | Streptomyces klenkii | A0A370VMY6 | Streptomyces sp. M7 |
| A0A0S3TWP6 | Leptolyngbya sp. NIES-3755 | A0A2A2DCF5 | Streptomyces albireticuli |
| J3ZVN4 | Bacillus thuringiensis HD-771 | A0A2A2DDU0 | Streptomyces albireticuli |
| A0A246JK44 | Roseateles aquatilis | A0A7K2RQP5 | Streptomyces sp. SID8367 |
| A0A3N1JHD8 | Granulicella sp. GAS466 | A0A4R3FQ43 | Streptomyces sp. BK042 |
| A0A1X1VXT2 | Mycobacterium gastri | A0A397RH39 | Streptomyces sp. 3211.1 |
| A0A1X1UTS6 | Mycobacterium gastri | A0A429DHD4 | Amycolatopsis sp. WAC 01416 |
| A0A3B0BCS9 | Streptomyces klenkii | E6UZA6 | Variovorax paradoxus (strain EPS) |
| A0A1W2AB89 | Kibdelosporangium aridum | A0A1Z2KWD2 | Streptomyces albireticuli |
| A0A428ZF34 | Kibdelosporangium aridum | A0A1V6MU25 | Streptomyces phaeoluteigriseus |
| A0A1Y5WUX7 | Kibdelosporangium aridum | A0A0M8YED7 | Saccharothrix sp. NRRL B-16348 |
| A0A428Z0R7 | Kibdelosporangium aridum | A0A397R2P1 | Streptomyces sp. 3211.1 |
| A0A1W2ACZ1 | Kibdelosporangium aridum | A0A243M3A1 | Bacillus thuringiensis serovar zhaodongensis |
| A0A4S4FTB6 | Glaciibacter sp. YIM 131861 | A0A0N0AQZ3 | Saccharothrix sp. NRRL B-16348 |
| A0A3B0AG24 | Streptomyces klenkii | A0A0N0TT97 | Saccharothrix sp. NRRL B-16348 |
| A0A3B0BBL9 | Streptomyces klenkii | A0A402AXW2 | Dictyobacter kobayashii |
| A0A1I6XDP9 | Actinopolyspora righensis | A0A6G7ZG30 | Sphingomonas sp. HDW15A |
| A0A7K2Y9D9 | Streptomyces sp. SID1046 | A0A1Z2L4G7 | Streptomyces albireticuli |
| A0A6I5C4S5 | Streptomyces sp. SID625 | A0A239GP97 | Azospirillum sp. RU38E |
| A0A6I5CD02 | Streptomyces sp. SID625 | A0A0L0KSB0 | Streptomyces stelliscabiei |
| A0A233SK10 | Streptomyces sp. 2R | A0A366M5Q1 | Sphaerisporangium sp. LHW63015 |
| A0A542JEE2 | Streptomyces sp. SLBN-31 | A0A7K2RNK1 | Streptomyces sp. SID8367 |
| A0A1A2SAA1 | Mycobacterium colombiense | A0A1B5CTB7 | Pseudomonas sp. 24 E 1 |
| A0AIR0UNA8 | Mycobacterium colombiense | A0A6G7SVL8 | Streptomyces sp. ID38640 |
| A0A2K8R627 | Streptomyces sp. M56 | A0A229R7X3 | Amycolatopsis alba DSM 44262 |
| A0A1A3RM31 | Mycobacterium colombiense | A0A371XWF2 | Streptomyces sp. AcE210 |
| A0A540VX96 | Kitasatospora sp. MMS16-CNU292 | A0A160IJ33 | Fictibacillus phosphorivorans |
| A0A495W1D2 | Saccharothrix australiensis | A0PNP6 | Mycobacterium ulcerans (strain Agy99) |
| A0A1A2RW84 | Mycobacterium colombiense | A0A2T7M9S9 | Streptomyces sp. CS014 |
| A0A329KVW6 | Mycobacterium colombiense | A0A0N1GNK3 | Actinobacteria bacterium OV320 |
| A0A6N8GFG5 | Streptomonospora sp. PA3 | A0A5C8Q625 | Streptomyces sp. IB2014 016-6 |
| A0A1A3SEC9 | Mycobacterium colombiense | A0A101J861 | Streptomyces regalis |
| A0A1A0VTF8 | Mycobacterium colombiense | A0A246HKI2 | Stenotrophomonas maltophilia |
| A0A329LNW6 | Mycobacterium colombiense | A0A4Q7ZTC1 | Krasilnikovia cinnamomea |
| A0A2N8BUB9 | Pseudomonas sp. FW306-2-11AA | A0A0X3VHA2 | Streptomyces regalis |
| A0A1H1L6Q6 | Pseudomonas sp. bs2935 | A0A2H1I3V5 | Brevibacterium antiquum |
| A0A4V6XB37 | Kribbella sp. NEAU-THZ 27 | A0A161U4B6 | Paenibacillus elgii |
| A0A1A3RNS5 | Mycobacterium colombiense | A0A495HSP0 | Acidovorax sp. 94 |
| A0A1A2SRQ5 | Mycobacterium colombiense | A0A4R8UXC0 | Cryobacterium sp. HLT2-23 |
| A0A4U3LN86 | Kribbella sp. NEAU-THZ 27 | W0F783 | Niabella soli DSM 19437 |
| V6UM19 | Streptomyces sp. HCCB10043 | A0A1H5QDF3 | Amycolatopsis pretoriensis |
| A0A445N4S6 | Streptomyces netropsis | A0A1G8GI82 | Sinosporangium album |
| A0A1V4E239 | Microbispora sp. GKU 823 | A0A3M2M222 | Streptomyces triticirhizae |
| A0A3G6UNS7 | Bacillus sp. FJAT-42376 | A0A1A2ZS62 | Mycobacterium sp. E787 |
| R8IR35 | Bacillus cereus K-5975c | A0A7I7SC82 | Mycolicibacillus koreensis |
| W9FP31 | Streptomyces filamentosus NRRL 11379 | A0A014N241 | Streptomyces sp. PRh5 |
| A0A222SYQ6 | Streptomyces sp. 11-1-2 | A0A0P7CCM2 | Frankia sp. R43 |
| A0A7L4Y429 | Streptomyces sp. QHH-9511 | V7JJY0 | Mycobacterium avium 10-5581 |
| A0A231PS24 | Streptomyces sp. XY006 | A0A5C8JQF6 | Nonomuraea sp. C10 |
| A0A2M9JQ97 | Streptomyces sp. CB01635 | A0A2T9JU73 | Caulobacter radicis |
| A0A231PE89 | Streptomyces sp. XY006 | A0A0Q8YGY3 | Pseudomonas sp. Root9 |
| A0A543N8Y2 | Haloactinospora alba | A0A2X1T653 | Mycobacterium xenopi |
| A0A6I3L667 | Nocardia sp. CT2-14 | A0A6G2TFN0 | Streptomyces sp. SID8366 |
| A0A3N1P921 | Streptomyces sp. PanSC19 | A0A1X7GFL6 | Sphingomonas indica |
| V6U9P7 | Streptomyces sp. HCCB10043 | A0A4Q7VY37 | Kribbella soli |
| A0A0C2AIL3 | Streptomyces sp. AcH 505 | A0A2S5TYE9 | Kaistia algarum |
| A0A429SUD6 | Streptomyces sp. WAC05292 | A0A223QK01 | Nocardiopsis dassonvillei |
| A0A1H8NFU8 | Streptomyces rubidus | A0A1X2BEI3 | Mycobacterium riyadhense |
| A0A1G6LCS1 | Streptomyces prasinopilosus | A0A1X2CDH2 | Mycobacterium riyadhense |
| A0A3D9WJ96 | Streptomyces sp. Ag82_O1-9 | A0A1H5R0I3 | Amycolatopsis pretoriensis |
| A0A0C2B296 | Streptomyces sp. AcH 505 | A0A559UVI1 | Streptomyces sp. CNZ289 |
| V6UAX9 | Streptomyces sp. HCCB10043 | A0A557XKF8 | Mycobacterium helveticum |
| A0A2G7F0L0 | Streptomyces sp. 70 | A0A2V2PW95 | Streptomyces sp. ZEA17I |
| A0A1G6LIH0 | Streptomyces prasinopilosus | A0A386ZGV1 | Nocardia yunnanensis |
| V6UM73 | Streptomyces sp. HCCB 10043 | A0A2L2Q5P2 | Streptomyces sp. WAC00288 |
| A0A6I3L6H1 | Nocardia sp. CT2-14 | A0A1N6HFD6 | Burkholderia sp. GAS332 |
| A0A1J4P0W0 | Streptomyces mangrovisoli | J8FK19 | Bacillus cereus MC67 |
| A0A0N0ADD8 | Streptomyces sp. NRRL WC-3723 | A0A2A3A3K3 | Mycobacterium avium |
| A0A1B6AE70 | Streptomyces sp. NBRC 110611 | A0A559URZ5 | Streptomyces sp. CNZ289 |
| M2ZJT6 | Amycolatopsis decaplanina DSM 44594 | A0A1I5VWG5 | Variovorax sp. 770b2 |
| A0A7K1UVM4 | Nocardia sp. ET3-3 | A0A2V3XXY9 | Hungatella effluvii |
| A0A0F3L061 | Luteibacter yeojuensis | A0A514JQS3 | Streptomyces asterosporus |
| A0A0F6L9C7 | Burkholderia pseudomallei MSHR4000 | A0A1E8CQL8 | Humibacillus sp. DSM 29435 |
| A0A1Q4ZEZ9 | Streptomyces sp. TSRI0281 | A0A1XIN7H7 | Streptomyces sp. CB03238 |
| A0A7I7KEB8 | Mycobacterium simiae | D9WNW9 | Streptomyces himastatinicus ATCC 53653 |
| A0A7I7KLK1 | Mycobacterium simiae | A0A1X1NCC2 | Streptomyces sp. CB03238 |
| A0A5M6CKA9 | Taibaiella sp. KVB11 | J8JYF0 | Bacillus cereus VD107 |
| A0A1S1NBH1 | Pseudoalteromonas byunsanensis | A0A4R1MFI1 | Paraburkholderia sp. BL912N2 |
| A0A4R4SJK2 | Actinomadura sp. GC306 | A0A6H9JYW3 | Bacillus sp. AY2-1 |
| A0A260ICH0 | Rhodococcus sp. 05-2256-B3 | A0A6H0CMK8 | Streptomyces sp. DSM 40868 |
| A0A7K1UPM9 | Nocardia sp. ET3-3 | A0A257BLY2 | Pseudomonas sp. PGPPP2 |
| A0A656TPB8 | Streptomyces sp. NRRL S-444 | A0A328GYP4 | Mycobacterium tuberculosis variant pinnipedii |
| A0A0A8ERS1 | Streptomyces sp. 769 | D9WUA0 | Streptomyces himastatinicus ATCC 53653 |
| A0A2S9QVG5 | Mycobacterium shigaense | A0A356TB51 | Myxococcales bacterium |
| A0A3S4D3M6 | Devosia sp. M6-77 | A0A1E5P917 | Streptomyces agglomeratus |
| A0A1X0YGE3 | Mycobacterium simiae | A0A4Y8UWR0 | Cellulomonas sp. HD19AZ1 |
| A0A0T9R4K0 | Yersinia similis | A0A286EZT9 | Streptomyces sp. 1222.2 |
| A0A1V2QV88 | Saccharothrix sp. ALI-22-I | A0A127EFB1 | Clostridium perfringens |
| A0A1V2QWH7 | Saccharothrix sp. ALI-22-I | A0A1B9EMS2 | Streptomyces sp. PTY08712 |
| A0A7I7NQI4 | Mycobacterium lacus | A0A6M1X2N6 | Clostridium perfringens |
| A0A1Q8CDQ0 | Actinophytocola xanthii | D9WKR0 | Streptomyces himastatinicus ATCC 53653 |
| A0A544ZAS9 | Microbispora sp. KK1-11 | A0A2N2ATU5 | Firmicutes bacterium HGW-Firmicutes-7 |
| A0A2G7ANQ4 | Streptomyces sp. 94 | A0A0H3B7W1 | Yersinia pseudotuberculosis serotype O:3 (strain YPIII) |
| A0A398C6X8 | Simplicispira hankyongi | A0A2N0GPL5 | Streptomyces sp. Ag109_G2-1 |
| A0AIY0KU31 | Pseudomonas sp. M30-35 | A0A2A3JBM9 | Streptomyces sp. Ag82_O1-15 |
| A0A4R4THK6 | Streptomyces hainanensis | A0A429G4M7 | Actinomadura sp. WAC 06369 |
| A0A4R4TR65 | Streptomyces hainanensis | A0A1W9Y8G5 | Mycobacterium alsense |
| A0A2Z5YAS3 | Mycobacterium marinum | A0A6M4WQW6 | Streptomyces cacaoi subsp. asoensis |
| J7L7L1 | Nocardiopsis alba (strain ATCC BAA-2165/BE74) | A0A1X2L8G9 | Mycobacterium vulneris |
| A0A4R4Q3R0 | Kribbella albertanoniae | A0A1V2KJ96 | Frankia sp. CcI49 |
| A0A2E0XWX6 | Phycisphaerae bacterium | A0A1A2K1V8 | Mycobacterium sp. E1214 |
| A0A5C6JVS9 | Streptomyces misionensis | A0A401VTY1 | Streptomyces rimosus subsp. paromomycinus |
| A0A3N1QKT1 | Frondihabitans sp. PhB188 | A0A2G9E0B2 | Streptomyces sp. JV178 |
| A0A1X1XU23 | Mycobacterium lacus | D2BES9 | Streptosporangium roseum (strain ATCC 12428/DSM 43021/JCM 3005/NI 9100) |
| A0A2U1ELJ6 | Promicromonospora sukumoe | D7WV18 | Lysinibacillus fusiformis ZC1 |
| A0A3N1KY93 | Streptomyces sp. 844.5 | A0A7G8P8J1 | Mycolicibacterium fluoranthenivorans |
| A0A4S3FS82 | Streptomyces sp. A1499 | A0A384HVA3 | Streptomyces sp. AC1-42W |
| A0A1R0M1K6 | Streptomyces sp. IMTB 2501 | A0A421B9E4 | Actinokineospora cianjurensis |
| A0A5N0EFZ7 | Nocardia colli | A0A6B2Z418 | Streptomyces sp. SID 14515 |
| A0A6N4UU35 | Mycolicibacterium alvei | A0A421BBY6 | Actinokineospora cianjurensis |
| A0A2S1IEM3 | Streptomyces nigra | A0A3D1FHM6 | Lachnoclostridium sp. |
| A0A7K2FQV8 | Streptomyces sp. SID4985 | A0A2A3J929 | Streptomyces sp. Ag82_O1-15 |
| A0A2T0M3Q4 | Prauserella shujinwangii | A0A7K0P2X4 | Actinobacteria bacterium |
| A0A2A2YWX0 | Streptomyces sp. SA15 | A0A7H8MV69 | Streptomyces fulvissimus |
| A0A1A0LAF4 | Mycobacterium sp. 1554424.7 | A0A1A2LZ41 | Mycobacterium sp. E3251 |
| A0A656RLI7 | Streptomyces griseorubens | A0A7H8MTS8 | Streptomyces fulvissimus |
| A0A2S1IDA9 | Streptomyces nigra | A0A538MUQ9 | Actinobacteria bacterium |
| A0A419HJZ3 | Amycolatopsis panacis | A0A0M9I899 | Alcaligenes xy losoxydans xylosoxydans |
| A0A1N5U5I2 | Micromonospora cremea | A0A5S3YRB2 | Pseudoalteromonas phenolica |
| A0A3R7GML0 | Paraburkholderia sediminicola | A0A5R9Q468 | Pseudoalteromonas phenolica |
| A0A7H8LTV4 | Actinomadura sp. NAK00032 | G2GAN0 | Streptomyces zinciresistens K42 |
| A0A560HQI8 | Nitrospirillum amazonense | A0A537Z4Y1 | Actinobacteria bacterium |
| A0A560F4U4 | Nitrospirillum amazonense | A0A538APH8 | Actinobacteria bacterium |
| A0A1V0TV83 | Streptomyces gilvosporeus | A0A4R1D1V9 | Streptomyces sp. KM273126 |
| A0A2T4NI37 | Streptomyces sp. A244 | A0A2Z5TCY0 | Mycobacterium pseudoshottsii JCM 15466 |
| A0A7L4Z1P6 | Streptomyces sp. HF10 | A0A100ICH2 | Mycobacterium pseudoshottsii JCM 15466 |
| G0Q026 | Streptomyces sp. ACT-1 | A0A1Q4HYE3 | Mycobacterium paraffinicum |
| A0A4Y8M0B6 | Frankia sp. B2 | A0A3D9SRM9 | Thermomonospora umbrina |
| A0A1D7VL31 | Streptomyces lydicus | A0A0S2K2Z9 | Pseudoalteromonas phenolica |
| A0A2M9KVH3 | Kitasatospora sp. CB02891 | A0A1Q4HQD2 | Mycobacterium paraffinicum |
| A0A2M9L212 | Kitasatospora sp. CB02891 | A0A4Q7IT42 | Pseudoalteromonas phenolica |
| A0A3N1H3T1 | Saccharothrix texasensis | A0A3D9T210 | Thermomonospora umbrina |
| D6K9D2 | Streptomyces sp. e14 | A0A5M7CBM8 | Pseudomonas chlororaphis |
| D6K5Q8 | Streptomyces sp. e14 | A0A2T9JSP1 | Caulobacter sp. 774 |
| D9T293 | Micromonospora aurantiaca (strain ATCC 27029/DSM 43813/BCRC 12538/CBS | A0A0X8NV74 | Alcaligenes xylosoxydans xylosoxydans |
| | 129.76/JCM 10878/NBRC 16125/NRRL B-16091/INA 9442) | | |
| B1KTK5 | Clostridium botulinum (strain Loch Maree/Type A3) | A0A6L6XAM4 | Streptomyces sp. p1417 |
| B1KTN9 | Clostridium botulinum (strain Loch Maree/Type A3) | G8NP97 | Granulicella mallensis (strain ATCC BAA-1857/DSM 23137/MP5ACTX8) |
| A0A0T6LYH0 | Streptomyces vitaminophilus | A0A7I8DPT3 | Anaerocolumna sp. CTTW |
| A0A7C4ZP27 | Anaerolineae bacterium | A0A1B9CUP1 | Mycobacterium malmoense |
| A0A6N7JJI8 | Niveispirillum sp. SYP-B3756 | A0A1S2WCN3 | Mycobacterium malmoense |
| A0A498CLS4 | Stenotrophomonas rhizophila | G8NWH4 | Granulicella mallensis (strain ATCC BAA-1857/DSM 23137/MP5ACTX8) |
| A0A2N3Z6B0 | Micromonospora sp. CNZ309 | A0A7H8JRZ6 | Streptomyces sp. NA04227 |
| A0A2M8M1V8 | Streptomyces carminius | H0B9X9 | Streptomyces sp. W007 |
| A0A2E0QXP2 | Verrucomicrobiales bacterium | A0A0Q6MMH5 | Rhizobacter sp. Root29 |
| A0A4P5XA20 | Planctomycetes bacterium | A0A6L6X404 | Streptomyces sp. p1417 |
| A0A542HPV8 | Streptomyces sp. SLBN-115 | A0A7H8JU47 | Streptomyces sp. NA04227 |
| A0A0F0DKA2 | Burkholderiaceae bacterium 26 | A0A7H8JU83 | Streptomyces sp. NA04227 |
| A0A362XYJ9 | Nitrosospira sp. Nsp22 | D3PVN7 | Stackebrandtia nassauensis (strain DSM 44728/CIP 108903/NRRL B-16338/NBRC |
| | | | 102104/LLR-40K-21) |
| A0A372ZN49 | Kitasatospora xanthocidica | A0A7H8JV64 | Streptomyces sp. NA04227 |
| A0A0D0HPC2 | Vibrio harveyi | D3Q9Z5 | Stackebrandtia nassauensis (strain DSM 44728/CIP 108903/NRRL B-16338/NBRC |
| | | | 102104/LLR-40K-21) |
| A0A352XK00 | Verrucomicrobiales bacterium | A0A167J9Q1 | Pseudoalteromonas luteoviolacea CPMOR-1 |
| A0A246RFT3 | Micromonospora wenchangensis | A0A132CAK7 | Burkholderia sp. ABCPW 14 |
| A0A6G3T6L7 | Streptomyces rubrogriseus | A0A5R9E088 | Streptomyces marianii |
| A0A259V207 | Sporomusa acidovorans DSM 3132 | L1KSH5 | Streptomyces ipomoeae 91-03 |
| A0A1H0RXB8 | Streptomyces sp. cf386 | K0K1H7 | Saccharothrix espanaensis (strain ATCC 51144/DSM 44229/JCM 9112/NBRC |
| | | | 15066/NRRL 15764) |
| A0A7K0CG81 | Streptomyces sp. RB5 | A0A0U0W9B9 | Mycobacterium bohemicum DSM 44277 |
| A0A658U1P7 | Mycobacteroides abscessus subsp. abscessus | A0A6I5BHE3 | Streptomyces sp. SID4941 |
| A0A1S2JDK0 | Curtobacterium sp. MMLR14 014 | A0A5E9G0Y8 | Cryobacterium flavum |
| A0A327UDT2 | Streptomyces sp. PsTaAH-130 | A0A4R5AA65 | Actinomadura rubrisoli |
| A0A1H0TDS3 | Streptomyces sp. cf386 | A0A5P2DJ63 | Streptomyces venezuelae |
| A0A0B2YIF5 | Mycolicibacterium setense | A0A0L1MAJ5 | Bacillus megaterium |
| A0A7K0CNT9 | Streptomyces sp. RB5 | A0A543ATT0 | Stackebrandtia endophytica |
| A0A421AV03 | Promicromonospora sp. YR516 | A0A5P2DW61 | Streptomyces venezuelae |
| A0A0N1FV32 | Actinobacteria bacterium OV450 | A0A613ZUM4 | Mycolicibacterium sp. CBMA 226 |
| A0ALA0U1N4 | Mycobacterium sp. 852014-50255_SCH5639931 | A0A4R8V423 | Cryobacterium flavum |
| A0A2G7DPC5 | Streptomyces sp. 1 | A0A3E0HEI7 | Kutzneria buriramensis |
| A0A0M0L4H2 | Priestia koreensis | A0A166XZW3 | Pseudoalteromonas luteoviolacea NCIMB 1942 |
| A0A6G3TCI9 | Streptomyces rubrogriseus | A0A5P2CV56 | Streptomyces venezuelae |
| A0A2T0MVU4 | Nonomuraea fuscirosea | A0A3D9VGH8 | Thermasporomyces composti |
| A0A6G3T8F6 | Streptomyces rubrogriseus | A0A5P2D313 | Streptomyces venezuelae |
| A0A423E3X2 | Pseudomonas protegens | A0A5P2BF58 | Streptomyces venezuelae |
| A0A1G4XFC9 | Nitrosospira sp. Nsp1 | A0A6G3BEU2 | Streptomyces sp. SID5594 |
| D6AAD2 | Streptomyces viridosporus (strain ATCC 14672/DSM 40746/JCM 4963/KCTC | A0A5P2BDK2 | Streptomyces venezuelae |
| | 9882/NRRL B-12104/FH1290) | | |
| A0A5N8VLC8 | Streptomyces adustus | A0A1W2BZK4 | Moheibacter sediminis |
| A0A242XQE3 | Bacillus thuringiensis serovar guiyangiensis | A0A6G7XKB0 | Nocardioides sp. HDW12B |
| A0A514WU25 | Bdellovibrio sp. NC01 | A0A7I7Z535 | Mycobacterium heidelbergense |
| A0A1N6MQF5 | Xenorhabdus innexi | A0A4Y8MFM2 | Streptomyces sp. ICN441 |
| A0A5N8VNG3 | Streptomyces adustus | A0A6I5H5D2 | Streptomyces sp. SID89 |
| A0A5N8VII4 | Streptomyces adustus | A0A3M8SXX8 | Streptomyces sp. NEAU-LD23 |
| A0A1Y2N182 | Streptomyces platensis | A0A6I2FZF5 | Nocardia sp. SYP-A9097 |
| A0A0S4QUH5 | Frankia irregularis | A0A0Q0TFG0 | Acidovorax sp. SD340 |
| A0A5N8VI95 | Streptomyces adustus | A0A6P1Q5Z9 | Mixta intestinalis |
| A0A429GWF9 | Streptomyces sp. WAC 05379 | A0A6S7ARZ6 | Achromobacter kerstersii |
| A0A233SIB1 | Streptomyces diastatochromogenes | A0A5R9EDU0 | Streptomyces marianii |
| A0A4Q6II27 | Streptomyces sp. F001 | A0A7I7QHZ5 | Mycobacterium stomatepiae |
| A0A3C1NDJ3 | Rhizobiales bacterium | A0A060ZPF2 | Streptomyces iranensis |
| A0A402BJ61 | Dictyobacter alpinus | A0A2R4K304 | Streptomyces sp. |
| A0A5N5W9F5 | Streptomyces mobaraensis | D1H012 | Streptomyces ravidus |
| A0A1R0KTV5 | Amycolatopsis coloradensis | A0A060ZD91 | Streptomyces iranensis |
| A0A3D0S6J8 | Nocardioides sp. | A0A1X9NW33 | Streptomyces sp. |
| A0A5N8X5M8 | Streptomyces acidicola | A0A1I9S3Q0 | Streptomyces sp. KIB-H033 |
| A0A1X1ZZ62 | Mycobacterium palustre | A0A6B2NNI7 | Ruegeria sp. PrR005 |
| A0A1A0WVS2 | Mycobacterium sp. 852013-50091_SCH5140682 | A0A3Q8VN90 | Streptomyces sp. |
| A0A2V2ANU6 | Streptomyces sp. CG 926 | A0A6B2NX87 | Ruegeria sp. PrR005 |
| A0A5N8WQT9 | Streptomyces acidicola | A0A6B1NB49 | Streptomyces sp. SID69 |
| A0A1R0KXR0 | Amycolatopsis coloradensis | A0A1W5VLE4 | Streptomyces sp. |
| A0A0R3IAH7 | Mycobacteroides sp. H001 | A0A1M4EAQ4 | Nonomuraea gerenzanensis |
| A0A1H9DYL4 | Lentzea albida | Q4JHQ9 | Saccharopolyspora pogona |
| A0A1X1Z8M3 | Mycobacterium palustre | A0A6G4CM00 | Clostridium botulinum |
| A0A2A5FRU3 | Rhizobiales bacterium | B5SP91 | Actinomyces sp. Lu 9419 |
| A0A2V2B361 | Streptomyces sp. CG 926 | A0A6G4DGE3 | Clostridium botulinum |
| A0A5N5VZ19 | Streptomyces mobaraensis | A0A0S4TQY4 | Ralstonia solanacearum |
| A0A4V1T4F4 | Rhizobiales bacterium | A0A653EHK8 | Mycobacterium kansasii |
| A0A1V2PCE9 | Actinosynnema sp. ALI-1.44 | B3TMP4 | Actinomadura kijaniata |
| A0A1H9KS63 | Lentzea albida | A0A1E3S1T3 | Mycobacterium intermedium |
| G8TUV2 | Sulfobacillus acidophilus (strain ATCC 700253/DSM 10332/NAL) | A0A222VLR6 | Prauserella marina |
| A0A0N0H3Y6 | Streptomyces chattanoogensis | A0A5P2X0Z6 | Streptomyces spectabilis |
| A0A6N9W6Q8 | Actinospica acidiphila | A0A5R9LQP6 | Streptomyces sp. So13.3 |
| A0A536GXG1 | Chloroflexi bacterium | A0A5P2XDJ8 | Streptomyces spectabilis |
| A0A5J6EWG6 | Streptomyces galilaeus | A0A1X2BZK1 | Mycobacterium saskatchewanense |
| A0A5J6EKB6 | Streptomyces galilaeus | A0A1X2C9U2 | Mycobacterium saskatchewanense |
| A0A1H9JQW9 | Lentzea xinjiangensis | A0A1E3SC10 | Mycobacterium intermedium |
| A0A535ZZC1 | Chloroflexi bacterium | A0A2G8B8K4 | Mycobacterium heckeshornense |
| A0A536B297 | Chloroflexi bacterium | A0A5P2X464 | Streptomyces spectabilis |
| A0A535IKB7 | Chloroflexi bacterium | A0A1R3XU80 | Mycobacterium bovis (strain ATCC BAA-935/AF2122/97) |
| A0A2S9PUU5 | Streptomyces sp. ST5x | A0A6B4J5N9 | Clostridium botulinum |
| A0A535QN89 | Chloroflexi bacterium | C0Z480 | Streptomyces violaceoruber |
| A0A536HPG2 | Chloroflexi bacterium | A0A6B4GQ66 | Clostridium botulinum |
| A0A7I7TMU7 | Mycobacterium paraintracellulare | A0A6G4F979 | Clostridium botulinum |
| A0A5J6F241 | Streptomyces galilaeus | A0A0S4V440 | Ralstonia solanacearum |
| A0A535AN77 | Chloroflexi bacterium | A0A6G4EDX2 | Clostridium botulinum |
| A0A535XJL0 | Chloroflexi bacterium | A0A6N3I5D0 | Hungatella hathewayi |
| A0A7K2IJK4 | Streptomyces sp. SID6137 | A0A6G4R043 | Caulobacter sp. 602-2 |
| A0A520R673 | Sandaracinaceae bacterium | A0A6B4N706 | Clostridium botulinum |
| A0A535C592 | Chloroflexi bacterium | A0A6B4G4M6 | Clostridium botulinum |
| A0A1V2Q035 | Actinosynnema sp. ALI-1.44 | A0A6G2P7K6 | Streptomyces sp. SID5466 |
| A1TCC5 | Mycolicibacterium vanbaalenii (strain DSM 7251/JCM | A0A6G4EDU2 | Clostridium botulinum |
| | 13017/BCRC 16820/KCTC 9966/NRRL B-24157/PYR-1) | | |
| A0A4R5C2C9 | Actinomadura darangshiensis | C4NYM5 | Micromonospora sp. Tu 6368 |
| A0A658VQ28 | Mycobacteroides abscessus subsp. massiliense | A0A6B4I7Z0 | Clostridium botulinum |
| A0A535EC47 | Chloroflexi bacterium | A0A6B4V5M5 | Clostridium botulinum |
| A0A535GMU1 | Chloroflexi bacterium | A0A1C6CAD3 | uncultured Clostridium sp. |
| A0A197SM72 | Streptomyces sp. ERV7 | A0A1S5Y1S6 | uncultured bacterium |
| A0A7K2G6P5 | Streptomyces sp. SID4950 | A0A653EEG8 | Mycobacterium riyadhense |
| A0A4U0NXT8 | Streptomyces piniterrae | A0A6M1VXW3 | Clostridium perfringens |
| A0A2G7HK14 | Clostridium combesii | A0A1L9DU80 | Streptomyces viridifaciens |
| A0A535EZI1 | Chloroflexi bacterium | A0A3G5KC10 | Yersinia pseudotuberculosis |
| A0A2T0IAA3 | Pseudomonas fluorescens | A0A2B9BVC4 | Bacillus cereus |
| A0A109KJN2 | Pseudomonas fluorescens | A0A5P2GL40 | Streptomyces seoulensis |
| A0A2N1EDP2 | Pseudomonas fluorescens | A0A2M9GUJ0 | Achromobacter ruhlandii |
| A0A2N0K2W5 | Kitasatospora sp. OK780 | A0A0J6ZMN7 | Bacillus cereus |
| A0A1H9S2L7 | Actinokineospora terrae | I2N599 | Streptomyces tsukubensis (strain DSM 42081/NBRC 108919/NRRL 18488/9993) |
| A0A5J4KRS0 | Dictyobacter vulcani | A0A1X1TAW1 | Mycobacterium conspicuum |
| A0A429ADA0 | Streptomyces sp. WAC 01420 | A0A4D4MXG2 | Streptomyces avermitilis |
| A0A7D7FMJ9 | Klebsiella aerogenes | I2N1B5 | Streptomyces tsukubensis (strain DSM 42081/NBRC 108919/NRRL 18488/9993) |
| A0A1W9ZP95 | Mycobacterium angelicum | A0A2Z5Y7R2 | Mycobacterium marinum |
| A0A401MHE6 | Streptomyces sp. NL15-2K | A0A1X1T3W7 | Mycobacterium conspicuum |
| L8PDF4 | Streptomyces viridochromogenes Tue57 | A0AIX1XZH4 | Mycobacterium kubicae |
| A0A1A2Z783 | Mycobacterium kyorinense | A0A142I755 | Streptomyces sp. FXJ1.172 |
| A0A0Q6L5K2 | Bacillus sp. Leaf406 | A0A1S8NZB0 | Clostridium beijerinckii |
| J1RQR1 | Streptomyces auratus AGR0001 | A0A0B8N335 | Nocardia seriolae |
| A0A543VMC0 | Streptomyces cavourensis | D7BRT5 | Streptomyces bingchenggensis (strain BCW-1) |
| N0CN45 | Streptomyces fulvissimus DSM 40593 | A0A4R5XMA7 | Jeotgalibacillus sp. S-D1 |
| A0A1S8XP43 | Micromonospora sp. Rc5 | A0A6G2BJJ6 | Streptomyces taklimakanensis |
| A0A6B2YN38 | Streptomyces sp. SID 14446 | D7C4Z8 | Streptomyces bingchenggensis (strain BCW-1) |
| A0A4R7FHU5 | Amnibacterium kyonggiense | D7BTB8 | Streptomyces bingchenggensis (strain BCW-1) |
| A0A429EAU2 | Streptomyces sp. WAC 04229 | A0A543NN12 | Haloactinospora alba |
| N0CVN7 | Streptomyces fulvissimus DSM 40593 | A0A4R1SHD0 | Curtobacterium sp. PhB128 |
| A0A2K4Y6J6 | Mycobacterium ahvazicum | A0A7L4Y8I5 | Streptomyces sp. QHH-9511 |
| A0A2L0RPJ0 | Pseudomonas orientalis | A0A3N1QAC0 | Streptomyces sp. PanSC19 |
| A0A1C4Y858 | Micromonospora purpureochromogenes | A0A0C2JJ03 | Streptomonospora alba |
| B1VRN7 | Streptomyces griseus subsp. griseus (strain JCM 4626/NBRC 13350) | A0A2G7A566 | Streptomyces sp. 1121.2 |
| B5GRN9 | Streptomyces clavuligerus | A0A1C4KGQ9 | Streptomyces sp. DvalAA-19 |
| A0A4Q7KL53 | Herbihabitans rhizosphaerae | A0A2G7A0A2 | Streptomyces sp. 1121.2 |
| A0A0L8NIL5 | Streptomyces antibioticus | A0A1M6G7S6 | Clostridium cavendishii DSM 21758 |
| A0A0L8NFP5 | Streptomyces antibioticus | A0A1I7LSZ8 | Methylobacterium sp. 174MFSha1.1 |
| A0A2K4YE25 | Mycobacterium ahvazicum | A0A537QH64 | Alphaproteobacteria bacterium |
| A0A4R9EG40 | Streptomyces sp. MZ04 | A0A6G2B7N1 | Streptomyces taklimakanensis |
| C7Q3B2 | Catenulispora acidiphila (strain DSM 44928/JCM 14897/NBRC 102108/NRRL B-24433/ | A0A117RDT0 | Streptomyces griseoruber |
| | ID139908) | | |
| A0A1Z1WDM2 | Streptomyces alboflavus | A0A6G2B784 | Streptomyces taklimakanensis |
| A0A385ZE33 | Streptomyces griseorubiginosus | A0A6G2BJN7 | Streptomyces taklimakanensis |
| B1V0U2 | Clostridium perfringens D str. JGS1721 | A0A2Z4J1H0 | Streptomyces cadmiisoli |
| A0A0E2WJ00 | Mycobacterium avium subsp. hominissuis A5 | A0A2S7UIA8 | Vibrio jasicida |
| A0A239BHH3 | Actinomadura mexicana | A0A1Q8UCJ7 | Streptomyces sp. MNU77 |
| A0A6I2FTD5 | Nocardia sp. SYP-A9097 | A0A222T963 | Streptomyces sp. 11-1-2 |
| A0A1X0DD00 | Mycobacterium heidelbergense | A0A2G7EMS5 | Streptomyces sp. 70 |
| A0A3M8X2E1 | Streptomyces sp. NEAU-LD23 | A0A1G6LCB7 | Streptomyces prasinopilosus |
| A0A2R3H967 | Pseudomonas sp. MYb193 | A0A1C4LF68 | Streptomyces sp. DvalAA-19 |
| A0A6I2G6U0 | Nocardia sp. SYP-A9097 | A0A4S2VNY2 | Streptomyces sp. A0592 |
| A0A4Q1S958 | Acidipila dinghuensis | A0A0NIK690 | Streptomyces sp. NRRL S-4 |
| A7G9Z1 | Clostridium botulinum (strain Langeland/NCTC 10281/Type F) | A0A614NBG8 | Streptomyces sp. BA2 |
| A7GA26 | Clostridium botulinum (strain Langeland/NCTC 10281/Type F) | A0AIH8YK50 | Amycolatopsis saalfeldensis |
| A0A1X0DWS2 | Mycobacterium heidelbergense | A0A1Q5AQ59 | Streptomyces sp. MJM1172 |
| A0A7I7Q8Z6 | Mycobacterium stomatepiae | A0A2R4WED0 | Methylobacterium currus |
| A0A1H0WK77 | Actinokineospora alba | A0A640TIT3 | Streptomyces libani subsp. libani |
| A0A429J3P6 | Streptomyces sp. WAC 06725 | A0A5A9G5J4 | Azospirillum lipoferum |
| A0A3L7A3V0 | Mycetocola tolaasinivorans | A0A0Q8VEZ2 | Nocardioides sp. Root224 |
| A0A1S1JBY0 | Flavobacterium spartansii | A0A101TLI7 | Streptomyces caeruleatus |
| A0A1W9ZFB6 | Mycobacterium angelicum | A0A7K2YSP1 | Streptomyces sp. SID3343 |
| D9R8K0 | Clostridium saccharolyticum (strain ATCC 35040/DSM 2544/NRCC 2533/WM1) | A0A1H8YHP8 | Amycolatopsis saalfeldensis |
| A0A3L7A9L3 | Mycetocola tolaasinivorans | A0A367HVB0 | Streptomyces sp. SDr-06 |
| A0A2S4ZVZ8 | Streptomyces sp. Ru62 | A0A3E2NBT1 | Clostridium indicum |
| A0A101S206 | Streptomyces griseorubiginosus | A0A367HP37 | Streptomyces sp. SDr-06 |
| A0A1H8YJM5 | Amycolatopsis saalfeldensis | A0A7K2YHV5 | Streptomyces sp. SID3343 |
| A0A6I5GIN8 | Streptomyces sp. SID89 | A0A367HLR5 | Streptomyces sp. SDr-06 |
| B5HT29 | Streptomyces sviceus ATCC 29083 | A0A385ZPS0 | Streptomyces griseorubiginosus |
| A0A101NA04 | Streptomyces cellostaticus | A0A1H2BY98 | Streptomyces sp. TLI_053 |
| A0A419XEA5 | Catellatospora citrea | A0A3N1N047 | Streptomyces griseorubiginosus |
| A0A717ZDU8 | Mycobacterium heidelbergense | A0A352A771 | Clostridiales bacterium |
| A0A3S0HDC9 | Variovorax sp. 679 | A0A640TUG6 | Streptomyces libani subsp. libani |
| A0A2S4YT83 | Streptomyces sp. Ru71 | A0A6G0FAD4 | Streptomyces sp. SS1-1 |
| F2JSW0 | Cellulosilyticum lentocellum (strain ATCC 49066/DSM 5427/NCIMB 11756/RHM5) | A0A4Q3D3S4 | Sphingobacteriales bacterium |
| L1KS92 | Streptomyces ipomoeae 91-03 | A0A1H2DI87 | Streptomyces sp. TLI_053 |
| A0A7K3GTV0 | Streptomyces sp. SID5789 | A0A640TSE5 | Streptomyces libani subsp. libani |
| A0A7D6VQ74 | Clostridium intestinale | A0A7C6LYF7 | Clostridiales bacterium |
| A0A429CTQ4 | Amycolatopsis sp. WAC 04169 | A0A6G0F8Y5 | Streptomyces sp. SS1-1 |
| A0A497WA44 | Streptomyces sp. 74 | A0A1Q5ANC4 | Streptomyces sp. MJM1172 |
| A0A1M7UTN5 | Bradyrhizobium erythrophlei | A0A124HW49 | Streptomyces griseorubiginosus |
| E8V420 | Terriglobus saanensis (strain ATCC BAA-1853/DSM 23119/SP1PR4) | A0A1H8YLV1 | Amycolatopsis saalfeldensis |
| A0A7D6ZW63 | Clostridium intestinale | A0AIQ5AQ00 | Streptomyces sp. MJM1 172 |
| A0A0F0GMW4 | Lentzea aerocolonigenes | A0A2H1IDJ5 | Brevibacterium linens ATCC 9172 |
| A0A7K1JXV1 | Mycobacteroides sp. CBMA 271 | A0A6P0GEG7 | Geodermatophilus normandii |
| A0A7I9ZCD2 | Mycobacterium timonense | A0A4S2THF4 | Streptomyces sp. A1136 |
| A0A1I4ELY3 | Methylorubrum salsuginis | A0A2E0Y160 | Phycisphaerae bacterium |
| A0A1H5AQN2 | Bradyrhizobium erythrophlei | A0A0M8WDM0 | Nocardia sp. NRRL S-836 |
| A0A378YB35 | Nocardia otitidiscaviarum | A0A2V0MX05 | Mycobacterium sp. MFM001 |
| A0A562TJ13 | Roseibium hamelinense | A0A1J9VDR3 | Bacillus paramycoides |
| A0A3L8R7D7 | Streptomyces rapamycinicus (strain ATCC 29253/DSM 41530/NRRL 5491/AYB-994) | A0A5M3X6K4 | Acrocarpospora macrocephala |
| A0A1M5W1C4 | Bradyrhizobium erythrophlei | A0A1X2EFY4 | Mycobacterium szulgai |
| A0A7I9Z9F7 | Mycobacterium timonense | A0A2U1FV73 | Pseudomonas sp. URIL14HWK12:I12 |
| A0A5D0UE88 | Actinomadura syzygii | A0A4V6PXA0 | Pseudomonas sp. OV184 |
| A0A1H4Q2I1 | Bradyrhizobium erythrophlei | A0A1X2DUG6 | Mycobacterium szulgai |
| A0A2S6GWK1 | Actinokineospora auranticolor | D2PVB9 | Kribbella flavida (strain DSM 17836/JCM 10339/NBRC 14399) |
| A3NEX0 | Burkholderia pseudomallei (strain 668) | A0A1L3LD87 | Sinorhizobium americanum CCGM7 |
| A0A1R1S777 | Streptomyces sparsogenes DSM 40356 | M3F528 | Streptomyces bottropensis ATCC 25435 |
| A0A1Q2GTB5 | Pseudoalteromonas aliena | A0A1H7Z476 | Luteibacter sp. UNCMF331Sha3.1 |
| C4KXQ0 | Burkholderia pseudomallei MSHR346 | A0A317QN54 | Geodermatophilus normandii |
| A0A1H5HD00 | Streptomyces sp. 2314.4 | A0A0M8VBD4 | Streptomyces sp. MMG1121 |
| A0A1S2QSA8 | Bacillus sp. MUM 13 | D2PVC1 | Kribbella flavida (strain DSM 17836/JCM 10339/NBRC 14399) |
| A0A124H5G2 | Streptomyces curacoi | W9FML0 | Streptomyces filamentosus NRRL 11379 |
| I0H620 | Actinoplanes missouriensis (strain ATCC 14538/DSM 43046/CBS 188.64/JCM 3121/ | A0A451F7I7 | Pseudoalteromonas sp. R3 |
| | NCIMB 12654/NBRC 102363/431) | | |
| E4N3G1 | Kitasatospora setae (strain ATCC 33774/DSM 43861/ | A0A1V4EFL0 | Microbispora sp. GKU 823 |
| | JCM 3304/KCC A-0304/NBRC 14216/KM-6054) | | |
| A0A3D9J954 | Streptomyces sp. 67 | A0A445N9I7 | Streptomyces netropsis |
| A0A2W6CE09 | Pseudonocardiales bacterium | M2QH44 | Amycolatopsis azurea DSM 43854 |
| A0A4R7S9N9 | Streptomyces sp. KS 21 | A0A7K3A7B4 | Streptomyces sp. SID8379 |
| A0A0M8SMU9 | Streptomyces sp. WM6368 | A0A4R7H761 | Streptomyces sp. BK161 |
| A0A1R1SK47 | Streptomyces sparsogenes DSM 40356 | A0A7K3A868 | Streptomyces sp. SID8379 |
| A0A1H3YC25 | Thiothrix caldifontis | A0A1E3ZRZ4 | Acidovorax sp. SCN 65-28 |
| A0A0X1TLF3 | Turicibacter sp. H121 | W9FN71 | Streptomyces filamentosus NRRL 11379 |
| A0A1R1S4J4 | Streptomyces sparsogenes DSM 40356 | A0A497V848 | Acidovorax sp. 106 |
| A0A2G5INR9 | Streptomyces sp. HG99 | M2Q9E1 | Amycolatopsis azurea DSM 43854 |
| A0A0M8SGX2 | Streptomyces sp. WM6368 | A0A1V2K8C3 | Pseudomonas cedrina subsp. cedrina |
| I0H335 | Actinoplanes missouriensis (strain ATCC 14538/DSM 43046/CBS 188.64/JCM 3121/ | A0A7G8BSP6 | Edaphobacter sp. 4G125 |
| | NCIMB 12654/NBRC 102363/431) | | |
| A0A378WDE4 | Mycolicibacterium fortuitum | L1Q5K3 | Clostridium celatum DSM 1785 |
| A0A126Z6Z6 | Frondihabitans sp. PAMC 28766 | A0A7G8BSW9 | Edaphobacter sp. 4G125 |
| A0A544XU60 | Microbispora bryophytorum | W9FZ52 | Streptomyces filamentosus NRRL 11379 |
| A0A2G5J6B6 | Streptomyces sp. HG99 | A0A0P8WWN9 | Oxobacter pfennigii |
| A0A117PE42 | Streptomyces curacoi | A0A6I6IVV3 | Roseovarius faecimaris |
| A0A1A3MU26 | Mycolicibacterium fortuitum | A0A6G4VJR5 | Streptomyces scabichelini |
| A0A124GZD9 | Streptomyces curacoi | A0A6G4VA08 | Streptomyces scabichelini |
| A0A516RK04 | Streptomyces spectabilis | A0A2T6GEP1 | Pseudomonas protegens |
| A0A2S4CDV2 | Pseudomonas sp. WP001 | A0A7G1NIEL | Streptomyces tuirus |
| A0A2U9P4B7 | Streptomyces actuosus | A0A6H3D319 | Pseudomonas fluorescens |
| A0A433UIE9 | Calothrix desertica PCC 7102 | A0A397PW44 | Streptomyces sp. 2233.2 |
| A0A285C3P0 | Streptomyces sp. TLI_55 | A0A3G2JGX4 | Streptomyces sp. Z022 |
| A0A285C3B0 | Streptomyces sp. TLI_55 | A0A1I2HNR6 | Acidovorax wautersii |
| A0A6P2BJC1 | Gammaproteobacteria bacterium | A0A544YH43 | Microbispora hainanensis |
| A0A4Q2ZQV4 | Sphingobacteriales bacterium | A0A5N6C0Q8 | Microbispora catharanthi |
| A0A101TWW9 | Streptomyces caeruleatus | A0A4Q8C620 | Nocardiopsis sp. CNZ304 |
| A0A124IAS9 | Streptomyces caeruleatus | C9ZFJ4 | Streptomyces scabiei (strain 87.22) |
| A0A4V1SF98 | Sphingobacteriales bacterium | A0A0N9VUM7 | Pseudomonas fluorescens |
| A0A4R5PFH0 | Mycobacteroides franklinii | A0A7K2LMV7 | Streptomyces sp. SID4920 |
| A0A3N4YQJ1 | Myceligenerans xiligouense | A0A120G838 | Pseudomonas fluorescens |
| A0A4R5BEG3 | Actinomadura rubrisoli | A0A6G3UK75 | Streptomyces sp. SID9124 |
| A0A2A8W1K2 | Bacillus megaterium | A0A2P8GVX3 | Labedella gwakjiensis |
| A0A5P2CL50 | Streptomyces venezuelae | A0A5E6PTS7 | Pseudomonas fluorescens |
| A0A5P2BYK1 | Streptomyces venezuelae | A0A0D0RVK7 | Pseudomonas fluorescens |
| I2A704 | Mycobacterium sp. MOTT36Y | A0A438MPZ8 | Nonomuraea polychroma |
| A0A4U2YHT2 | Lysinibacillus varians | A0A6I6N4L4 | Streptomyces broussonetiae |
| A0A1U9ZWN7 | Nonomuraea sp. ATCC 55076 | A0A5B2XNC8 | Goodfellowiella sp. AN110305 |
| A0A1Y0BWY8 | Mycobacterium dioxanotrophicus | A0A0H2ZS20 | Mycobacterium avium (strain 104) |
| A0A1V0A8P9 | Nonomuraea sp. ATCC 55076 | A0A1A2E6W2 | Mycobacterium sp. 852014-52450_SCH5900713 |
| A0A1Y0C965 | Mycobacterium dioxanotrophicus | A0A1X1N1C6 | Frankia sp. KB5 |
| A0A0B5I4W1 | Streptomyces vietnamensis | A0A561C182 | Kribbella amoyensis |
| A0A3N4ZGF3 | Myceligenerans xiligouense | A0A0J6L6S7 | Bacillus sp. LK2 |
| A0A327UEC3 | Streptomyces sp. Amel2xB2 | A0A7L4ZED4 | Kordia antarctica |
| A0A2S4YVZ0 | Streptomyces sp. Ru71 | Q8CJU6 | Streptomyces coelicolor (strain ATCC BAA-471/A3(2)/M145) |
| A0A0U0WFY2 | Mycobacterium bohemicum DSM 44277 | A0A2S1T0M2 | Streptomyces tirandamycinicus |
| A0A1M5S7H9 | Bradyrhizobium erythrophlei | S3ZF37 | Streptomyces aurantiacus JA 4570 |
| A0A231RNG9 | Bacillus sp. DSM 27956 | A0A402A382 | Tengunoibacter tsumagoiensis |
| A0A1U9ZX47 | Nonomuraea sp. ATCC 55076 | E2Q897 | Streptomyces clavuligerus |
| A0A327UJI4 | Streptomyces sp. Amel2xB2 | A0A4R5AJF8 | Jiangella aurantiaca |
| A0A327UGF3 | Streptomyces sp. Amel2xB2 | Q93S08 | Streptomyces coelicolor (strain ATCC BAA-471/A3(2)/M145) |
| A0A7K2W4A9 | Streptomyces sp. SID2888 | A0A559U2H2 | Streptomyces sp. CNZ289 |
| A0A2A2Y762 | Verrucomicrobiae bacterium AMD-G2 | A0A1R1WKH2 | Streptomyces sp. M1013 |
| A0A1C4W024 | Micromonospora chaiyaphumensis | A9E0H6 | Kordia algicida OT-1 |
| A0A7G6XBW9 | Streptomyces sp. INR7 | A9DZL8 | Kordia algicida OT-1 |
| A0A4P8UUY1 | Mycobacterium avium subsp. hominissuis | A0A557WWY6 | Mycobacterium helveticum |
| A0A238KW02 | Ruegeria arenilitoris | A0A1H4CL41 | Mycobacterium sp. 283mftsu |
| A0A143C1S7 | Streptomyces qaidamensis | A0A367E8U1 | Streptomyces diacarni |
| A0A1H9CA45 | Litorimicrobium taeanense | A0A5P0YJ08 | Fictibacillus phosphorivorans |
| A0A1H4V5S0 | Streptomyces sp. PAN_FS17 | A0A0R3HCX4 | Mycobacteroides sp. H054 |
| A0A1I4VRD8 | Saccharopolyspora antimicrobica | A0A101JCX1 | Streptomyces regalis |
| A0A7H8HIF4 | Amycolatopsis sp. Hca4 | V7JWZ0 | Mycobacterium avium subsp. silvaticum ATCC 49884 |
| A0A7H8HIJ7 | Amycolatopsis sp. Hca4 | A0ASN7YLY9 | Starkeya sp. |
| A0A5A4W414 | Mycobacterium avium subsp. hominissuis | A0A7I7W1G3 | Mycobacterium branderi |
| A0A1Q3U3Q7 | Sphingobacteriales bacterium 39-19 | A0A4V3RJ16 | Stenotrophomonas maltophilia |
| A0A318AM70 | Caulobacter sp. D5 | A0A5C4J5P2 | Actinomadura sp. 14C53 |
| A0A5C8UST1 | Lacisediminihabitans profunda | A0A5C4J9J4 | Actinomadura sp. 14C53 |
| A0A437QM09 | Rheinheimera riviphila | A0A5C8QHU6 | Streptomyces sp. IB2014 016-6 |
| A0A0H3HQ59 | Burkholderia pseudomallei (strain 1026b) | A0A0N1GXX8 | Actinobacteria bacterium OV320 |
| A0A1A2GF50 | Mycobacterium sp. 852002-51057_SCH5723018 | A0A2A2ZP34 | Mycobacterium avium |
| A0A4R2WG59 | Massilia sp. GV090 | A0A229RB73 | Amycolatopsis alba DSM 44262 |
| A0A059WBE2 | Streptomyces albulus | A0A1B1BAY7 | Streptomyces griseochromogenes |
| A0A5D0PH36 | Microbispora tritici | A0A1H5UUF0 | Bryocella elongata |
| C5T975 | Acidovorax delafieldii 2AN | D8IZD2 | Herbaspirillum seropedicae (strain SmR1) |
| A0A2T5JM43 | Nitrosospira sp. Nsp5 | A0A2T7MA46 | Streptomyces sp. CS014 |
| A0A4R8R0Q0 | Mycobacteroides franklinii | A0A2T6FZ86 | Paenibacillus elgii |
| A0A2U0EQI0 | Pseudomonas sp. HMWF011 | A0A1I5J2J2 | Actinomadura madurae |
| C1FQX3 | Clostridium botulinum (strain Kyoto/Type A2) | A0A1M4E1H0 | Nonomuraea gerenzanensis |
| A0A1S1L6E2 | Mycobacteroides franklinii | A0A1M4ECZ1 | Nonomuraea gerenzanensis |
| A0A401QRN7 | Streptomyces albulus | A0A6B1PB91 | Streptomyces sp. SID2955 |
| A0A399G1I4 | Thermobifida halotolerans | A0A7L8VZ30 | Bradyrhizobium sp. SEMIA |
| A0A4V2XI74 | Nonomuraea longispora | A0A291NN01 | Streptomyces sp. SCSIO 1666 |
| A0A5S4GKC1 | Actinomadura geliboluensis | Q8VWA5 | Streptomyces galilaeus |
| A8ZKV4 | Acaryochloris marina (strain MBIC 11017) | A0A6N3EX60 | Clostridium tertium |
| A0A399FVL7 | Thermobifida halotolerans | A0A6N3AY55 | Clostridium tertium |
| A0A6G2VK03 | Streptomyces sp. SID161 | A0A6B1TPN9 | Clostridium beijerinckii |
| A0A2T6JAW1 | Acidovorax sp. 107 | FIDI30 | Streptomyces sp. SCSIO 1666 |
| A0A0L8NQZ4 | Streptomyces griseoflavus | A0A6B3QPV7 | Streptomyces tendae |
| A0A1A2GIJ1 | Mycobacterium sp. 852002-51057_SCH5723018 | I2N2K9 | Streptomyces tsukubensis (strain DSM 42081/NBRC 108919/NRRL 18488/9993) |
| A0A2N0JMW9 | Kitasatospora sp. OK780 | U6A1G7 | Streptomyces sp. CNH287 |
| M1MKZ8 | Clostridium saccharoperbutylacetonicum N1-4(HMT) | A0A6B1NWH1 | Streptomyces sp. SID1034 |
| A0A4S2J304 | Streptomyces sp. S816 | A0A6N3EQ76 | Clostridium tertium |
| A0A261CXI1 | Williamsia sp. 1138 | H8Y6P5 | Actinoalloteichus sp. WH1-2216-6 |
| A0A6G2VI00 | Streptomyces sp. SID161 | V5RN11 | Goodfellowiella coeruleoviolacea |
| A0A1A9QWM5 | Streptomyces albulus | G2ZP41 | blood disease bacterium R229 |
| A0A4R1KN59 | Curtobacterium sp. PhB136 | Q2HR11 | Streptomyces sp. SCC 2136 |
| A0A255D5F1 | Mycolicibacterium sphagni | A0A1B4Z982 | Actinomadura fulva subsp. indica |
| A0A387BHF5 | Gryllotalpicola protaetiae | A5A3J1 | Streptomyces coeruleorubidus |
| A0A7H8HVR8 | Amycolatopsis sp. Hca4 | A0A6A8REJ9 | Nocardia seriolae |
| A0A5P2W9U8 | Streptomyces nodosus | HIZYV5 | Streptomyces sp. CS40 |
| A0A640S1V3 | Streptomyces caniferus | A0A6B1QVB3 | Streptomyces sp. SID335 |
| A0A401R576 | Streptomyces albulus | A0A653ESW9 | Mycobacterium riyadhense |
| A0A2T6KU99 | Promicromonospora sp. AC04 | A0A652LGM9 | Streptomyces sp. or43 |
| A0A7K3E2D6 | Streptomyces sp. SID5476 | B5L6M0 | Micromonospora chalcea |
| A0A0G9LA35 | Clostridium sp. C8 | A0A6G3SUW5 | Streptomyces anulatus |
| A0A506VEE1 | Mixta tenebrionis | D3Y1I2 | Streptomyces sp. 307-9 |
| A0A179V931 | Mycobacteroides immunogenum | MIF4V9 | Actinoalloteichus cyanogriseus |
| A0A6G2RHH7 | Streptomyces sp. SID4951 | A0A6B1QMK5 | Streptomyces sp. SID335 |
| A0A1I4VDM0 | Algoriella xinjiangensis | A0A1C6KYM4 | uncultured Clostridium sp. |
| A0A0B5DQY0 | Streptomyces nodosus | A0A652KL55 | Streptomyces sp. gb1(2016) |
| A0A1H9KQB8 | Lentzea flaviverrucosa | A0A6B1QG21 | Streptomyces sp. SID335 |
| A0A1H2FZ86 | Amycolatopsis keratiniphila | A0A6D1T3U1 | Bacillus sp. BH32 |
| A0A6G2RB16 | Streptomyces sp. SID4951 | A0A652LZK7 | Streptomyces sp. or43 |
| A0A1G9GSP2 | Arthrobacter sp. ov407 | A0A6S6UHV3 | uncultured Thiotrichaceae bacterium |
| S6FKK0 | Clostridium chauvoei JF4335 | A0A6B3N5I8 | Symploca sp. SIO1C4 |
| A0A0K9XKT1 | Streptomyces caatingaensis | U5XNI5 | Streptomyces sp. Ls2151 |
| A0A0C1DMN2 | Nocardia vulneris | A0A7G8VAV7 | Variovorax sp. PAMC26660 |
| A0A0K9XD97 | Streptomyces caatingaensis | A0A193PKW1 | Streptomyces sp. TK08046 |
| A0A0M9ZN09 | Streptomyces sp. AS58 | A1C189 | Streptomyces echinatus |
| A0A3A4KA37 | Nocardia panacis | E2D2L8 | uncultured soil bacterium |
| A0A168KKJ3 | Frankia sp. EI5c | S4VAU1 | Streptomyces lusitanus |
| A0A2U3H0L9 | Streptomyces sp. NWU49 | A0A1B1V591 | Streptomyces albogriseolus |
| A0A4Q7Q0S6 | Kribbella sp. VKM Ac-2569 | A0A6BITR92 | Clostridium beijerinckii |
| A0A1G9HDE0 | Streptomyces indicus | Q9ALN1 | Saccharopolyspora spinosa |
| A0A177HR17 | Streptomyces jeddahensis | A0A6B0DNP7 | Acidovorax citrulli |
| A0A066YI47 | Kitasatospora cheerisanensis KCTC 2395 | A0A6B1NSB4 | Streptomyces sp. SID 1034 |
| A0A4P7YNK7 | Azospirillum sp. TSH100 | A0A6B1NND5 | Streptomyces sp. SID 1034 |
| A0A066Z739 | Kitasatospora cheerisanensis KCTC 2395 | A0A0T9MN63 | Mycobacterium tuberculosis |
| A0A428ZP53 | Streptomyces sp. WAC 01325 | A0A0E8NSA7 | Mycobacterium tuberculosis |
| A0A0M9Z354 | Streptomyces sp. H021 | A0A7J0C9R2 | Streptomyces fulvorobeus |
| D7C564 | Streptomyces bingchenggensis (strain BCW-1) | A0A6B4YAN7 | Clostridium botulinum |
| A0A2K9NL77 | Niveispirillum cyanobacteriorum | A0A2N8NV12 | Streptomyces eurocidicus |
| A0A2M8X5T3 | Acidovorax sp. 69 | A0A1E3T4D4 | Mycobacterium sherrisii |
| A0A6L6QCD8 | Pseudoduganella eburnea | A0A6S7AIJ8 | Achromobacter deleyi |
| A0A066YQP0 | Kitasatospora cheerisanensis KCTC 2395 | A0A7I7MPV8 | Mycobacterium shinjukuense |
| A0A516PXG4 | Microlunatus sp. KUDC0627 | A0A1M6Y0B3 | Bradyrhizobium lablabi |
| A0A101SUK9 | Streptomyces griseoruber | A0A519ML61 | Acidovorax sp. |
| A0A2U3C9T5 | Streptomyces sp. ICBB 8177 | A0A3G7UTE3 | Pseudomonas synxantha |
| A0A193C0Z9 | Amycolatopsis orientalis | A0A1M6T2I4 | Bradyrhizobium lablabi |
| A0A6G2X4C4 | Streptomyces sp. SID8375 | A0A7G8KF13 | Streptomyces buecherae |
| A0A1M5C2T0 | Tenacibaculum mesophilum | A0A517YJ58 | Anatilimnocola aggregata |
| A0A4V3T9X6 | Streptomyces sp. A0592 | A0A7G7XJD8 | Pseudomonas protegens |
| A0A193BUN6 | Amycolatopsis orientalis | A0A3N4FSJ6 | Burkholderia mallei |
| A0A193C8E1 | Amycolatopsis orientalis | A0A0S4QPA5 | Frankia irregularis |
| A0A4Q7PZ57 | Kribbella sp. VKM Ac-2569 | A0A1G7JYE2 | Dyella sp. 333MFSha |
| A0A3N7AA39 | Taibaiella sp. KBW10 | A0A0L8N1R4 | Streptomyces virginiae |
| A0A2M9B2T7 | Streptomyces sp. CNZ306 | A0A4R7V0M1 | Actinophytocola oryzae |
| A0A348Y3G0 | Flavobacteriaceae bacterium | A0A5N5ZAH4 | Kordia sp. TARA_039_SRF |
| A0A0Q8Q0N3 | Kitasatospora sp. Root187 | A0A1C0TJ76 | Pseudoalteromonas luteoviolacea |
| A0A3D1D8T8 | Flavobacteriaceae bacterium | A0A0CIQFQ2 | Pseudoalteromonas luteoviolacea |
| A0A286FQR9 | Streptomyces sp. Ag109_G2-15 | A0A541BI86 | Pseudoalteromonas luteoviolacea |
| A0A4Q7XZX7 | Streptomyces sp. BK022 | A0A1A2GT56 | Mycobacterium sp. E3198 |
| A0A0D8BEY4 | Frankia torreyi | A0A3G8C321 | Pseudomonas sp. R4-34-07 |
| A0A2M9B3E1 | Streptomyces sp. CNZ306 | A0A2H9VJY5 | Bradyrhizobium lablabi |
| A0A1H9FJ94 | Loktanella sp. DSM 29012 | A0A3L8J7N4 | Streptomyces griseocarneus |
| A0A160BTL4 | Ralstonia mannitolilytica | A0A0L8M5Z0 | Streptomyces virginiae |
| A0A7H9DNX3 | Empedobacter falsenii | A0A3G7U0Y9 | Pseudomonas synxantha |
| A0A7H5I9A3 | Streptomyces sp. S1D4-14 | A0A2M9LFY8 | Streptomyces sp. CB02613 |
| A0A5E8PLZ3 | Mycolicibacterium porcinum | A0A1E4EET7 | Niastella sp. SCN 39-18 |
| R1GAL1 | Amycolatopsis vancoresmycina DSM 44592 | A0A1V9KC09 | Streptomyces sp. M41(2017) |
| A0A2V8DJ34 | Acidobacteria bacterium | A0A1Y2MS83 | Streptomyces platensis |
| A0A075K6F4 | Pelosinus sp. UFO1 | A0A2T0ME42 | Nonomuraea fuscirosea |
| A0A5M3W6P1 | Acrocarpospora corrugata | A0A519ITV7 | Acidovorax sp. |
| A0A1H6D4T7 | Nonomuraea solani | A0A1Y2NAS9 | Streptomyces platensis |
| A0A1X1QWJ2 | Mycobacterium bohemicum | A0A7H8N866 | Streptomyces buecherae |
| A0A1E8FUR8 | Arthrobacter sp. SW1 | A0A386WEJ5 | Micromonospora tulbaghiae |
| A0A1H6BYV6 | Nonomuraea solani | A0A0J6KG58 | Chromobacterium sp. LK1 |
| A0A2V7YZ10 | Acidobacteria bacterium | A0A5N8VR67 | Streptomyces adustus |
| A0A6S7CFN9 | Achromobacter dolens | A0A2T0M277 | Nonomuraea fuscirosea |
| A0A0Q8TQH5 | Acidovorax sp. Root219 | A0A1Y2NH94 | Streptomyces platensis |
| A0A4Q3KKQ4 | Oxalobacteraceae bacterium | A0A7V8NJY5 | Streptomyces sp. AJS327 |
| A0A6F8YAS6 | Phytohabitans suffuscus | WP_030877494.1 | Streptomyces varsoviensis |
| A0A7X0U8H0 | Acidovorax soli | A0A7W9W5M9 | Armatimonas rosea |
| A0A7W0ZNZ9 | Chloroflexia bacterium | A0A7S7QUS2 | Bradyrhizobium sp. CCBAU 51753 |
| A0A7W1GLS8 | Actinobacteria bacterium | A0A7V9N582 | Chloroflexia bacterium |
| A0A7W9KIS2 | Kutzneria kofuensis | A0A841AV07 | Amycolatopsis umgeniensis |
| A0A7Y5XF41 | Streptomycetaceae bacterium | A0A7W3W2T5 | Amycolatopsis dendrobii |
| WP_235454663.1 | Streptomyces olivochromogenes | WP_211768552.1 | Kutzneria sp. CA-103260 |

In some embodiments, the flavin-dependent oxidase is not EncM from Streptomyces maritimus or Clz9 from Streptomyces sp. CNH-287 (SEQ ID NO:15). Flavin-dependent oxidases known as EncM from Streptomyces maritimus or Clz9 from Streptomyces sp. CNH-287, as well as entire genomes of bacterial and fungal species, were sequenced previously, which in some embodiments may be described as comprising the peptide motif of Formula 1. However, prior disclosures of proteins that may, in some embodiments, comprise the peptide motif of Formula I, did not recognize the criticality of the conserved regions of peptide's motif and the binding of the Cys in that motif with an FAD cofactor at the indicated positions. Likewise, prior disclosures did not recognize that bivalent binding of the FAD included not only the Cys of the motif of Formula I, but also a His residue that is also present in the flavin-dependent oxidase. Thus, the present disclosure provides for novel flavin-dependent oxidases, as well as a method of identifying a bacterial protein or a fungal protein useful for flavin-dependent oxidation, e.g., a flavin-dependent oxidase capable of oxidative cyclization of a prenylated aromatic compound into a cannabinoid.

In some embodiments, the flavin-dependent oxidase does not comprise a disulfide bond. In the context of a protein or polypeptide, a disulfide bond (sometimes called a “S—S bond” or “disulfide bridge”) refers to a covalent bond between two cysteine residues, typically formed through oxidation of the thiol groups on the cysteines. Proteins comprising disulfide bonds, e.g., endogenous to plants, can be unstable in bacterial host cells as the disulfide bonds are often disrupted due to the reducing environment in bacterial cells. In some embodiments, cannabinoid synthases from C. sativa are substantially unstable in a bacterial cell, e.g., an E. coli cell. As used herein, “unstable” protein can refer to proteins that are non-functional, denatured, and/or degraded rapidly, resulting in catalytic activity that is greatly reduced relative to the activity found in its native host cell, e.g., C. sativa plants. In some embodiments, the lack of a disulfide bond in the flavin-dependent oxidase described herein advantageously allows for its soluble and active expression by a bacterial host cell. In some embodiments, a bacterial host cell produces at least 1.5 times, at least 1.6 times, at least 1.7 times, at least 1.8 times, at least 1.9 times, at least 2 times, at least 3 times, at least 4 times, at least 5 times, at least 6 times, at least 7 times, at least 8 times, at least 9 times, at least 10 times, at least 20 times, at least 50 times, or at least 100 times more of the flavin-dependent oxidase that does not comprise a disulfide bond as compared with a flavin-dependent oxidase that comprises a disulfide bond, e.g., a wild-type cannabinoid synthase from C. sativa. In some embodiments, the flavin-dependent oxidase comprises at least 70%, at least 75%, at least 80%, at least 85%, at least 90%, at least 95%, at least 96%, at least 97% at least 98%, at least 99%, or 100% sequence identity to a protein with UniProt IDs A0A1H4CL41, A0A7X₀U8H0, A0A1Q5S5E2, A0A0Q7FI10, A0A2E0XWX6, D9XHS6, A0A0K3BN04, and A0A1U9QQ65. In some embodiments, the flavin-dependent oxidase comprises a motif of any one of SEQ ID NOs:1-14.

In some embodiments, the flavin-dependent oxidase is not glycosylated. As used herein, glycosylation refers to the addition of one or more sugar molecules to another biomolecule, e.g., a protein or polypeptide. Glycosylation can play an important role in the folding, secretion, and stability of proteins (see, e.g., Drickamer and Taylor, Introduction to Glycobiology (2^{nd} ed.), Oxford University Press, USA). Glycosylation mechanisms and patterns in bacteria and eukaryotes are distinct from one another. Moreover, the most common type of glycosylation, N-linked glycosylation, occurs in eukaryotes but not in bacteria. Thus, bacterial cells are generally not suitable for the production of eukaryotic proteins that are glycosylated, e.g., the cannabinoid synthases from C. sativa. In some embodiments, the lack of glycosylation in the flavin-dependent oxidase further advantageously allows for its soluble and active expression by a bacterial host cell. In some embodiments, a bacterial host cell produces at least 1.5 times, at least 1.6 times, at least 1.7 times, at least 1.8 times, at least 1.9 times, at least 2 times, at least 3 times, at least 4 times, at least 5 times, at least 6 times, at least 7 times, at least 8 times, at least 9 times, at least 10 times, at least 20 times, at least 50 times, or at least 100 times more (e.g., by weight) of the flavin-dependent oxidase that is not glycosylated, compared with a flavin-dependent oxidase that is glycosylated, e.g., a wild-type cannabinoid synthase from C. sativa.

In some embodiments, a bacterial host cell produces at least 1.5 times, at least 1.6 times, at least 1.7 times, at least 1.8 times, at least 1.9 times, at least 2 times, at least 3 times, at least 4 times, at least 5 times, at least 6 times, at least 7 times, at least 8 times, at least 9 times, at least 10 times, at least 20 times, at least 50 times, or at least 100 times (e.g., by weight) more of the flavin-dependent oxidase that does not comprise a disulfide bond and is not glycosylated, compared with a flavin-dependent oxidase that comprises a disulfide bond and is glycosylated, e.g., a wild-type cannabinoid synthase from C. sativa.

In some embodiments, the flavin-dependent oxidase described herein is capable of converting a prenylated aromatic compound to a cannabinoid. Prenylated aromatic compounds and cannabinoids are described herein. In some embodiments, the prenylated aromatic compound is cannabigerolic acid (CBGA), cannabigerorcinic acid (CBGOA), cannabigerovarinic acid (CBGVA), cannabigerorcinol (CBGO), cannabigerivarinol (CBGV), or cannabigerol (CBG). In some embodiments, the cannabinoid is CBCA, CBCVA, CBCOA, CBC, CBCV, CBCO, THCA, THCVA, THCOA, THC, THCV, THCO, CBDA, CBDVA, CBDOA, CBD, CBDV, CBDO, or an isomer, analog, or derivative thereof. In some embodiments, the flavin-dependent oxidase comprises at least 70%, at least 75%, at least 80%, at least 85%, at least 90%, at least 95%, at least 96%, at least 97% at least 98%, at least 99%, or 100% sequence identity to a protein with UniProt IDs A0A1H4CL41, A0A7X0U8H0, A0A1Q5S5E2, A0A0Q7FI10. A0A2E0XWX6, D9XHS6, A0A0K3BN04, and A0A1U9QQ65. In some embodiments, the flavin-dependent oxidase comprises a motif of any one of SEQ ID NOs:1-14.

In some embodiments, the disclosure provides a non-natural flavin-dependent oxidase. As described herein, a “non-natural” protein or polypeptide refers to a protein or polypeptide sequence having at least one variation at an amino acid position as compared to a wild-type polypeptide sequence. In some embodiments, the flavin-dependent oxidase has at least one variation at an amino acid position as compared to a wild-type flavin-dependent oxidase.

In some embodiments, the at least one amino acid variation comprises a substitution, deletion, insertion, or combinations thereof. In some embodiments, the variation comprises an amino acid substitution. In some embodiments, the variation comprises a deletion of one or more amino acids e.g., about 1 to about 100, about 2 to about 80, about 5 to about 50, about 10 to about 40, about 12 to about 35, about 13 to about 32, or about 14 to about 30 amino acids. In some embodiments, the variation comprises an insertion of one or more amino acids. In some embodiments, the at least one amino acid variation in the flavin-dependent oxidase is not in an active site of the flavin-dependent oxidase. In some embodiments, the active site of the flavin-dependent oxidase comprises one or more amino acid residues involved in binding the substrate, e.g., CBGA, CBGOA, CBGVA, CBG, CBGO, and/or CBGV. In some embodiments, the active site of the flavin-dependent oxidase comprises one or more amino acid residues involved in binding FAD cofactor. In some embodiments, the active site of the flavin-dependent oxidase comprises one or more amino acid residues involved for catalysis, e.g., the oxidative cyclization of CBGA into CBCA.

In some embodiments, the flavin-dependent oxidase is capable of converting a prenylated aromatic compound into a cannabinoid at about pH 4 to about pH 9, or about pH 4.5 to about pH 8.5, or about pH 5 to about pH 8, or about pH 5.5 to about pH 7.5, or about pH 5 to about pH 7. In some embodiments, catalytic activity of the flavin-dependent oxidase is substantially the same from about pH 4 to about pH 9. In some embodiments, catalytic activity of the flavin-dependent oxidase is substantially the same from about pH 4.5 to about pH 8.5. In some embodiments, catalytic activity of the flavin-dependent oxidase is substantially the same from about pH 5 to about pH 8. In some embodiments, catalytic activity of the flavin-dependent oxidase is substantially the same from about pH 5.5 to about pH 7.5. In some embodiments, catalytic activity of the flavin-dependent oxidase is substantially the same from about pH 5 to about pH 7. In some embodiments, catalytic activity of the flavin-dependent oxidase is substantially the same at about pH 5 and at about pH 7. As referred to throughout the application, when comparing the catalytic activity of at least two enzymes, it will be understood by one of ordinary skill in the art that the enzymes can be subjected to the same or substantially the same reaction conditions or the enzymes can be subjected to the optimal reaction conditions for each enzyme, and catalytic activity is assessed using the same or substantially the same methods and/or equipment. Optimal reaction conditions for the enzymes described herein can be determined by one of ordinary skill in the art. As used herein, the term “substantially” when referring to enzyme activity at different pH conditions means that the flavin-dependent oxidase enzyme activity does not vary (increase or decrease) by more than 20%, more than 15%, more than 10%, more than 5%, or more than 1% under the different pH conditions. In some embodiments, catalytic activity of the flavin-dependent oxidase does not vary more than 20%, more than 15%, more than 10%, more than 5%, or more than 1% from about pH 5 to about pH 8. As described herein, cannabinoid synthases from C. sativa generally require low pH (around 5 to 5.5) for optimal activity and are less active at neutral pH (see, e.g., Zirpel et al. (2018), J Biotechnol 284:17-26). The catalytic activity of the flavin-dependent oxidase does not vary substantially over a wide range of pH (e.g., from about pH 5 to about pH 8), which is beneficial for microbial production of cannabinoids.

In some embodiments, the flavin-dependent oxidase has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to a natural, i.e., wild-type, flavin-dependent oxidase. As described herein, the terms “natural” or “wild-type” flavin-dependent oxidase can refer to any known flavin-dependent oxidase, e.g., the flavin-dependent oxidases in Table 1. In some embodiments, the flavin-dependent oxidase comprises at least 70%, at least 75%, at least 80%, at least 85%, at least 90%, at least 95%, at least 96%, at least 97% at least 98%, at least 99%, or 100% sequence identity to a protein with UniProt IDs A0A1H4CL41, A0A7X0U8H0, A0A1Q5S5E2, A0A0Q7FI10, A0A2E0XWX6, D9XHS6, A0A0K3BN04, and A0A1U9QQ65. In some embodiments, the flavin-dependent oxidase comprises a motif of any one of SEQ ID NOs:1-14.

In some embodiments, the disclosure provides a flavin-dependent oxidase with about 70%, 75%, 80%, 85%, 90%, 95%, 99% or greater identity to at least about 25, 50, 75, 100, 125, 150, 200, 250, 300, 350, 400, 450, 500, or more contiguous amino acids of a flavin-dependent oxidase in Table 1. In some embodiments, the flavin-dependent oxidase further comprises at least one amino acid variation as compared to a wild type flavin-dependent oxidase. In some embodiments, the flavin-dependent oxidase comprises about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, or about 100 amino acid variations as compared to a wild-type flavin-dependent oxidase of Table 1. In some embodiments, the amino acid variation is an amino acid substitution, deletion, or insertion. In some embodiments, the variation is a substitution of one or more amino acids in the polypeptide sequence of a flavin-dependent oxidase in Table 1.

In some embodiments, the flavin-dependent oxidase herein is capable of converting CBGA to CBCA, THCA, CBDA, or combinations thereof. In some embodiments, the flavin-dependent oxidase herein is capable of converting CBGOA to CBCOA, THCOA, CBDOA, or combinations thereof. In some embodiments, the flavin-dependent oxidase herein is capable of converting CBGVA to CBCVA, THCVA, CBDVA, or combinations thereof. In some embodiments, the flavin-dependent oxidase herein is capable of converting CBG to CBC, THC, CBD, or combinations thereof. In some embodiments, the flavin-dependent oxidase herein is capable of converting CBGO to CBCO, THCO, CBDO, or combinations thereof. In some embodiments, the flavin-dependent oxidase herein is capable of converting CBGV to CBCV, THCV, CBDV, or combinations thereof. In some embodiments, the conversion is performed at about pH 4 to about pH 9, or about pH 4.5 to about pH 8.5, or about pH 5 to about pH 8, or about pH 5.5 to about pH 7.5. In some embodiments, the conversion is performed at about pH 4, about pH 4.5 about pH 5, about pH 5.5, about pH 6, about pH 6.5, about pH 7, about pH 7.5, about pH 8, about pH 8.5, or about pH 9. In some embodiments, the conversion is performed at about pH 5. In some embodiments, the conversion is performed at about pH 7.4 or about pH 7.5. In some embodiments, the flavin-dependent oxidase has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least or about 99%, or at least about 100% of the catalytic activity of a wild-type cannabinoid synthase, e.g., wild-type CBCAS. THCAS, or CBDAS from C. sativa. In some embodiments, the flavin-dependent oxidase comprises at least 70%, at least 75%, at least 80%, at least 85%, at least 90%, at least 95%, at least 96%, at least 97% at least 98%, at least 99%, or 100% sequence identity to a protein with UniProt IDs A0A1H4CL41, A0A7X0U8H0, A0A1Q5S5E2, A0A0Q7FI10, A0A2E0XWX6, D9XHS6, A0A0K3BN04, and A0A1U9QQ65. In some embodiments, the flavin-dependent oxidase comprises a motif of any one of SEQ ID NOs:1-14.

In some embodiments, the flavin-dependent oxidase described herein further comprises an affinity tag, a purification tag, a solubility tag, or combinations thereof. As used in the context of proteins and polypeptides, a “tag” can refer to a short polypeptide sequence, typically about 5 to about 50 amino acids in length, that is covalently attached to the protein of interest, e.g., the flavin-dependent oxidase. Additionally or alternatively, a tag can also comprise a polypeptide that is greater than 50 amino acids in length and that provides a desired property, e.g., increases solubility, to the tagged protein of interest. In some embodiments, the tag is attached to the protein such that it in the same reading frame as the protein, i.e., “in-frame.” In general, the tag allows a specific chemical or enzymatic modification to the protein of interest. Solubility tags increases the solubility of the tagged protein and include, e.g., thioredoxin (TRX), poly(NANP), maltose-binding protein (MBP), and glutathione S-transferase (GST). Affinity tags allow the protein to bind to a specific molecule. Examples of affinity tags include chitin binding protein (CBP), Strep-tag, poly(His) tag, and the like; in addition, certain solubility tags such as MBP and GST can also serve as an affinity tag. Purification tags, also termed chromatography tags, allow the protein to be separated from other components in a particular purification or separation technique and are typically comprise polyanionic amino acids, such as the FLAG-tag. Further examples of tags that can be included on the flavin-dependent oxidases provided herein include, without limitation, epitope tags such as ALFA-tag, V5-tag, Myc-tag, HA-tag, Spot-tag, T7-tag, and NE-tag, which can be useful in western blotting or immunoprecipitation; and fluorescence tags such as GFP and its variants for visualization of the tagged protein. One of ordinary skill in the art would understand that the flavin-dependent oxidase provided herein can comprise a single tag, or combinations of tags including multiple functions. Methods of producing tagged proteins, e.g., a tagged flavin-dependent oxidase, are known in the field. See, e.g., Kimple et al. (2013), Curr Protoc Protein Sci 73: Unit-9.9.

In some embodiments, the disclosure further provides a polynucleotide comprising a nucleic acid sequence encoding the flavin-dependent oxidase described herein. In some embodiments, the disclosure further provides a polynucleotide comprising a nucleic acid sequence encoding the flavin-dependent oxidase in Table 1. In some embodiments, the disclosure further provides a polynucleotide comprising: (a) a nucleic acid sequence encoding a polypeptide comprising at least 80% sequence identity to a flavin-dependent oxidase described herein, e.g., in Table 1; and (b) a heterologous regulatory element operably linked to the nucleic acid sequence. In some embodiments, the nucleic acid sequence encodes a polypeptide comprising at least 85%, at least 90%, at least 91%, at least 92%, at least 93%, at least 94%, at least 95%, at least 96%, at least 97%, at least 98%, at least 99%, or 100% sequence identity to the amino acid sequence of any of the flavin-dependent oxidases in Table 1. In some embodiments, the flavin-dependent oxidase comprises at least 70%, at least 75%, at least 80%, at least 85%, at least 90%, at least 95%, at least 96%, at least 97% at least 98%, at least 99%, or 100% sequence identity to a protein with UniProt IDs A0A1H4CL41, A0A7X0U8H0, A0A1Q5S5E2, A0A0Q7FI10, A0A2E0XWX6, D9XHS6, A0A0K3BN04, and A0A1U9QQ65. In some embodiments, the flavin-dependent oxidase comprises a motif of any one of SEQ ID NOs:1-14.

In some embodiments, the nucleic acid sequence encoding the flavin-dependent oxidase is codon optimized. An example of a codon optimized sequence is, in one instance, a sequence optimized for expression in a bacterial host cell, e.g., E. coli. In some embodiments, one or more codons in a nucleic acid sequence encoding the flavin-dependent oxidase described herein corresponds to the most frequently used codon for a particular amino acid in the bacterial host cell.

In some embodiments, the heterologous regulatory element of the polynucleotide comprises a promoter, an enhancer, a silencer, a response element, or combinations thereof. In some embodiments, the heterologous regulatory element of (b) is a bacterial regulatory element. Non-limiting examples of bacterial regulatory elements include the T7 promoter, Sp6 promoter, lac promoter, araBad promoter, trp promoter, and Ptac promoter. Further examples of regulatory elements can be found, e.g., using the PRODORIC2 database (Eckweiler et al. (2018), Nucleic Acids Res 46(D1):D320-D326).

In some embodiments, the disclosure provides an expression construct comprising the polynucleotide provided herein. Expression constructs are described herein and include, e.g., pQE vectors (Qiagen), pBluescript plasmids, pNH vectors, lambda-ZAP vectors (Stratagene): pTrc99a, pKK223-3, pDR540, and pRIT2T (Pharmacia). In some embodiments, the expression construct comprises a regulatory element. Regulatory elements are provided herein.

In some embodiments, the disclosure provides an engineered cell comprising a heterologous polynucleotide encoding the flavin-dependent oxidase described herein. In some embodiments, the disclosure provides an engineered cell comprising a heterologous polynucleotide encoding a flavin-dependent oxidase of Table 1. In some embodiments, the disclosure provides an engineered cell comprising the flavin-dependent oxidase described herein, the polynucleotide described herein, the expression construct described herein, or combinations thereof. In some embodiments, the flavin-dependent oxidase is any of the flavin-dependent oxidases in Table 1. In some embodiments, the flavin-dependent oxidase is a non-natural flavin-dependent oxidase described herein.

In some embodiments, the disclosure provides a method of making an isolated flavin-dependent oxidase, comprising isolating the flavin-dependent oxidase from the engineered cell provided herein. In some embodiments, the disclosure provides an isolated flavin-dependent oxidase, wherein the isolated flavin-dependent oxidase is expressed, e.g., overexpressed, and isolated from the engineered cell. In some embodiments, the flavin-dependent oxidase is any of the flavin-dependent oxidases in Table 1. In some embodiments, the flavin-dependent oxidase is a non-natural flavin-dependent oxidase described herein. Methods of expressing and isolating heterologous proteins are known to one of ordinary skill in the art. In some embodiments, the flavin-dependent oxidase comprises at least 70%, at least 75%, at least 80%, at least 85%, at least 90%, at least 95%, at least 96%, at least 97% at least 98%, at least 99%, or 100% sequence identity to a protein with UniProt IDs A0A1H4CL41, A0A7X0U8H0, A0A1Q5S5E2, A0A0Q7FI10. A0A2E0XWX6, D9XHS6, A0A0K3BN04, and A0A1U9QQ65. In some embodiments, the flavin-dependent oxidase comprises a motif of any one of SEQ ID NOs:1-14.

In some embodiments, the engineered cell described herein is capable of making a cannabinoid. Cannabinoids are further described herein. In some embodiments, the cannabinoid is CBCA. CBDA, THCA, CBCOA, CBDOA, THCOA, CBCVA, CBDVA, THCVA, CBC, CBD, THC, CBCO, CBDO, THCO, CBCV, CBDV, THCV, or combinations thereof. Methods of making cannabinoids in cells, e.g., by fermentation, are further described herein.

In some embodiments, the engineered cell further comprises a cannabinoid biosynthesis pathway enzyme. An exemplary cannabinoid biosynthesis pathway starts from the conversion of hexanoate to hexanoyl-CoA (Hex-CoA) via hexanoyl-CoA synthetase. Hex-CoA is then converted to 3-oxooctanoyl-CoA, then 3,5-dioxodecanoyl-CoA, then 3,5,7-trioxododecanoyl-CoA by olivetol synthase (OLS; also known as tetraketide synthase or TKS). The 3,5,7-trioxododecanoyl-CoA is subsequently converted to olivetolic acid by olivetolic acid cyclase (OAC). A prenyltransferase then catalyzes the reaction between olivetolic acid and geranyldiphosphate (GPP) to produce CBGA, which can be converted to CBG via non-enzymatic decarboxylation. In an analogous manner, CBGOA is produced from the prenyltransferase-catalyzed reaction between orsellinic acid and GPP; CBGVA is produced from the prenyltransferase-catalyzed reaction between divarinic acid and GPP. In some embodiments, the CBGA, CBG, CBGOA, and/or CBGVA produced from the cannabinoid biosynthesis pathways are further converted into a cannabinoid by the flavin-dependent oxidases provided herein. Cannabinoid biosynthesis pathways are further described, e.g., in Degenhardt et al., Chapter 2—The Biosynthesis of Cannabinoids. Handbook of Cannabis and Related Pathologies, pp. 13-23; Elsevier Academic Press, 2017. In some embodiments, the cannabinoid biosynthesis pathway enzyme comprises an enzyme from Cannabis sativa, e.g., OLS, OAC, a GPP biosynthesis pathway enzyme, and/or prenyltransferase. In some embodiments, the cannabinoid biosynthesis pathway enzyme comprises a homolog of a C. sativa enzyme, e.g., a homolog of OLS, OAC, GPP pathway enzyme, and/or prenyltransferase. It will be understood by one of ordinary skill in the art that a homolog of a cannabinoid biosynthesis pathway enzyme can be a sequence homolog, a structural homolog, and/or an enzyme activity homolog.

In some embodiments, the engineered cell further comprises an enzyme in the CBGA biosynthesis pathway. In some embodiments, the engineered cell further comprises an enzyme in the CBG biosynthesis pathway. In some embodiments, the engineered cell comprises an enzyme in the CBGOA biosynthesis pathway. In some embodiments, the engineered cell comprises an enzyme in the CBGVA biosynthesis pathway. In some embodiments, the engineered cell comprises an enzyme in the CBGO biosynthesis pathway. In some embodiments, the engineered cell comprises an enzyme in the CBGV biosynthesis pathway.

In some embodiments, CBGA is produced from olivetolic acid (OA) and geranyldiphosphate (GPP). In some embodiments, CBG is produced from CBGA. In some embodiments, CBGOA is produced from orsellinic acid (OSA) and GPP. In some embodiments, CBGVA is produced from divarinic acid (DA) and GPP. In some embodiments, the engineered cells of the disclosure have higher levels of available GPP, OA, OSA, DA, CBGA, CBG, CBGOA, and/or CBGVA (and derivatives or analogs thereof) as compared to a naturally-occurring, non-engineered cell.

### OLS

In some embodiments, the engineered cell of the disclosure further comprises an enzyme in the olivetolic acid pathway. In some embodiments, the enzyme in the olivetolic acid pathway is olivetol synthase (OLS). OLS catalyzes the addition of two malonyl-CoA (Mal-CoA) and hexanoyl-CoA (Hex-CoA) to form 3,5-dioxodecanoyl-CoA, which can be further converted by OLS to 3,5,7-trioxododecanoyl-CoA with the addition of a third Mal-CoA. 3,5,7-trioxododecanoyl-CoA can subsequently be converted to OA by OAC.

Although the metabolic pathway is discussed herein with reference to certain precursors and intermediates, it is understood that analogs may be substituted in essentially the same reactions. For example, it is understood that Hex-CoA analogs, including other acyl-CoAs, can be used in place of Hex-CoA. Exemplary analogs include, but are not limited to any C₂-C₂₀ acyl-CoA such as acetyl-CoA, propionyl-CoA, butyryl-CoA, pentanoyl-CoA, heptanoyl-CoA, octanoyl-CoA, nonanoyl-CoA, decanoyl-CoA, and aromatic acid CoA such as benzoic, chorismic, phenylacetic, and phenoxyacetic acid-CoA.

In some embodiments, the engineered cells of the disclosure have increased production of one or more precursors (e.g., Mal-CoA, Hex-CoA or other acyl-CoA, OA. OSA, DA, CBGA, CBGOA, and/or CBGVA) of the cannabinoids provided herein, e.g., CBCA, CBDA, THCA, CBCOA, CBDOA, THCOA, CBCVA, CBDVA, THCVA, CBC, CBD, THC, CBCO, CBDO, THCO, CBCV, CBDV, and/or THCV. In some embodiments, the engineered cells of the disclosure have increased production of one or more precursors (e.g., Mal-CoA, Hex-CoA or other acyl-CoA, OA, OSA, DA, CBGA, CBGOA, and/or CBGVA) of THCA, CBCA, CBCOA, CBCVA, CBC, CBCO, and/or CBCV.

In some embodiments, the engineered cells of the disclosure have increased production of OA precursors, e.g., Mal-CoA and/or acyl-CoA (such as, e.g., Hex-CoA or any other acyl-CoA described herein). In some embodiments, a non-natural OLS preferentially catalyzes the condensation of Mal-CoA and acyl-CoA (such as, e.g., Hex-CoA or any other acyl-CoA described herein) to form a polyketide (such as, e.g., 3,5,7-trioxododecanoyl-CoA and 3,5,7-trioxododecanoate and their analogs) over the reaction side products, e.g., pentyl diacetic acid lactone (PDAL), hexanoyl triacetic acid lactone (HTAL), or other lactone analogs compared with a wild-type OLS.

In some embodiments, the engineered cell expresses an exogenous or overexpresses an exogenous or endogenous OLS. In some embodiments, the OLS is a natural OLS, e.g., a wild-type OLS. In some embodiments, the OLS is a non-natural OLS. In some embodiments, the OLS comprises one or more amino acid substitutions relative to a wild-type OLS. In some embodiments, the one or more amino acid substitutions in the non-natural OLS increases the activity of the OLS as compared to a wild-type OLS.

In some embodiments, the OLS has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:16.

In some embodiments, the OLS comprises a variation at amino acid position A125, S126, D185, M187, L190, G204, G209, D210. G211, G249, G250, L257, F259, M331, S332, or combinations thereof, wherein the position corresponds to SEQ ID NO:16. In some embodiments, the variation is an amino acid substitution. OLS and non-natural variants thereof are further discussed in, e.g., WO2020/214951.

In some embodiments, the non-natural OLS comprises an amino acid substitution selected from A125G, A125S, A125T, A125C, A125Y, A125H, A125N, A125Q, A125D, A125E, A125K, A125R, S126G, S126A, D185G, D185G, D185A, D185S, D185P, D185C, D185T, D185N, M187G, M187A, M187S, M187P, M187C, M187T, M187D, M187N, M187E, M187Q, M187H, M187H, M187V, M187L, M187I, M187K, M187R, L190G, L190A, L190S, L190P, L190C, L190T, L190D, L190N, L190E, L190Q, L190H, L190V, L190M, L190I, L190K, L190R, G204A, G204C. G204P, G204V, G204L, G2041, G204M, G204F, G204W, G204S, G204T, G204Y, G204H, G204N, G204Q. G204D, G204E, G204K, G204R, G209A, G209C, G209P, G209V, G209L, G2091, G209M, G209F, G209W, G209S, G209T, G209Y, G209H, G209N, G209Q, G209D. G209E, G209K, G209R, D210A, D210C, D210P, D210V, D210L, D210I, D210M, D210F, D210W, D210S, D210T, D210Y, D210H, D210N, D210Q, D210E, D210K, D210R, G211A. G211C. G211P, G211V, G211L, G2111, G211M, G211F, G211W, G211S, G211T. G211Y, G211H, G211N, G211Q, G211D, G211E, G211K, G211R, G249A, G249C, G249P, G249V, G249L, G2491, G249M, G249F, G249W, G249S, G249T, G249Y, G249H, G249N, G249Q, G249D, G249E, G249K, G249R, G249S, G249T, G249Y, G250A, G250C, G250P, G250V, G250L, G2501, G250M, G250F, G250W, G250S, G250T, G250Y, G250H, G250N, G250Q, G250D, G250E, G250K, G250R, L257V, L257M, L2571, L257K, L257R, L257F, L257Y, L257W, L257S, L257T, L257C, L257H, L257N, L257Q, L257D, L257E, F259G, F259A, F259C, F259P, F259V, F259L, F259I. F259M, F259Y, F259W, F259S, F259T, F259Y, F259H, F259N, F259Q, F259D, F259E, F259K, F259R, M331G, M331A, M331S, M331P, M331C, M331T, M331D, M331N, M331E, M331Q, M331H, M331V, M331L, M3311, M331K, M331R, S332G, S332A, or combinations thereof, wherein the position corresponds to SEQ ID NO:16.

In some embodiments, the disclosure provides a composition comprising the flavin-dependent oxidase described herein and the OLS described herein. In some embodiments, the disclosure provides an engineered cell comprising the flavin-dependent oxidase described herein and the OLS described herein. In some embodiments, the disclosure provides one or more polynucleotides comprising one or more nucleic acid sequences encoding the flavin-dependent oxidase described herein and the OLS described herein. In some embodiments, the OLS is a non-natural OLS. In some embodiments, the flavin-dependent oxidase is any of the flavin-dependent oxidases in Table 1. In some embodiments, the flavin-dependent oxidase is a non-natural flavin-dependent oxidase described herein. In some embodiments, the disclosure provides an expression construct comprising the one or more polynucleotides. In some embodiments, the expression construct comprises a single expression vector. In some embodiments, the expression construct comprises more than one expression vector. In some embodiments, the disclosure provides an engineered cell comprising the one or more polynucleotides. In some embodiments, the disclosure provides an engineered cell comprising the expression construct. In some embodiments, the engineered cell produces CBCA, CBDA, THCA, CBCOA, CBDOA, THCOA, CBCVA, CBDVA, THCVA, CBCO, CBDO, THCO. CBCV, CBDV, THCV, CBC, CBD, and/or THC.

In some embodiments, the OLS described herein is enzymatically capable of at least about 1.1, 1.2, 1.5, 2, 3, 4, 5, 6, 7, 8, 9, 10, 12, 15, 20, or greater rate of formation of OA and/or olivetol from Mal-CoA and Hex-CoA in the presence of an excess of the OAC described herein, as compared to a wild type OLS.

### OAC

In some embodiments, the engineered cell of the disclosure further comprises an enzyme in the olivetolic acid pathway. In some embodiments, the enzyme in the olivetolic acid pathway is olivetolic acid cyclase (OAC). As discussed herein, OAC catalyzes the conversion of 3,5,7-trioxododecanoyl-CoA to OA.

In some embodiments, the engineered cell expresses an exogenous or overexpresses an exogenous or endogenous OAC. In some embodiments, the OAC is a natural OAC, e.g., a wild-type OAC. In some embodiments, the OAC is a non-natural OAC. In some embodiments, the OAC comprises one or more amino acid substitutions relative to a wild-type OAC. In some embodiments, the one or more amino acid substitutions in the non-natural OAC increases the activity of the OAC as compared to a wild-type OAC. OAC and non-natural variants thereof are further discussed in, e.g., WO2020/247741.

In some embodiments, the OAC has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:17.

In some embodiments, the OAC comprises a variation at amino acid position L9, F23, V59, V61, V66, E67, 169, Q70, 173, 174, V79, G80, F81, G82, D83, R86, W89, L92, or 194, V46, T47, Q48, K49, N50, K51, V46, T47, Q48, K49, N50, K51, or combinations thereof, wherein the position corresponds to SEQ ID NO: 17. In some embodiments, the variation is an amino acid substitution. In some embodiments, the variation is in a first peptide (e.g., a first monomer) of an OAC dimer. In some embodiments, the variation is in a second peptide (e.g., a second monomer) of an OAC dimer. In some embodiments, the variation is in a first peptide and in a second peptide (e.g., a OAC dimer comprising mutations in each peptide).

In some embodiments, the OAC forms a dimer, wherein a first peptide of the dimer (e.g., a first monomer) of the dimer comprises a variation at amino acid position H5, l7, L9, F23, F24, Y27, V59, V61, V66, E67, 169, Q70, 173, 174, V79, G80, F81, G82, D83, R86, W89, L92, 194, D96, V46, T47, Q48, K49, N50, K51, or combination thereof, and wherein a second peptide (e.g., a second monomer) of the dimer comprises a variation at amino acid position V46, T47, Q48, K49, N50, K51, or combination thereof, wherein the position corresponds to SEQ ID NO:17. In some embodiments, the OAC forms a dimer, wherein a first peptide of the dimer comprises a variation at amino acid position L9, F23, V59, V61, V66, E67, 169, Q70, 173, I74, V79, G80, F81, G82, D83, R86, W89, L92, 194, V46, T47, Q48, K49, N50, K51, or combination thereof, and a second peptide of the dimer comprises a variation at amino acid position V46, T47, Q48, K49, N50, K51, or combination thereof, wherein the position corresponds to SEQ ID NO:17.

In some embodiments, the OAC comprises an amino acid substitution selected from H5X¹, wherein X¹ is G, A, C. P, V, L, I, M, F, Y, W, Q, E, K, R, S, T, Y, N, Q, D, E, K, or R; 17X², wherein X² is G, A, C, P, V, L, M, F, Y, W, K, R, S, T, H, N, Q, D, or E; L9X³, wherein X³ is G, A, C, P, V, I, M, F, Y, W. K, R, S, T, Y, H, N, Q, D, E, K, or R; F23X⁴, wherein X⁴ is G, A, C, P, V, L, I, M, Y, W, S, T, H, N, Q, D, E, K, or R; F24X⁵, wherein X⁵ is G, A, C, P, V, I, M, Y, S, T, H, N, Q, D, E, K, R, or W; Y27X⁶, wherein X⁶ is G, A, C, P, V, L, I, M, F, W, S, T, H, N, Q, D, E, K, or R; V59X⁷, wherein X⁷ is G, A, C, P, L, I, M, F, Y, W, H, Q, E, K, or R; V61X⁸, wherein X⁸ is G, A, C, P, L, I, M, F, Y, W, H, Q, E, K, R, S, T, N, or D; V66X⁹, wherein X⁹ is G, A, C, P, L, I, M, F, Y, or W; E67X¹⁰, wherein X¹⁰ is G, A, C, P, V, L, I, M, F, Y, or W; I69X¹¹, wherein X¹¹ is G, A, C, P, V, L, M, F, Y, or W; Q70X¹², wherein X¹² is S, T, H, N, D, E, R, K, or Y; 173X¹³, wherein X¹³ is G, A, C, P. V, L, M, F, Y, or W; 174X¹⁴, wherein X¹⁴ is G, A, C, P, V, L, M, F, Y, or W; V79X¹⁵, wherein X¹⁵ is G, A, C, P, L, I, M, F, Y, or W; G80X¹⁶, wherein X¹⁶ is A, C. P, V, L, I, M, F, Y, W, S, T, H, N, Q, D, E, K, or R; F81X¹⁷, wherein X¹⁷ is G, A, C, P, V, L, I, M, Y, W, S, T, H, N, Q, D, E, R, or K; G82X¹⁸, wherein X¹⁸ is A, C, P, V, L, I, M, F, Y, W. S, T, H, N, Q, E, K, or R; D83X¹⁹, wherein X¹⁹ is S, T, H, Q, N, E, R, K, or Y; R86X²⁰, wherein X²⁰ is S, T, H, Q, N, D, E, K, or Y; W89X²¹, wherein X²1 is G, A, C, P, V, L, I, M, F, Y, W, S, T, H, N, Q, D, E, K, or R; L92X²², wherein X²² is G, A, C, P, V, I, M, F, Y, or W; 194X²³, wherein X²³ is G, A, C, P, V, L, M, F, Y, W, K, R, S, T, Y, H, N, Q, D, or E; D96X²⁴, wherein X²⁴ is S, T, H, Q, N, E, R, K, or Y; V46X²⁵, wherein X²⁵ is G, A, C, P, L, I, M, F, Y, or W; T47X²⁶, wherein X²⁶ is S, H, Q, N, D, E, R, K, or Y; Q48X²⁷, wherein X²⁷ is S, T, H, N, D, E, R, K, or Y; K49X²⁸, wherein X²⁸ is S, T, H, Q, N, D, E, R, or Y; N50X²⁹, wherein X²⁹ is G, A, C, P, V, L, I, M, F, Y, or W; K51X³⁰, wherein X³⁰ is S, T, H, Q, N, D, E, R, or Y; V46*X³1, wherein X³¹ is G, A, C, P, L, I, M, F, Y, or W; T47*X³², wherein X³² is S, H, Q, N, D, E, R, K, or Y; Q48*X³³, wherein X³³ is S, T, H, N, D, E, R, K, or Y; K49*X³⁴, wherein X³⁴ is S, T, H, Q, N, D, E, R, or Y; N50*X³⁵, wherein X³⁵ is G, A, C, P, V, L, I, M, F, Y, or W; K51*X³⁶, wherein X³⁶ is S, T, H, Q, N, D, E, R, or Y; and combinations thereof; wherein the amino acid position corresponds to SEQ ID NO:17, and wherein the “*” following the amino acid position indicates amino acid residues from a second peptide of a OAC dimer (e.g., monomer B) and corresponding to SEQ ID NO:17.

In some embodiments, the OAC comprises more than one amino acid variations. In some embodiments, the OAC is not a single substitution at position K4A, H5A, H5L, H5Q, H5S, H5N, H5D, I7L, I7F, L9A, L9W, K12A, F23A, F23I, F23W, F23L, F24L, F24W, F24A, Y27F, Y27M, Y27W, V28F, V29M, K38A, V40F, D45A, H57A, V59M, V59A, V59F, Y72F, H75A, H78A, H78N, H78Q, H78S, H78D, or D96A, wherein the amino acid position corresponds to SEQ ID NO:17.

In some embodiments, the OAC described herein is capable of producing olivetolic acid at a faster rate compared with a wild-type OAC. In some embodiments, the OAC has increased affinity for a polyketide (e.g., 3,5,7-trioxododecanoyl-CoA or an analog thereof, as produced by an OLS described herein) compared with a wild-type OAC. In some embodiments, the rate of formation of olivetolic acid from 3,5,7-trioxododecanoyl-CoA or analog thereof by the OAC described herein is about 1.2 times to about 300 times, about 1.5 times to about 200 times, or about 2 times to about 30 times as compared to a wild-type OAC. The rate of formation of olivetolic acid from 3,5,7-trioxododecanoyl-CoA or an analog thereof can be determined in an in vitro enzymatic reaction using a purified OAC. Methods of determining enzyme kinetics and product formation rate are known in the field.

In some embodiments, the OAC is present in molar excess of the OLS in the engineered cell. In some embodiments, the molar ratio of the OLS to the OAC is about 1:1.1, 1:1.2, 1:1.5, 1:1.8, 1:2, 1:3, 1:4, 1:5, 1:10, 1:20, 1:25, 1:50, 1:75, 1:100, 1:125, 1:150, 1:200, 1:250, 1:300, 1:350, 1:400, 1:450, 1:500, 1:1000, 1:1250, 1:1500, 1:2000, 1:2500, 1:5000, 1:7500, 1:10,000, or 1 to more than 10,000. In some embodiments, the molar ratio of the OLS to the OAC is about 1000:1, 500:1, 100:1, 10:1, 5:1, 2.5:1, 1.5:1, 1.2:1, 1.1:1, 1:1, or less than 1 to 1. In some embodiments, the enzyme turnover rate of the OAC is greater than OLS. As used herein, “turnover rate” refers to the rate at which an enzyme can catalyze a reaction (e.g., turn substrate into product). In some embodiments, the higher turnover rate of OAC compared to OLS provides a greater rate of formation of OA than olivetol.

In some embodiments, the total byproducts (e.g., olivetol and analogs thereof, PDAL, HTAL, and other lactone analogs) of the OLS reaction products in the presence of molar excess of OAC, are in an amount (w/w) of less than about 50%, 45%, 40%, 35%, 30%, 25%, 20%, 15%, 12.5%, 10%, 9%, 8%, 7%. 6%, 5%, 4%, 3%, 2%, 1%, 0.9%, 0.8%, 0.7%, 0.6%, 0.5%, 0.4%, 0.3%, 0.2%, 0.1%, 0.05%, 0.025%, or 0.01% of the total weight of the products formed by the combination of individual OLS and OAC enzyme reactions.

In some embodiments, the disclosure provides a composition comprising the flavin-dependent oxidase described herein and one or both of the OLS described herein and the OAC described herein. In some embodiments, the disclosure provides an engineered cell comprising the flavin-dependent oxidase described herein and one or both of the OLS described herein and the OAC described herein. In some embodiments, the disclosure provides one or more polynucleotides comprising one or more nucleic acid sequences encoding the flavin-dependent oxidase described herein and one or both of the OLS described herein and the OAC described herein. In some embodiments, the flavin-dependent oxidase is any of the flavin-dependent oxidases in Table 1. In some embodiments, the flavin-dependent oxidase is a non-natural flavin-dependent oxidase described herein. In some embodiments, the disclosure provides an expression construct comprising the one or more exogenous polynucleotides. In some embodiments, the expression construct comprises a single expression vector. In some embodiments, the expression construct comprises more than one expression vector. In some embodiments, the disclosure provides an engineered cell comprising the one or more polynucleotides. In some embodiments, the disclosure provides an engineered cell comprising the expression construct. In some embodiments, the engineered cell produces CBCA, CBDA, THCA, CBCOA, CBDOA, THCOA, CBCVA, CBDVA, THCVA, CBCO, CBDO, THCO, CBCV, CBDV, THCV, CBC. CBD, and/or THC or analogs or derivatives thereof.

### GPP

In some embodiments, the engineered cell of the disclosure further comprises an enzyme in the geranyl pyrophosphate (GPP) pathway. GPP pathways are further provided, e.g., in WO 2017/161041. In some embodiments, the GPP pathway comprises a mevalonate (MVA) pathway, a non-mevalonate methylerythritol-4-phosphate (MEP) pathway, an alternative non-MEP, non-MVA geranyl pyrophosphate pathway, or combinations thereof. In some embodiments, the GPP pathway comprises an enzyme selected from geranyl pyrophosphate (GPP) synthase, farnesyl pyrophosphate synthase, isoprenyl pyrophosphate synthase, geranylgeranyl pyrophosphate synthase, alcohol kinase, alcohol diphosphokinase, phosphate kinase, isopentenyl diphosphate isomerase, or combinations thereof. In some embodiments, the alternative non-MEP, non-MVA geranyl pyrophosphate pathway comprises alcohol kinase, alcohol diphosphokinase, phosphate kinase, isopentenyl disphosphate isomerase, geranyl pyrophosphate synthase, or combinations thereof.

GPP and its precursors may be produced from several pathways within a host cell, including the mevalonate pathway (MVA) or a non-mevalonate, methylerythritol-4-phosphate (MEP) pathway (also known as the deoxyxylulose-5-phosphate pathway), which produce isopentenyl pyrophosphate (IPP) and dimethylallyl pyrophosphate (DMAPP), which are isomerized by isopentenyl-diphosphate delta-isomerase (IDI) and converted GPP using geranyl pyrophosphate synthase (GPPS). As described herein, prenyltransferase can convert GPP and OA into CBGA, which can then be converted into CBCA and/or THCA by the flavin-dependent oxidase described herein. Prenyltransferase can also convert GPP and OSA into CBGOA, which can then be converted in CBCOA by the flavin-dependent oxidase described herein. Prenyltransferase can further convert GPP and DA into CBGVA, which can then be converted into CBCVA by the flavin-dependent oxidase described herein.

In some embodiments, the engineered cell produces GPP from a MVA pathway. In some embodiments, the engineered cell produces GPP from a MEP pathway. In some embodiments, the engineered cell expresses an exogenous or overexpresses an exogenous or endogenous gene that encodes any one of the enzymes in the MVA pathway or the MEP pathway, thereby increasing the production of GPP. In some embodiments, the MVA pathway enzyme is acetoacetyl-CoA thiolase (AACT); HMG-CoA synthase (HMGS); HMG-CoA reductase (HMGR); mevalonate-3-kinase (MVK); phosphomevalonate kinase (PMK); mevalonate-5-pyrophosphate decarboxylase (MVD); isopentenyl pyrophosphate isomerase (IDI), or geranyl pyrophosphate synthase (GPPS). In some embodiments, the MEP pathway enzyme is 1-deoxy-D-xylulose 5-phosphate synthase (DXS), 1-deoxy-D-xylulose 5-phosphate reductoisomerase (DXR); 2-C-methyl-D-erythritol 4-phosphate cytidylyltransferase (CMS); 4-diphosphocytidyl-2-C-methyl-D-erythritol kinase (CMK); 2-C-methyl-D-erythritol 2,4-cyclodiphosphate synthase (MECS); 4-hydroxy-3-methyl-but-2-enyl pyrophosphate synthase (HDS); 4-hydroxy-3-methyl-but-2-enyl pyrophosphate reductase (HDR); isopentenyl pyrophosphate isomerase (IDI), or geranyl pyrophosphate synthase (GPPS). In some embodiments, the MVA pathway enzyme is mevalonate 3-phosphate-5-kinase, isopentenyl-5-phosphate kinase, mevalonate-5-phosphate decarboxylase, or mevalonate-5-kinase. In some embodiments, the increased production of GPP results in increased production of the cannabinoids described herein, e.g., CBCA, CBDA, THCA, CBCOA, CBDOA, THCOA, CBCVA, CBDVA, THCVA, CBCO, CBDO, THCO, CBCV, CBDV, THCV, CBC, CBD, and/or THC, by the flavin-dependent oxidase described herein. In some embodiments, the increased production of GPP results in increased production of CBCA, THCA, CBCOA, CBCVA, CBCO, CBCV, and/or CBC, by the flavin-dependent oxidase described herein.

In some embodiments, the engineered cell produces GPP from an alternative non-MEP, non-MVA geranyl pyrophosphate pathway. In some embodiments, GPP is produced from a precursor selected from isoprenol, prenol, and geraniol. In some embodiments, the engineered cell expresses an exogenous or overexpresses an exogenous or endogenous gene that encodes any one of the enzymes in a non-MVA, non-MEP pathways, thereby increasing the production of GPP. In some embodiments, the non-MVA, non-MEP pathway enzyme is alcohol kinase, alcohol diphosphokinase, phosphate kinase, isopentenyl diphosphate isomerase, or geranyl pyrophosphate synthase (GPPS). In some embodiments, the increased production of GPP results in increased production of the cannabinoids described herein, e.g., CBCA, CBDA, THCA, CBCOA, CBDOA, THCOA, CBCVA, CBDVA, THCVA, CBCO, CBDO, THCO, CBCV, CBDV, THCV, CBC, CBD, and/or THC, by the flavin-dependent oxidase described herein.

In some embodiments, the engineered cell an exogenous or overexpresses an exogenous or endogenous GPP synthase. Non-limiting examples of GPP synthases include E. coli IspA (NP_414955), C. glutamicum IdsA (WP_011014931.1), and the enzymes listed in Table 2.

TABLE 2
Exemplary GPP Synthases

| | GenBank | | GenBank |
|---|---|---|---|
| Species | Accession No. | Species | Accession No. |
| Abies grandis | AAN01133.1 and | Corynebacterium | WP_035105251.1 |
| | AAN01134.1 | camporealensis | |
| Corynebacterium crudilactis | WP 074025495.1 | Corynebacterium | WP 005328932.1 |
| | | tuberculostearicum | |
| Corynebacterium | WP_096457048.1 | Corynebacterium | WP_005324491.1 |
| glutamicum | | pseudogenitalium | |
| Corynebacterium deserti | WP_053545301.1 | Corynebacterium testudinoris | WP_083985528.1 |
| Corynebacterium callunae | WP_015651699.1 | Corynebacterium stationis | WP_066793135.1 |
| Corynebacterium efficiens | WP_006768068.1 | Corynebacterium sp. J010B- | WP_105324112.1 |
| | | 136 | |
| Corynebacterium sp. | WP_080794061.1 | Corynebacterium sp. CCUG | WP_123047545.1 |
| Marseille-P2417 | | 69366 | |
| Corynebacterium | WP_040086238.1 | Corynebacterium sp. | WP_023030480.1 |
| humireducens | | KPL1818 | |
| Corynebacterium | WP_015401326.1 | Corynebacterium accolens | WP_005283903.1 |
| halotolerans | | | |
| Corynebacterium marinum | WP_042621772.1 | Corynebacterium | WP_126319428.1 |
| | | segmentosum | |
| Corynebacterium singulare | WP_042531577.1 | Corynebacterium macginleyi | WP_121911356.1 |
| Corynebacterium | WP 115022907.1 | Pseudomonas aeruginosa | SQG59150.1 |
| minutissimum | | | |
| Corynebacterium pollutisoli | WP_143337494.1 | Streptococcus thermophilus | VDG63248.1 |
| Corynebacterium lubricantis | WP_018297093.1 | Nocardia vermiculata | WP_084473733.1 |
| Corynebacterium | WP_092284621.1 | Rhodococcus sp. 1168 | WP_088945631.1 |
| spheniscorum | | | |
| Corynebacterium | WP_018020857.1 | Clostridium paraputrificum | WP_113570111.1 |
| doosanense | | | |
| Corynebacterium flavescens | WP_075731219.1 | Nocardia cyriacigeorgica | WP_036535265.1 |
| Corynebacterium | WP_143334899.1 | Nocardia concava | WP_040806894.1 |
| aurimucosum | | | |
| Corynebacterium | WP_003845210.1 | Rhodococcus yunnanensis | WP_072806331.1 |
| ammoniagenes | | | |
| Corynebacterium | WP_086587718.1 | | |
| kefirresidentii | | | |

In some embodiments, the disclosure provides a composition comprising the flavin-dependent oxidase described herein and one or more of the OLS described herein, the QAC described herein, and the GPP pathway enzyme described herein. In some embodiments, the disclosure provides an engineered cell comprising the flavin-dependent oxidase described herein and one or more of the OLS described herein, the OAC described herein, and the GPP pathway enzyme described herein. In some embodiments, the disclosure provides one or more polynucleotides comprising one or more nucleic acid sequences encoding the flavin-dependent oxidase described herein and one or more of the OLS described herein, the OAC described herein, and the GPP pathway enzyme described herein. In some embodiments, the GPP pathway enzyme comprises geranyl pyrophosphate (GPP) synthase, farnesyl pyrophosphate synthase, isoprenyl pyrophosphate synthase, geranylgeranyl pyrophosphate synthase, alcohol kinase, alcohol diphosphokinase, phosphate kinase, isopentenyl diphosphate isomerase, geranyl pyrophosphate synthase, or combinations thereof. In some embodiments, the flavin-dependent oxidase is any of the flavin-dependent oxidases in Table 1. In some embodiments, the flavin-dependent oxidase is a non-natural flavin-dependent oxidase described herein. In some embodiments, the disclosure provides an expression construct comprising the one or more polynucleotides. In some embodiments, the expression construct comprises a single expression vector. In some embodiments, the expression construct comprises more than one expression vector. In some embodiments, the disclosure provides an engineered cell comprising the one or more polynucleotides. In some embodiments, the disclosure provides an engineered cell comprising the expression construct. In some embodiments, the engineered cell produces CBCA, CBDA, THCA, CBCOA, CBDOA, THCOA, CBCVA, CBDVA, THCVA, CBCO, CBDO, THCO, CBCV, CBDV, THCV, CBC, CBD, and/or THC.

### Prenyltransferase

In some embodiments, the engineered cell of the disclosure further comprises a prenyltransferase.

In general, the conversion of OA+GPP to CBGA (and the analogous conversions of OSA+GPP to CBGOA and DA+GPP to CBGVA) is performed by a prenyltransferase. In C. sativa, prenyltransferase is a transmembrane protein belonging to the UbiA superfamily of membrane proteins. Other prenyltransferases, e.g., aromatic prenyltransferases such as NphB from Streptomyces, which are non-transmembrane and soluble, can also catalyze conversion of OA to CBGA, OSA to CBGOA, and/or DA to CBGVA.

In some embodiments, the prenyltransferase is a natural prenyltransferase, e.g., wild-type prenyltransferase. In some embodiments, the prenyltransferase is a non-natural prenyltransferase. In some embodiments, the prenyltransferase comprises one or more amino acid substitutions relative to a wild-type prenyltransferase. In some embodiments, the one or more amino acid substitutions in the non-natural prenyltransferase increases the activity of the prenyltransferase as compared to a wild-type prenyltransferase.

In some embodiments, the prenyltransferase has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:18. In some embodiments, the prenyltransferase is a non-natural prenyltransferase comprising at least 1, at least 2, at least 3, at least 4, at least 5, at least 6, at least 7, at least 8, at least 9, or at least 10 amino acid variations at positions corresponding to SEQ ID NO:18.

Although the amino acid positions of prenyltransferase described herein are with reference to the corresponding amino acid sequence of SEQ ID NO:18, it is understood that the amino acid sequence of a non-natural prenyltransferase can include an amino acid variation at an equivalent position corresponding to a variant of SEQ ID NO:18. One of the skill in the art would understand that alignment methods can be used to align variations of SEQ ID NO:18 to identify the position in the prenyltransferase variant that corresponds to a position in SEQ ID NO:18. In some embodiments, SEQ ID NO:18 corresponds to the amino acid sequence of Streptomyces antibioticus AQJ23_4042 prenyltransferase.

In some embodiments, the prenyltransferase comprises an amino acid substitutions at position V45, F121, T124, Q159, M160, Y173, S212, V213, A230, T267, Y286, Q293, R294, L296, F300, or combinations thereof, wherein the position corresponds to SEQ ID NO:18. In some embodiments, the prenyltransferase comprises two or more amino acid substitutions at positions V45, F121, T124, Q159, M160, Y173, S212, V213, A230, T267, Y286, Q293, R294, L296, F300, or combinations thereof. In some embodiments, the prenyltransferase comprises two or more amino acid substitutions at positions V45, F121, T124, Q159, M160, Y173, S212, V213, A230, T267, Y286, Q293, R294, L296, F300, or combinations thereof. Prenyltransferase and non-natural variants thereof are further discussed, e.g., in WO2019/173770 and WO2021/046367.

In some embodiments, the amino acid substitution is selected from V45I, V45T, F121V, T124K, T124L, Q159S, M160L, M160S, Y173D, Y173K, Y173P, Y173Q, S212H, A230S, T267P, Y286V, Q293H, R294K, L296K, L296L, L296M, L296Q, F300Y, and combinations thereof.

In some embodiments, the prenyltransferase comprising an amino acid substitution as described herein is capable of a greater rate of formation of CBGA from GPP and OA, CBGOA from GPP and OSA, and/or CBGVA from GPP and DA as compared with wild-type prenyltransferase.

In some embodiments, the disclosure provides a composition comprising the flavin-dependent oxidase described herein and one or more of the OLS described herein, the OAC described herein, the GPP pathway enzyme described herein, and the prenyltransferase described herein. In some embodiments, the disclosure provides an engineered cell comprising the flavin-dependent oxidase described herein and one or more of the OLS described herein, the OAC described herein, the GPP pathway enzyme described herein, and the prenyltransferase described herein. In some embodiments, the disclosure provides one or more polynucleotides comprising one or more nucleic acid sequences encoding the flavin-dependent oxidase described herein and one or more of the OLS described herein, the OAC described herein, the GPP pathway enzyme described herein, and the prenyltransferase described herein. In some embodiments, the flavin-dependent oxidase is any of the flavin-dependent oxidases in Table 1. In some embodiments, the flavin-dependent oxidase is a non-natural flavin-dependent oxidase described herein. In some embodiments, the disclosure provides an expression construct comprising the one or more polynucleotides. In some embodiments, the expression construct comprises a single expression vector. In some embodiments, the expression construct comprises more than one expression vector. In some embodiments, the disclosure provides an engineered cell comprising the one or more polynucleotides. In some embodiments, the disclosure provides an engineered cell comprising the expression construct. In some embodiments, the engineered cell produces CBCA, CBDA, THCA, CBCOA, CBDOA, THCOA, CBCVA, CBDVA, THCVA, CBCO, CBDO, THCO, CBCV, CBDV, THCV, CBC, CBD, and/or THC.

### Additional Strain Modifications

In some embodiments, the engineered cell of the disclosure further comprises a modification that facilitates the production of the cannabinoids described herein, e.g., CBCA, CBDA, THCA, CBCOA, CBDOA, THCOA, CBCVA, CBDVA, THCVA, CBCO, CBDO, THCO, CBCV, CBDV, THCV, CBC, CBD, and/or THC. In some embodiments, the modification increases production of a cannabinoid in the engineered cell compared with a cell not comprising the modification. In some embodiments, the modification increases efflux of a cannabinoid in the engineered cell compared with a cell not comprising the modification. In some embodiments, the modification comprises expressing or upregulating the expression of an endogenous gene that facilitates production of a cannabinoid. In some embodiments, the modification comprises introducing and/or overexpression an exogenous and/or heterologous gene that facilitates production of a cannabinoid. In some embodiments, the modification comprises downregulating, disrupting, or deleting an endogenous gene that hinders production of a cannabinoid. Expression and/or overexpression of endogenous and exogenous genes, and downregulation, disruption and/or deletion of endogenous genes are described in embodiments herein.

In some embodiments, the engineered cell of the disclosure comprises one or more of the following modifications:
i) express one or more exogenous nucleic acid sequences or overexpress one or more endogenous genes encoding a protein having an ABC transporter permease activity;
ii) express one or more exogenous nucleic acid sequences or overexpress one or more endogenous genes encoding a protein having an ABC transporter ATP-binding protein activity;
iii) express one or more exogenous nucleic acids sequences or overexpress one or more endogenous genes selected from blc, ydhC, ydhG, or a homolog thereof;
iv) express one or more exogenous nucleic acids sequences or overexpress one or more endogenous genes selected from mlaD, mlaE, mlaF, or a homolog thereof;
v) express one or more exogenous nucleic acid sequences or overexpress one or more endogenous genes encoding a protein having a siderophore receptor protein activity or overexpress one or more endogenous genes encoding a protein having a siderophore receptor protein activity;
vi) comprise a disruption of or downregulation in the expression of a regulator of expression of one or more endogenous genes encoding a protein having an ABC transporter permease activity, a protein having an ABC transporter ATP-binding protein activity, a ble gene, a ybhG protein, a ydhC protein, a mlaD protein, mlaE protein, mlaF protein, or a protein having a siderophore receptor protein activity;
vii) express one or more exogenous nucleic acids sequences or overexpress one or more endogenous genes encoding a multi-domain protein having acetyl-CoA carboxylase activity (MD-ACC);
viii) express one or more exogenous nucleic acids sequences or overexpress one or more endogenous genes encoding acetyl-CoA carboxyltransferase subunit α, biotin carboxyl carrier protein, biotin carboxylase, or acetyl-CoA carboxyltransferase subunit β, or express one or more exogenous nucleic acids or overexpress one or more endogenous genes encoding acetyl-CoA carboxyltransferase, biotin carboxyl carrier protein, or biotin carboxylase activities;
ix) disruption of or downregulation in the expression of an endogenous gene encoding a protein having (acyl-carrier-protein) S-malonyltransferase activity, an endogenous gene encoding a protein having 3-hydroxypalmityl-(acyl-carrier-protein) dehydratase activity, or both;
x) express an exogenous nucleic acid sequence or overexpress an endogenous gene encoding a protein having fatty acyl-CoA ligase activity, or both;
xi) disruption of or downregulation in the expression of at least one endogenous gene encoding a protein having acyl-CoA dehydrogenase activity or enoyl-CoA hydratase activity;
xii) comprise a disruption of or downregulation in the expression of at least one endogenous gene encoding a protein having acyl-CoA esterase/thioesterase activity;
xiii) comprise a disruption of or downregulation in the expression of at least one endogenous gene encoding a repressor of transcription of one or more genes required for fatty acid beta-oxidation or an upregulator of fatty acid biosynthesis in combination with disruption or downregulation of one or more endogenous genes encoding one or more proteins of fatty acid beta-oxidation pathway;
xiv) express an exogenous nucleic acid sequence or overexpress an endogenous gene encoding a protein having geranyl pyrophosphate synthase (GPPS), farnesyl pyrophosphate synthase, isoprenyl pyrophosphate synthase, geranylgeranyl pyrophosphate synthase, alcohol kinase, alcohol diphosphokinase, phosphate kinase, isopentenyl diphosphate isomerase, geranyl pyrophosphate synthase, prenol kinase activity, prenol diphosphokinase activity, isoprenol kinase activity, isoprenol diphosphokinase activity, dimethylallyl phosphate kinase activity, isopentenyl phosphate kinase activity, or isopentenyl diphosphate isomerase activity;
xv) express an exogenous nucleic acid sequence or overexpress an endogenous gene encoding a protein having GPP synthase activity;
xvi) express an exogenous nucleic acid sequence encoding an olivetol synthase;
xvii) express an exogenous nucleic acid sequence encoding an olivetolic acid cyclase;
xviii) express an exogenous nucleic acid sequence encoding a prenyltransferase;
xix) express one or more exogenous nucleic acid sequences or overexpressing one or more endogenous genes encoding one or more enzymes of MVA pathway, MEP pathway, or a non-MVA, non-MEP pathway;
xx) express an exogenous nucleic acid sequence or overexpress an endogenous gene encoding a biotin-(acetyl-CoA carboxylase) ligase;
xxi) express an exogenous nucleic acid sequence or overexpress an endogenous gene encoding a isopentenyl-diphosphate delta-isomerase;
xxii) express an exogenous nucleic acid sequence or overexpress an endogenous gene encoding a hydroxyethylthiazole kinase or both;
xxiii) express an exogenous nucleic acid sequence or overexpress an endogenous gene encoding a Type III pantothenate kinase; and
xxiv) comprise a disruption of or downregulation in the expression of at least one endogenous gene encoding a phosphatase selected from the group consisting of ADP-sugar pyrophosphatase, dihydroneopterin triphosphate diphosphatase, pyrimidine deoxynucleotide diphosphatase, pyrimidine pyrophosphate phosphatase, and Nudix hydrolase.

In some embodiments, the disclosure provides an engineered cell comprising the flavin-dependent oxidase described herein and one or more of the OLS described herein, the OAC described herein, the GPP pathway enzyme described herein, the prenyltransferase described herein, and an additional modification described herein. In some embodiments, the disclosure provides one or more polynucleotides comprising one or more nucleic acid sequences encoding the flavin-dependent oxidase described herein and one or more of the OLS described herein, the OAC described herein, the GPP pathway enzyme described herein, the prenyltransferase described herein, and an additional modification described herein. In some embodiments, the flavin-dependent oxidase is any of the flavin-dependent oxidases in Table 1. In some embodiments, the flavin-dependent oxidase is a non-natural flavin-dependent oxidase described herein. In some embodiments, the disclosure provides an expression construct comprising the one or more polynucleotides. In some embodiments, the expression construct comprises a single expression vector. In some embodiments, the expression construct comprises more than one expression vector. In some embodiments, the disclosure provides an engineered cell comprising the one or more polynucleotides. In some embodiments, the disclosure provides an engineered cell comprising the expression construct. In some embodiments, the engineered cell produces CBCA, CBDA. THCA, CBCOA, CBDOA, THCOA, CBCVA, CBDVA, THCVA, CBCO, CBDO, THCO, CBCV, CBDV, THCV, CBC, CBD, and/or THC.

### Host Cells

A variety of microorganisms may be suitable as the engineered cell described herein. Such organisms include both prokaryotic and eukaryotic organisms including, but not limited to, bacteria, including archaea and eubacteria, and eukaryotes, including yeast, plant, and insect. Nonlimiting examples of suitable microbial hosts for the bio-production of a cannabinoid include, but are not limited to, any Gram negative organisms, more particularly a member of the family Enterobacteriaceae, such as E. coli, or Oligotropha carboxidovorans, or a Pseudomonas sp.; any Gram positive microorganism, for example Bacillus subtilis, Lactobacillus sp. or Lactococcus sp.; a yeast, for example Saccharomyces cerevisiae, Pichia pastoris or Pichia stipitis; and other groups or microbial species. In some embodiments, the microbial host is a member of the genera Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Pichia, Candida, Hansenula, or Saccharomyces. In some embodiments, the microbial host is Oligotropha carboxidovorans (such as strain OM5), Escherichia coli, Alcaligenes eutrophus (Cupriavidus necator), Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis or Saccharomyces cerevisiae.

Further exemplary species are reported in U.S. Pat. No. 9,657,316 and include, for example, Escherichia coli, Saccharomyces cerevisiae, Saccharomyces kluyveri, Candida boidinii, Clostridium kluyveri, Clostridium acetobutylicum, Clostridium beijerinckii, Clostridium saccharoperbutylacetonicum, Clostridium perfringens, Clostridium difficile, Clostridium botulinum, Clostridium tyrobutyricum, Clostridium tetanomorphum, Clostridium tetani, Clostridium propionicum, Clostridium aminobutyricum. Clostridium subterminale, Clostridium sticklandii, Ralstonia eutropha, Mycobacterium bovis, Mycobacterium tuberculosis, Porphyromonas gingivalis, Arabidopsis thaliana, Thermus thermophilus, Pseudomonas species, including Pseudomonas aeruginosa, Pseudomonas putida, Pseudomonas stutzeri, Pseudomonas fluorescens, Homo sapiens, Oryctolagus cuniculus, Rhodobacter spaeroides, Thermoanaerobacter brockii, Metallosphaera sedula, Leuconostoc mesenteroides, Chloroflexus aurantiacus, Roseiflexus castenholzii, Erythrobacter, Simmondsia chinensis, Acinetobacter species, including Acinetobacter calcoaceticus and Acinetobacter baylyi, Porphyromonas gingivalis, Sulfolobus tokodaii, Sulfolobus solfataricus, Sulfolobus acidocaldarius, Bacillus subtilis, Bacillus cereus, Bacillus megaterium, Bacillus brevis, Bacillus pumilus, Rattus norvegicus, Klebsiella pneumonia, Klebsiella oxytoca, Euglena gracilis, Treponema denticola, Moorella thermoacetica, Thermotoga maritima, Halobacterium salinarum, Geobacillus stearothermophilus, Aeropyrum pernix, Sus scrofa, Caenorhabditis elegans, Corynebacterium glutamicum, Acidaminococcus fermentans, Lactococcus lactis, Lactobacillus plantarum, Streptococcus thermophilus, Enterobacter aerogenes, Candida, Aspergillus terreus, Pedicoccus pentosaceus, Zymomonas mobilus, Acetobacter pasteurians, Kluyveromyces lactis, Eubacterium barkeri, Bacteroides capillosus, Anaerotruncus colihominis, Natranaerobius thermophilum, Campylobacter jejuni, Hacmophilus influenzac, Serratia marcescens, Citrobacter amalonaticus, Myxococcus xanthus, Fusobacterium nuleatum, Penicillium chrysogenum, marine gamma proteobacterium, butyrate-producing bacterium, Nocardia iowensis, Nocardia farcinica, Streptomyces griseus, Schizosaccharomyces pombe, Geobacillus thermoglucosidasius, Salmonella typhimurium, Vibrio cholera, Heliobacter pylori, Nicotiana tabacum. Oryza sativa, Haloferax mediterranei, Agrobacterium tumefaciens, Achromobacter denitrificans, Fusobacterium nucleatum, Streptomyces clavuligenus, Acinetobacter baumanii, Mus musculus, Lachancea kluyveri, Trichomonas vaginalis, Trypanosoma brucei, Pseudomonas stutzeri, Bradyrhizobium japonicum, Mesorhizobium loti, Bos taurus, Nicotiana glutinosa, Vibrio vulnificus, Selenomonas ruminantium, Vibrio parahaemolyticus, Archaeoglobus fulgidus, Haloarcula marismortui, Pyrobaculum aerophilum, Mycobacterium smegmatis MC2 155, Mycobacterium avium subsp. paratuberculosis K-10, Mycobacterium marinum M, Tsukamurella paurometabola DSM 20162, Cyanobium PCC7001, Dictyostelium discoideum AX4, as well as other exemplary species disclosed herein or available as source organisms for corresponding genes.

In some embodiments, the engineered cell is a bacterial cell or a fungal cell. In some embodiments, the engineered cell is a bacterial cell. In some embodiments, the engineered cell is a yeast cell. In some embodiments, the engineered cell is an algal cell. In some embodiments, the engineered cell is a cyanobacterial cell. In some embodiments, the bacteria is Escherichia, Corynebacterium, Bacillus, Ralstonia, Zymomonas, or Staphylococcus. In some embodiments, the bacterial cell is an Escherichia coli cell.

In some embodiments, the engineered cell is an organism selected from Acinetobacter baumannii Naval-82, Acinetobacter sp. ADP1, Acinetobacter sp. strain M-1, Actinobacillus succinogenes 130Z, Allochromatium vinosum DSM 180, Amycolatopsis methanolica, Arabidopsis thaliana, Atopobium parvulum DSM 20469, Azotobacter vinelandii DJ, Bacillus alcalophilus ATCC 27647, Bacillus azotoformans LMG 9581, Bacillus coagulans 36D1, Bacillus megaterium, Bacillus methanolicus MGA3, Bacillus methanolicus PB1, Bacillus selenitireducens MLS10, Bacillus smithii, Bacillus subtilis, Burkholderia cenocepacia, Burkholderia cepacia, Burkholderia multivorans, Burkholderia pyrrocinia, Burkholderia stabilis, Burkholderia thailandensis E264, Burkholderiales bacterium Joshi_001, Butyrate-producing bacterium L2-50, Campylobacter jejuni, Candida albicans, Candida boidinii, Candida methylica, Carboxydothermus hydrogenoformans, Carboxydothermus hydrogenoformans Z-2901, Caulobacter sp. AP07, Chloroflexus aggregans DSM 9485, Chloroflexus aurantiacus J-10-fl, Citrobacter freundii, Citrobacter koseri ATCC BAA-895, Citrobacter youngae, Clostridium, Clostridium acetobutylicum, Clostridium acetobutylicum ATCC 824, Clostridium acidurici, Clostridium aminobutyricum, Clostridium asparagiforme DSM 15981, Clostridium beijerinckii, Clostridium beijerinckii NCIMB 8052, Clostridium bolteae ATCC BAA-613, Clostridium carboxidivorans P7, Clostridium cellulovorans 743B, Clostridium difficile, Clostridium hiranonis DSM 13275, Clostridium hylemonae DSM 15053, Clostridium kluyveri, Clostridium kluyveri DSM 555, Clostridium ljungdahlii, Clostridium ljungdahlii DSM 13528, Clostridium methylpentosum DSM 5476, Clostridium pasteurianum, Clostridium pasteurianum DSM 525, Clostridium perfringens, Clostridium perfringens ATCC 13124, Clostridium perfringens str. 13. Clostridium phytofermentans ISDg, Clostridium saccharobutylicum, Clostridium saccharoperbutylacetonicum, Clostridium saccharoperbutylacetonicum N1-4, Clostridium tetani, Corynebacterium glutamicum ATCC 14067, Corynebacterium glutamicum R, Corynebacterium sp. U-96. Corynebacterium variabile, Cupriavidus necator N−1, Cyanobium PCC7001, Desulfatibacillum alkenivorans AK-01, Desulfitobacterium hafniense, Desulfitobacterium metallireducens DSM 15288, Desulfotomaculum reducens MI-1, Desulfovibrio africanus str. Walvis Bay, Desulfovibrio fructosovorans JJ, Desulfovibrio vulgaris str. Hildenborough, Desulfovibrio vulgaris str. ‘Miyazaki F’, Dictyostelium discoideum AX4, Escherichia coli, Escherichia coli K-12, Escherichia coli K-12 MG1655, Eubacterium hallii DSM 3353, Flavobacterium frigoris, Fusobacterium nucleatum subsp. polymorphum ATCC 10953, Geobacillus sp. Y4.1MC1, Geobacillus themodenitrificans NG80-2, Geobacter bemidjiensis Bem, Geobacter sulfurreducens, Geobacter sulfurreducens PCA, Geobacillus stearothermophilus DSM 2334. Haemophilus influenzae, Helicobacter pylori, Homo sapiens, Hydrogenobacter thermophilus, Hydrogenobacter thermophilus TK-6, Hyphomicrobium denitrificans ATCC 51888, Hyphomicrobium zavarzinii, Klebsiella pneumoniae, Klebsiella pneumoniae subsp. pneumoniae MGH 78578, Lactobacillus brevis ATCC 367, Leuconostoc mesenteroides, Lysinibacillus fusiformis, Lysinibacillus sphaericus, Mesorhizobium loti MAFF303099, Metallosphaera sedula, Methanosarcina acetivorans, Methanosarcina acetivorans C2A, Methanosarcina barkeri, Methanosarcina mazei Tuc01, Methylobacter marinus, Methylobacterium extorquens, Methylobacterium extorquens AM1, Methylococcus capsulatas, Methylomonas aminofaciens, Moorella thermoacetica, Mycobacter sp. strain JC1 DSM 3803, Mycobacterium avium subsp. paratuberculosis K-10, Mycobacterium bovis BCG, Mycobacterium gastri, Mycobacterium marinum M, Mycobacterium smegmatis, Mycobacterium smegmatis MC2 155, Mycobacterium tuberculosis, Nitrosopumilus salaria BD31, Nitrososphaera gargensis Ga9.2, Nocardia farcinica IFM 10152, Nocardia iowensis (sp. NRRL 5646). Nostoc sp. PCC 7120, Ogataea angusta, Ogataea parapolymorpha DL-1 (Hansenula polymorpha DL-1), Paenibacillus peoriae KCTC 3763, Paracoccus denitrificans, Penicillium chrysogenum, Photobacterium profundum 3TCK, Phytofermentans ISDg, Pichia pastoris, Picrophilus torridus DSM9790, Porphyromonas gingivalis, Porphyromonas gingivalis W83, Pseudomonas aeruginosa PA01, Pseudomonas denitrificans, Pseudomonas knackmussii, Pseudomonas putida, Pseudomonas sp, Pseudomonas syringae pv. syringae B728a, Pyrobaculum islandicum DSM 4184, Pyrococcus abyssi, Pyrococcus furiosus, Pyrococcus horikoshii OT3, Ralstonia eutropha, Ralstonia eutropha H16, Rhodobacter capsulatus, Rhodobacter sphaeroides, Rhodobacter sphaeroides ATCC 17025, Rhodopseudomonas palustris, Rhodopseudomonas palustris CGA009, Rhodopseudomonas palustris DX-1, Rhodospirillum rubrum, Rhodospirillum rubrum ATCC 11170, Ruminococcus obeum ATCC 29174, Saccharomyces cerevisiae, Saccharomyces cerevisiae S288c, Salmonella enterica, Salmonella enterica subsp. enterica serovar Typhimurium str. LT2, Salmonella enterica typhimurium, Salmonella typhimurium, Schizosaccharomyces pombe, Sebaldella termitidis ATCC 33386, Shewanella oneidensis MR-1, Sinorhizobium meliloti 1021, Streptomyces coelicolor, Streptomyces griseus subsp. griseus NBRC 13350, Sulfolobus acidocaldarius, Sulfolobus solfataricus P-2, Synechocystis str. PCC 6803, Syntrophobacter fumaroxidans, Thaucra aromatica, Thermoanaerobacter sp. X514, Thermococcus kodakaraensis, Thermococcus litoralis, Thermoplasma acidophilum, Thermoproteus neutrophilus, Thermotoga maritima, Thiocapsa roseopersicina, Tolumonas auensis DSM 9187, Trichomonas vaginalis G3, Trypanosoma brucei, Tsukamurella paurometabola DSM 20162, Vibrio cholera, Vibrio harveyi ATCC BAA-1116, Xanthobacter autotrophicus Py2, Yersinia intermedia, and Zea mays.

Algae that can be engineered for cannabinoid production include, but are not limited to, unicellular and multicellular algae. Examples of such algae can include a species of rhodophyte, chlorophyte, heterokontophyte (including diatoms), tribophyte, glaucophyte, chlorarachniophyte, euglenoid, haptophyte, cryptomonad, dinoflagellum, phytoplankton, and the like, and combinations thereof. In one embodiment, algae can be of the classes Chlorophyceae and/or Haptophyta.

Microalgae (single-celled algae) produce natural oils that can contain the synthesized cannabinoids. Specific species that are considered for cannabinoid production include, but are not limited to, Neochloris oleoabundans, Scenedesmus dimorphus, Euglena gracilis, Phaeodactylum tricornutum, Pleurochrysis carterae, Prymnesium parvum, Tetraselmis chui, Nannochloropsis gaditana, Dunaliella salina. Dunaliella tertiolecta, Chlorella vulgaris, Chlorella variabilis, and Chlamydomonas reinhardtii. Additional or alternate algal sources can include one or more microalgae of the Achnanthes, Amphiprora, Amphora, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrsosphaera, Cricosphaera, Crypthecodinium, Cryptomonas, Cyclotella, Dunaliella, Ellipsoidon, Emiliania. Fremosphaera, Ernodesmius, Euglena, Franceia, Fragilaria, Gloeolhamnion, Haematococcus, Halocafeteria, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus. Platymonas, Pleurochrsis, Pleurococcus, Prototheca, Pseudo chlorella, Pyramimonas, Pvrobotrys, Scenedesmus, Skeletonema, Spirogyra, Stichococcus, Tetraselmis, Thalassiosira, Viridiella, and Volvox species, and/or one or more cyanobacteria of the Agmenellum, Anabaena, Anabaenopsis, Anacystis, Aphanizomenon, Arthrospira, Asterocapsa, Borzia, Calothrix, Chamaesiphon, Chlorogloeopsis, Chroococcidiopsis, Chroococcus, Crinalium, Cyanobacterium, Cyanobium, Cyanocystis, Cyanospira, Cyanothece, Cylindrospermopsis, Cylindrospermum, Dactylcoccopsis, Dermocarpella, Fischerella, Fremyella, Geitleria, Geitlerinema, Gloeobacter, Gloeocapsa, Gloeothece, Halospirulina, Ivengariella, Leptolyngbya, Limnothrix, Lyngbya, Microcoleus, Microcystis, Mxosarcina, Nodularia, Nostoc, Nostochopsis, Oscillatoria, Phormidium, Planktothrix, Pleurocapsa, Prochlorococcus, Prochloron, Prochlorothrix, Pseudanabaena, Rivularia, Schizothrix, Scvtonema, Spirulina, Stanieria, Starria, Stigonema, Symploca, Synechococcus, Svnechocystis, Tolipothrix, Trichodesmium. Tychonema, and Xenococcus species.

The host cell may be genetically modified for a recombinant production system, e.g., to produce CBCA, CBDA, THCA, CBCOA, CBDOA, THCOA, CBCVA, CBDVA, THCVA, CBCO, CBDO, THCO, CBCV, CBDV, THCV, CBC, CBD, and/or THC as described herein. The mode of gene transfer technology may be by electroporation, conjugation, transduction or natural transformation as described herein.

To genetically modify a host cell of the disclosure, one or more heterologous nucleic acids disclosed herein is introduced stably or transiently into a host cell, using established techniques. Such techniques may include, but are not limited to, electroporation, calcium phosphate precipitation, DEAE-dextran mediated transfection, liposome-mediated transfection, particle bombardment, and the like. For stable transformation, a heterologous nucleic acid will generally further include a selectable marker, e.g., any of several well-known selectable markers such as neomycin resistance, ampicillin resistance, tetracycline resistance, chloramphenicol resistance, kanamycin resistance, hygromycin resistance, G418 resistance, bleomycin resistance, zeocin resistance, and the like. A broad range of plasmids and drug resistance markers are available and described in embodiments herein. The cloning vectors are tailored to the host organisms based on the nature of antibiotic resistance markers that can function in that host cell. In some embodiments, the host cell is genetically modified using CRISPR/Cas9 to produce the engineered cell of the disclosure.

### Fermentation

In some embodiments, the disclosure provides a method of producing a cannabinoid or precursor thereof, e.g., CBCA, CBDA, THCA, CBCOA, CBDOA, THCOA, CBCVA, CBDVA, THCVA, CBCO, CBDO, THCO, CBCV, CBDV, THCV, CBC, CBD, and/or THC, as described herein, comprising culturing an engineered cell provided herein to provide the cannabinoid. In some embodiments, the method further comprises recovering the cannabinoid, e.g., CBCA, CBDA, THCA, CBCOA, CBDOA, THCOA, CBCVA, CBDVA, THCVA, CBCO, CBDO, THCO, CBCV, CBDV, THCV, CBC, CBD, and/or THC from the cell, cell extract, culture medium, whole culture, or combinations thereof.

In some embodiments, the culture medium of the engineered cells further comprises at least one carbon source. In embodiments where the cells are heterotrophic cells, the culture medium comprises at least one carbon source that is also an energy source, also known as a “feed molecule.” In some embodiments, the culture medium comprises one, two, three, or more carbon sources that are not primary energy sources. Non-limiting examples of feed molecules that can be included in the culture medium include acetate, malonate, oxaloacetate, aspartate, glutamate, beta-alanine, alpha-alanine, butyrate, hexanoate, hexanol, prenol, isoprenol, and geraniol. Further examples of compounds that can be provided in the culture medium include, without limitation, biotin, thiamine, pantethine, and 4-phosphopantetheine.

In some embodiments, the culture medium comprises acetate. In some embodiments, the culture medium comprises acetate and hexanoate. In some embodiments, the culture medium comprises malonate and hexanoate. In some embodiments, the culture medium comprises prenol, isoprenol, and/or geraniol. In some embodiments, the culture medium comprises aspartate, hexanoate, prenol, isoprenol, and/or geraniol.

Depending on the desired microorganism or strain to be used, the appropriate culture medium may be used. For example, descriptions of various culture media may be found in “Manual of Methods for General Bacteriology” of the American Society for Bacteriology (Washington D.C., USA, 1981). As used herein, culture medium, or simply “medium” as it relates to the growth source, refers to the starting medium, which may be in a solid or liquid form. “Cultured medium” as used herein refers to medium (e.g. liquid medium) containing microbes that have been fermentatively grown and can include other cellular biomass. The medium generally includes one or more carbon sources, nitrogen sources, inorganic salts, vitamins and/or trace elements. “Whole culture” as used herein refers to cultured cells plus the culture medium in which they are cultured. “Cell extract” as used herein refers to a lysate of the cultured cells, which may include the culture medium and which may be crude (unpurified), purified or partially purified. Methods of purifying cell lysates are known to the skilled artisan and described in embodiments herein.

Exemplary carbon sources include sugar carbons such as sucrose, glucose, galactose, fructose, mannose, isomaltose, xylose, maltose, arabinose, cellobiose and 3-, 4-, or 5-oligomers thereof. Other carbon sources include carbon sources such as methanol, ethanol, glycerol, formate and fatty acids. Still other carbon sources include carbon sources from gas such as synthesis gas, waste gas, methane, CO, CO₂ and any mixture of CO, CO₂ with H₂. Other carbon sources can include renewal feedstocks and biomass. Exemplary renewal feedstocks include cellulosic biomass, hemicellulosic biomass and lignin feedstocks.

In some embodiments, the engineered cell is sustained, cultured, or fermented under aerobic, microaerobic, anaerobic or substantially anaerobic conditions. Exemplary aerobic, microaerobic, and anaerobic conditions have been described previously and are known in the art. Briefly, anaerobic conditions refer to an environment devoid of oxygen. Substantially anaerobic conditions include, for example, a culture, batch fermentation or continuous fermentation such that the dissolved oxygen concentration in the medium remains between 0 and 10% of saturation, or higher. Substantially anaerobic conditions also include growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases. Exemplary anaerobic conditions for fermentation processes are described, for example, in US2009/0047719. Any of these conditions can be employed with the microbial organisms described herein as well as other anaerobic conditions known in the field. The culture conditions can include, for example, liquid culture procedures as well as fermentation and other large scale culture procedures.

The culture conditions can be scaled up and grown continuously for manufacturing the cannabinoid products described herein. Exemplary growth procedures include, for example, fed-batch fermentation and batch separation; fed-batch fermentation and continuous separation, or continuous fermentation and continuous separation. Fermentation procedures can be particularly useful for the biosynthetic production of commercial quantities of cannabinoids, e.g., CBCA, CBDA, THCA, CBCOA, CBDOA, CBCVA, CBDVA, THCVA, THCOA, CBCO, CBDO, THCO, CBCV, CBDV, THCV, CBC, CBD, and/or THC. Generally, and as with non-continuous culture procedures, the continuous and/or near-continuous production of cannabinoid product can include culturing a cannabinoid-producing organism with sufficient nutrients and medium to sustain and/or nearly sustain growth in an exponential phase. Continuous culture under such conditions can include, for example, 1 day, 2, 3, 4, 5, 6 or 7 days or more. Additionally, continuous culture can include 1 week, 2, 3, 4 or 5 or more weeks and up to several months. Alternatively, the desired microorganism can be cultured for hours, if suitable for a particular application. It is to be understood that the continuous and/or near-continuous culture conditions also can include all time intervals in between these exemplary periods. It is further understood that the time of culturing the microbial organism is for a sufficient period of time to produce a sufficient amount of product for a desired purpose.

Fermentation procedures are known to the skilled artisan. Briefly, fermentation for the biosynthetic production of a cannabinoid, e.g., CBCA, CBDA, THCA, CBCOA, CBDOA. THCOA, CBCVA, CBDVA, THCVA, CBCO, CBDO, THCO, CBCV, CBDV, THCV, CBC, CBD, and/or THC, can be utilized in, for example, fed-batch fermentation and batch separation; fed-batch fermentation and continuous separation, or continuous fermentation and continuous separation. Examples of batch and continuous fermentation procedures are known in the field. Typically, cells are grown at a temperature in the range of about 25° C. to about 40° C. in an appropriate medium, as well as up to 70° C. for thermophilic microorganisms.

The culture medium at the start of fermentation may have a pH of about 4 to about 7. The pH may be less than 11, less than 10, less than 9, or less than 8. In some embodiments, the pH is at least 2, at least 3, at least 4, at least 5, at least 6, or at least 7. In some embodiments, the pH of the medium is about 6 to about 9.5; 6 to about 9, about 6 to 8 or about 8 to 9.

In some embodiments, upon completion of the cultivation period, the fermenter contents are passed through a cell separation unit, for example, a centrifuge, filtration unit, and the like, to remove cells and cell debris. In embodiments where the desired product is expressed intracellularly, the cells are lysed or disrupted enzymatically or chemically prior to or after separation of cells from the fermentation broth, as desired, in order to release additional product. The fermentation broth can be transferred to a product separations unit. Isolation of product can be performed by standard separations procedures employed in the art to separate a desired product from dilute aqueous solutions. Such methods include, but are not limited to, liquid-liquid extraction using a water immiscible organic solvent (e.g., toluene or other suitable solvents, including but not limited to diethyl ether, ethyl acetate, methylene chloride, chloroform, benzene, pentane, hexane, heptane, petroleum ether, methyl tertiary butyl ether (MTBE), and the like) to provide an organic solution of the product, if appropriate, standard distillation methods, and the like, depending on the chemical characteristics of the product of the fermentation process.

Suitable purification and/or assays to test a cannabinoid, e.g., CBCA, CBDA, THCA, CBCOA, CBDOA, THCOA, CBCVA, CBDVA, THCVA, CBCO, CBDO, THCO, CBCV, CBDV, THCV, CBC, CBD, and/or THC, produced by the methods herein can be performed using known methods. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al. (2005), Biotechnol. Bioeng. 90:775-779), or other suitable assay and detection methods well known in the art. The individual enzyme or protein activities from the exogenous DNA sequences can also be assayed using methods known in the art.

The cannabinoids produced using methods described herein can be separated from other components in the culture using a variety of methods well known in the art. Such separation methods include, for example, extraction procedures as well as methods that include liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, and ultrafiltration. For example, the amount of cannabinoid or other product(s), including a polyketide, produced in a bio-production media generally can be determined using any of methods such as, for example, high performance liquid chromatography (HPLC), gas chromatography (GC), GC/Mass Spectroscopy (MS), or spectrometry.

In some embodiments, the cell extract or cell culture medium described herein comprises a cannabinoid. In some embodiments, the cannabinoid is cannabichromene (CBC) type (e.g. cannabichromenic acid), cannabigerol (CBG) type (e.g. cannabigerolic acid), cannabidiol (CBD) type (e.g. cannabidiolic acid), Δ⁹-trans-tetrahydrocannabinol (Δ⁹-THC) type (e.g. Δ⁹-tetrahydrocannabinolic acid), Δ⁸-trans-tetrahydrocannabinol (Δ⁸-THC) type, cannabicyclol (CBL) type, cannabielsoin (CBE) type, cannabinol (CBN) type, cannabinodiol (CBND) type, cannabitriol type, or combinations thereof. In some embodiments, the cannabinoid is cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C4 (CBD-C4), cannabidivarinic acid (CBDVA), cannabidivarin (CBDV), cannabidiorcol (CBD-C1), Δ⁹-tetrahydrocannabinolic acid A (THCA-A), Δ⁹-tetrahydrocannabinolic acid B (THCA-B), Δ⁹-tetrahydrocannabinol (THC), Δ⁹-tetrahydrocamiabinolic acid-C4 (THCA-C4), Δ⁹-tetrahydrocannabinol-C4 (THC-C4), Δ⁹-tetrahydrocannabivarinic acid (THCVA), Δ⁹-tetrahydrocannabivarin (THCV), Δ⁹-tetrahydrocannabiorcolic acid (THCA-C1), Δ⁹-tetrahydrocannabiorcol (THC-C1), Δ⁷-cis-iso-tetrahydrocannabivarin, Δ⁸-tetrahydrocannabinolic acid (Δ⁸-THCA), Δ⁸-tetrahydrocannabinol (Δ⁸-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabielsoinic acid, cannabicitranic acid, cannabinolic acid (CBNA), cannabinol (CBN), cannabinol methylether (CBNM), cannabinol-C4, (CBN—C4), cannabivarin (CBV), cannabinol-C2 (CNB—C2), cannabiorcol (CBN—C1), cannabinodiol (CBND), cannabidivarin (CBVD), cannabitriol, 10-ethyoxy-9-hydroxy-delta-6a-tetrahydrocannabinol, 8,9-dihydroxyl-delta-6a-tetrahydrocannabinol, cannabitriolvarin (CBTVE), dehydrocannabifuran (DCBF), cannabifuran (CBF), cannabichromanon (CBCN), cannabicitran, 10-oxo-delta-6a-tetrahydrocannabinol (OTHC), Δ⁹-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), trihydroxy-Δ⁹-tetrahydrocannabinol (triOH-THC), or combinations thereof.

In some embodiments, the disclosure provides a cell extract or cell culture medium comprising cannabigerolic acid (CBGA), cannabichromenic acid (CBCA), cannabidiolic acid (CBDA), tetrahydrocannabinolic acid (THCA), cannabigerol (CBG), cannabichromene (CBC), cannabidiol (CBD), tetrahydrocannabinol (THC), cannabigerorcinic acid (CBGOA), cannabiorcichromenic acid (CBCOA), cannabidiorcinic acid (CBDOA), tetrahydrocannabiorcolic acid (THCOA), cannabigerovarinic acid (CBGVA), cannabichromevarinic acid (CBCVA), cannabidivarinic acid (CBDVA), tetrahydrocannabivarin acid (THCVA), cannabigerorcinol (CBGO), cannabichromeorcin (CBCO), cannabidiorcin (CBDO), tetrahydrocannabiorcin (THCO), cannabigerivarinol (CBGV), cannabichromevarin (CBCV), cannabidivarin (CBDV), tetrahydrocannabivarin (THCV), an isomer, analog or derivative thereof, or combinations thereof derived from the engineered cell described herein. In some embodiments, a derivative of a cannabinoid described herein, e.g., CBGA, CBCA, CBDA, THCA, CBGOA, CBCOA, CBDOA, THCOA, CBGVA, CBCVA, CBDVA, and/or THCVA, is a decarboxylated form of the cannabinoid.

### Method of Making or Isolating

In some embodiments, the disclosure provides a method of making a cannabinoid selected from CBCA, CBC, CBCOA, CBCVA, CBCO, CBCV, CBDA, CBD, CBDOA, CBDVA, CBCO, CBDV, THCA, THC, THCOA, THCVA, THCO, THCV, an isomer, analog or derivative thereof, or combinations thereof, comprising culturing the engineered cell as described herein. In some embodiments, the engineered cell comprises a flavin-dependent oxidase in Table 1. In some embodiments, the engineered cell comprises a non-natural flavin-dependent oxidase described herein. In some embodiments, the engineered cell comprises a heterologous polynucleotide encoding a flavin-dependent oxidase of Table 1. In some embodiments, the flavin-dependent oxidase comprises at least 70%, at least 75%, at least 80%, at least 85%, at least 90%, at least 95%, at least 96%, at least 97% at least 98%, at least 99%, or 100% sequence identity to a protein with UniProt IDs A0A1H4CL41, A0A7X0U8H0, A0A1Q5S5E2, A0A0Q7FI10, A0A2E0XWX6, D9XHS6, A0A0K3BN04, and A0A1U9QQ65. In some embodiments, the flavin-dependent oxidase comprises a motif of any one of SEQ ID NOs:1-14. In some embodiments, the engineered cell comprises a heterologous polynucleotide encoding a non-natural flavin-dependent oxidase described herein. In some embodiments, the engineered cell comprises an expression construct comprising the polynucleotide.

In some embodiments, the disclosure provides a method of isolating CBCA, CBC, CBCOA, CBCVA, CBCO, CBCV, CBDA, CBD, CBDOA, CBDVA, CBDO, CBCV, THCA, THC, THCOA, THCVA, THCO, THCV, an isomer, analog or derivative thereof, or combinations thereof, from the cell extract or cell culture medium of the engineered cell.

Methods of culturing cells, e.g., the engineered cell of the disclosure, are provided herein. Methods of isolating a cannabinoid, e.g., CBCA, CBC, CBCOA, CBCVA, CBCO, CBCV, CBDA, CBD, CBDOA, CBDVA, CBDO, CBDV, THCA, THC, THCOA, THCVA, THCO, THCV, an isomer, analog or derivative thereof, are also provided herein. In some embodiments, the isolating comprises liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, ultrafiltration, or combinations thereof.

In some embodiments, the disclosure provides a method of making CBCA, CBDA, THCA, or an isomer, analog or derivative thereof, or combinations thereof, comprising contacting CBGA with the flavin-dependent oxidase described herein. In some embodiments, the flavin-dependent oxidase is a non-natural flavin-dependent oxidase described herein. In some embodiments, the disclosure provides a method of making CBCA, CBDA, THCA, or an isomer, analog or derivative thereof, or combinations thereof, comprising contacting CBGA with a flavin-dependent oxidase of Table 1. In some embodiments, the flavin-dependent oxidase comprises a motif of any one of SEQ ID NOs:1-14.

In some embodiments, the disclosure provides a method of making CBCOA, CBDOA, THCOA, or an isomer, analog or derivative thereof, or combinations thereof, comprising contacting CBGOA with the flavin-dependent oxidase described herein. In some embodiments, the flavin-dependent oxidase is a non-natural flavin-dependent oxidase described herein. In some embodiments, the disclosure provides a method of making CBCOA, CBDOA, THCOA, or an isomer, analog or derivative thereof, or combinations thereof, comprising contacting CBGOA with a flavin-dependent oxidase of Table 1. In some embodiments, the flavin-dependent oxidase comprises a motif of any one of SEQ ID NOs:1-14.

In some embodiments, the disclosure provides a method of making CBCVA, CBDVA, THCVA, or an isomer, analog or derivative thereof, or combinations thereof, comprising contacting CBGVA with the flavin-dependent oxidase described herein. In some embodiments, the flavin-dependent oxidase is a non-natural flavin-dependent oxidase described herein. In some embodiments, the disclosure provides a method of making CBCVA, CBDVA, THCVA, or an isomer, analog or derivative thereof, or combinations thereof, comprising contacting CBGVA with a flavin-dependent oxidase of Table 1. In some embodiments, the flavin-dependent oxidase comprises a motif of any one of SEQ ID NOs:1-14.

In some embodiments, the disclosure provides a method of making CBC, CBD, THC, or an isomer, analog or derivative thereof, or combinations thereof, comprising contacting CBG with the flavin-dependent oxidase described herein. In some embodiments, the flavin-dependent oxidase is a non-natural flavin-dependent oxidase described herein. In some embodiments, the disclosure provides a method of making CBC, CBD, THC, or an isomer, analog or derivative thereof, or combinations thereof, comprising contacting CBG with a flavin-dependent oxidase of Table 1. In some embodiments, the flavin-dependent oxidase comprises a motif of any one of SEQ ID NOs:1-14.

In some embodiments, the disclosure provides a method of making CBCO, CBDO, THCO, or an isomer, analog or derivative thereof, or combinations thereof, comprising contacting CBGO with the flavin-dependent oxidase described herein. In some embodiments, the flavin-dependent oxidase is a non-natural flavin-dependent oxidase described herein. In some embodiments, the disclosure provides a method of making CBCO, CBDO, THCO, or an isomer, analog or derivative thereof, or combinations thereof, comprising contacting CBGO with a flavin-dependent oxidase of Table 1. In some embodiments, the flavin-dependent oxidase comprises a motif of any one of SEQ ID NOs:1-14.

In some embodiments, the disclosure provides a method of making CBCV, CBDV, THCV, or an isomer, analog or derivative thereof, or combinations thereof, comprising contacting CBGV with the flavin-dependent oxidase described herein. In some embodiments, the flavin-dependent oxidase is a non-natural flavin-dependent oxidase described herein. In some embodiments, the disclosure provides a method of making CBCV, CBDV, THCV, or an isomer, analog or derivative thereof, or combinations thereof, comprising contacting CBGV with a flavin-dependent oxidase of Table 1. In some embodiments, the flavin-dependent oxidase comprises a motif of any one of SEQ ID NOs:1-14.

In some embodiments, the contacting occurs at about pH 4 to about pH 9, about pH 4.5 to about pH 8.5, about pH 5 to about pH 8, about pH 5.5 to about pH 7.5, or about pH 5 to about pH 7. In some embodiments, the method is performed in an in vitro reaction medium, e.g., an aqueous reaction medium.

In some embodiments, the reaction medium further comprises a buffer, a salt, a surfactant, or combinations thereof. In some embodiments, the surfactant is about 0.005% (v/v) to about 5% (v/v) of the in vitro reaction medium. In some embodiments, the surfactant is about 0.01% (v/v) to about 1% (v/v) of the in vitro reaction medium. In some embodiments, the surfactant is about 0.05% (v/v) to about 0.5% (v/v) of the in vitro reaction medium. In some embodiments, the surfactant is about 0.08% (v/v) to about 0.2% (v/v) of the in vitro reaction medium. In some embodiments, the surfactant is a nonionic surfactant. Non-limiting examples of nonionic surfactants include TRITON™ X-100, TWEEN®, IGEPAL® CA-630, NONIDET™ P-40, and the like. In some embodiments, the surfactant is 2-[4-(2,4,4-trimethylpentan-2-yl)phenoxy]ethanol (also known as TRITON™ X-100). In some embodiments, the in vitro reaction medium comprises about 0.1% (v/v) 2-[4-(2,4,4-trimethylpentan-2-yl)phenoxy]ethanol.

In some embodiments, the flavin-dependent oxidase is produced by an engineered cell. In some embodiments, the flavin-dependent oxidase is overexpressed, e.g., on an exogenous nucleic acid such as a plasmid, by an inducible or constitutive promoter, in an engineered cell. In some embodiments, the disclosure provides a method of making an isolated flavin-dependent oxidase, comprising isolating the flavin-dependent oxidase expressed in the engineered cell. Methods of culturing cells, e.g., the engineered cell of the disclosure, are provided herein. In some embodiments, the disclosure provides an isolated flavin-dependent oxidase made by the methods provided herein. In some embodiments, the flavin-dependent oxidase is any of the flavin-dependent oxidases in Table 1. In some embodiments, the flavin-dependent oxidase is a non-natural flavin-dependent oxidase described herein. In some embodiments, the flavin-dependent oxidase comprises at least 70%, at least 75%, at least 80%, at least 85%, at least 90%, at least 95%, at least 96%, at least 97% at least 98%, at least 99%, or 100% sequence identity to a protein with UniProt IDs A0A1H4CL41, A0A7X0U8H0, A0A1Q5S5E2, A0A0Q7FI10, A0A2E0XWX6, D9XHS6, A0A0K3BN04, and A0A1U9QQ65. In some embodiments, the flavin-dependent oxidase comprises a motif of any one of SEQ ID NOs:1-14.

Methods of isolating proteins (e.g., the flavin-dependent oxidase) from cells are known in the art. For example, the cells can be lysed to form a crude lysate, and the crude lysate can be further purified using filtration, centrifugation, chromatography, buffer exchange, or combinations thereof. The cell lysate is considered partially purified when about 10% to about 60%, or about 20% to about 50%, or about 30% to about 50% of the total proteins in the lysate is the desired protein of interest, e.g., the non-natural flavin-dependent oxidase. A protein can also be isolated from the cell lysate as a purified protein when greater than 60%, greater than 70%, greater than 80%, greater than 90%, greater than 95%, or greater than 99% of total proteins in the lysate is the desired protein of interest, e.g., the flavin-dependent oxidase. In some embodiments, the flavin-dependent oxidase comprises a motif of any one of SEQ ID NOs:1-14.

In some embodiments, the crude lysate comprising the flavin-dependent oxidase is capable of converting CBGA to CBCA, CBDA, THCA, or an isomer, analog or derivative thereof; or CBGOA to CBCOA, CBDOA, THCOA, or an isomer, analog or derivative thereof; or CBGVA to CBCVA, CBDVA, THCVA, or an isomer, analog or derivative thereof; or CBG to CBC, CBD, THC, or an isomer, analog or derivative thereof; or CBGO to CBCO, CBDO, THCO, or an isomer, analog or derivative thereof; or CBGV to CBCV, CBDV, THCV, or an isomer, analog or derivative thereof. In some embodiments, an analog or derivative of CBGA, CBGOA, and CBGVA known in the art is used as a substrate for conversion of the flavin-dependent oxidase. In some embodiments, the CBGA. CBGOA, CBGVA, CBG, CBGO, and/or CBGV is contacted with crude lysate comprising the flavin-dependent oxidase to form CBCA, CBDA, THCA, CBCOA, CBDOA, THCOA, CBCVA, CBDVA, THCVA, CBC, CBD, THC, CBCO, CBDO, THCO, CBCV, CBDV, THCV, or an isomer, analog or derivative thereof. In some embodiments, the flavin-dependent oxidase is any of the flavin-dependent oxidases in Table 1. In some embodiments, the flavin-dependent oxidase is a non-natural flavin-dependent oxidase described herein. In some embodiments, the flavin-dependent oxidase comprises a motif of any one of SEQ ID NOs:1-14.

In some embodiments, a partially purified lysate comprising the flavin-dependent oxidase is capable of converting CBGA to CBCA, CBDA, THCA, or an isomer, analog or derivative thereof; or CBGOA to CBCOA, CBDOA, THCOA, or an isomer, analog or derivative thereof; or CBGVA to CBCVA, CBDVA, THCVA, or an isomer, analog or derivative thereof; or CBG to CBC, CBD, THC, or an isomer, analog or derivative thereof; or CBGO to CBCO, CBDO, THCO, or an isomer, analog or derivative thereof; or CBGV to CBCV, CBDV, THCV, or an isomer, analog or derivative thereof. In some embodiments, the CBGA, CBGOA, CBGVA, CBG, CBGO, and/or CBGV is contacted with the partially purified lysate comprising the flavin-dependent oxidase to form CBCA, CBDA, THCA, CBCOA, CBDOA, THCOA, CBCVA, CBDVA, THCVA, CBC, CBD, THC, CBCO, CBDO, THCO, CBCV, CBDV, THCV, or an isomer, analog or derivative thereof. In some embodiments, the flavin-dependent oxidase is any of the flavin-dependent oxidases in Table 1. In some embodiments, the flavin-dependent oxidase is a non-natural flavin-dependent oxidase described herein. In some embodiments, the flavin-dependent oxidase comprises a motif of any one of SEQ ID NOs:1-14.

In some embodiments, a purified flavin-dependent oxidase is capable of converting CBGA to CBCA, CBDA, THCA, or an isomer, analog or derivative thereof; or CBGOA to CBCOA, CBDOA, THCOA, or an isomer, analog or derivative thereof; or CBGVA to CBCVA, CBDVA, THCVA, or an isomer, analog or derivative thereof; or CBG to CBC, CBD, THC, or an isomer, analog or derivative thereof; or CBGO to CBCO, CBDO, THCO, or an isomer, analog or derivative thereof; or CBGV to CBCV, CBDV, THCV, or an isomer, analog or derivative thereof. In some embodiments, the CBGA, CBGOA, CBGVA, CBG, CBGO, and/or CBGV is contacted with the purified flavin-dependent oxidase to form CBCA, CBDA, THCA, CBCOA, CBDOA, THCOA, CBCVA, CBDVA, THCVA, CBC, CBD, THC, CBCO, CBDO, THCO, CBCV, CBDV, THCV, or an isomer, analog or derivative thereof. In some embodiments, the flavin-dependent oxidase is any of the flavin-dependent oxidases in Table 1. In some embodiments, the flavin-dependent oxidase is a non-natural flavin-dependent oxidase described herein. In some embodiments, the flavin-dependent oxidase comprises a motif of any one of SEQ ID NOs:1-14.

### Compositions

In some embodiments, the disclosure provides a composition comprising a cannabinoid or an isomer, analog or derivative thereof obtained from the engineered cell, cell extract, or method described herein. In some embodiments, the cannabinoid is CBCA, CBDA. THCA, CBCOA, CBDOA, THCOA, CBCVA, CBDVA, THCVA, CBC, CBD, THC, CBCO, CBDO, THCO, CBCV, CBDV, THCV, or an isomer, analog or derivative thereof, or combinations thereof. In some embodiments, the cannabinoid is 10% or greater, 20% or greater, 30% or greater, 40% or greater, 50% or greater, 60% or greater, 70% or greater, 80% or greater, 85% or greater, 90% or greater, 91% or greater, 92% or greater, 93% or greater, 94% or greater, 95% or greater, 96% or greater, 97% or greater, 98% or greater, 99% or greater, 99.2% or greater, 99.4% or greater, 99.5% or greater, 99.6% or greater, 99.7% or greater, 99.8% or greater, or 99.9% or greater of total cannabinoid compound(s) in the composition.

In some embodiments, the composition is a therapeutic or medicinal composition. In some embodiments, the composition further comprises a pharmaceutically acceptable excipient. In some embodiments, the composition is a topical composition. In some embodiments, the composition is in the form of a cream, a lotion, a paste, or an ointment.

In some embodiments, the composition is an edible composition. In some embodiments, the composition is provided in a food or beverage product. In some embodiments, the composition is an oral unit dosage composition. In some embodiments, the composition is provided in a tablet or a capsule.

In some embodiments, the disclosure provides a composition comprising (a) a flavin-dependent oxidase as described herein; and (b) a cannabinoid, a prenylated aromatic compound, or both. In some embodiments, the cannabinoid is CBCA, CBDA, THCA, CBCOA, CBDOA, THCOA, CBCVA, CBDVA, THCVA, CBC, CBD, THC, CBCO, CBDO, THCO, CBCV, CBDV, THCV, or an isomer, analog, or derivative thereof, or combinations thereof. In some embodiments, the flavin-dependent oxidase is any of the flavin-dependent oxidases in Table 1. In some embodiments, the flavin-dependent oxidase is a non-natural flavin-dependent oxidase described herein. In some embodiments, the flavin-dependent oxidase comprises a motif of any one of SEQ ID NOs:1-14.

In some embodiments, the compositions herein comprising a flavin-dependent oxidase and a cannabinoid, a prenylated aromatic compound, or both, further comprise an enzyme in a cannabinoid biosynthesis pathway. Cannabinoid biosynthesis pathways are described herein. In some embodiments, the cannabinoid biosynthesis pathway enzyme comprises olivetol synthase (OLS), olivetolic acid cyclase (OAC), prenyltransferase, or combinations thereof. In some embodiments, the flavin-dependent oxidase is any of the flavin-dependent oxidases in Table 1. In some embodiments, the flavin-dependent oxidase is a non-natural flavin-dependent oxidase described herein. In some embodiments, the flavin-dependent oxidase comprises a motif of any one of SEQ ID NOs:1-14.

All references cited herein, including patents, patent applications, papers, textbooks and the like, and the references cited therein, to the extent that they are not already, are hereby incorporated herein by reference in their entirety.

### EXAMPLES

### Example 1. Identification of Enzymes with Peptide Motif

A search in the UniProt public database using the InterPro code for the berberine-bridge enzyme (BBE) family, IPR012951, yielded 31,898 enzyme sequences. Restricting the taxonomy to bacteria yielded 13,398 enzyme sequences. The FASTA amino acid sequences for 13,398 enzymes were analyzed. One key feature of the BBE family is the covalent attachment of the catalytically required FAD cofactor. A histidine residue is essentially universally conserved among every enzyme in this family and provides one covalent attachment to the FAD. Enzymes known to oxidize CBGA to a cannabinoid (Clz9, THCAS, CBDAS) generally require a second covalent attachment to the FAD for full activity, which is achieved by a cysteine residue. A sequence comparison of the region around this Cys residue yielded a string of highly conserved amino acids: xGxCxxxxxxGxxxGGGxG, where x is any amino acid (see FIG. 1). This amino acid string was used to restrict the 13,398 bacterial BBE-like enzymes to ones which contain that string in their sequence. This reduces the number of bacterial BBE-like enzymes to 3,844

### Example 2. Identification of Bacterial BBE-Like Enzymes with Cannabinoid Synthase Activity

Plasmids for select sequences were codon optimized, synthesized, constructed and tested for cannabinoid synthase activity. Overnight cultures of E. coli BL21(DE3) containing plasmids expressing sequence-verified enzymes were grown in 0.5 mL of LB media overnight at 35° C. in a 96-deep-well plate. On the following day, 10 μL of overnight culture was added to 1000 μL of LB media containing 100 μg/mL of carbenicillin in a 96-deep-well plate. The cultures were grown at 35° C. for 3 hours until OD₆₀₀ reached approximately 0.4 to 0.6, and 0.5 mM IPTG and 0.2 mM cumate were added to induce protein expression. Protein was expressed for approximately 18 to 20 hours at room temperature. Cells were pelleted by centrifugation at 4000×g for 10 minutes. Cell pellets were resuspended to OD₆₀₀=10 and lysed by sonication in 50 mM Tris-HCl buffer, pH 7.4 and protease inhibitor cocktail. Cell lysates were clarified by centrifugation at 4000×g for 10 minutes. 20 μL of clarified lysate was mixed with 80 μL of 240 RM CBGA in 100 mM Tris-HCl buffer. pH 7.4, with 0.1% TRITON™ X-100 or 100 mM Citrate buffer, pH 5.0 with 0.1% TRITON™ X-100 in 96-well plates. The plates were then sealed, and the reactions were incubated at 37° C. for 24 hours and then quenched with 300 μL of 75% acetonitrile solution containing 0.1% formic acid and 1.2 RM diclofenac and 2 μM ibuprofen as internal standards. Precipitated protein and cell debris were removed by vacuum filtration using a 0.2 μm 96-well filter plate (PALL).

Analysis Method: The flow through was directly injected into an LC/MS system for analysis. The spectra were monitored by LC/MS at 357/191 multiple reaction monitoring (MRM) transitions. Cannabinoid products were identified by retention time to authentic cannabinoid standards and quantified by relative peak area versus peak area of known concentrations of cannabinoid standards.

### Example 3. Analysis of Cannabinoid Products

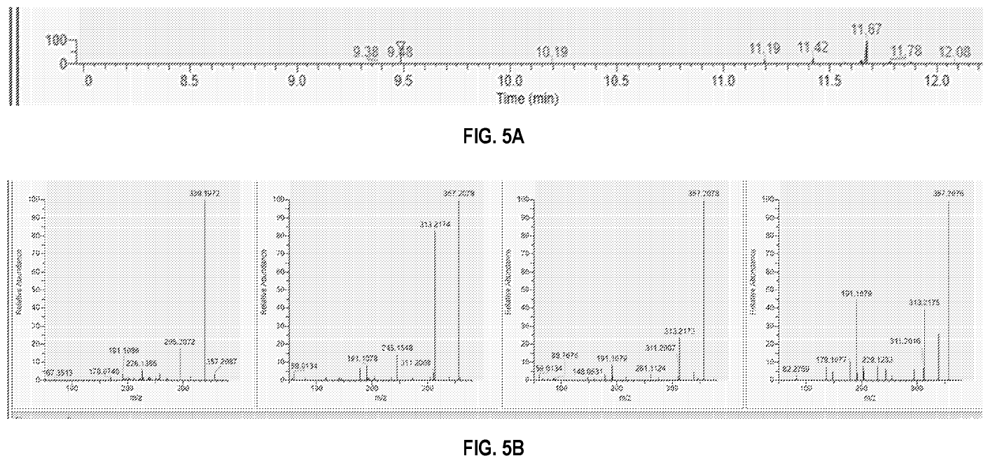

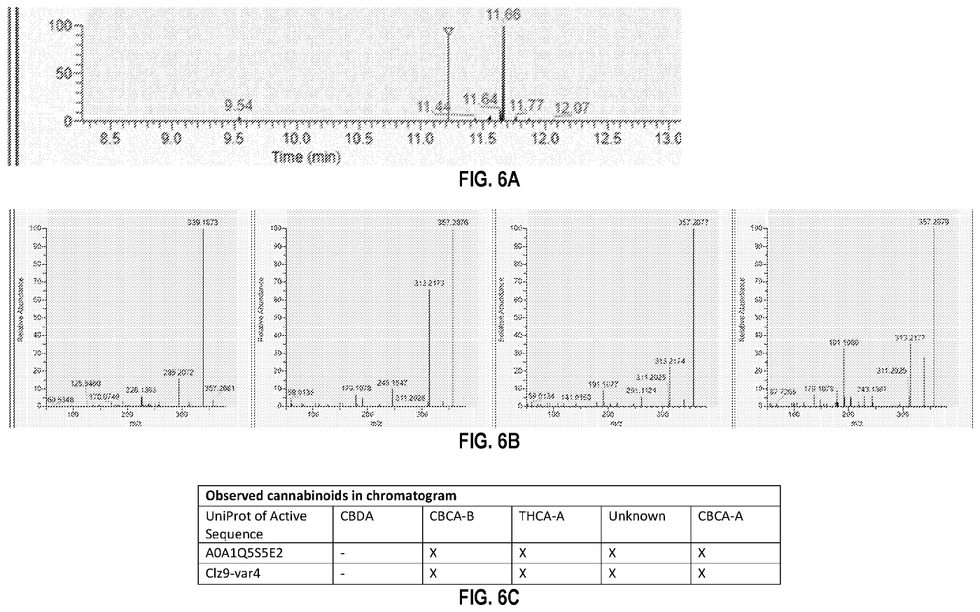

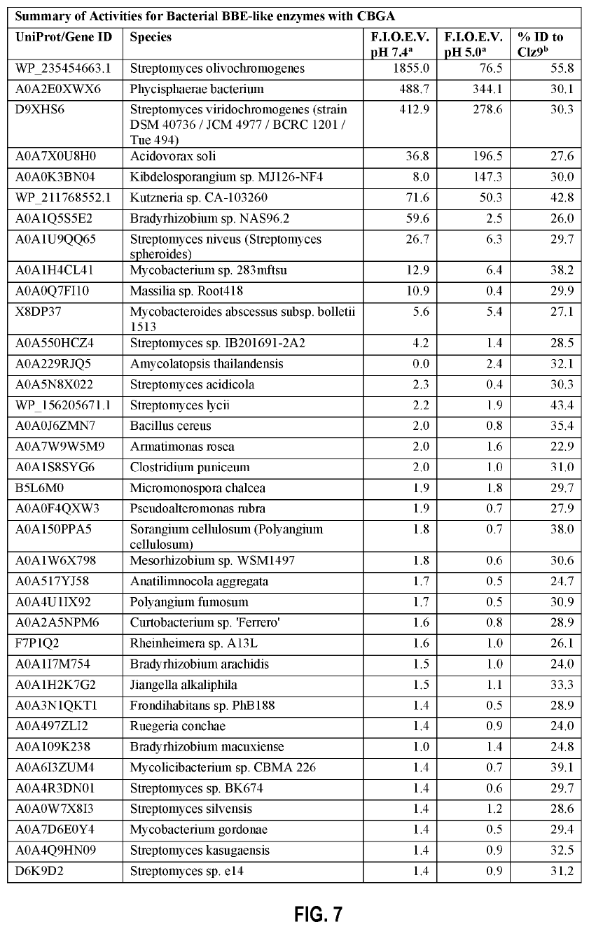

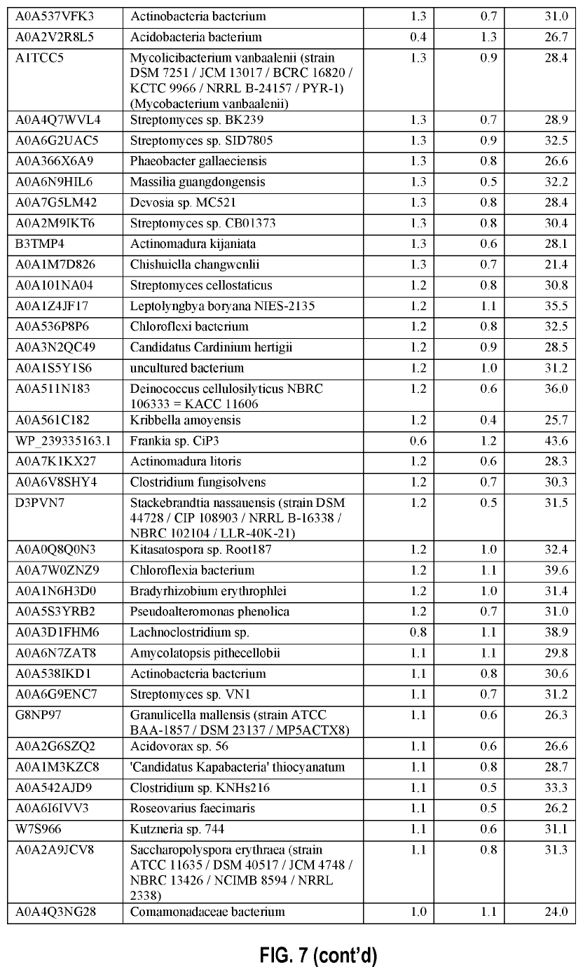

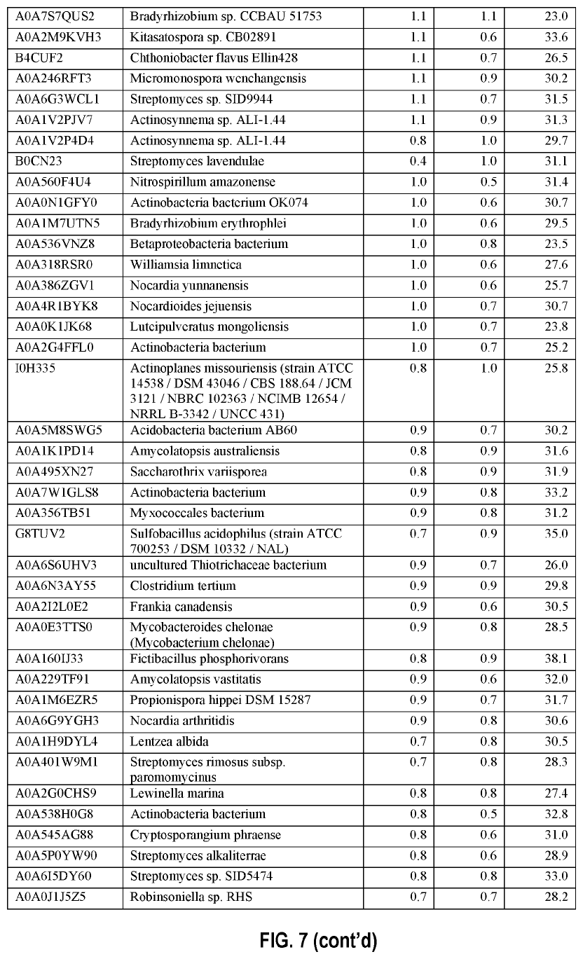

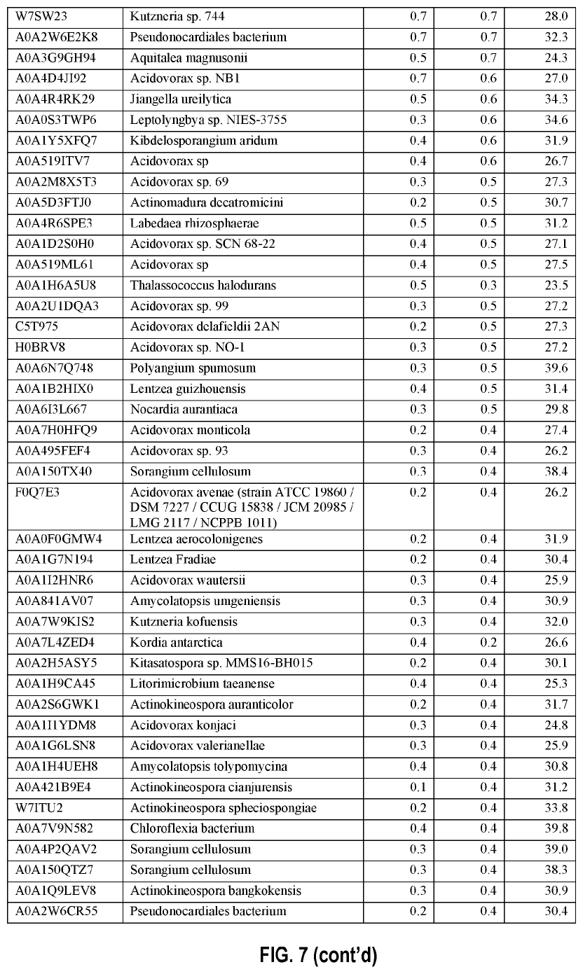

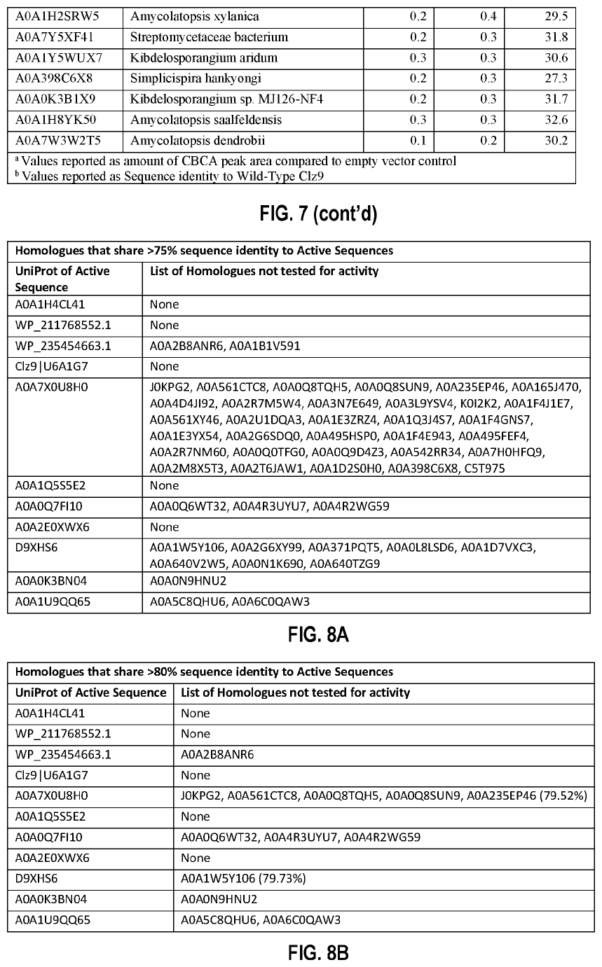

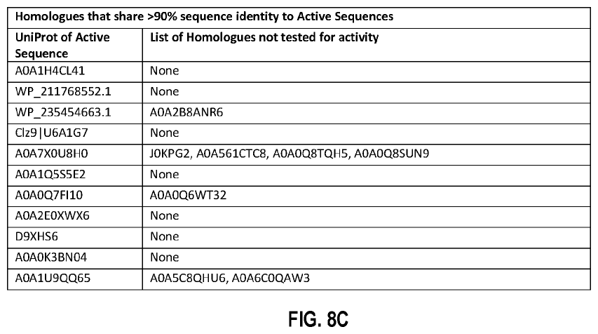

The protein with UniProt ID A0A1Q5S5E2 from Bradyrhizobium sp. NAS96 (“A0A1Q5S5E2”) was evaluated for activity using a similar assay as described in Example 2. Briefly, A0A1Q5S5E2 was contacted with CBGA in citrate buffer, pH 5.0, and the reaction was allowed to proceed for 96 hours. The reaction products were subjected to LC/MS/MS to identify the cannabinoid products. The resulting chromatogram of the products is shown in FIG. 5A. FIG. 5B shows the LC/MS/MS fragmentation patterns of the cannabinoid products. FIG. 6A shows the chromatogram of the reaction products from the same assay performed with a Clz9 variant comprising the amino acid mutations D404A T438F N400W V323Y Q275R C285L E370Q V372I L296M I271H A338N A272C E159A T442D (“Clz9-var4”), and FIG. 6B shows the LC/MS/MS fragmentation patterns of the cannabinoid products produced by Clz9-var4. In each of FIGS. 5B and 6B, the panels show, from left to right. CBCA-B, THCA-A, an unknown cannabinoid, and CBCA-A. FIG. 6C shows a summary of the cannabinoids observed in the chromatograms.

What is claimed is:

1. A flavin-dependent oxidase comprising:
(i) a first amino acid sequence comprising a His residue, wherein an FAD cofactor is covalently attached to the His residue; and
(ii) a second amino acid sequence comprising a peptide motif of Formula I:
[Formula I]
X₁-Gly-X₂-Cys-X₃-X₄-X₅-X₆-X₇-X₈-Gly-X₉-X₁₀-X₁₁-Gly-
Gly-Gly-X₁₂-Gly
wherein each X is any amino acid; and wherein the FAD cofactor is covalently attached to the Cys residue,
wherein the flavin-dependent oxidase is capable of oxidative cyclization of a prenylated aromatic compound into a cannabinoid, and
wherein the flavin-dependent oxidase is a bacterial protein or a fungal protein.

2. The flavin-dependent oxidase of claim 1, comprising:
Ala, Gly, Ser, Thr, or His at position X₁;
Thr, Ser, Arg, Val, Gly, Phe, or Asn at position X₂;
Pro, Ala, Gly, Tyr, or Phe at position X₃;
Thr, Ser, Ala, Asp, Gly, Asn, or Arg at position X₄;
Val or Ile at position X₅;
Gly, Ala, Cys, Arg, or Asn at position X₆;
lie, Val, Ala, Leu, Met, or Pro at position X₇;
Ala, Gly, Ser, Thr, or Tyr at position X₈;
Leu, His, Phe, Tyr, Ile, Val, or Trp at position X₉;
Thr, Val, Leu, Ile, or Ala at position X₁₀;
Leu, Gln, Ser, Thr, Cys, or Met at position X₁₁;
Ile, Tyr, Leu, Trp, Val, Phe, Met, His, or Gln at position X₂; or
any combination thereof.

3. The flavin-dependent oxidase of claim 1 or 2, wherein the peptide motif comprises:
X₁-Gly-X₂-Cys-Pro-Thr-Val-Gly-X₇-X₈-Gly-Leu-Thr-
Leu-Gly-Gly-Gly-X₁₂-Gly.

4. The flavin-dependent oxidase of claim 3, wherein:
X₂ is Thr or Ser; X₇ is Ile or Val; X₈ is Ala, Gly, or Ser; and X₁₂ is Ile, Tyr, or Leu.

5. The flavin-dependent oxidase of any one of claims 1 to 4, wherein the peptide motif comprises any one of SEQ ID NOs:1-14.

6. The flavin-dependent oxidase of any one of claims 1 to 5, wherein the flavin-dependent oxidase is isolated or derived from an organism according to Table 1.

7. The flavin-dependent oxidase of any one of claims 1 to 6, wherein the flavin-dependent oxidase is not glycosylated.

8. The flavin-dependent oxidase of any one of claims 1 to 7, wherein the flavin-dependent oxidase does not comprise a disulfide bond.

9. The flavin-dependent oxidase of any one of claims 1 to 8, wherein the prenylated aromatic compound is cannabigerolic acid (CBGA), cannabigerorcinic acid (CBGOA), cannabigerovarinic acid (CBGVA), cannabigerorcinol (CBGO), cannabigerivarinol (CBGV), cannabigerol (CBG), or analog or derivative thereof.

10. The flavin-dependent oxidase of any one of claims 1 to 9, wherein the flavin-dependent oxidase comprises at least one amino acid variation as compared to a wild-type flavin-dependent oxidase.

11. An engineered cell comprising a heterologous polynucleotide encoding the flavin-dependent oxidase of any one of claims 1 to 10.

12. The engineered cell of claim 11, wherein the engineered cell is capable of producing a cannabinoid.

13. The engineered cell of claim 12, wherein the cannabinoid comprises CBCA, CBDA, THCA, CBCOA, CBDOA, THCOA, CBCVA, CBDVA, THCVA, CBC, CBD, THC, CBCO, CBDO, THCO, CBCV, CBDV, THCV, analog or derivative thereof or combinations thereof.

14. The engineered cell of any one of claims 11 to 13, further comprising a cannabinoid biosynthesis pathway enzyme.

15. The engineered cell of claim 14, wherein the cannabinoid biosynthesis pathway enzyme comprises olivetol synthase (OLS), olivetolic acid cyclase (OAC), prenyltransferase, a geranyl pyrophosphate (GPP) biosynthesis pathway enzyme, or combinations thereof.

16. The engineered cell of any of claims 11 to 15, wherein the cell is a bacterial cell or a fungal cell.

17. The engineered cell of claim 16, wherein the cell is an Escherichia coli cell.

18. A cell extract or cell culture medium comprising cannabigerolic acid (CBGA), cannabichromenic acid (CBCA), cannabidiolic acid (CBDA), tetrahydrocannabinolic acid (THCA), cannabigerol (CBG), cannabichromene (CBC), cannabidiol (CBD), tetrahydrocannabinol (THC), cannabigerorcinic acid (CBGOA), cannabiorcichromenic acid (CBCOA), cannabidiorcinic acid (CBDOA), tetrahydrocannabiorcolic acid (THCOA), cannabigerovarinic acid (CBGVA), cannabichromevarinic acid (CBCVA), cannabidivarinic acid (CBDVA), tetrahydrocannabivarinic acid (THCVA), cannabigerorcinol (CBGO), cannabichromeorcin (CBCO), cannabidiorcin (CBDO), tetrahydrocannabiorcin (THCO), cannabigerivarinol (CBGV), cannabichromevarin (CBCV), cannabidivarin (CBDV), tetrahydrocannabivarin (THCV), an isomer, analog or derivative thereof, or combinations thereof, derived from the engineered cell of any one of claims 11 to 17.

19. A method of making a cannabinoid comprising: contacting a prenylated aromatic compound with the flavin-dependent oxidase of any one of claims 1 to 10; culturing the engineered cell of any one of claims 11 to 17; isolating the cannabinoid from the cell extract or cell culture medium of claim 18; or a combination thereof.

20. The method of claim 19, wherein the prenylated aromatic compound comprises CBGA, CBG, CBGOA, CBGO, CBGVA, CBGV, or analog or derivative thereof or a combination thereof.

21. The method of claim 19 or 20, wherein the cannabinoid comprises CBCA, CBC, CBCOA, CBCO, CBCVA, CBCV, CBDA, CBD, CBDOA, CBDO, CBDVA, CBDV, THCA, THC, THCOA, THCO, THCVA, THCV, an isomer, analog or derivative thereof, or combinations thereof.

22. A composition comprising a cannabinoid or an isomer, analog or derivative thereof obtained from the engineered cell of any one of claims 11 to 17, the cell extract or cell culture medium of claim 18, or the method of any one of claims 19 to 21.

23. The composition of claim 22, wherein the cannabinoid is CBCA, CBC, CBCOA, CBCO, CBCVA, CBCV, CBDA, CBD, CBDOA, CBDO, CBDVA, CBDV, THCA, THC, THCOA, THCO, THCVA, THCV, an isomer, analog or derivative thereof, or combinations thereof.

24. The composition of claim 23, wherein the cannabinoid is 50% or greater, 60% or greater, 70% or greater, 80% or greater, 85% or greater, 90% or greater, 91% or greater, 92% or greater, 93% or greater, 94% or greater, 95% or greater, 96% or greater, 97% or greater, 98% or greater, 99% or greater, 99.2% or greater, 99.4% or greater, 99.5% or greater, 99.6% or greater, 99.7% or greater, 99.8% or greater, or 99.9% or greater of total cannabinoid compound(s) in the composition.

25. The composition of any one of claims 22 to 24, wherein the composition is a therapeutic or medicinal composition; a topical composition; an edible composition; or combinations thereof.

26. A composition comprising: (a) the flavin-dependent oxidase of any one of claims 1 to 10; and (b) a prenylated aromatic compound, a cannabinoid, or both.

27. The composition of claim 26, wherein the prenylated aromatic compound comprises CBGA, CBG, CBGOA, CBGO, CBGVA, CBGV, or a combination thereof; and wherein the cannabinoid comprises CBCA, CBC, CBCOA, CBCO, CBCVA, CBCV, CBDA, CBD, CBDOA, CBDO, CBDVA, CBDV, THCA, THC, THCOA, THCO, THCVA, THCV, an isomer, analog or derivative thereof, or combinations thereof.

28. The composition of claim 26 or 27, further comprising an enzyme in a cannabinoid biosynthesis pathway.

29. The composition of claim 28, wherein the cannabinoid biosynthesis pathway enzyme comprises olivetol synthase (OLS), olivetolic acid cyclase (OAC), an enzyme in a geranyl pyrophosphate (GPP) pathway, prenyltransferase, or combinations thereof.


SEQUENCE LISTING (18 sequences)

SEQ ID NO: 1 - protein, 19 aa, synthetic construct
AGSCPTVGVAGLTLGGGFG

SEQ ID NO: 2 - protein, 19 aa, synthetic construct
AGSCGTVAIGGLTLGGGVG

SEQ ID NO: 3 - protein, 19 aa, synthetic construct
AGSCPTVGIAGLTLGGGIG

SEQ ID NO: 4 - protein, 19 aa, synthetic construct
AGSCFTVGVAGVTLGGGIG

SEQ ID NO: 5 - protein, 19 aa, synthetic construct
GGTCPRVAVGGLVLGGGYG

SEQ ID NO: 6 - protein, 19 aa, synthetic construct
AGVCPDIRIGGHVLGGGVG

SEQ ID NO: 7 - protein, 19 aa, synthetic construct
AGTCPRIGIGGHVLGGGMG

SEQ ID NO: 8 - protein, 19 aa, synthetic construct
AGFCPEIGIAGHVLGGGAG

SEQ ID NO: 9 - protein, 19 aa, synthetic construct
TGACGSVCVGGFVQGGGYG

SEQ ID NO: 10 - protein, 19 aa, synthetic construct
GGSCHDVCVAGFMQGGGFG

SEQ ID NO: 11 - protein, 19 aa, synthetic construct
SGRCPTVGTSGLVLGGGWG

SEQ ID NO: 12 - protein, 19 aa, synthetic construct
GGSCPSVGIAGYLLGGGVG

SEQ ID NO: 13 - protein, 19 aa, synthetic construct
TGNCPTVGMGGYLQGGGVG

SEQ ID NO: 14 - protein, 19 aa, synthetic construct
GGYCPTVAAGGYFAGGGMG

SEQ ID NO: 15 - protein, 506 aa, Streptomyces sp.
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 16 - protein, 385 aa, synthetic construct
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 17 - protein, 101 aa, synthetic construct
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 18 - protein, 305 aa, synthetic construct
MSGAADVERVYAAMEEAAGLLGVTCAREKIYPLLTEFQDTLTDGVVVFSMASGRRSTELDFSISVPTSQGDPYATVVDKGLFPATGHPVDDLLADTQKHLPVSMFAIDGEVTGGFKKTYAFFPTDDMPGVAQLSAIPSMPSSVAENAELFARYGLDKVQMTSMDYKKRQVNLYFSELSEQTLAPESVLALVRELGLHVPTELGLEFCKRSFSVYPTLNWDTGKIDRLCFAVISTDPTLVPSTDERDIEQFRHYGTKAPYAYVGENRTLVYGLTLSPTEEYYKLGAYYHITDIQRRLLKAFDALED